(19) United States
(12) Patent Application Publication
(10) Pub. No.: US 2022/0033862 A1
(43) Pub. Date: Feb. 3, 2022

(54) ENGINEERED BIOSYNTHETIC PATHWAYS FOR PRODUCTION OF 2-OXOADIPATE BY FERMENTATION

(71) Applicant: Zymergen Inc., Emeryville, CA (US)

(72) Inventors: Anupam Chowdhury, Emeryville, CA (US); Steven M. Edgar, Albany, CA (US); Alexander Glennon Shearer, San Francisco, CA (US); Cara Ann Tracewell, Walnut Creek, CA (US); Stepan Tymoshenko, Emeryville, CA (US); Zhihao Wang, Emeryville, CA (US)

(73) Assignee: Zymergen Inc., Emeryville, CA (US)

(21) Appl. No.: 17/297,371

(22) PCT Filed: Nov. 25, 2019
(86) PCT No.: PCT/US2019/063107

§ 371 Date: May 26, 2021

Related U.S. Application Data
(60) Provisional application No. 62/773,118, filed on Nov. 29, 2018.

(51) Int. Cl. C12P 7/50; C12N 9/10; C12N 15/81; C12N 15/77; C12N 1/16; C12N 1/20

(52) U.S. Cl. CPC C12P 7/50; C12N 9/1025; C12N 15/81; C12R 2001/865; C12N 1/16; C12N 1/20; C12Y 203/03014; C12N 15/77

(57) ABSTRACT

The present disclosure describes the engineering of microbial cells for fermentative production of 2-oxoadipate and provides novel engineered microbial cells and cultures, as well as related 2-oxoadipate production methods.

### CROSS-REFERENCE TO RELATED APPLICATIONS

This application claims priority to and benefit of U.S. provisional application No. 62/773,118, filed on Nov. 29, 2018, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Nov. 20, 2019, is named ZMGNP009WO_Seq_List_ST25.txt and is 334,915 bytes in size.

### FIELD OF THE DISCLOSURE

The present disclosure relates generally to the area of engineering microbes for production of 2-oxoadipate by fermentation.

### BACKGROUND

2-Oxoadipate is produced biosynthetically from 2-oxoglutarate and acetyl-CoA by three enzymatic steps. 2-Oxoadipate (α-ketoadipate) is also a metabolite in the degradation pathway of lysine.

### SUMMARY

The disclosure provides engineered microbial cells, cultures of the microbial cells, and methods for the production of 2-oxoadipate, including the following:

Embodiment 1: An engineered microbial cell that expresses a heterologous homocitrate synthase, wherein the engineered microbial cell produces 2-oxoadipate.

Embodiment 2: The engineered microbial cell of embodiment 1, wherein the engineered microbial cell also expresses a heterologous homoaconitase.

Embodiment 3: The engineered microbial cell of embodiment 1 or embodiment 2, wherein the engineered microbial cell also expresses a heterologous homoisocitrate dehydrogenase.

Embodiment 4: The engineered microbial cell of any one of embodiments 1-3, wherein the engineered microbial cell expresses one or more additional enzyme(s) selected from an additional heterologous homocitrate synthase, an additional heterologous homoaconitase, or an additional heterologous homoisocitrate dehydrogenase.

Embodiment 5: An engineered microbial cell that expresses a non-native homocitrate synthase, wherein the engineered microbial cell produces 2-oxoadipate.

Embodiment 6: The engineered microbial cell of embodiment 5, wherein the engineered microbial cell also expresses a non-native homoaconitase.

Embodiment 7: The engineered microbial cell of embodiment 5 or embodiment 6, wherein the engineered microbial cell also expresses a non-native homoisocitrate dehydrogenase.

Embodiment 8: The engineered microbial cell of any one of embodiments 5-7, wherein the engineered microbial cell expresses one or more additional enzyme(s) selected from an additional non-native homocitrate synthase, an additional non-native homoaconitase, or an additional non-native homoisocitrate dehydrogenase.

Embodiment 9: The engineered microbial cell of 8, wherein the additional enzyme(s) are from a different organism than the corresponding enzyme in embodiments 5-7.

Embodiment 10: The engineered microbial cell of any of embodiments 5-9, wherein the engineered microbial cell includes increased activity of one or more upstream 2-oxoadipate pathway enzyme(s), said increased activity being increased relative to a control cell.

Embodiment 11: The engineered microbial cell of any one of embodiments 5-10, wherein the engineered microbial cell includes reduced activity of one or more enzyme(s) that consume one or more 2-oxoadipate pathway precursors, said reduced activity being reduced relative to a control cell.

Embodiment 12: The engineered microbial cell of embodiment 11, wherein the one or more enzyme(s) that consume one or more 2-oxoadipate pathway precursors comprise alpha-ketoglutarate dehydrogenase or citrate synthase.

Embodiment 13: The engineered microbial cell of embodiment 11 or embodiment 12, wherein the reduced activity is achieved by replacing a native promoter of a gene for the one or more enzymes that consume one or more 2-oxoadipate pathway precursors with a less active promoter.

Embodiment 14: An engineered microbial cell, wherein the engineered microbial cell includes means for expressing a heterologous homocitrate synthase, wherein the engineered microbial cell produces 2-oxoadipate.

Embodiment 15: The engineered microbial cell of embodiment 14, wherein the engineered microbial cell also includes means for expressing a heterologous homoaconitase.

Embodiment 16: The engineered microbial cell of embodiment 14 or embodiment 15, wherein the engineered microbial cell also includes means for expressing a non-native homoisocitrate dehydrogenase.

Embodiment 17: An engineered microbial cell, wherein the engineered microbial cell includes means for expressing a non-native homocitrate synthase, wherein the engineered microbial cell produces 2-oxoadipate.

Embodiment 18: The engineered microbial cell of embodiment 17, wherein the engineered microbial cell also includes means for expressing a non-native homoaconitase.

Embodiment 19: The engineered microbial cell of embodiment 17 or embodiment 18, wherein the engineered microbial cell also includes means for expressing a non-native homoisocitrate dehydrogenase.

Embodiment 20: The engineered microbial cell of any one of embodiments 14-19, wherein the engineered microbial cell includes means for increasing the activity of one or more upstream 2-oxoadipate pathway enzyme(s), said increased activity being increased relative to a control cell.

Embodiment 21: The engineered microbial cell of any one of embodiments 14-20, wherein the engineered microbial cell includes means for reducing the activity of one or more enzyme(s) that consume one or more 2-oxoadipate pathway precursors, said reduced activity being reduced relative to a control cell.

Embodiment 22: The engineered microbial cell of embodiment 21, wherein the one or more enzyme(s) that consume one or more 2-oxoadipate pathway precursors comprise alpha-ketoglutarate dehydrogenase or citrate synthase.

Embodiment 23: The engineered microbial cell of embodiment 21 or embodiment 22, wherein the reduced activity is achieved by means for replacing a native promoter of a gene for said one or more enzymes with a less active promoter.

Embodiment 24: The engineered microbial cell of any one of embodiments 5-23, wherein the engineered microbial cell includes a fungal cell.

Embodiment 25: The engineered microbial cell of embodiment 24, wherein the engineered microbial cell includes a yeast cell.

Embodiment 26: The engineered microbial cell of embodiment 25, wherein the yeast cell is a cell of the genus Saccharomyces.

Embodiment 27: The engineered microbial cell of embodiment 26, wherein the yeast cell is a cell of the species cerevisiae.

Embodiment 28: The engineered microbial cell of any one of embodiments 5-27, wherein the non-native homocitrate synthase includes a homocitrate synthase having at least 70% amino acid sequence identity with a homocitrate synthase from Komagataella pastoris or Thermus thermophiles.

Embodiment 29: The engineered microbial cell of embodiment 28, wherein the engineered microbial cell includes a non-native homocitrate synthase having at least 70% amino acid sequence identity with the homocitrate synthase from Komagataella pastoris and a non-native homocitrate synthase having at least 70% amino acid sequence identity with the homocitrate synthase from Thermus thermophilus.

Embodiment 30: The engineered microbial cell of embodiment 25, wherein the engineered microbial cell includes a homocitrate synthase having at least 70 percent amino acid sequence identity to a homocitrate synthase from Schizosaccharomyces pombe (strain 972/ATCC 24843) (Fission yeast) (Uniprot ID No. Q9Y823; SEQ ID NO:90), having amino acid substitution D123N; a homoaconitase having at least 70 percent amino acid sequence identity to a homoaconitase from Saccharomyces cerevisiae (strain ATCC 204508/S288c) (Baker's yeast) (Uniprot ID No. P49367; SEQ ID NO:33); and a homoisocitrate dehydrogenase having at least 70 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11).

Embodiment 31: The engineered microbial cell of embodiment 30, wherein the engineered microbial cell is a Saccharomyces cerevisiae cell or a Yarrowia lipolytica cell.

Embodiment 32: The engineered microbial cell of any one of embodiments 7-23, wherein the engineered microbial cell is a bacterial cell.

Embodiment 33: The engineered microbial cell of embodiment 32, wherein the bacterial cell is a cell of the genus Corynebacterium.

Embodiment 34: The engineered microbial cell of embodiment 33, wherein the bacterial cell is a cell of the species glutamicum.

Embodiment 35: The engineered microbial cell of embodiment 34, wherein the non-native homocitrate synthase includes a homocitrate synthase having at least 70% amino acid sequence identity with a homocitrate synthase selected from the group consisting of Thermus thermophilus, Saccharomyces cerevisiae, Candida dubliniensis, Ustilaginoidea virens, Schizosaccharomyces cryophilus, and Komagataella pastoris.

Embodiment 36: The engineered microbial cell of embodiment 35, wherein the non-native homocitrate synthase includes a homocitrate synthase having at least 70% amino acid sequence identity with a homocitrate synthase from Thermus thermophilus or Saccharomyces cerevisiae.

Embodiment 37: The engineered microbial cell of embodiment 36, wherein the engineered microbial cell includes a non-native homocitrate synthase having at least 70% amino acid sequence identity with the homocitrate synthase from Thermus thermophilus and a non-native homocitrate synthase having at least 70% amino acid sequence identity with the homocitrate synthase from Saccharomyces cerevisiae.

Embodiment 38: The engineered microbial cell of any one of embodiments 34-37, wherein the engineered microbial cell also expresses a non-native homoaconitase having at least 70% amino acid sequence identity with a homoaconitase selected from the group consisting of Ogataea parapolymorpha, Komagataella pastoris, Ustilaginoidea virens, Ceratocystis fimbriata f. sp. Platani, and Gibberella moniliformis.

Embodiment 39: The engineered microbial cell of embodiment 38, wherein the non-native homoaconitase includes a homoaconitase having at least 70% amino acid sequence identity with a homoaconitase from Ogataea parapolymorpha.

Embodiment 40: The engineered microbial cell of any one of embodiments 34-39, wherein the wherein the engineered microbial cell also expresses a non-native homoisocitrate dehydrogenase having at least 70% amino acid sequence identity with a homoisocitrate dehydrogenase selected from the group consisting of Ogataea parapolymorpha, Candida dubliniensis, and Saccharomyces cerevisiae.

Embodiment 41: The engineered microbial cell of any one of embodiments 1-40, wherein the wherein the engineered microbial cell also expresses a non-native homoisocitrate dehydrogenase having at least 70% amino acid sequence identity with a homoisocitrate dehydrogenase from Ogataea parapolymorpha.

Embodiment 42: The engineered microbial cell of embodiment 34, wherein the engineered microbial cell includes a homocitrate synthase having at least 70 percent amino acid sequence identity to a homocitrate synthase from Schizosaccharomyces pombe (strain 972/ATCC 24843) (Fission yeast) (Uniprot ID No. Q9Y823; SEQ ID NO:90), having amino acid substitution D123N; a homoaconitase having at least 70 percent amino acid sequence identity to a homoaconitase from Saccharomyces cerevisiae (strain ATCC 204508/S288c) (Baker's yeast) (Uniprot ID No. P49367; SEQ ID NO:33); and a homoisocitrated dehydrogenase having at least 70 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11).

Embodiment 43: The engineered microbial cell of embodiment 32, wherein the bacterial cell is a Bacillus subtilis cell.

Embodiment 44: The engineered microbial cell of embodiment 43, wherein the engineered microbial cell includes a homocitrate synthase having at least 70 percent amino acid sequence identity to a homocitrate synthase from Saccharomyces cerevisiae (strain ATCC 204508/S288c) (Baker's yeast) (Uniprot ID No. P48570; SEQ ID NO:35); a homoaconitase having at least 70 percent amino acid sequence identity to a homoaconitase from Neosartorya fumigata (strain ATCC MYA-4609/Af293/CBS 101355/FGSC A1100) (Aspergillus fumigatus) (Uniprot ID No. Q4WUL6; SEQ ID NO:83), which includes a deletion of amino acid residues 2-41 and 721-777, relative to the full-length sequence; and a homoisocitrate dehydrogenase having at least 70 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11).

Embodiment 45: The engineered microbial cell of any one of embodiments 5-41, wherein, when cultured, the engineered microbial cell produces 2-oxoadipate at a level at least 100 μg/L of culture medium.

Embodiment 46: The engineered microbial cell of embodiment 45, wherein, when cultured, the engineered microbial cell produces 2-oxoadipate at a level at least 20 mg/L of culture medium.

Embodiment 47: The engineered microbial cell of embodiment 46, wherein, when cultured, the engineered microbial cell produces 2-oxoadipate at a level at least 75 mg/L of culture medium.

Embodiment 48: A culture of engineered microbial cells according to any one of embodiments 5-46.

Embodiment 49: The culture of embodiment 48, wherein the substrate includes a carbon source and a nitrogen source selected from the group consisting of urea, an ammonium salt, ammonia, and any combination thereof.

Embodiment 50: The culture of embodiment 48 or embodiment 49, wherein the engineered microbial cells are present in a concentration such that the culture has an optical density at 600 nm of 10-500.

Embodiment 51: The culture of any one of embodiments 48-50, wherein the culture includes 2-oxoadipate.

Embodiment 52: The culture of any one of embodiments 48-51, wherein the culture includes 2-oxoadipate at a level at least 100 μg/L of culture medium.

Embodiment 53: A method of culturing engineered microbial cells according to any one of embodiments 5-46, the method including culturing the cells under conditions suitable for producing 2-oxoadipate.

Embodiment 54: The method of embodiment 53, wherein the method includes fed-batch culture, with an initial glucose level in the range of 1-100 g/L, followed controlled sugar feeding.

Embodiment 55: The method of embodiment 53 or embodiment 54, wherein the fermentation substrate includes glucose and a nitrogen source selected from the group consisting of urea, an ammonium salt, ammonia, and any combination thereof.

Embodiment 56: The method of any one of embodiments 53-55, wherein the culture is pH-controlled during culturing.

Embodiment 57: The method of any one of embodiments 53-56, wherein the culture is aerated during culturing.

Embodiment 58: The method of any one of embodiments 53-57, wherein the engineered microbial cells produce 2-oxoadipate at a level at least 100 μg/L of culture medium.

Embodiment 59: The method of any one of embodiments 53-58, wherein the method additionally includes recovering 2-oxoadipate from the culture.

Embodiment 60: A method for preparing 2-oxoadipate using microbial cells engineered to produce 2-oxoadipate, the method including: (a) expressing a non-native homocitrate synthase in microbial cells; (b) cultivating the microbial cells in a suitable culture medium under conditions that permit the microbial cells to produce 2-oxoadipate, wherein the 2-oxoadipate is released into the culture medium; and (c) isolating 2-oxoadipate from the culture medium.

### BRIEF DESCRIPTION OF THE DRAWINGS

FIG. 1: Biosynthetic pathway for 2-oxoadipate. Step 1 is catalyzed by homocitrate synthase. Step 2 is catalyzed by homoaconitase. Step 3 is catalyzed by homoisocitrate dehydrogenase.

FIG. 2: 2-oxoadipate titers measured in the extracellular broth following fermentation by the first-round engineered host Corynebacterium glutamicum. (See also Example 1, Table 1.)

FIG. 3: 2-oxoadipate titers measured in the extracellular broth following fermentation by the first-round engineered host Saccharomyces cerevisiae. (See also Example 1, Table 1.)

FIG. 4: 2-oxoadipate titers measured in the extracellular broth following fermentation by the second-round engineered host Corynebacterium glutamicum. (See also Example 1, Table 2.)

FIG. 5: 2-oxoadipate titers measured in the extracellular broth following fermentation by the second-round engineered host Saccharomyces cerevisiae. (See also Example 1, Table 2.)

FIG. 6: Integration of Promoter-Gene-Terminator into Saccharomyces cerevisiae and Yarrowia lipolytica.

FIG. 7: Promoter replacement in Saccharomyces cerevisiae and Yarrowia lipolytica.

FIG. 8: Targeted gene deletion in Saccharomyces cerevisiae and Yarrowia lipolytica.

FIG. 9: Integration of Promoter-Gene-Terminator into Corynebacterium glutamicum and Bacillus subtilis.

FIG. 10: 2-oxoadipate titers measured in the extracellular broth following fermentation by the engineered host Yarrowia lipolytica. (See also Example 2, Table 4.)

FIG. 11: 2-oxoadipate titers measured in the extracellular broth following fermentation by the engineered host Bacillus subtilis. (See also Example 2, Table 5.)

FIG. 12: 2-oxoadipate titers measured in the extracellular broth following fermentation by the further engineered host Saccharomyces cerevisiae. (See also Example 2, Table 6.)

FIG. 13: 2-oxoadipate titers measured in the extracellular broth following fermentation by the host-evaluation-round engineered host Corynebacterium glutamicum. (See also Example 2, Table 7.)

FIG. 14: 2-oxoadipate titers measured in the extracellular broth following fermentation by the improvement-round engineered host Corynebacterium glutamicum.

(See also Example 2, Table 8.)

FIG. 15: “Loop-in, loop-out, double-crossover” genomic integration strategy used to engineer Bacillus subtilis in Example 2.

### DETAILED DESCRIPTION

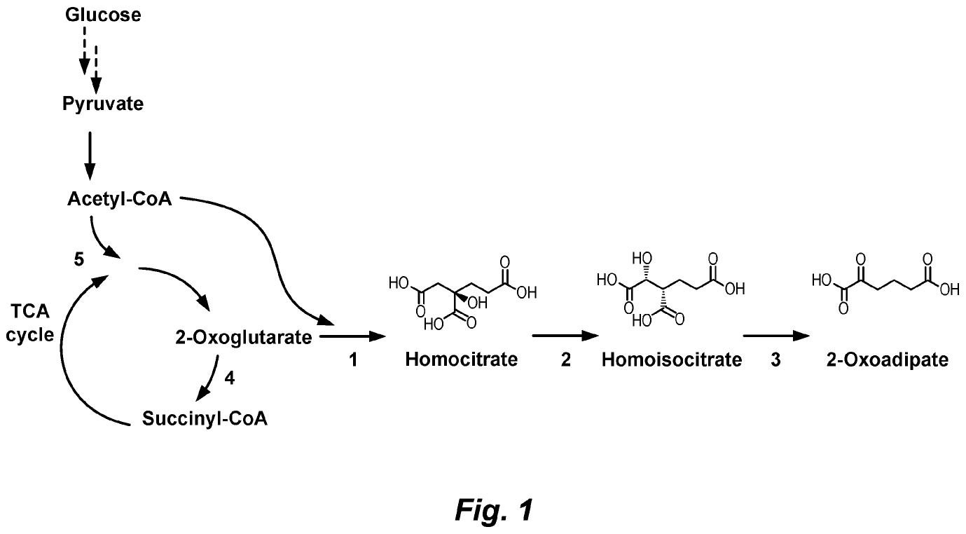

This disclosure describes a method for the production of the small molecule 2-oxoadipate via fermentation by a microbial host from simple carbon and nitrogen sources, such as glucose and urea, respectively. This objective can be achieved by enhancing a native pathway and/or introducing a non-native metabolic pathway into a suitable microbial host for industrial fermentation of chemical products. Illustrative hosts include Saccharomyces cerevisiae, Yarrowia lipolytica, Corynebacterium glutamicum, and Bacillus subtilis. The engineered metabolic pathway links the central metabolism of the host to a non-native pathway to enable the production of 2-oxoadipate. The simplest embodiment of this approach is the expression of an enzyme, such as a homocitrate synthase enzyme, in a microbial host strain that has the other enzymes necessary for 2-oxoadipate production (see FIG. 1), such as S. cerevisiae. In some hosts, such as C. glutamicum, two additional enzymes must be expressed with the homocitrate synthase: homoaconitase and homoisocitrate dehydrogenase.

The following disclosure describes how to engineer a microbe with the necessary characteristics to produce industrially feasible titers of 2-oxoadipate from simple carbon and nitrogen sources. Active homocitrate synthases, as well as active homoaconitases and homoisocitrate dehydrogenases, have been identified that enable S. cerevisiae and C. glutamicum to produce significant levels of 2-oxoadipate, and it has been found that the expression of an additional copy of homocitrate synthase improves the 2-oxoadipate titers. Expression and/or over-expression of heterologous pathway enzymes in the work described herein enabled titers of 28.5 mg/L 2-oxoadipate in C. glutamicum and 0.5 mg/L 2-oxoadipate in S. cerevisiae (Example 1). Further engineering gave titers of 97 mg/L and 80 mg/L in C. glutamicum and S. cerevisiae, respectively, and demonstrated the feasibility of engineering Bacillus subtilis and Yarrowia lipolytica to produce 2-oxoadipate.

### Definitions

Terms used in the claims and specification are defined as set forth below unless otherwise specified.

The term “fermentation” is used herein to refer to a process whereby a microbial cell converts one or more substrate(s) into a desired product (such as 2-oxoadipate) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the native enzyme native to the cell. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native enzyme. In some embodiments, the feedback-deregulated enzyme shows no feedback-inhibition in the microbial cell.

The term “2-oxoadipate” refers to 2-oxohexanedioic acid (CAS #3184-35-8).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., 2-oxoadipate) produced by a culture of microbial cells divided by the culture volume.

As used herein with respect to recovering 2-oxoadipate from a cell culture, “recovering” refers to separating the 2-oxoadipate from at least one other component of the cell culture medium.

### Engineering Microbes for 2-Oxoadipate Production

2-Oxoadipate Biosynthesis Pathway

2-oxoadipate is typically derived from 2-oxoglutarate and acetyl-CoA by three enzymatic steps, requiring the enzymes homocitrate synthase, homoaconitase, and homoisocitrate dehydrogenase. The 2-oxoadipate biosynthesis pathway is shown in FIG. 1. Significant 2-oxoadipate production is enabled by the addition of a single non-native enzyme in Saccharomyces cerevisiae, namely, homocitrate synthase. Some microbial species do not have activities for homocitrate synthase, homoaconitase, or homoisocitrate dehydrogenase natively. To enable 2-oxoadipate production in Corynebacterium glutamicum, for example, three non-native enzymes having these activities are introduced.

Engineering for Microbial 2-Oxoadipate Production

Any homocitrate synthase that is active in the microbial cell being engineered may be introduced into the cell, typically by introducing and expressing the gene(s) encoding the enzyme(s) using standard genetic engineering techniques. Suitable homocitrate synthases may be derived from any source, including plant, archaeal, fungal, gram-positive bacterial, and gram-negative bacterial sources. Exemplary sources include, but are not limited to: Candida dubliniensis, Komagataella pastoris, Saccharomyces cerevisiae, Schizosaccharomyces cryophilus, Thermus thermophilus, and Ustilaginoidea virens.

Any homoaconitase that is active in the microbial cell being engineered may be introduced into the cell, typically by introducing and expressing the gene(s) encoding the enzyme(s)s using standard genetic engineering techniques. Suitable homoaconitases may be derived from any source, including plant, archaeal, fungal, gram-positive bacterial, and gram-negative bacterial sources. Exemplary sources include, but are not limited to: Ceratocystis fimbriata f. sp. Platani, Gibberella moniliformis, Komagataella pastoris, Ogataea parapolymorpha, and Ustilaginoidea virens.

Any homoisocitrate dehydrogenase that is active in the microbial cell being engineered may be introduced into the cell, typically by introducing and expressing the gene(s) encoding the enzyme(s) using standard genetic engineering techniques. Suitable homoisocitrate dehydrogenases may be derived from any source, including plant, archaeal, fungal, gram-positive bacterial, and gram-negative bacterial sources. Exemplary sources include, but are not limited to: Candida dubliniensis, Ogataea parapolymorpha, and Saccharomyces cerevisiae.

One or more copies of any of these genes can be introduced into a selected microbial host cell. If more than one copy of a gene is introduced, the copies can have the same or different nucleotide sequences. In some embodiments, one or both (or all) of the heterologous gene(s) is/are expressed from a strong, constitutive promoter. In some embodiments, the heterologous gene(s) is/are expressed from an inducible promoter. The heterologous gene(s) can optionally be codon-optimized to enhance expression in the selected microbial host cell.

Example 1 shows that, in Corynebacterium glutamicum, a 28 mg/L titer of 2-oxoadipate was achieved in a first round of engineering after integration of the three necessary non-native enzymes. Nearly all of the engineered C. glutamicum strains in this first round give a similar titer. (See Table 1.) One strain, which contains constitutively expressed homocitrate synthase from Thermus thermophilus (UniProt ID 087198), homoaconitase from Ogataea parapolymorpha (UniProt ID W1QJE4), and homoisocitrate dehydrogenase from Ogataea parapolymorpha (UniProt ID W1QLF1), was chosen to be the parent strain for additional engineering.

Example 1 shows that, in Saccharomyces cerevisiae, a titer of 128 μg/L was achieved in a first round of engineering after integration of homocitrate synthase from Komagataella pastoris (UniProt ID F2QPL2). (See Table 1.) This strain was chosen to be the parent strain for additional engineering.

A second round of engineering was carried out in the C. glutamicum and S. cerevisiae parent strains from the first round. For the second round, plasmids designed to integrate an additional copy of various, different homocitrate synthases expressed from a strong constitutive promoter were introduced. (See Table 2).

In S. cerevisiae, a titer of 553 μg/L was achieved by integration of homocitrate synthase from Thermus thermophilus (UniProt ID 087198).

Designs for a third round of engineering in C. glutamicum are shown in Table 3.

Example 2 shows that, in Corynebacterium glutamicum, a 97 mg/L titer of 2-oxoadipate was achieved after integration of: a homocitrate synthase from Schizosaccharomyces pombe (strain 972/ATCC 24843) (Fission yeast) (Uniprot ID No. Q9Y823; SEQ ID NO:90), having amino acid substitution D123N, a homoaconitase from Saccharomyces cerevisiae (strain ATCC 204508/S288c) (Baker's yeast) (Uniprot ID No. P49367; SEQ ID NO:33), and a homoisocitrate dehydrogenase from Saccharomyces cerevisiae (strain ATCC 204508/S288c) (Baker's yeast) (Uniprot ID No. P40495; SEQ ID NO:11). (See Table 7.)

Also in Example 2, an 80 mg/L titer of 2-oxoadipate was achieved in S. cerevisiae after integration of: a homocitrate synthase from Schizosaccharomyces pombe (strain 972/ATCC 24843) (Fission yeast) (Uniprot ID No. Q9Y823; SEQ ID NO:90), having amino acid substitution D123N, a homoaconitase from Saccharomyces cerevisiae (strain ATCC 204508/S288c) (Baker's yeast) (Uniprot ID No. P49367; SEQ ID NO:33), and a homoisocitrate dehydrogenase from Saccharomyces cerevisiae (strain ATCC 204508/S288c) (Baker's yeast) (Uniprot ID No. P40495; SEQ ID NO:11). (See Table 6.)

In Example 2, two additional hosts were engineered for 2-oxoadipate production: Yarrowia lipolytica and Bacillus subtilis. In Y. lipolytica, a 238 μg/L titer of 2-oxoadipate was achieved in a first round of engineering after integration of: a homocitrate synthase from Schizosaccharomyces pombe (strain 972/ATCC 24843) (Fission yeast) (Uniprot ID No. Q9Y823; SEQ ID NO:90), having amino acid substitution D123N, a homoaconitase from Saccharomyces cerevisiae (strain ATCC 204508/S288c) (Baker's yeast) (Uniprot ID No. P49367; SEQ ID NO:33), and a homoisocitrate dehydrogenase from Saccharomyces cerevisiae (strain ATCC 204508/S288c) (Baker's yeast) (Uniprot ID No. P40495; SEQ ID NO:11). (See Table 4.) In B. subtilis, a 7 μg/L titer of 2-oxoadipate was achieved in a first round of engineering after integration of: a homocitrate synthase from Saccharomyces cerevisiae (strain ATCC 204508/S288c) (Baker's yeast) (Uniprot ID No. P48570; SEQ ID NO:35), a homoaconitase from Neosartorya fumigata (strain ATCC MYA-4609/Af293/CBS 101355/FGSC A1100) (Aspergillus fumigatus) (Uniprot ID No. Q4WUL6; SEQ ID NO:83), which includes a deletion of amino acid residues 2-41 and 721-777, relative to the full-length sequence, and a homoisocitrate dehydrogenase from Saccharomyces cerevisiae (strain ATCC 204508/S288c) (Baker's yeast) (Uniprot ID No. P40495; SEQ ID NO:11). (See Table 5.)

### Increasing the Activity of Upstream Enzymes

One approach to increasing 2-oxoadipate production in a microbial cell that is capable of such production is to increase the activity of one or more upstream enzymes in the 2-oxoadipate biosynthesis pathway. Upstream pathway enzymes include all enzymes involved in the conversions from a feedstock all the way to into the last native metabolite. Illustrative enzymes for use in this embodiment include citrate synthase (E.C. 2.3.3.1), aconitase (E.C. 4.2.1.3), isocitrate dehydrogenase (E.C. 1.1.1.42 or E.C. 1.1.1.41), pyruvate dehydrogenase (E.C. 1.2.4.1), dihydrolipoyl transacetylase (E.C. 2.3.1.12), dihydrolipoyl dehydrogenase (E.C. 1.8.1.4), and isoforms, paralogs, or orthologs having these enzymatic activities (which as those of skill in the art readily appreciate may be known by different names). Suitable upstream pathway genes encoding these enzymes may be derived from any source, including, for example, those discussed above as sources for a homocitrate synthase, homoaconitase, or homoisocitrate dehydrogenase genes.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

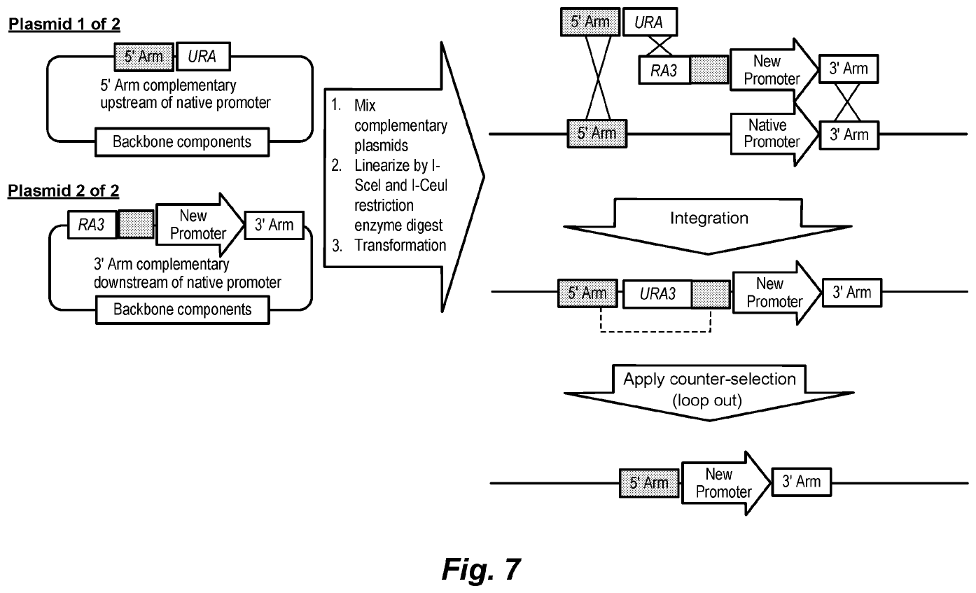

Alternatively, or in addition, one or more promoters can be substituted for native promoters using, for example, a technique such as that illustrated in FIG. 7. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of 2-oxoadipate production and expressed from a strong constitutive promoter and/or can optionally be codon-optimized to enhance expression in the selected microbial host cell.

In various embodiments, the engineering of a 2-oxoadipate-producing microbial cell to increase the activity of one or more upstream pathway enzymes increases the 2-oxoadipate titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in 2-oxoadipate titer is in the range of 10 percent to 100-fold, 2-fold to 50-fold, 5-fold to 40-fold, 10-fold to 30-fold, or any range bounded by any of the values listed above. (Ranges herein include their endpoints.) These increases are determined relative to the 2-oxoadipate titer observed in a 2-oxoadipate-producing microbial cell that lacks any increase in activity of upstream pathway enzymes. This reference cell may have one or more other genetic alterations aimed at increasing 2-oxoadipate production, e.g., the cell may express a feedback-deregulated enzyme.

In various embodiments, the 2-oxoadipate titers achieved by increasing the activity of one or more upstream pathway genes are at least 1, 10, 20, 30, 40, 50, 75, 100, 200, 300, 400, 500, 600, 700, 800, or 900 mg/L or at least 1, 1.5, 2, 2.5, 3, 3.5, 4, 4.5, 5, or 10 gm/L. In various embodiments, the titer is in the range of 10 mg/L to 10 gm/L, 20 mg/L to 5 gm/L, 50 mg/L to 4 gm/L, 100 mg/L to 3 gm/L, 500 mg/L to 2 gm/L or any range bounded by any of the values listed above.

### Reduction of Precursor Consumption

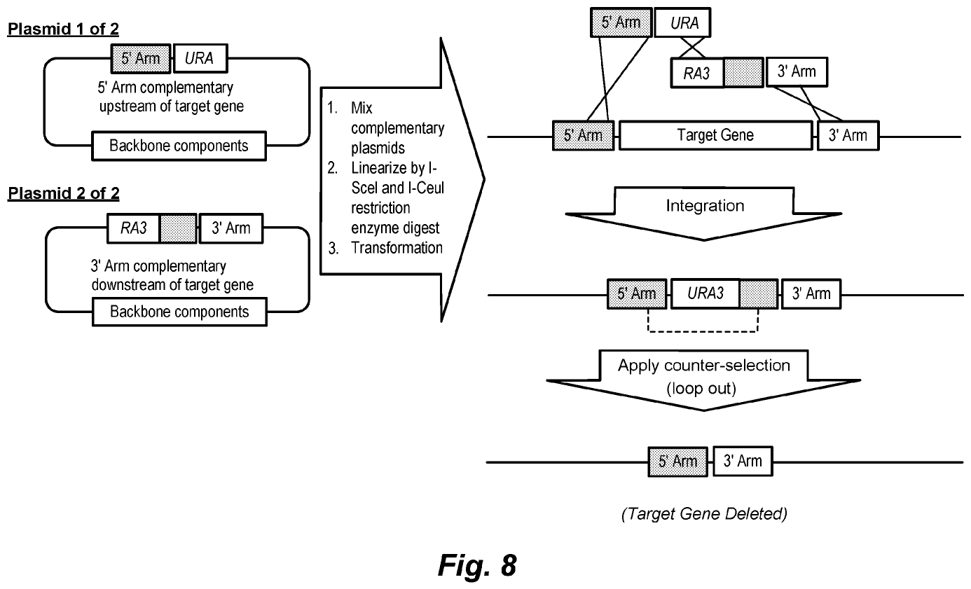

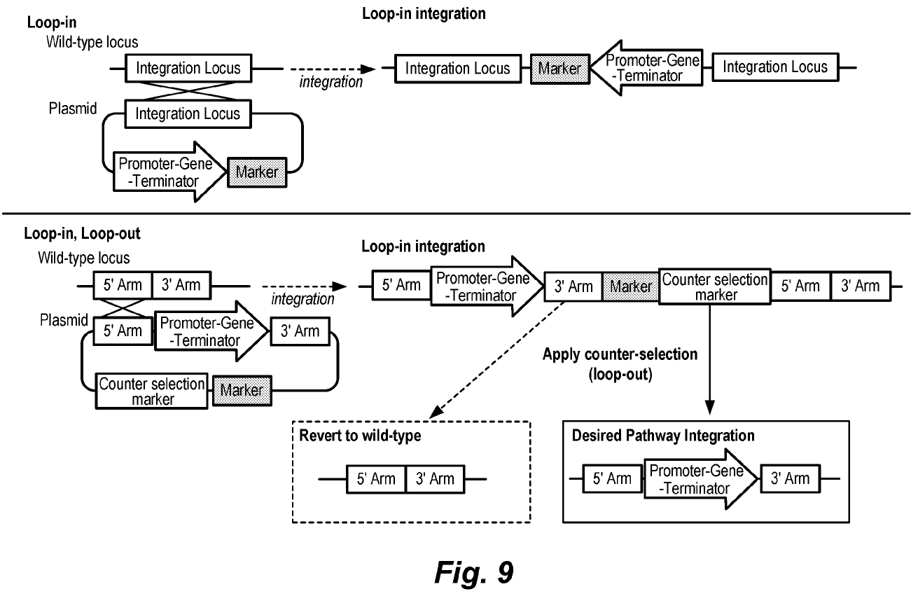

Another approach to increasing 2-oxoadipate production in a microbial cell that is capable of such production is to decrease the activity of one or more enzymes that consume one or more 2-oxoadipate pathway precursors. In some embodiments, the activity of one or more such enzymes is reduced by modulating the expression or activity of the native enzyme(s). Illustrative enzymes of this type include alpha-ketoglutarate dehydrogenase and citrate synthase. Lower expression of alpha-ketoglutarate dehydrogenase will decrease consumption of alpha-ketoglutarate (2-oxoglutarate), a substrate for the 2-oxoadipate pathway (FIG. 1 shows this enzyme as a step “4” that converts 2-oxoglutarate to succinyl-CoA). Decreased citrate synthase activity will decrease shunting of acetyl-CoA into the citric acid cycle. The activity of such enzymes can be decreased, for example, by substituting the native promoter of the corresponding gene(s) with a less active or inactive promoter or by deleting the corresponding gene(s). See FIGS. 7 and 8 for examples of schemes for promoter replacement and targeted gene deletion, respectively, in S. cervisiae and Y. lipolytica.

In various embodiments, the engineering of a 2-oxoadipate-producing microbial cell to reduce precursor consumption by one or more side pathways increases the 2-oxoadipate titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in 2-oxoadipate titer is in the range of 10 percent to 100-fold, 2-fold to 50-fold, 5-fold to 40-fold, 10-fold to 30-fold, or any range bounded by any of the values listed above. These increases are determined relative to the 2-oxoadipate titer observed in a 2-oxoadipate-producing microbial cell that does not include genetic alterations to reduce precursor consumption. This reference cell may (but need not) have other genetic alterations aimed at increasing 2-oxoadipate production, i.e., the cell may have increased activity of an upstream pathway enzyme.

In various embodiments, the 2-oxoadipate titers achieved by reducing precursor consumption by one or more side pathways are at least 100, 200, 300, 400, 500, 600, 700, 800, or 900 μg/L, or at least 1, 10, 50, 75, 100, 200, 300, 400, 500, 600, 700, 800, or 900 mg/L or at least 1, 1.5, 2, 2.5, 3, 3.5, 4, 4.5, 5, 10, 20, 50 g/L. In various embodiments, the titer is in the range of 50 μg/L to 50 g/L, 75 μg/L to 20 g/L, 100 μg/L to 10 g/L, 200 μg/L to 5 g/L, 500 μg/L to 4 g/L, 1 mg/L to 3 g/L, 500 mg/L to 2 g/L or any range bounded by any of the values listed above.

The approaches of increasing the activity of one or more native enzymes and/or introducing one or more feedback-deregulated enzymes and/or reducing precursor consumption by one or more side pathways can be combined to achieve even higher 2-oxoadipate production levels.

Illustrative Amino Acid and Nucleotide Sequences

The following table identifies amino acid and nucleotide sequences used in Example 1. The corresponding sequences are shown in the Sequence Listing.

SEQ ID NO Cross-Reference Table

| SEQ | | | | |
|---|---|---|---|---|
| ID | Sequence Type with | Uniprot | | |
| NO | Modifications | ID | Activity name | Source organism |
| 1 | AA seq for enzyme P49367 | P49367 | Homoisocitrate hydro-lyase | Saccharomyces cerevisiae (strain ATCC 204508/ |
| | | | | S288c) (Baker's yeast) |
| 2 | DNA seq for enzyme P49367 | P49367 | Homoisocitrate hydro-lyase | Saccharomyces cerevisiae (strain ATCC 204508/ |
| | | | | S288c) (Baker's yeast) |
| 3 | AA seq for enzyme P40495 | P40495 | (1R,25)-1-hydroxybutane-1,2,4- | Saccharomyces cerevisiae (strain ATCC 204508/ |
| | | | tricarboxylate: NAD+ oxidoreductase | S288c) (Baker's yeast) |
| 4 | DNA seq for enzyme P40495 | P40495 | (1R,25)-1-hydroxybutane-1,2,4- | Saccharomyces cerevisiae (strain ATCC 204508/ |
| | | | tricarboxylate: NAD+ oxidoreductase | S288c) (Baker's yeast) |
| 5 | AA seq for enzyme Q5KIZ5 | Q5KIZ5 | Homocitrate synthase, putative | Cryptococcus neoformans var. neoformans serotype |
| | | | | D (strain JEC21/ATCC MYA-565) (Filobasidiella |
| | | | | neoformans) |
| 6 | DNA seq for enzyme Q5KIZ5 | Q5KIZ5 | Homocitrate synthase, putative | Cryptococcus neoformans var. neoformans serotype |
| | | | | D (strain JEC21/ATCC MYA-565) (Filobasidiella |
| | | | | neoformans) |
| 7 | AA seq for enzyme A0A150JKI3 | A0A150JKI3 | Putative homocitrate synthase AksA (EC | Arc I group archaeon ADurb1113_Bin01801 |
| | | | 2.3.3.14) | |
| 8 | DNA seq for enzyme | A0A150JKI3 | Putative homocitrate synthase AksA (EC | Arc I group archaeon ADurb1113_Bin01801 |
| | A0A150JKI3 | | 2.3.3.14) | |
| 9 | AA seq for enzyme J8Q3V7 | J8Q3V7 | Lys12p | Saccharomyces arboricola (strain H-6/AS 2.3317/ |
| | | | | CBS 10644) (Yeast) |
| 10 | DNA seq for enzyme J8Q3V7 | J8Q3V7 | Lys12p | Saccharomyces athoricola (strain H-6/AS 2.3317/ |
| | | | | CBS 10644) (Yeast) |
| 11 | AA seq for enzyme P40495 | P40495 | Homoisocitrate dehydrogenase, | Saccharomyces cerevisiae (strain ATCC 204508/ |
| | | | mitochondrial (HIcDH) (EC 1.1.1.87) | S288c) (Baker's yeast) |
| 12 | DNA seq for enzyme P40495 | P40495 | Homoisocitrate dehydrogenase, | Saccharomyces cerevisiae (strain ATCC 204508/ |
| | | | mitochondrial (HIcDH) (EC 1.1.1.87) | S288c) (Baker's yeast) |
| 13 | AA seq for enzyme A4G035 | A4G035 | 2-isopropylmalate synthase (EC 2.3.3.13) | Methanococcus maripaludis (strain C5/ATCC BAA- |
| | | | | 1333) |
| 14 | DNA seq for enzyme A4G035 | A4G035 | 2-isopropylmalate synthase (EC 2.3.3.13) | Methanococcus maripaludis (strain C5/ATCC BAA- |
| | | | | 1333) |
| 15 | AA seq for enzyme E4V1M0 | E4V1M0 | Homocitrate synthase | Arthroderma gypseum (strain ATCC MYA-4604/ |
| | | | | CBS 118893) (Microsporum gypseum) |
| 16 | DNA seq for enzyme E4V1M0 | E4V1M0 | Homocitrate synthase | Arthroderma gypseum (strain ATCC MYA-4604/ |
| | | | | CBS 118893) (Microsporum gypseum) |
| 17 | AA seq for enzyme Q2IHS7 | Q2IHS7 | Homocitrate synthase (EC 2.3.3.14) | Anaeromyxobacter dehalogenans (strain 2CP-C) |
| 18 | DNA seq for enzyme Q2IHS7 | Q2IHS7 | Homocitrate synthase (EC 2.3.3.14) | Anaeromyxobacter dehalogenans (strain 2CP-C) |
| 19 | AA seq for enzyme Q9Y823 | Q9Y823 | Homocitrate synthase, mitochondrial (EC | Schizosaccharomyces pombe (strain 972/ATCC |
| | containing AA substitution | | 2.3.3.14) | 24843) (Fission yeast) |
| | E222Q | | | |
| 22 | DNA seq for enzyme Q9Y823 | Q9Y823 | Homocitrate synthase, mitochondrial (EC | Schizosaccharomyces pombe (strain 972/ATCC |
| | containing AA substitution | | 2.3.3.14) | 24843) (Fission yeast) |
| | E222Q | | | |
| 20 | AA seq for enzyme | A0A117DXK2 | Homocitrate synthase | Aspergillus niger |
| | A0A117DXK2 | | | |
| 21 | DNA seq for enzyme | A0A117DXK2 | Homocitrate synthase | Aspergillus niger |
| | A0A117DXK2 | | | |
| 23 | AA seq for enzyme F2NL20 | F2NL20 | Homocitrate synthase (EC 2.3.3.14) | Marinithermus hydrothermalis (strain DSM 14884/ |
| | | | | JCM 11576/T1) |
| 24 | DNA seq for enzyme F2NL20 | F2NL20 | Homocitrate synthase (EC 2.3.3.14) | Marinithermus hydrothermalis (strain DSM 14884/ |
| | | | | JCM 11576/11) |
| 25 | AA seq for enzyme Q9Y823 | Q9Y823 | Homocitrate synthase, mitochondrial (EC | Schizosaccharomyces pombe (strain 972/ATCC |
| | containing AA substitution | | 2.3.3.14) | 24843) (Fission yeast) |
| | R288K | | | |
| 26 | DNA seq for enzyme Q9Y823 | Q9Y823 | Homocitrate synthase, mitochondrial (EC | Schizosaccharomyces pombe (strain 972/ATCC |
| | containing AA substitution | | 2.3.3.14) | 24843) (Fission yeast) |
| | R288K | | | |
| 27 | AA seq for enzyme | B3LTU1 | B3LTU1 Homo-isocitrate dehydrogenase | Saccharomyces cerevisiae (strain RM11-1a) (Baker's |
| | | | | yeast) |
| 28 | DNA seq for enzyme B3LTU1 | B3LTU1 | Homo-isocitrate dehydrogenase | Saccharomyces cerevisiae (strain RM11-1a) (Baker's |
| | | | | yeast) |
| 30 | AA seq for enzyme F2PSY4 | F2PSY4 | Homocitrate synthase | Trichophyton equinum (strain ATCC MYA-4606/ |
| | | | | CBS 127.97) (Horse ringworm fungus) |
| 29 | DNA seq for enzyme F2PSY4 | F2PSY4 | Homocitrate synthase | Trichophyton equinum (strain ATCC MYA-4606/ |
| | | | | CBS 127.97) (Horse ringworm fungus) |
| 31 | AA seq for enzyme | A0A0F7TVK2 | Homocitrate synthase, mitochondrial | Penicillium brasilianum |
| | A0A0F7TVK2 | | (Putative Homocitrate synthase) | |
| 32 | DNA seq for enzyme | A0A0F7TVK2 | Homocitrate synthase, mitochondrial | Penicillium brasilianum |
| | A0A0F7TVK2 | | (Putative Homocitrate synthase) | |
| 33 | AA seq for enzyme P49367 | P49367 | Homoaconitase, mitochondrial (EC | Saccharomyces cerevisiae (strain ATCC 204508/ |
| | | | 4.2.1.36) (Homoaconitate hydratase) | S288c) (Baker's yeast) |
| 34 | DNA seq for enzyme P49367 | P49367 | Homoaconitase, mitochondrial (EC | Saccharomyces cerevisiae (strain ATCC 204508/ |
| | | | 4.2.1.36) (Homoaconitate hydratase) | S288c) (Baker's yeast) |
| 35 | AA seq for enzyme P48570 | P48570 | Homocitrate synthase, cytosolic isozyme | Saccharomyces cerevisiae (strain ATCC 204508/ |
| | | | (EC 2.3.3.14) | S288c) (Baker's yeast) |
| 36 | DNA seq for enzyme P48570 | P48570 | Homocitrate synthase, cytosolic isozyme | Saccharomyces cerevisiae (strain ATCC 204508/ |
| | | | (EC 2.3.3.14) | S288c) (Baker's yeast) |
| 37 | AA seq for enzyme | A0A0L1I0C1 | Homocitrate synthase (EC 2.3.3.14) | Stemphylium lycopersici |
| | A0A0L1I0C1 | | | |
| 38 | DNA seq for enzyme | A0A0L1I0C1 | Homocitrate synthase (EC 2.3.3.14) | Stemphylium lycopersici |
| | A0A0L1I0C1 | | | |
| 39 | AA seq for enzyme P40495 | P40495 | Homoisocitrate dehydrogenase, | Saccharomyces cerevisiae (strain ATCC 204508/ |
| | | | mitochondrial (HIcDH) (EC 1.1.1.87) | S288c) (Baker's yeast) |
| 40 | DNA seq for enzyme P40495 | P40495 | Homoisocitrate dehydrogenase, | Saccharomyces cerevisiae (strain ATCC 204508/ |
| | | | mitochondrial (HIcDH) (EC 1.1.1.87) | S288c) (Baker's yeast) |
| 41 | DNA seq for enzyme Q9Y823 | Q9Y823 | Homocitrate synthase, mitochondrial (EC | Schizosaccharomyces pombe (strain 972/ATCC |
| | containing AA substitution | | 2.3.3.14) | 24843) (Fission yeast) |
| | D123N | | | |
| 42 | AA seq for enzyme | A0A0E4HH64 | Homocitrate synthase 1 (EC 2.3.3.14) | Paenibacillus riograndensis SBR5 |
| | A0A0E4HH64 | | | |
| 43 | DNA seq for enzyme | A0A0E4HH64 | Homocitrate synthase 1 (EC 2.3.3.14) | Paenibacillus riograndensis SBR5 |
| | A0A0E4HH64 | | | |
| 44 | AA seq for enzyme Q4WUL6 | Q4WUL6 | Homoaconitase, mitochondrial (EC | Neosartorya fumigata (strain ATCC MYA-4609/ |
| | | | 4.2.1.36) (Homoaconitate hydratase) | Af293/CBS 101355/FGSC A1100) (Aspergillus |
| | | | | fumigatus) |
| 45 | DNA seq for enzyme Q4WUL6 | Q4WUL6 | Homoaconitase, mitochondrial (EC | Neosartorya fumigata (strain ATCC MYA-4609/ |
| | | | 4.2.1.36) (Homoaconitate hydratase) | Af293/CBS 101355/FGSC A1100) (Aspergillus |
| | | | | fumigatus) |
| 46 | AA seq for enzyme | A0A1F8TP88 | Homocitrate synthase | Chloroflexi bacterium |
| | A0A1F8TP88 | | | RIFCSPLOWO2_12_FULL_71_12 |
| 47 | DNA seq for enzyme | A0A1F8TP88 | Homocitrate synthase | Chloroflexi bacterium |
| | A0A1F8TP88 | | | RIFCSPLOWO2_12_FULL_71_12 |
| 48 | AA seq for enzyme Q75A20 | Q75A20 | ADR107VVp | Ashbya gossypii (strain ATCC 10895/CBS 109.51/ |
| | | | | FGSC 9923/NRRL Y-1056) (Yeast) (Eremothecium |
| | | | | gossypii) |
| 49 | DNA seq for enzyme Q75A20 | Q75A20 | ADR107VVp | Ashbya gossypii (strain ATCC 10895/CBS 109.51/ |
| | | | | FGSC 9923/NRRL Y-1056) (Yeast) (Eremothecium |
| | | | | gossypii) |
| 50 | AA seq for enzyme S6 KZZ1 | S6KZZ1 | Nth/protein, encodes a homocitrate | Pseudomonas stutzeri B1SMN1 |
| | | | synthase | |
| 51 | DNA seq for enzyme S6KZZ1 | S6KZZ1 | Nth/protein, encodes a homocitrate | Pseudomonas stutzeri B1SMN1 |
| | | | synthase | |
| 52 | AA seq for enzyme G8NBZ9 | G8NBZ9 | Homocitrate synthase | Thermus sp. CCB_US3_UF1 |
| 53 | DNA seq for enzyme G8NBZ9 | G8NBZ9 | Homocitrate synthase | Thermus sp. CCB_US3_UF1 |
| 54 | AA seq for enzyme A5UL49 | A5UL49 | 2-isopropylmalate synthase, LeuA (EC | Methanobrevibacter smithii (strain ATCC 35061/ |
| | | | 2.3.3.13) | DSM 861/OCM 144/PS) |
| 55 | DNA seq for enzyme A5UL49 | A5UL49 | 2-isopropylmalate synthase, LeuA (EC | Methanobrevibacter smithii (strain ATCC 35061/ |
| | | | 2.3.3.13) | DSM 861/OCM 144/PS) |
| 56 | AA seq for enzyme Q4WUL6 | Q4WUL6 | Homoaconitase, mitochondrial (EC | Neosartorya fumigata (strain ATCC MYA-4609/ |
| | | | 4.2.1.36) (Homoaconitate hydratase) | Af293/CBS 101355/FGSC A1100) (Aspergillus |
| | | | | fumigatus) |
| 57 | DNA seq for enzyme Q4VVUL6 | Q4WUL6 | Homoaconitase, mitochondrial (EC | Neosartorya fumigata (strain ATCC MYA-4609/ |
| | | | 4.2.1.36) (Homoaconitate hydratase) | Af293/CBS 101355/FGSC A1100) (Aspergillus |
| | | | | fumigatus) |
| 58 | AA seq for enzyme I2DYU9 | I2DYU9 | Homocitrate synthase | Burkholderia sp. KJ006 |
| 59 | DNA seq for enzyme I2DYU9 | I2DYU9 | Homocitrate synthase | Burkholderia sp. KJ006 |
| 60 | AA seq for enzyme P05342 | P05342 | Homocitrate synthase (EC 2.3.3.14) | Azotobacter vinelandii |
| 61 | DNA seq for enzyme P05342 | P05342 | Homocitrate synthase (EC 2.3.3.14) | Azotobacter vinelandii |
| 62 | AA seq for enzyme | A0A126T608 | Homocitrate synthase | Methylomonas denitrificans |
| | A0A126T608 | | | |
| 63 | DNA seq for enzyme | A0A126T608 | Homocitrate synthase | Methylomonas denitrificans |
| | A0A126T608 | | | |
| 64 | AA seq for enzyme Q9Y823 | Q9Y823 | Homocitrate synthase, mitochondrial (EC | Schizosaccharomyces pombe (strain 972/ATCC |
| | containing AA substitution | | 2.3.3.14) | 24843) (Fission yeast) |
| | R275K | | | |
| 65 | DNA seq for enzyme Q9Y823 | Q9Y823 | Homocitrate synthase, mitochondrial (EC | Schizosaccharomyces pombe (strain 972/ATCC |
| | containing AA substitution | | 2.3.3.14) | 24843) (Fission yeast) |
| | R275K | | | |
| 66 | AA seq for enzyme V5IKX8 | V5IKX8 | Homocitrate synthase (Homocitrate | Neurospora crassa (strain ATCC 24698/74-0R23- |
| | | | synthase, variant 1) | 1A/CBS 708.71/DSM 1257/FGSC 987) |
| 67 | DNA seq for enzyme V5IKX8 | V5IKX8 | Homocitrate synthase (Homocitrate | Neurospora crassa (strain ATCC 24698/74-0R23- |
| | | | synthase, variant 1) | 1A/CBS 708.71/DSM 1257/FGSC 987) |
| 68 | AA seq for enzyme D5Q163 | D5Q163 | Homocitrate synthase (EC 2.3.3.14) | Clostridioides difficile NAP08 |
| 69 | DNA seq for enzyme D5Q163 | D5Q163 | Homocitrate synthase (EC 2.3.3.14) | Clostridioides difficile NAP08 |
| 70 | AA seq for enzyme P12683 | P12683 | 3-hydroxy-3-methylglutaryl-coenzyme A | Saccharomyces cerevisiae (strain ATCC 204508/ |
| | containing dell- | | reductase 1 (HMG-CoA reductase 1) (EC | S288c) (Baker's yeast) |
| | 527;Y528M;T529A | | 1.1.1.34) | |
| 71 | DNA seq for enzyme P12683 | P12683 | 3-hydroxy-3-methylglutaryl-coenzyme A | Saccharomyces cerevisiae (strain ATCC 204508/ |
| | containing dell- | | reductase 1 (HMG-CoA reductase 1) (EC | S288c) (Baker's yeast) |
| | 527;Y528M;T529A | | 1.1.1.34) | |
| 72 | DNA seq for enzyme P49367 | P49367 | Homoaconitase, mitochondrial | Saccharomyces cerevisiae (strain ATCC 204508/ |
| | | | | S288c) (Baker's yeast) |
| 73 | AA seq for enzyme W1QJE4 | W1QJE4 | Homoaconitase, mitochondrial | Ogataea parapolymorpha (strain ATCC 26012/ |
| | | | | BCRC 20466/JCM 22074/NRRL Y-7560/DL-1) |
| | | | | (Yeast) (Hansenula polymorpha) |
| 74 | DNA seq for enzyme W1QJE4 | W1QJE4 | Homoaconitase, mitochondrial | Ogataea parapolymorpha (strain ATCC 26012/ |
| | | | | BCRC 20466/JCM 22074/NRRL Y-7560/DL-1) |
| | | | | (Yeast) (Hansenula polymorpha) |
| 75 | DNA seq for enzyme P49367 | P49367 | Homoaconitase, mitochondrial | Saccharomyces cerevisiae (strain ATCC 204508/ |
| | | | | S288c) (Baker's yeast) |
| 76 | DNA seq for enzyme | A0A0G9LF37 | Trans-homoaconitate synthase | Clostridium sp. C8 |
| | A0A0G9LF37 | | | |
| 77 | DNA seq for enzyme P48570 | P48570 | Homocitrate synthase, cytosolic isozyme | Saccharomyces cerevisiae (strain ATCC 204508/ |
| | | | | S288c) (Baker's yeast) |
| 78 | DNA seq for enzyme P40495 | P40495 | Homoisocitrate dehydrogenase, | Saccharomyces cerevisiae (strain ATCC 204508/ |
| | | | mitochondrial | S288c) (Baker's yeast) |
| 79 | DNA seq for enzyme P40495 | P40495 | Homoisocitrate dehydrogenase, | Saccharomyces cerevisiae (strain ATCC 204508/ |
| | | | mitochondrial | S288c) (Baker's yeast) |
| 80 | DNA seq for enzyme P48570 | P48570 | Homocitrate synthase, cytosolic isozyme | Saccharomyces cerevisiae (strain ATCC 204508/ |
| | | | | S288c) (Baker's yeast) |
| 81 | DNA seq for enzyme P40495 | P40495 | Homoisocitrate dehydrogenase, | Saccharomyces cerevisiae (strain ATCC 204508/ |
| | | | mitochondrial | S288c) (Baker's yeast) |
| 82 | DNA seq for enzyme P49367 | P49367 | Homoaconitase, mitochondrial | Saccharomyces cerevisiae (strain ATCC 204508/ |
| | | | | S288c) (Baker's yeast) |
| 83 | AA seq for enzyme Q4WUL6 | Q4WUL6 | Homoaconitase, mitochondrial | Neosartorya fumigata (strain ATCC MYA-4609/ |
| | with AA residues 2-41 and 721- | | | Af293/CBS 101355/FGSC A1100) (Aspergillus |
| | 777 truncated | | | fumigatus) |
| 84 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 85 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 86 | DNA seq for enzyme Q9Y823 | Q9Y823 | Homocitrate synthase, mitochondrial | Schizosaccharomyces pombe (strain 972/ATCC |
| | containing AA substitution | | | 24843) (Fission yeast) |
| | D123N | | | |
| 87 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 88 | AA seq for enzyme Q72IW9 | Q72IW9 | Homoisocitrate dehydrogenase | Thermus thermophilus (strain HB27/ATCC |
| | | | | BAA-163/DSM 7039) |
| 89 | DNA seq for enzyme Q72IW9 | Q72IW9 | Homoisocitrate dehydrogenase | Thermus thermophilus (strain HB27/ATCC |
| | | | | BAA-163/DSM 7039) |
| 90 | AA seq for enzyme Q9Y823 | Q9Y823 | Homocitrate synthase, mitochondrial | Schizosaccharomyces pombe (strain 972/ATCC |
| | containing AA substitution | | | 24843) (Fission yeast) |
| | D123N | | | |
| 91 | DNA seq for enzyme Q9Y823 | Q9Y823 | Homocitrate synthase, mitochondrial | Schizosaccharomyces pombe (strain 972/ATCC |
| | containing AA substitution | | | 24843) (Fission yeast) |
| | D123N | | | |
| 92 | DNA seq for enzyme 087198 | O87198 | Homocitrate synthase | Thermus thermophilus (strain HB27/ATCC |
| | | | | BAA-163/DSM 7039) |
| 93 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 94 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 95 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 96 | AA seq for enzyme | A0A0G9LF37 | Trans-homoaconitate synthase | Clostridium sp. C8 |
| | A0A0G9LF37 | | | |
| 97 | DNA seq for enzyme | A0A0G9LF37 | Trans-homoaconitate synthase | Clostridium sp. C8 |
| | A0A0G9LF37 | | | |
| 98 | DNA seq for enzyme Q72IW9 | Q72IW9 | Homoisocitrate dehydrogenase | Thermus thermophilus (strain HB27/ATCC |
| | | | | BAA-163/DSM 7039) |
| 99 | DNA seq for enzyme P49367 | P49367 | Homoaconitase, mitochondrial | Saccharomyces cerevisiae (strain ATCC 204508/ |
| | | | | S288c) (Baker's yeast) |
| 100 | DNA seq for enzyme | A0A0G9LF37 | Trans-homoaconitate synthase | Clostridium sp. C8 |
| | A0A0G9LF37 | | | |
| 101 | DNA seq for enzyme | A0A0G9LF37 | Trans-homoaconitate synthase | Clostridium sp. C8 |
| | A0A0G9LF37 | | | |
| 102 | DNA seq for enzyme Q72IW9 | Q72IW9 | Homoisocitrate dehydrogenase | Thermus thermophilus (strain HB27/ATCC |
| | | | | BAA-163/DSM 7039) |
| 103 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 104 | DNA seq for enzyme Q9Y823 | Q9Y823 | Homocitrate synthase, mitochondrial | Schizosaccharomyces pombe (strain 972/ATCC |
| | containing AA substitution | | | 24843) (Fission yeast) |
| | D123N | | | |
| 105 | DNA seq for enzyme Q9Y823 | Q9Y823 | Homocitrate synthase, mitochondrial | Schizosaccharomyces pombe (strain 972/ATCC |
| | containing AA substitution | | | 24843) (Fission yeast) |
| | D123N | | | |
| 106 | DNA seq for enzyme P49367 | P49367 | Homoaconitase, mitochondrial | Saccharomyces cerevisiae (strain ATCC 204508/ |
| | | | | S288c) (Baker's yeast) |
| 107 | AA seq for enzyme W1QLF1 | W1QLF1 | Homoisocitrate dehydrogenase, | Ogataea parapolymorpha (strain ATCC 26012/ |
| | | | mitochondrial | BCRC 20466/JCM 22074/NRRL Y-7560/DL-1) |
| | | | | (Yeast) (Hansenula polymorpha) |
| 108 | DNA seq for enzyme W1QLF1 | W1QLF1 | Homoisocitrate dehydrogenase, | Ogataea parapolymorpha (strain ATCC 26012/ |
| | | | mitochondrial | BCRC 20466/JCM 22074/NRRL Y-7560/DL-1) |
| | | | | (Yeast) (Hansenula polymorpha) |
| 109 | DNA seq for enzyme P48570 | P48570 | Homocitrate synthase, cytosolic isozyme | Saccharomyces cerevisiae (strain ATCC 204508/ |
| | | | | S288c) (Baker's yeast) |
| 110 | DNA seq for enzyme P48570 | P48570 | Homocitrate synthase, cytosolic isozyme | Saccharomyces cerevisiae (strain ATCC 204508/ |
| | | | | S288c) (Baker's yeast) |
| 111 | DNA seq for enzyme P49367 | P49367 | Homoaconitase, mitochondrial | Saccharomyces cerevisiae (strain ATCC 204508/ |
| | | | | S288c) (Baker's yeast) |
| 112 | DNA seq for enzyme O87198 | O87198 | Homocitrate synthase | Thermus thermophilus (strain HB27/ATCC |
| | | | | BAA-163/DSM 7039) |
| 113 | DNA seq for enzyme P49367 | P49367 | Homoaconitase, mitochondrial | Saccharomyces cerevisiae (strain ATCC 204508/ |
| | | | | S288c) (Baker's yeast) |
| 114 | DNA seq for enzyme Q4WUL6 | Q4WUL6 | Homoaconitase, mitochondrial | Neosartorya fumigata (strain ATCC MYA-4609/ |
| | | | | Af293/CBS 101355/FGSC A1100) (Aspergillus |
| | | | | fumigatus) |
| 115 | DNA seq for enzyme O87198 | O87198 | Homocitrate synthase | Thermus thermophilus (strain HB27/ATCC |
| | | | | BAA-163/DSM 7039) |
| 116 | AA seq for enzyme O87198 | O87198 | Homocitrate synthase | Thermus thermophilus (strain HB27/ATCC |
| | | | | BAA-163/DSM 7039) |
| 117 | DNA seq for enzyme O87198 | O87198 | Homocitrate synthase | Thermus thermophilus (strain HB27/ATCC |
| | | | | BAA-163/DSM 7039) |
| 118 | DNA seq for enzyme O87198 | O87198 | Homocitrate synthase | Thermus thermophilus (strain HB27/ATCC |
| | | | | BAA-163/DSM 7039) |
| 119 | DNA seq for enzyme Q72IW9 | Q72IW9 | Homoisocitrate dehydrogenase | Thermus thermophilus (strain HB27/ATCC |
| | | | | BAA-163/DSM 7039) |
| 120 | AA seq for enzyme F2QPL2 | F2QPL2 | Homocitrate synthase | Komagataella pastoris |
| 121 | DNA seq for enzyme F2QPL2 | F2QPL2 | Homocitrate synthase | Komagataella pastoris |

Microbial Host Cells

Any microbe that can be used to express introduced genes can be engineered for fermentative production of 2-oxoadipate as described above. In certain embodiments, the microbe is one that is naturally incapable of fermentative production of 2-oxoadipate. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacilis spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T reesei, T viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Genetic Engineering Methods

Microbial cells can be engineered for fermentative 2-oxoadipate production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum and S. cerevisiae cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, 2-oxoadipate. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for 2-oxoadipate production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous homocitrate synthase, such as in the case of a microbial host cell that does not naturally produce 2-oxoadipate. In various embodiments, the microbial cell can include and express, for example: (1) a single heterologous homocitrate synthase gene, (2) two or more heterologous homocitrate synthase genes, which can be the same or different (in other words, multiple copies of the same heterologous 2 homocitrate synthase genes can be introduced or multiple, different heterologous homocitrate synthase genes can be introduced), (3) a single heterologous homocitrate synthase gene that is not native to the cell and one or more additional copies of an native homocitrate synthase gene, or (4) two or more non-native homocitrate synthase genes, which can be the same or different, and one or more additional copies of an native homocitrate synthase gene.

This engineered host cell can include at least one additional genetic alteration that increases flux through the pathway leading to the production of homoisocitrate (the immediate precursor of 2-oxoadipate). These “upstream” enzymes in the pathway include: citrate synthase (E.C. 2.3.3.1), aconitase (E.C. 4.2.1.3), isocitrate dehydrogenase (E.C. 1.1.1.42 or E.C. 1.1.1.41), pyruvate dehydrogenase (E.C. 1.2.4.1), dihydrolipoyl transacetylase (E.C. 2.3.1.12), dihydrolipoyl dehydrogenase (E.C. 1.8.1.4), including any isoforms, paralogs, or orthologs having these enzymatic activities (which as those of skill in the art readily appreciate may be known by different names). The at least one additional alteration can increase the activity of the upstream pathway enzyme(s) by any available means, e.g., by: (1) modulating the expression or activity of the native enzyme(s), (2) expressing one or more additional copies of the genes for the native enzymes, and/or (3) expressing one or more copies of the genes for one or more non-native enzymes.

The engineered microbial cells can contain introduced genes that have a native nucleotide sequence or that differ from native. For example, the native nucleotide sequence can be codon-optimized for expression in a particular host cell. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

In some embodiments, increased availability of precursors to 2-oxoadipate can be achieved by reducing the expression or activity of enzymes that consume one or more 2-oxoadipate pathway precursors, such as alpha-ketoglutarate dehydrogenase and citrate synthase. For example, the engineered host cell can include one or more promoter swaps to down-regulate expression of any of these enzymes and/or can have their genes deleted to eliminate their expression entirely.

The approach described herein has been carried out in bacterial cells, namely C. glutamicum (prokaryotes), and in fungal cells, namely the yeast S. cerevisiae (eukaryotes). (See Examples 1 and 2.) Other microbial hosts of particular interest included B. subtilis and Y. lypolytica. (See Example 2.)

Illustrative Engineered Yeast Cells

In certain embodiments, the engineered yeast (e.g., S. cerevisiae) cell expresses a heterologous (e.g., non-native) homocitrate synthase having at least 70 percent, 75 percent, 80 percent, 85 percent, 90 percent, 95 percent, or 100 percent amino acid sequence identity to a homocitrate synthase from Komagataella pastoris (UniProt ID F2QPL2; e.g., SEQ ID NO:(SEQ ID NO:120). In particular embodiments, the Komagataella pastoris homocitrate synthase can include SEQ ID NO:120. The engineered yeast (e.g., S. cerevisiae) cell can alternatively or additionally express a heterologous homocitrate synthase having at least 70 percent 75 percent, 80 percent, 85 percent, 90 percent, 95 percent or 100 percent amino acid sequence identity to a homocitrate synthase from Thermus thermophilus (UniProt ID 087198; SEQ ID NO:116). In particular embodiments, the Thermus thermophilus homocitrate synthase includes SEQ ID NO:116.

In certain embodiments, the engineered yeast (e.g., S. cerevisiae or Y. lipolytica) cell expresses heterologous (e.g., non-native) enzymes including: a homocitrate synthase having at least 70 percent, 75 percent, 80 percent, 85 percent, 90 percent, 95 percent, or 100 percent amino acid sequence identity to a homocitrate synthase from Schizosaccharomyces pombe (strain 972/ATCC 24843) (Fission yeast) (Uniprot ID No. Q9Y823; SEQ ID NO:90), having amino acid substitution D123N (in particular embodiments, the S. pombe homocitrate synthase can include the sequence resulting from incorporation of the amino acid substitution D123N into SEQ ID NO:90); a homoaconitase having at least 70 percent, 75 percent, 80 percent, 85 percent, 90 percent, 95 percent, or 100 percent amino acid sequence identity to a homoaconitase from Saccharomyces cerevisiae (strain ATCC 204508/S288c) (Baker's yeast) (Uniprot ID No. P49367; SEQ ID NO:33) (in particular embodiments, the S. cerevisiae homoaconitase can include SEQ ID NO:33); and a homoisocitrate dehydrogenase having at least 70 percent, 75 percent, 80 percent, 85 percent, 90 percent, 95 percent, or 100 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11) (in particular embodiments, the S. cerevisiae homoisocitrate dehydrogenase can include SEQ ID NO:11).

These may be the only genetic alterations of the engineered yeast cell, or the yeast cell can include one or more additional genetic alterations, as discussed more generally above.

Illustrative Engineered Bacterial Cells

In certain embodiments, the engineered bacterial (e.g., C. glutamicum) cell expresses a heterologous homocitrate synthase having at least 70 percent, 75 percent, 80 percent, 85 percent, 90 percent, 95 percent or 100 percent amino acid sequence identity with a homocitrate synthase from Thermus thermophilus (UniProt ID 087198; SEQ ID NO:116). In particular embodiments, the Thermus thermophilus homocitrate synthase includes SEQ ID NO:116. The engineered bacterial (e.g., C. glutamicum) cell can also express a heterologous homoaconitase having at least 70 percent, 75 percent, 80 percent, 85 percent, 90 percent, 95 percent or 100 percent amino acid sequence identity with a homoaconitase from Ogataea parapolymorpha (UniProt ID W1QJE4; SEQ ID NO:73). In particular embodiments, the Ogataea parapolymorpha homoaconitase includes SEQ ID NO:73. In some embodiments, the engineered bacterial (e.g., C. glutamicum) cell also expresses a heterologous homoisocitrate dehydrogenase having at least 70 percent, 75 percent, 80 percent, 85 percent, 90 percent, 95 percent or 100 percent amino acid sequence identity to a homoisocitrate dehydrogenase from Ogataea parapolymorpha (UniProt ID W1QLF1; SEQ ID NO:107). In particular embodiments, the Ogataea parapolymorpha (UniProt ID W1QLF1; homoisocitrate dehydrogenase includes SEQ ID NO:107.

In certain embodiments, the engineered bacterial (e.g., C. glutamicum) cell expresses heterologous (e.g., non-native) enzymes including: a homocitrate synthase having at least 70 percent, 75 percent, 80 percent, 85 percent, 90 percent, 95 percent, or 100 percent amino acid sequence identity to a homocitrate synthase from Schizosaccharomyces pombe (strain 972/ATCC 24843) (Fission yeast) (Uniprot ID No. Q9Y823; SEQ ID NO:90), having amino acid substitution D123N (in particular embodiments, the S. pombe homocitrate synthase can include the sequence resulting from incorporation of the amino acid substitution D123N into SEQ ID NO:90); a homoaconitase having at least 70 percent, 75 percent, 80 percent, 85 percent, 90 percent, 95 percent, or 100 percent amino acid sequence identity to a homoaconitase from Saccharomyces cerevisiae (strain ATCC 204508/S288c) (Baker's yeast) (Uniprot ID No. P49367; SEQ ID NO:33) (in particular embodiments, the S. cerevisiae homoaconitase can include SEQ ID NO:33); and a homoisocitrate dehydrogenase having at least 70 percent, 75 percent, 80 percent, 85 percent, 90 percent, 95 percent, or 100 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11) (in particular embodiments, the S. cerevisiae homoisocitrate dehydrogenase can include SEQ ID NO:11).

In certain embodiments, the engineered bacterial (e.g., B. subtilis) cell expresses heterologous (e.g., non-native) enzymes including: a homocitrate synthase having at least 70 percent, 75 percent, 80 percent, 85 percent, 90 percent, 95 percent, or 100 percent amino acid sequence identity to a homocitrate synthase from Saccharomyces cerevisiae (strain ATCC 204508/S288c) (Baker's yeast) (Uniprot ID No. P48570; SEQ ID NO:35) (in particular embodiments, the S. cerevisiae homocitrate synthase can include SEQ ID NO:35); a homoaconitase having at least 70 percent, 75 percent, 80 percent, 85 percent, 90 percent, 95 percent, or 100 percent amino acid sequence identity to a homoaconitase from Neosartorya fumigata (strain ATCC MYA-4609/Af293/CBS 101355/FGSC A1100) (Aspergillus fumigatus) (Uniprot ID No. Q4WUL6; SEQ ID NO:83), which includes a deletion of amino acid residues 2-41 and 721-777, relative to the full-length sequence (in particular embodiments, the N. fumigata homoaconitase can include SEQ ID NO:83); and a homoisocitrate dehydrogenase having at least 70 percent, 75 percent, 80 percent, 85 percent, 90 percent, 95 percent, or 100 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11) (in particular embodiments, the S. cerevisiae homoisocitrate dehydrogenase can include SEQ ID NO:11).

### Culturing of Engineered Microbial Cells

Any of the microbial cells described herein can be cultured, e.g., for maintenance, growth, and/or 2-oxoadipate production.

In some embodiments, the cultures are grown to an optical density at 600 nm of 10-500, such as an optical density of 50-150.

In various embodiments, the cultures include produced 2-oxoadipate at titers of at least 10, 25, 50, 75, 100, 200, 300, 400, 500, 600, 700, 800, or 900 μg/L, or at least 1, 10, 50, 75, 100, 200, 300, 400, 500, 600, 700, 800, or 900 mg/L or at least 1, 1.5, 2, 2.5, 3, 3.5, 4, 4.5, 5, 10, 20, 50 g/L. In various embodiments, the titer is in the range of 10 μg/L to 10 g/L, 25 μg/L to 20 g/L, 100 μs/L to 10 g/L, 200 μg/L to 5 g/L, 500 μs/L to 4 g/L, 1 mg/L to 3 g/L, 500 mg/L to 2 g/L or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (laevulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce 2-oxoadipate, the culture medium can include, and/or is supplemented during culture with, glucose and/or a nitrogen source such as urea, an ammonium salt, ammonia, or any combination thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO₂, and a pH between about 5 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels fall within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

Illustrative materials and methods suitable for the maintenance and growth of the engineered microbial cells described herein can be found below in Example 1.

### 2-Oxoadipate Production and Recovery

Any of the methods described herein may further include a step of recovering 2-oxoadipate. In some embodiments, the produced 2-oxoadipate contained in a so-called harvest stream is recovered/harvested from the production vessel. The harvest stream may include, for instance, cell-free or cell-containing aqueous solution coming from the production vessel, which contains 2-oxoadipate as a result of the conversion of production substrate by the resting cells in the production vessel. Cells still present in the harvest stream may be separated from the 2-oxoadipate by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced 2-oxoadipate from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify 2-oxoadipate. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### Example 1—Construction and Selection of Strains of Corynebacterium glutamicum and Saccharomyces cerevisiae Engineered to Produce 2-Oxoadipate

Plasmid/DNA Design

All strains tested for this work were transformed with plasmid DNA designed using proprietary software. Plasmid designs were specific to each of the host organisms engineered in this work. The plasmid DNA was physically constructed by a standard DNA assembly method. This plasmid DNA was then used to integrate metabolic pathway inserts by one of two host-specific methods, each described below.

C. glutamicum Pathway Integration

A “loop-in, single-crossover” genomic integration strategy has been developed to engineer C. glutamicum strains. FIG. 9 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum chromosome. Integration events are stably maintained in the genome by growth in the presence of antibiotic (25 μg/ml kanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome of C. glutamicum. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

S. cerevisiae Pathway Integration

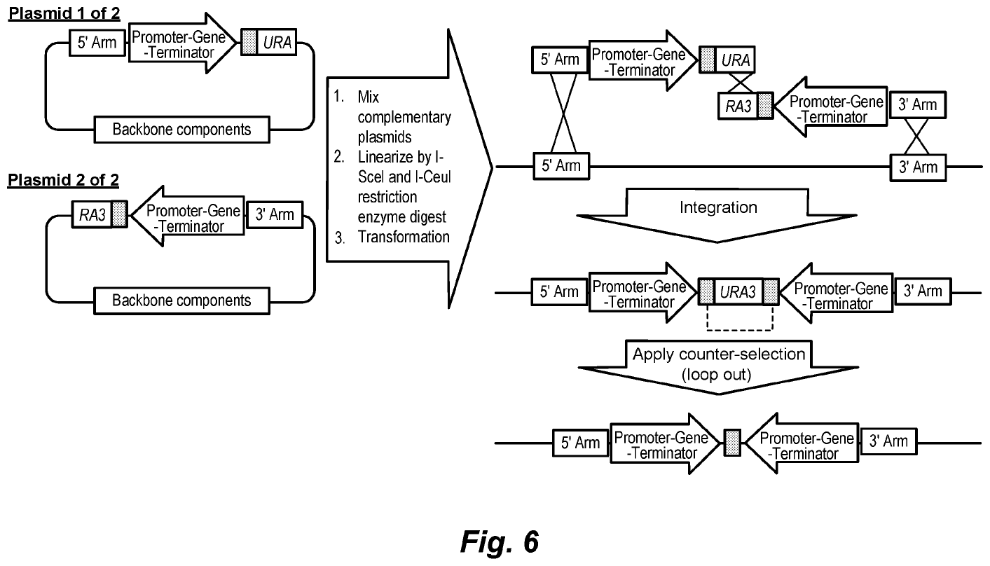

A “split-marker, double-crossover” genomic integration strategy has been developed to engineer S. cerevisiae strains. FIG. 6 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

Cell Culture

The workflow established for S. cerevisiae involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (SD-ura for S. cerevisiae) and cultivated for two days until saturation and then frozen with 16.6% glycerol at −80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 μL of supernatant was transferred to each plate, with one stored at 4° C., and the second stored at 80° C. for long-term storage.

First-Round Genetic Engineering Results in Corynebacterium glutamicum and Saccharomyces cerevisiae

A library approach was taken to screen heterologous pathway enzymes to establish the 2-oxoadipate pathway. For homocitrate synthase, five heterologous sequences from fungi and one heterologous sequence from bacteria were tested from sources listed in Table 1. The homocitrate synthases were codon-optimized and expressed in both Saccharomyces cerevisiae and Corynebacterium glutamicum hosts. For homoaconitase, six heterologous sequences from fungi were tested from sources listed in Table 1. The homoaconitases were codon-optimized and expressed in the C. glutamicum host. For homoisocitrate dehydrogenase, three heterologous sequences from fungi were tested from the sources listed in Table 1. The homoisocitrate dehydrogenases were codon-optimized and expressed in the C. glutamicum host.

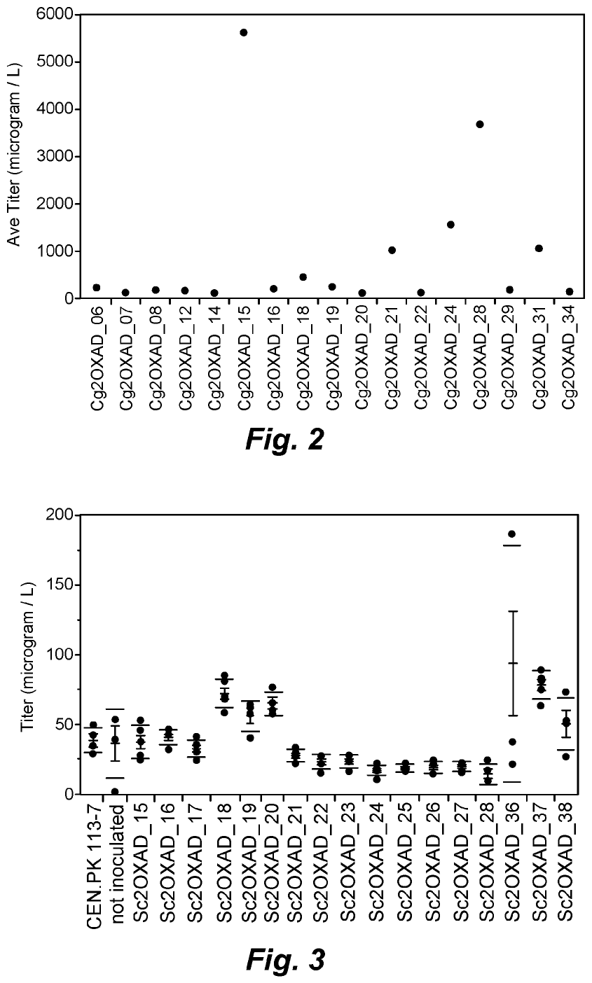

First-round genetic engineering results are shown in Table 1 and FIGS. 2 (C. glutamicum) and 3 (S. cerevisiae). In Corynebacterium glutamicum, a 28 mg/L titer of 2-oxoadipate was achieved in a first round of engineering after integration of the three necessary non-native enzymes. In Saccharomyces cerevisiae, a titer of 128 μg/L was achieved in a first round of engineering after integration of a homocitrate synthase.

TABLE 1
First-round genetic engineering results in Corynebacterium glutamicum and Saccharomyces cerevisiae

| | | | | | | | |
|---|---|---|---|---|---|---|---|
| | Titer | E1 | Enzyme 1- | Enzyme 1- | E1 Codon | E2 | Enzyme 2- |
| Strain name | (μg/L) | Uniprot ID | activity name | source organism | Optimization | Uniprot ID | activity name |

Corynebacterium glutamicum

| Cg2OXAD_06 | 24988.4 | B9W7P6 | homocitrate | Candida dubliniensis | Cg | F2QY53 | homoaconitase |
|---|---|---|---|---|---|---|---|
| | | | synthase | | | | |
| Cg2OXAD_07 | 25622.6 | B9W7P6 | homocitrate | Candida dubliniensis | Cg | E9L3N1 | homoaconitase |
| | | | synthase | | | | |
| Cg2OXAD_08 | 26845.7 | B9W7P6 | homocitrate | Candida dubliniensis | Cg | F8DCX2 | homoaconitase |
| | | | synthase | | | | |
| Cg2OXAD_12 | 27166.4 | 63CBV0 | homocitrate | Ustilaginoidea virens | Cg | E9L3N1 | homoaconitase |
| | | | synthase | | | | |
| Cg2OXAD_14 | 24969.6 | 63CBV0 | homocitrate | Ustilaginoidea virens | Cg | E9L3N1 | homoaconitase |
| | | | synthase | | | | |
| Cg2OXAD_15 | 27130.9 | O87198 | homocitrate | Thermus thermophilus | Cg | W1QJE4 | homoaconitase |
| | | | synthase | | | | |
| Cg2OXAD_16 | 24327.2 | S9W189 | homocitrate | Schizosaccharomyces | Cg | W1QJE4 | homoaconitase |
| | | | synthase | cryophilus | | | |
| Cg2OXAD_18 | 28512.3 | F2QPL2 | homocitrate | Komagataella | Cg | W1QJE4 | homoaconitase |
| | | | synthase | pastoris | | | |
| Cg2OXAD_19 | 25598.7 | B9W7P6 | homocitrate | Candida dubliniensis | Cg | W1QJE4 | homoaconitase |
| | | | synthase | | | | |
| Cg2OXAD_20 | 26456.3 | 63CBV0 | homocitrate | Ustilaginoidea virens | Cg | W1QJE4 | homoaconitase |
| | | | synthase | | | | |
| Cg2OXAD_24 | 28564.4 | P48570 | homocitrate | Saccharomyces | Cg | W7MZD4 | homoaconitase |
| | | | synthase | cerevisiae | | | |
| Cg2OXAD_29 | 25875.8 | F2QPL2 | homocitrate | Komagataella | Cg | F2QY53 | homoaconitase |
| | | | synthase | pastoris | | | |
| Cg2OXAD_31 | 26366.3 | F2QPL2 | homocitrate | Komagataella | Cg | F2QY53 | homoaconitase |
| | | | synthase | pastoris | | | |
| Cg2OXAD_34 | 27713.5 | 63CBV0 | homocitrate | Ustilaginoidea virens | Cg | E9L3N1 | homoaconitase |
| | | | synthase | | | | |
| | | Enzyme 2- | E2 Codon | E3 | Enzyme 3- | Enzyme 3- | E3 Codon |
| | Strain name | source organism | Optimization | Uniprot ID | activity name | source organism | Optimization |
| | Cg2OXAD_06 | Komagataella | Cg | B9WKX4 | homoisocitrate | Candida | Cg |
| | | pastoris | | | dehydrogenase | dubliniensis | |
| | Cg2OXAD_07 | Ustilaginoidea | Cg | B9WKX4 | homoisocitrate | Candida | Cg |
| | | virens | | | dehydrogenase | dubliniensis | |
| | Cg2OXAD_08 | Ceratocystis | Cg | B9WKX4 | homoisocitrate | Candida | Cg |
| | | fimbriata f. sp. | | | dehydrogenase | dubliniensis | |
| | | Platani | | | | | |
| | Cg2OXAD_12 | Ustilaginoidea | Cg | P40495 | homoisocitrate | Saccharomyces | Cg |
| | | virens | | | dehydrogenase | cerevisiae | |
| | Cg2OXAD_14 | Ustilaginoidea | Cg | W1QLF1 | homoisocitrate | Ogataea | Cg |
| | | virens | | | dehydrogenase | parapolymorpha | |
| | Cg2OXAD_15 | Ogataea | Cg | W1QLF1 | homoisocitrate | Ogataea | Cg |
| | | parapolymorpha | | | dehydrogenase | parapolymorpha | |
| | Cg2OXAD_16 | Ogataea | Cg | W1QLF1 | homoisocitrate | Ogataea | Cg |
| | | parapolymorpha | | | dehydrogenase | parapolymorpha | |
| | Cg2OXAD_18 | Ogataea | Cg | W1QLF1 | homoisocitrate | Ogataea | Cg |
| | | parapolymorpha | | | dehydrogenase | parapolymorpha | |
| | Cg2OXAD_19 | Ogataea | Cg | W1QLF1 | homoisocitrate | Ogataea | Cg |
| | | parapolymorpha | | | dehydrogenase | parapolymorpha | |
| | Cg2OXAD_20 | Ogataea | Cg | W1QLF1 | homoisocitrate | Ogataea | Cg |
| | | parapolymorpha | | | dehydrogenase | parapolymorpha | |
| | Cg2OXAD_24 | Gibberella | Cg | P40495 | homoisocitrate | Saccharomyces | Cg |
| | | moniliformis | | | dehydrogenase | cerevisiae | |
| | Cg2OXAD_29 | Komagataella | Cg | B9WKX4 | homoisocitrate | Candida | Cg |
| | | pastoris | | | dehydrogenase | dubliniensis | |
| | Cg2OXAD_31 | Komagataella | Cg | B9WKX4 | homoisocitrate | Candida | Cg |
| | | pastoris | | | dehydrogenase | dubliniensis | |
| | Cg2OXAD_34 | Ustilaginoidea | Cg | B9WKX4 | homoisocitrate | Candida | Cg |
| | | virens | | | dehydrogenase | dubliniensis | |
| | Titer | E1 | Enzyme 1- | Enzyme 1- | E1 Codon | E2 | Enzyme 2- |
| Strain name | (μg/L) | Uniprot ID | activity name | source organism | Optimization | Uniprot ID | activity name |

Saccharomyces cerevisiae

| Sc2OXAD_15 | 37.5 | O87198 | homocitrate | Thermus thermophilus | Cg | | |
|---|---|---|---|---|---|---|---|
| | | | synthase | | | | |
| Sc2OXAD_16 | 40.8 | S9W189 | homocitrate | Schizosaccharomyces | Cg | | |
| | | | synthase | cryophilus | | | |
| Sc2OXAD_17 | 32.6 | P48570 | homocitrate | Saccharomyces | Cg | | |
| | | | synthase | cerevisiae | | | |
| Sc2OXAD_18 | 128.6 | F2QPL2 | homocitrate | Komagataella | Cg | | |
| | | | synthase | pastoris | | | |
| Sc2OXAD_19 | 55.9 | B9W7P6 | homocitrate | Candida dubliniensis | Cg | | |
| | | | synthase | | | | |
| Sc2OXAD_20 | 64.8 | 63CBV0 | homocitrate | Ustilaginoidea virens | Cg | | |
| | | | synthase | | | | |
| Sc2OXAD_22 | 23.1 | O87198 | homocitrate | Thermus thermophilus | Cg | | |
| | | | synthase | | | | |
| Sc2OXAD_23 | 23.9 | S9W189 | homocitrate | Schizosaccharomyces | Cg | | |
| | | | synthase | cryophilus | | | |
| Sc2OXAD_24 | 17.0 | P48570 | homocitrate | Saccharomyces | Cg | | |
| | | | synthase | cerevisiae | | | |
| Sc2OXAD_25 | 18.8 | F2QPL2 | homocitrate | Komagataella | Cg | | |
| | | | synthase | pastoris | | | |
| Sc2OXAD_26 | 19.1 | B9W7P6 | homocitrate | Candida | Cg | | |
| | | | synthase | dubliniensis | | | |
| Sc2OXAD_27 | 19.8 | 63CBV0 | homocitrate | Ustilaginoidea virens | Cg | | |
| | | | synthase | | | | |
| Sc2OXAD_36 | 93.4 | O87198 | homocitrate | Thermus thermophilus | Cg | | |
| | | | synthase | | | | |
| Sc2OXAD_37 | 78.2 | S9W189 | homocitrate | Schizosaccharomyces | Cg | | |
| | | | synthase | cryophilus | | | |
| Sc2OXAD_38 | 50.6 | P48570 | homocitrate | Saccharomyces | Cg | | |
| | | | synthase | cerevisiae | | | |
| | | Enzyme 2- | E2 Codon | E3 | Enzyme 3- | Enzyme 3- | E3 Codon |
| | Strain name | source organism | Optimization | Uniprot ID | activity name | source organism | Optimization |
| | Sc2OXAD_15 | | | | | | |
| | Sc2OXAD_16 | | | | | | |
| | Sc2OXAD_17 | | | | | | |
| | Sc2OXAD_18 | | | | | | |
| | Sc2OXAD_19 | | | | | | |
| | Sc2OXAD_20 | | | | | | |
| | Sc2OXAD_22 | | | | | | |
| | Sc2OXAD_23 | | | | | | |
| | Sc2OXAD_24 | | | | | | |
| | Sc2OXAD_25 | | | | | | |
| | Sc2OXAD_26 | | | | | | |
| | Sc2OXAD_27 | | | | | | |
| | Sc2OXAD_36 | | | | | | |
| | Sc2OXAD_37 | | | | | | |
| | Sc2OXAD_38 | | | | | | |

Note:
“Cg” refers to codon optimization for Corynebacterium glutamicum.

Second-Round Genetic Engineering Results in Corynebacterium glutamicum and Saccharomyces cerevisiae

In an effort to improve 2-oxoadipate production, an additional homocitrate synthase gene was expressed from a constitutive promoter in the best-performing strains from the first round of genetic engineering. The enzymes and results are listed in Table 2. In addition to the enzymes in Table 2, the strains contained the best enzymes from first round. The Corynebacterium glutamicum host contained a homocitrate synthase from Thermus thermophilus (UniProt ID 087198; SEQ ID NO:116), a homoaconitase from Ogataea parapolymorpha (UniProt ID W1QJE4; SEQ ID NO:73), and a homoisocitrate dehydrogenase from Ogataea parapolymorpha (UniProt ID W1QLF1; SEQ ID NO:107). The Saccharomyces cerevisiae host contained a homocitrate synthase from Komagataella pastoris (UniProt ID F2QPL2; e.g., SEQ ID NO:(SEQ ID NO:120).

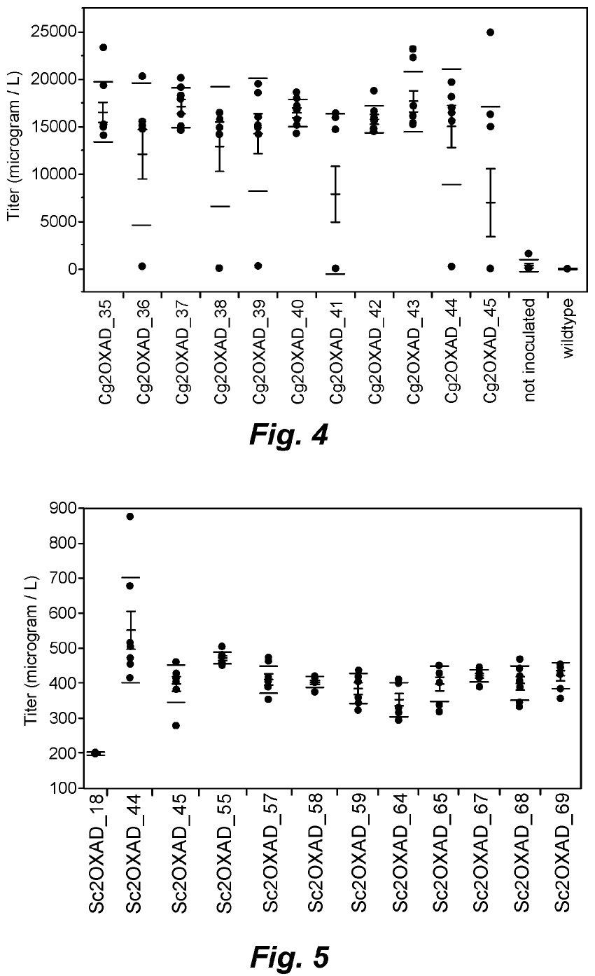

Second-round genetic engineering results are shown in Table 2 and FIGS. 4 (C. glutamicum) and 5 (S. cerevisiae). No improvement was seen in the C. glutamicum strains. In S. cerevisiae, a titer of 553 μg/L was achieved by integration of homocitrate synthase from Thermus thermophilus UniProt ID 087198; SEQ ID NO:116).

TABLE 2
Second-round genetic engineering results in genetic engineering results in Corynebacterium glutamicum and
Saccharomyces cerevisiae

| | Titer | E1 | Enzyme 1- | | |
|---|---|---|---|---|---|
| Strain name | (μg/L) | Uniprot ID | activity name | Enzyme 1-source organism | E1 Codon Optimization |
| Corynebacterium glutamicum | | | | | |
| Cg2OXAD_35 | 11443.0 | O87198 | homocitrate synthase | Thermus thermophilus | Corynebacterium glutamicum |
| Cg2OXAD_36 | 8344.5 | S9W189 | homocitrate synthase | Schizosaccharomyces cryophilus | Corynebacterium glutamicum |
| Cg2OXAD_37 | 9908.4 | P48570 | homocitrate synthase | Saccharomyces cerevisiae | Corynebacterium glutamicum |
| Cg2OXAD_38 | 8398.7 | F2QPL2 | homocitrate synthase | Komagataella pastoris | Corynebacterium glutamicum |
| Cg2OXAD_39 | 10381.7 | B9W7P6 | homocitrate synthase | Candida dubliniensis | Corynebacterium glutamicum |
| Cg2OXAD_40 | 14806.6 | F2QPL2 | homocitrate synthase | Komagataella pastoris | Corynebacterium glutamicum |
| Cg2OXAD_41 | 6061.4 | B9W7P6 | homocitrate synthase | Candida dubliniensis | Corynebacterium glutamicum |
| Cg2OXAD_42 | 9388.3 | O87198 | homocitrate synthase | Thermus thermophilus | Corynebacterium glutamicum |
| Cg2OXAD_43 | 13567.3 | S9W189 | homocitrate synthase | Schizosaccharomyces cryophilus | Corynebacterium glutamicum |
| Cg2OXAD_44 | 17888.1 | P48570 | homocitrate synthase | Saccharomyces cerevisiae | Corynebacterium glutamicum |
| Cg2OXAD_45 | 4068.4 | F2QPL2 | homocitrate synthase | Komagataella pastoris | Corynebacterium glutamicum |
| Saccharomyces cerevisiae | | | | | |
| Sc2OXAD_44 | 553.4 | O87198 | homocitrate synthase | Thermus thermophilus | Corynebacterium glutamicum |
| Sc2OXAD_45 | 400.0 | S9W189 | homocitrate synthase | Schizosaccharomyces cryophilus | Corynebacterium glutamicum |
| Sc2OXAD_55 | 472.7 | 63CBV0 | homocitrate synthase | Ustilaginoidea virens | Corynebacterium glutamicum |
| Sc2OXAD_57 | 412.1 | O87198 | homocitrate synthase | Thermus thermophilus | Corynebacterium glutamicum |
| Sc2OXAD_58 | 405.0 | S9W189 | homocitrate synthase | Schizosaccharomyces cryophilus | Corynebacterium glutamicum |
| Sc2OXAD_59 | 385.8 | P48570 | homocitrate synthase | Saccharomyces cerevisiae | Corynebacterium glutamicum |
| Sc2OXAD_64 | 355.1 | S9W189 | homocitrate synthase | Schizosaccharomyces cryophilus | Corynebacterium glutamicum |
| Sc2OXAD_65 | 399.0 | P48570 | homocitrate synthase | Saccharomyces cerevisiae | Corynebacterium glutamicum |
| Sc2OXAD_67 | 423.2 | F2QPL2 | homocitrate synthase | Komagataella pastoris | Corynebacterium glutamicum |
| Sc2OXAD_68 | 401.0 | O87198 | homocitrate synthase | Thermus thermophilus | Corynebacterium glutamicum |

Third-Round Genetic Engineering Designs in Corynebacterium glutamicum

2-oxoadipate production was further pursued in Corynebacterium glutamicum, and the strain designs are shown in Table 3, below). Because the best-performing C. glutamicum strain from the two previous rounds of engineering had two antibiotic selection markers integrated and cannot be used for additional builds, the strains shown in Table 3 expressed no additional heterologous enzymes (i.e., the Table 3 enzymes were expressed in wild-type C. glutamicum).

### Example 2—Construction and Selection of Strains Engineered to Produce 2-Oxoadipate in Various Hosts

Genetic Engineering Results in Yarrowia lipolytica

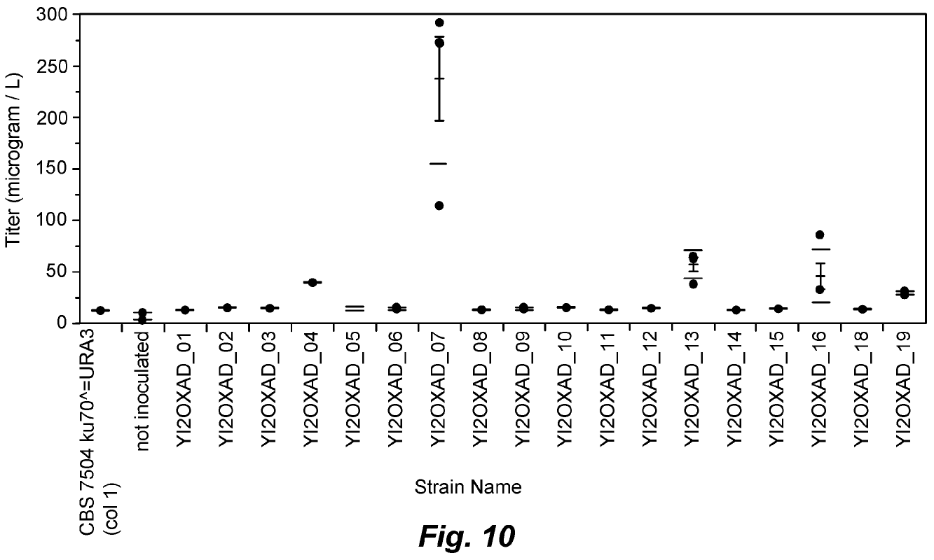

Yarrowia lipolytica was engineered to produce 2-oxoadipate using the same general approach as described above for Saccharomyces cerevisiae (see FIG. 6). First-round genetic engineering results are shown in Table 4 and FIG. 10. In Y. lipolytica, a 238 μg/L titer of 2-oxoadipate was achieved in a first round of engineering after integration of: a homocitrate synthase from Schizosaccharomyces pombe (strain 972/ATCC 24843) (Fission yeast) (Uniprot ID No. Q9Y823; SEQ ID NO:90), having amino acid substitution D123N, a homoaconitase from Saccharomyces cerevisiae (strain ATCC 204508/S288c) (Baker's yeast) (Uniprot ID No. P49367; SEQ ID NO:33), and a homoisocitrate dehydrogenase from Saccharomyces cerevisiae (strain ATCC 204508/S288c) (Baker's yeast) (Uniprot ID No. P40495; SEQ ID NO:11).

Genetic Engineering Results in Bacillus subtilis

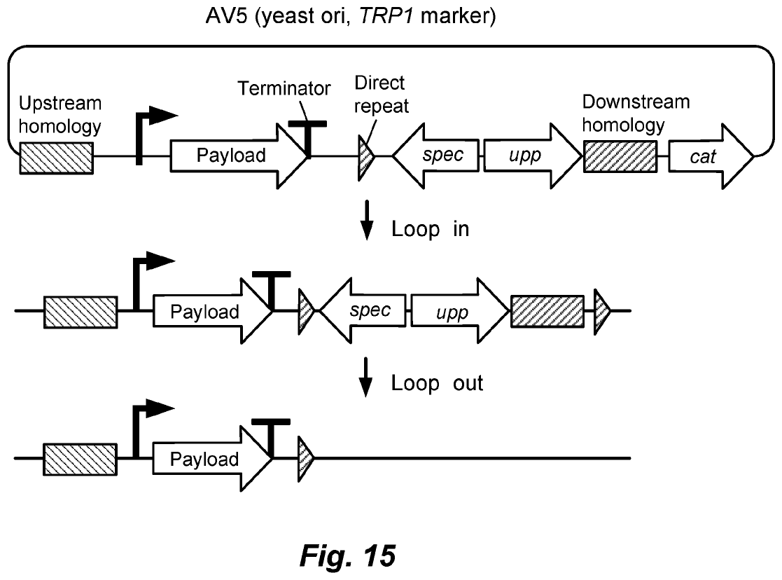

Bacillus subtilis was engineered to produce 2-oxoadipate using a “loop-in, loop-out, double-crossover” genomic integration strategy illustrated schematically in FIG. 15. FIG. 15 shows the double-crossover construct, genomic integration resulting in loop-in, and the loop-out genomic state. The plasmid construct contained the two 2-kb homology arms (denoted as “upstream homology” and “downstream homology”), a positive selection marker (denoted here as “spec”), a counter-selection marker (denoted here as “upp”) and gene(s) of interest (denoted as “payload”) and a short “direct repeat” homologous to a region in the chromosome following the downstream homology arm. A double-crossover event integrated the plasmid into the B. subtilis chromosome. Integration events are stably maintained in the genome by growth in the presence of antibiotic (25 μg/ml spectinomycin). Correct genomic integration in colonies derived from loop-in integration were confirmed by colony PCR with UF/IR and DR/IF PCR primers.

“Loop-out” is achieved by a single crossover event between the direct repeats in the chromosome of B. subtilis. Correct genomic integration was confirmed by colony PCR and counter-selection was applied so that the selection and counter-selection markers could be excised. This results in the desired pathway integration. Again, correct genomic loop-out is confirmed by colony PCR. (Abbreviations: Primers: UF=upstream forward, DR=downstream reverse, IR=internal reverse, IF=internal forward.)

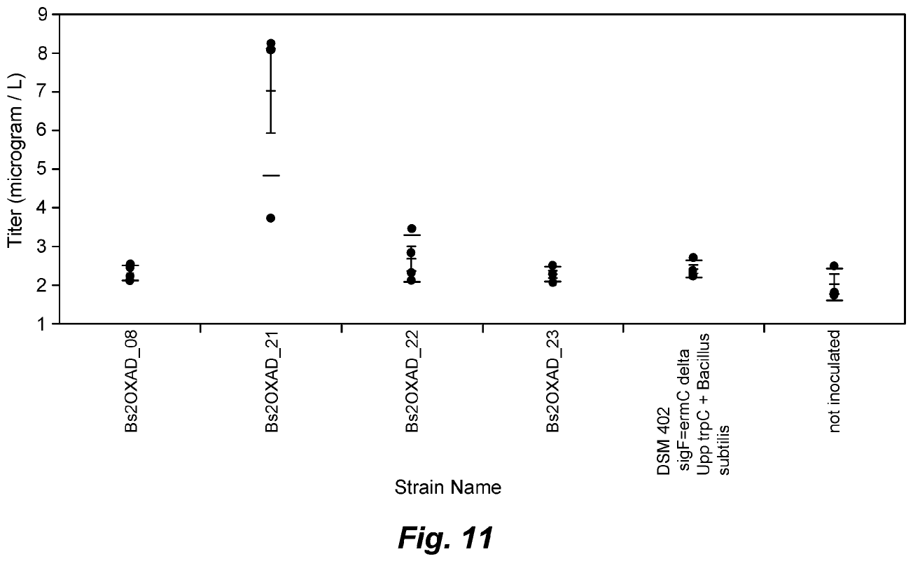

First-round genetic engineering results are shown in Table 5 and FIG. 11. In B. subtilis, a 7 μg/L titer of 2-oxoadipate was achieved in a first round of engineering after integration of: a homocitrate synthase from Saccharomyces cerevisiae (strain ATCC 204508/S288c) (Baker's yeast) (Uniprot ID No. P48570; SEQ ID NO:35), a homoaconitase from Neosartorya fumigata (strain ATCC MYA-4609/Af293/CBS 101355/FGSC A1100) (Aspergillus fumigatus) (Uniprot ID No. Q4WUL6; SEQ ID NO:83), which includes a deletion of amino acid residues 2-41 and 721-777, relative to the full-length sequence, and a homoisocitrate dehydrogenase from Saccharomyces cerevisiae (strain ATCC 204508/S288c) (Baker's yeast) (Uniprot ID No. P40495; SEQ ID NO:11).

Additional Genetic Engineering Results in Saccharomyces cerevisiae

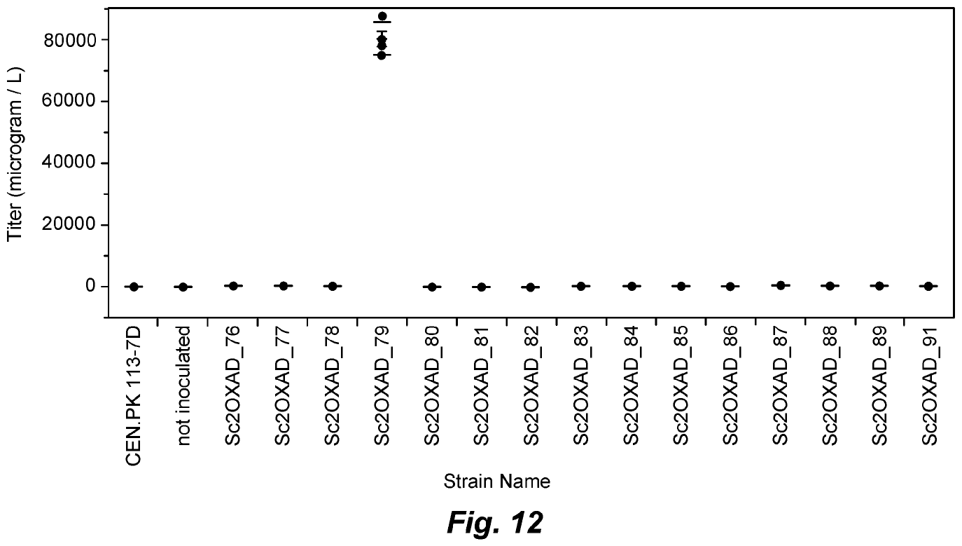

An additional round of engineering for 2-oxoadipate production was carried out in Saccharomyces cerevisiae. Results are shown in Table 6 and FIG. 12. In this round, an 80 mg/L titer of 2-oxoadipate was achieved after integration of: a homocitrate synthase from Schizosaccharomyces pombe (strain 972/ATCC 24843) (Fission yeast) (Uniprot ID No. Q9Y823; SEQ ID NO:90), having amino acid substitution D123N, a homoaconitase from Saccharomyces cerevisiae (strain ATCC 204508/S288c) (Baker's yeast) (Uniprot ID No. P49367; SEQ ID NO:33), and a homoisocitrate dehydrogenase from Saccharomyces cerevisiae (strain ATCC 204508/S288c) (Baker's yeast) (Uniprot ID No. P40495; SEQ ID NO:11).

Host evaluation-round genetic engineering results for Corynebacterium glutamicum

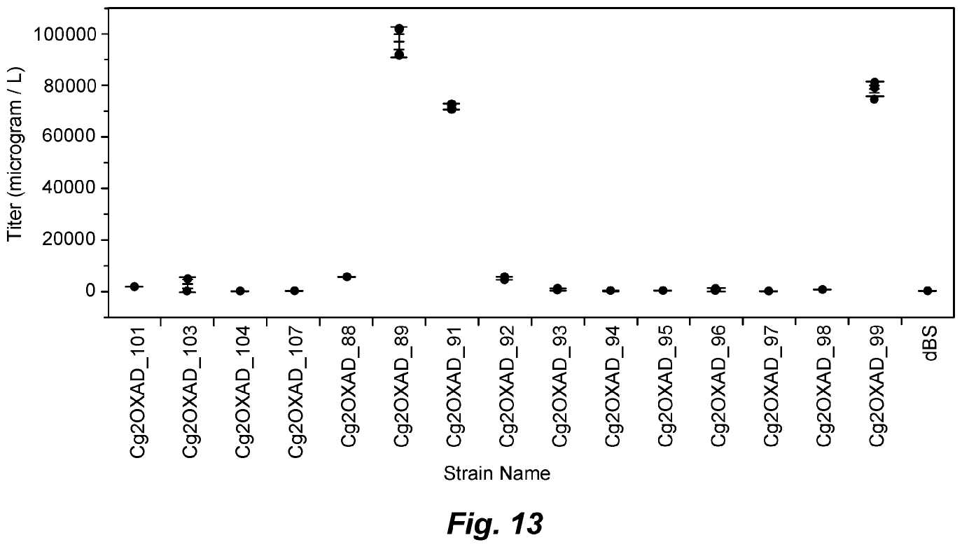

In a host evaluation-round of genetic engineering for 2-oxoadipate production (Table 7; FIG. 13), a titer of 97 mg/L was achieved in Corynebacterium glutamicum after integration of: a homocitrate synthase from Schizosaccharomyces pombe (strain 972/ATCC 24843) (Fission yeast) (Uniprot ID No. Q9Y823; SEQ ID NO:90), having amino acid substitution D123N, a homoaconitase from Saccharomyces cerevisiae (strain ATCC 204508/S288c) (Baker's yeast) (Uniprot ID No. P49367; SEQ ID NO:33), and a homoisocitrate dehydrogenase from Saccharomyces cerevisiae (strain ATCC 204508/S288c) (Baker's yeast) (Uniprot ID No. P40495; SEQ ID NO:11).

Improvement-round genetic engineering results for Corynebacterium glutamicum

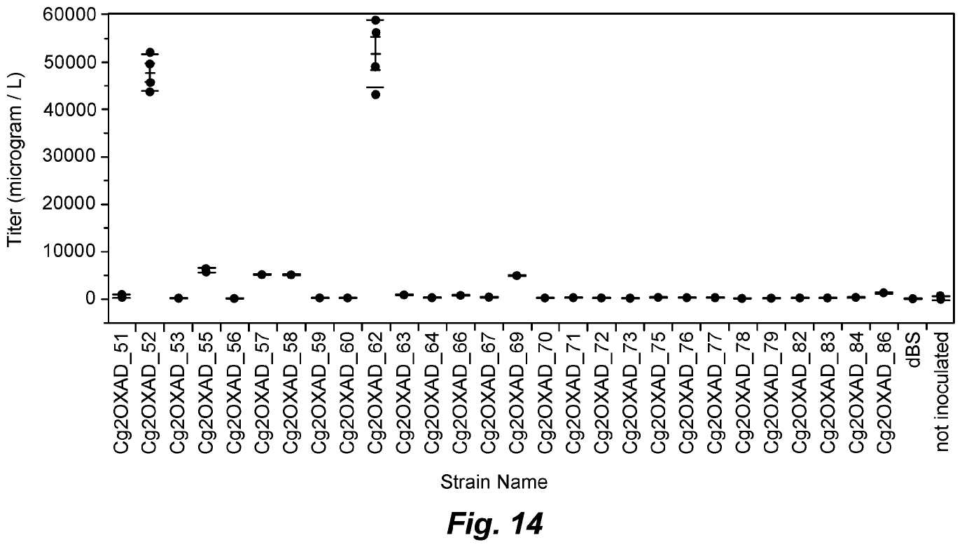

An “improvement-round” of genetic engineering was carried out in Corynebacterium glutamicum. The results are shown in Table 8 and FIG. 14. The highest titer achieved in this round of engineering was 51.7 mg/L.

### TABLES 3-8

TABLE 3
Third-round genetic engineering strain designs in Corynebacterium glutamicum

| | | | | | | |
|---|---|---|---|---|---|---|
| | Enzyme 1- | E1 | Enzyme 1- | E1 Codon | | Enzyme 2- |
| E1 Uniprot ID | activity name | Modifications | source organism | Optimization | E2 Uniprot ID | activity name |

Corynebacterium glutamicum

| Q9Y823 | No Activity | D123N | Schizosaccharomyces | Cg/Sc | P49367 | Homoisocitrate |
|---|---|---|---|---|---|---|
| | Name Found | | pombe ATCC 24843 | | | hydrolyase |
| Q9Y823 | No Activity | E222Q | Schizosaccharomyces | Cg/Sc | P49367 | Homoisocitrate |
| | Name Found | | pombe ATCC 24843 | | | hydrolyase |
| Q9Y823 | No Activity | R288K | Schizosaccharomyces | Cg/Sc | P49367 | Homoisocitrate |
| | Name Found | | pombe ATCC 24843 | | | hydrolyase |
| Q9Y823 | No Activity | Q364R | Schizosaccharomyces | Cg/Sc | P49367 | Homoisocitrate |
| | Name Found | | pombe ATCC 24843 | | | hydrolyase |
| Q9Y823 | No Activity | R275K | Schizosaccharomyces | Cg/Sc | P49367 | Homoisocitrate |
| | Name Found | | pombe ATCC 24843 | | | hydrolyase |
| P48570 | No Activity | 0 | Saccharomyces | Cg/Sc | P49367 | Homoisocitrate |
| | Name Found | | cerevisiae 24843 | | | hydrolyase |
| Q9Y823 | No Activity | D123N | Schizosaccharomyces | Cg/Sc | P49367 | Homoisocitrate |
| | Name Found | | pombe ATCC 24843 | | | hydrolyase |
| P48570 | No Activity | 0 | Saccharomyces | Cg/Sc | P49367 | No Activity |
| | Name Found | | cerevisiae 24843 | | | hydrolyase |
| P48570 | No Activity | 0 | Saccharomyces | Cg/Sc | P49367 | No Activity |
| | Name Found | | cerevisiae 24843 | | | Name Found |
| P48570 | No Activity | 0 | Saccharomyces | Cg/Sc | Q4WUL6 | No Activity |
| | Name Found | | cerevisiae S288c | | | Name Found |
| P48570 | No Activity | 0 | Saccharomyces | Cg/Sc | Q4WUL6 | No Activity |
| | Name Found | | cerevisiae S288c | | | Name Found |
| P48570 | No Activity | 0 | Saccharomyces | Cg/Sc | Q4WUL6 | No Activity |
| | Name Found | | cerevisiae S288c | | | Name Found |
| P48570 | No Activity | 0 | Saccharomyces | Cg/Sc | P49367 | No Activity |
| | Name Found | | cerevisiae S288c | | | Name Found |
| P48570 | No Activity | 0 | Saccharomyces | Cg/Sc | P49367 | No Activity |
| | Name Found | | cerevisiae S288c | | | Name Found |
| P48570 | No Activity | 0 | Saccharomyces | Cg/Sc | P49367 | Homoisocitrate |
| | Name Found | | cerevisiae S288c | | | hydrolyase |
| P48570 | No Activity | 0 | Saccharomyces | Cg/Sc | P49367 | Homoisocitrate |
| | Name Found | | cerevisiae S288c | | | hydrolyase |
| P48570 | No Activity | 0 | Saccharomyces | Cg/Sc | A0A0G9LF37 | No Activity |
| | Name Found | | cerevisiae S288c | | | Name Found |
| P48570 | No Activity | 0 | Saccharomyces | Cg/Sc | | |
| | Name Found | | cerevisiae S288c | | | |
| P48570 | No Activity | 0 | Saccharomyces | Cg/Sc | | |
| | Name Found | | cerevisiae S288c | | | |
| Q57926 | No Activity | 0 | Methanocaldococcus | Cg/Sc | | |
| | Name Found | | jannaschii | | | |
| | | | ATCC 43067 | | | |
| D5Q163 | No Activity | 0 | Clostridioides | Cg/Sc | | |
| | Name Found | | difficile NAP08 | | | |
| Q57926 | No Activity | 0 | Methanocaldococcus | Cg/Sc | P49367 | Homoisocitrate |
| | Name Found | | jannaschii | | | hydrolyase |
| | | | ATCC 43067 | | | |
| O27667 | No Activity | 0 | Methanothermobacter | Cg/Sc | P49367 | Homoisocitrate |
| | Name Found | | thermautotrophicus | | | hydrolyase |
| | | | ATCC 29096 | | | |
| O87198 | No Activity | 0 | Thermus | Cg/Sc | P49367 | Homoisocitrate |
| | Name Found | | thermophilus | | | hydrolyase |
| | | | ATCC BAA-163 | | | |
| G8NBZ9 | No Activity | 0 | Thermus sp. | Cg/Sc | P49367 | Homoisocitrate |
| | Name Found | | CCB_US3_UF1 | | | hydrolyase |
| F2NL20 | No Activity | 0 | Marinithermus | Cg/Sc | P49367 | Homoisocitrate |
| | Name Found | | hydrothermalis | | | hydrolyase |
| | | | DSM 14884 | | | |
| E4U9R8 | No Activity | 0 | Oceanithermus | Cg/Sc | P49367 | Homoisocitrate |
| | Name Found | | profundus DSM | | | hydrolyase |
| A0A0F7TVK2 | No Activity | 0 | Penicillium | Cg/Sc | P49367 | Homoisocitrate |
| | Name Found | | brasilianum | | | hydrolyase |
| A0A0L1I0C1 | No Activity | 0 | Stemphylium | Cg/Sc | P49367 | Homoisocitrate |
| | Name Found | | lycopersici | | | hydrolyase |
| C1CVX4 | No Activity | 0 | Deinococcus | Cg/Sc | P49367 | Homoisocitrate |
| | Name Found | | deserti DSM 17065 | | | hydrolyase |
| Q9RUZ2 | No Activity | 0 | Deinococcus | Cg/Sc | P49367 | Homoisocitrate |
| | Name Found | | radiodurans | | | hydrolyase |
| | | | ATCC 13939 | | | |
| Q2IHS7 | No Activity | 0 | Anaeromyxobacter | Cg/Sc | P49367 | Homoisocitrate |
| | Name Found | | dehalogenans | | | hydrolyase |
| | | | (strain 2CP-C) | | | |
| A0A1F8TP88 | No Activity | 0 | Chloroflexi bacterium | Cg/Sc | Q4WUL6 | No Activity |
| | Name Found | | RIFCSPLOWO2 | | | Name Found |
| | | | _12_FULL_71_12 | | | |
| Q9Y823 | No Activity | 0 | Schizosaccharomyces | Cg/Sc | Q4WUL6 | No Activity |
| | Name Found | | pombe ATCC 24843 | | | Name Found |
| P48570 | No Activity | 0 | Saccharomyces | Cg/Sc | Q4WUL6 | No Activity |
| | Name Found | | cerevisiae S288c | | | Name Found |
| Q75A20 | No Activity | 0 | Ashbya gossypii | Cg/Sc | Q4WUL6 | No Activity |
| | Name Found | | ATCC | | | Name Found |
| M7X1E3 | Homocitrate | 0 | Rhodosporidium | Cg/Sc | Q4WUL6 | No Activity |
| | synthase | | toruloides NP11 | | | Name Found |
| E4V1M0 | No Activity | 0 | Arthroderma | Cg/Sc | Q4WUL6 | No Activity |
| | Name Found | | gypseum ATCC | | | Name Found |
| | | | MYA-4604 | | | |
| F2PSY4 | No Activity | 0 | Trichophyton | Cg/Sc | Q4WUL6 | No Activity |
| | Name Found | | equinum ATCC | | | Name Found |
| | | | MYA-4606 | | | |
| F2S364 | No Activity | 0 | Trichophyton | Cg/Sc | Q4WUL6 | No Activity |
| | Name Found | | tonsurans CBS | | | Name Found |
| | | | 112818 | | | |
| P12683 | 3-hydroxy-3- | 0 | Saccharomyces | Cg/Sc | Q4WUL6 | No Activity |
| | methylglutaryl- | | cerevisiae S288c | | | Name Found |
| | coenzyme A | | | | | |
| | reductase 1 | | | | | |
| | (HMG-CoA | | | | | |
| | reductase 1) | | | | | |
| | (EC 1.1.1.34) | | | | | |
| A0A117DXK2 | No Activity | 0 | Aspergillus niger | Cg/Sc | Q4WUL6 | No Activity |
| | Name Found | | | | | Name Found |
| | Enzyme 2- | E2 Codon | | Enzyme 3- | Enzyme 3- | E3 Codon |
| E1 Uniprot ID | source organism | Optimization | E3 Uniprot ID | activity name | source organism | Optimization |
| Q9Y823 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| Q9Y823 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| Q9Y823 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| Q9Y823 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| Q9Y823 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| P48570 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| Q9Y823 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| P48570 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| P48570 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| P48570 | Neosartorya | Cg/Sc | P40495 | (1R,2S)-1- | Saccharomyces | Cg/Sc |
| | fumigata ATCC | | | hydroxybutane- | cerevisiae | |
| | MYA-4609 | | | 1,2,4-tricarboxylate: | S288c | |
| | | | | NAD + oxidoreductase | | |
| P48570 | Neosartorya | Cg/Sc | P40495 | (1R,2S)-1- | Saccharomyces | Cg/Sc |
| | fumigata ATCC | | | hydroxybutane- | cerevisiae | |
| | MYA-4609 | | | 1,2,4-tricarboxylate: | S288c | |
| | | | | NAD + oxidoreductase | | |
| P48570 | Neosartorya | Cg/Sc | P40495 | (1R,2S)-1- | Saccharomyces | Cg/Sc |
| | fumigata ATCC | | | hydroxybutane- | cerevisiae | |
| | MYA-4609 | | | 1,2,4-tricarboxylate: | S288c | |
| | | | | NAD + oxidoreductase | | |
| P48570 | Saccharomyces | Cg/Sc | P40495 | No Activity | Saccharomyces | Cg/Sc |
| | cerevisiae S288c | | | Name Found | cerevisiae S288c | |
| P48570 | Saccharomyces | Cg/Sc | P40495 | No Activity | Saccharomyces | Cg/Sc |
| | cerevisiae S288c | | | Name Found | cerevisiae S288c | |
| P48570 | Saccharomyces | Cg/Sc | P40495 | No Activity | Saccharomyces | Cg/Sc |
| | cerevisiae S288c | | | Name Found | cerevisiae S288c | |
| P48570 | Saccharomyces | Cg/Sc | P40495 | No Activity | Saccharomyces | Cg/Sc |
| | cerevisiae S288c | | | Name Found | cerevisiae S288c | |
| P48570 | Clostridium sp. | Cg/Sc | P40495 | (1R,2S)-1- | Saccharomyces | Cg/Sc |
| | C8 | | | hydroxybutane- | cerevisiae | |
| | | | | 1,2,4-tricarboxylate: | S288c | |
| | | | | NAD + oxidoreductase | | |
| P48570 | | | | | | |
| P48570 | | | | | | |
| Q57926 | | | | | | |
| D5Q163 | | | | | | |
| Q57926 | Saccharomyces | Cg/Sc | | | | |
| | cerevisiae S288c | | | | | |
| O27667 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| O87198 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| G8NBZ9 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| F2NL20 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| E4U9R8 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| A0A0F7TVK2 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| A0A0L1I0C1 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| C1CVX4 | Saccharomyces | Cg/Sc | P40495 | (1R,2S)-1- | Saccharomyces | Cg/Sc |
| | cerevisiae | | | hydroxybutane- | cerevisiae | |
| | S288c | | | 1,2,4-tricarboxylate: | S288c | |
| | | | | NAD + oxidoreductase | | |
| Q9RUZ2 | Saccharomyces | Cg/Sc | B3LTU1 | No Activity | Saccharomyces | Cg/Sc |
| | cerevisiae | | | Name Found | cerevisiae | |
| | S288c | | | | RM11-1a | |
| Q2IHS7 | Saccharomyces | Cg/Sc | B3LTU1 | No Activity | Saccharomyces | Cg/Sc |
| | cerevisiae | | | Name Found | cerevisiae | |
| | S288c | | | | RM11-la | |
| A0A1F8TP88 | Neosartorya | Cg/Sc | B3LTU1 | No Activity | Saccharomyces | Cg/Sc |
| | fumigata ATCC | | | Name Found | cerevisiae | |
| | MYA-4609 | | | | RM11-1a | |
| Q9Y823 | Neosartorya | Cg/Sc | B3L1U1 | No Activity | Saccharomyces | Cg/Sc |
| | fumigata ATCC | | | Name Found | cerevisiae | |
| | MYA-4609 | | | | RM11-1a | |
| P48570 | Neosartorya | Cg/Sc | B3LTU1 | No Activity | Saccharomyces | Cg/Sc |
| | fumigata ATCC | | | Name Found | cerevisiae | |
| | MYA-4609 | | | | RM11-1a | |
| Q75A20 | Neosartorya | Cg/Sc | B3LTU1 | No Activity | Saccharomyces | Cg/Sc |
| | fumigata ATCC | | | Name Found | cerevisiae | |
| | MYA-4609 | | | | RM11-1a | |
| M7X1E3 | Neosartorya | Cg/Sc | B3LTU1 | No Activity | Saccharomyces | Cg/Sc |
| | fumigata ATCC | | | Name Found | cerevisiae | |
| | MYA-4609 | | | | RM11-la | |
| E4V1M0 | Neosartorya | Cg/Sc | B3LTU1 | No Activity | Saccharomyces | Cg/Sc |
| | fumigata ATCC | | | Name Found | cerevisiae | |
| | MYA-4609 | | | | RM11-1a | |
| F2PSY4 | Neosartorya | Cg/Sc | J8Q3V7 | No Activity | Saccharomyces | Cg/Sc |
| | fumigata ATCC | | | Name Found | arboricola CBS | |
| | MYA-4609 | | | | 10644 | |
| F2S364 | Neosartorya | Cg/Sc | J8Q3V7 | No Activity | Saccharomyces | Cg/Sc |
| | fumigata ATCC | | | Name Found | arboricola CBS | |
| | MYA-4609 | | | | 10644 | |
| P12683 | Neosartorya | Cg/Sc | J8Q3V7 | No Activity | Saccharomyces | Cg/Sc |
| | fumigata ATCC | | | Name Found | arboricola CBS | |
| | MYA-4609 | | | | 10644 | |
| A0A117DXK2 | Neosartorya | Cg/Sc | J8Q3V7 | No Activity | Saccharomyces | Cg/Sc |
| | fumigata ATCC | | | Name Found | arboricola CBS | |
| | MYA-4609 | | | | 10644 | |

Note:
Cg/SC = codon-optimized according to modified codon usage for Cg and Sc

TABLE 4
Genetic engineering results in Yarrowia lipolytica

| | Titer | E1 | Enzyme 1- | E1 | Enzyme 1- |
|---|---|---|---|---|---|
| Strn | (μ/L) | Uniprot ID | activity name | Modifications | source organism |
| Yl2OXAD_01 | 13.4 | P48570 | Homocitrate | | Saccharomyces |
| | | | synthase, | | cerevisiae (strain |
| | | | cytosolic isozyme | | ATCC 204508/S288c) |
| | | | | | (Baker's yeast) |
| Yl2OXAD_02 | 15.4 | P48570 | Homocitrate | | Saccharomyces |
| | | | synthase, | | cerevisiae (strain |
| | | | cytosolic isozyme | | ATCC 204508/S288c) |
| | | | | | (Baker's yeast) |
| Yl2OXAD_03 | 14.9 | P48570 | Homocitrate | | Saccharomyces |
| | | | synthase, | | cerevisiae (strain |
| | | | cytosolic isozyme | | ATCC 204508/S288c) |
| | | | | | (Baker's yeast) |
| Yl2OXAD_04 | 40.1 | P48570 | Homocitrate | | Saccharomyces |
| | | | synthase, | | cerevisiae (strain |
| | | | cytosolic isozyme | | ATCC 204508/S288c) |
| | | | | | (Baker's yeast) |
| Yl2OXAD_05 | 14.2 | Q9Y823 | Homocitrate | D123N | Schizosaccharomyces |
| | | | synthase, | | pombe |
| | | | mitochondrial | | (strain 972/ATCC 24843) |
| | | | | | (Fission yeast) |
| Yl2OXAD_06 | 14.5 | Q9Y823 | Homocitrate | D123N | Schizosaccharomyces |
| | | | synthase, | | pombe |
| | | | mitochondrial | | (strain 972/ATCC 24843) |
| | | | | | (Fission yeast) |
| Yl2OXAD_07 | 237.8 | Q9Y823 | Homocitrate | D123N | Schizosaccharomyces |
| | | | synthase, | | pombe |
| | | | mitochondrial | | (strain 972/ATCC 24843) |
| | | | | | (Fission yeast) |
| Yl2OXAD_08 | 13.6 | A0A0G9LF37 | Transhomoaconitate | | Clostridium sp. C8 |
| | | | synthase | | |
| Yl2OXAD_09 | 14.4 | A0A0G9LF37 | Transhomoaconitate | | Clostridium sp. C8 |
| | | | synthase | | |
| Yl2OXAD_10 | 15.9 | A0A0G9LF37 | Transhomoaconitate | | Clostridium sp. C8 |
| | | | synthase | | |
| Yl2OXAD_11 | 13.5 | O87198 | Homocitrate | | Thermus thermophilus |
| | | | synthase | | (strain HB27/ATCC BAA-163/ |
| | | | | | DSM 7039) |
| Yl2OXAD_12 | 14.6 | O87198 | Homocitrate | | Thermus thermophilus |
| | | | synthase | | (strain HB27/ATCC BAA-163/ |
| | | | | | DSM 7039) |
| Yl2OXAD_13 | 57.8 | O87198 | Homocitrate | | Thermus thermophilus |
| | | | synthase | | (strain HB27/ATCC BAA-163/ |
| | | | | | DSM 7039) |
| Yl2OXAD_14 | 13.5 | P48570 | Homocitrate | | Saccharomyces |
| | | | synthase | | cerevisiae (strain |
| | | | cytosolic isozyme | | ATCC 204508/S288c) |
| | | | | | (Baker's yeast) |
| Yl2OXAD15_ | 14.7 | P48570 | Homocitrate | | Saccharomyces |
| | | | synthase | | cerevisiae (strain |
| | | | cytosolic isozyme | | ATCC 204508/S288c) |
| | | | | | (Baker's yeast) |
| Yl2OXAD_16 | 46.4 | P48570 | Homocitrate | | Saccharomyces |
| | | | synthase | | cerevisiae (strain |
| | | | cytosolic isozyme | | ATCC 204508/S288c) |
| | | | | | (Baker's yeast) |
| Yl2OXAD_17 | | P48570 | Homocitrate | | Saccharomyces |
| | | | synthase | | cerevisiae (strain |
| | | | cytosolic isozyme | | ATCC 204508/S288c) |
| | | | | | (Baker's yeast) |
| Yl2OXAD_18 | 14.0 | P48570 | Homocitrate | | Saccharomyces |
| | | | synthase | | cerevisiae (strain |
| | | | cytosolic isozyme | | ATCC 204508/S288c) |
| | | | | | (Baker's yeast) |
| Yl2OXAD_19 | 29.3 | P48570 | Homocitrate | | Saccharomyces |
| | | | synthase | | cerevisiae (strain |
| | | | cytosolic isozyme | | ATCC 204508/S288c) |
| | | | | | (Baker's yeast) |
| | E1 Codon | E2 | Enzyme 2- | E2 | Enzyme 2- |
| Strn | Optimization | Uniprot ID | activity name | Modifications | source organism |
| Yl2OXAD_01 | Bacillus subtilis | P49367 | Homoaconitase, | | Saccharomyces |
| | | | mitochondrial | | cerevisiae (strain |
| | | | | | ATCC 204508/S288c) |
| | | | | | (Baker's yeast) |
| Yl2OXAD_02 | modified codon | P49367 | Homoaconitase, | | Saccharomyces |
| | usage for | | mitochondrial | | cerevisiae (strain |
| | Corynebacterium | | | | ATCC 204508/S288c) |
| | glutamicum and | | | | (Baker's yeast) |
| | Saccharomyces | | | | |
| | cerevisiae | | | | |
| Yl2OXAD_03 | Saccharomyces | P49367 | Homoaconitase, | | Saccharomyces |
| | cerevisiae | | mitochondrial | | cerevisiae (strain |
| | | | | | ATCC 204508/S288c) |
| | | | | | (Baker's yeast) |
| Yl2OXAD_04 | Yarrowia | P49367 | Homoaconitase, | | Saccharomyces |
| | lipolytica | | mitochondrial | | cerevisiae (strain |
| | | | | | ATCC 204508/S288c) |
| | | | | | (Baker's yeast) |
| Yl2OXAD_05_ | Bacillus | P49367 | Homoaconitase, | | Saccharomyces |
| | subtilis | | mitochondrial | | cerevisiae (strain |
| | | | | | ATCC 204508/S288c) |
| | | | | | (Baker's yeast) |
| Yl2OXAD_06 | Saccharomyces | P49367 | Homoaconitase, | | Saccharomyces |
| | cerevisiae | | mitochondrial | | cerevisiae (strain |
| | | | | | ATCC 204508/S288c) |
| | | | | | (Baker's yeast) |
| Yl2OXAD_07 | Yarrowia | P49367 | Homoaconitase, | | Saccharomyces |
| | lipolytica | | mitochondrial | | cerevisiae (strain |
| | | | | | ATCC 204508/S288c) |
| | | | | | (Baker's yeast) |
| Yl2OXAD_08 | Bacillus | P49367 | Homoaconitase, | | Saccharomyces |
| | subtilis | | mitochondrial | | cerevisiae (strain |
| | | | | | ATCC 204508/S288c) |
| | | | | | (Baker's yeast) |
| Yl2OXAD_09 | Saccharomyces | P49367 | Homoaconitase, | | Saccharomyces |
| | cerevisiae | | mitochondrial | | cerevisiae (strain |
| | | | | | ATCC 204508/S288c) |
| | | | | | (Baker's yeast) |
| Yl2OXAD_10 | Yarrowia | P49367 | Homoaconitase, | | Saccharomyces |
| | lipolytica | | mitochondrial | | cerevisiae (strain |
| | | | | | ATCC 204508/S288c) |
| | | | | | (Baker's yeast) |
| Yl2OXAD_11 | Bacillus | P49367 | Homoaconitase, | | Saccharomyces |
| | subtilis | | mitochondrial | | cerevisiae (strain |
| | | | | | ATCC 204508/S288c) |
| | | | | | (Baker's yeast) |
| Yl2OXAD_12 | Saccharomyces | P49367 | Homoaconitase, | | Saccharomyces |
| | cerevisiae | | mitochondrial | | cerevisiae (strain |
| | | | | | ATCC 204508/S288c) |
| | | | | | (Baker's yeast) |
| Yl2OXAD_13 | Yarrowia | P49367 | Homoaconitase, | | Saccharomyces |
| | lipolytica | | mitochondrial | | cerevisiae (strain |
| | | | | | ATCC 204508/S288c) |
| | | | | | (Baker's yeast) |
| Yl2OXAD_14 | Bacillus | Q4WUL6 | Homoaconitase, | | Neosartorya fumigata |
| | subtilis | | mitochondrial | | (strain ATCC MYA- |
| | | | | | 4609/Af293/CBS |
| | | | | | 101355/FGSCA1100) |
| | | | | | (Aspergillus fumigatus) |
| Yl2OXAD15_ | Saccharomyces | Q4WUL6 | Homoaconitase, | | Neosartorya fumigata |
| | cerevisiae | | mitochondrial | | (strain ATCC MYA- |
| | | | | | 4609/Af293/CBS |
| | | | | | 101355/FGSCA1100) |
| | | | | | (Aspergillus fumigatus) |
| Yl2OXAD_16 | Yarrowia | Q4WUL6 | Homoaconitase, | | Neosartorya fumigata |
| | lipolytica | | mitochondrial | | (strain ATCC MYA- |
| | | | | | 4609/Af293/CBS |
| | | | | | 101355/FGSCA1100) |
| | | | | | (Aspergillus fumigatus) |
| Yl2OXAD_17 | Bacillus | Q4WUL6 | Homoaconitase, | | Neosartorya fumigata |
| | subtilis | | mitochondrial | | (strain ATCC MYA- |
| | | | | | 4609/Af293/CBS |
| | | | | | 101355/FGSCA1100) |
| | | | | | (Aspergillus fumigatus) |
| Yl2OXAD_18 | Saccharomyces | Q4WUL6 | Homoaconitase, | del 2-41 and | Neosartorya fumigata |
| | cerevisiae | | mitochondrial | del 721-777 | (strain ATCC MYA- |
| | | | | | 4609/Af293/CBS |
| | | | | | 101355/FGSCA1100) |
| | | | | | (Aspergillus fumigatus) |
| Yl2OXAD_19 | Yarrowia | Q4WUL6 | Homoaconitase, | del 2-41 and | Neosartorya fumigata |
| | lipolytica | | mitochondrial | del 721-777 | (strain ATCC MYA- |
| | | | | | 4609/Af293/CBS |
| | | | | | 101355/FGSCA1100) |
| | | | | | (Aspergillus fumigatus) |
| | E2 Codon | E3 | Enzyme 3- | Enzyme 3- | E3 Codon |
| Strn | Optimization | Uniprot ID | activity name | source organism | Optimization |
| Yl2OXAD_01 | Bacillus | P40495 | Homoisocitrate | Saccharomyces | Bacillus |
| | subtilis | | dehydrogenase, | cerevisiae (strain | subtilis |
| | | | mitochondrial | ATCC 204508/S288c) | |
| | | | | (Baker's yeast) | |
| Yl2OXAD_02 | modified codon | P40495 | Homoisocitrate | Saccharomyces | modified codon |
| | usage for | | dehydrogenase, | cerevisiae (strain | usage for Corynebacterium |
| | Corynebacterium | | mitochondrial | ATCC 204508/S288c) | glutamicum and |
| | glutamicum and | | | (Baker's yeast) | Saccharomyces |
| | Saccharomyces | | | | cerevisiae |
| | cerevisiae | | | | |
| Yl2OXAD_03 | Saccharomyces | P40495 | Homoisocitrate | Saccharomyces | Saccharomyces |
| | cerevisiae | | dehydrogenase, | cerevisiae (strain | cerevisiae |
| | | | mitochondrial | ATCC 204508/S288c) | cerevisiae |
| | | | | (Baker's yeast) | |
| Yl2OXAD_04 | Yarrowia | P40495 | Homoisocitrate | Saccharomyces | Yarrowia |
| | lipolytica | | dehydrogenase, | cerevisiae (strain | lipolytica |
| | | | mitochondrial | ATCC 204508/S288c) | |
| | | | | (Baker's yeast) | |
| Yl2OXAD_05_ | Bacillus | P40495 | Homoisocitrate | Saccharomyces | Bacillus subtilis |
| | subtilis | | dehydrogenase, | cerevisiae (strain | |
| | | | mitochondrial | ATCC 204508/S288c) | |
| | | | | (Baker's yeast) | |
| Yl2OXAD_06 | Saccharomyces | P40495 | Homoisocitrate | Saccharomyces | Saccharomyces |
| | cerevisiae | | dehydrogenase, | cerevisiae (strain | cerevisiae |
| | | | mitochondrial | ATCC 204508/S288c) | |
| | | | | (Baker's yeast) | |
| Yl2OXAD_07 | Yarrowia | P40495 | Homoisocitrate | Saccharomyces | Yarrowia lipolytica |
| | lipolytica | | dehydrogenase, | cerevisiae (strain | |
| | | | mitochondrial | ATCC 204508/S288c) | |
| | | | | (Baker's yeast) | |
| Yl2OXAD_08 | Bacillus | P40495 | Homoisocitrate | Saccharomyces | Bacillus subtilis |
| | subtilis | | dehydrogenase, | cerevisiae (strain | |
| | | | mitochondrial | ATCC 204508/S288c) | |
| | | | | (Baker's yeast) | |
| Yl2OXAD_09 | Saccharomyces | P40495 | Homoisocitrate | Saccharomyces | Saccharomyces |
| | cerevisiae | | dehydrogenase, | cerevisiae (strain | cerevisiae |
| | | | mitochondrial | ATCC 204508/S288c) | |
| | | | | (Baker's yeast) | |
| Yl2OXAD_10 | Yarrowia | P40495 | Homoisocitrate | Saccharomyces | Yarrowia lipolytica |
| | lipolytica | | dehydrogenase, | cerevisiae (strain | |
| | | | mitochondrial | ATCC 204508/S288c) | |
| | | | | (Baker's yeast) | |
| Yl2OXAD_11 | Bacillus | Q72IW9 | Homoisocitrate | Thermus thermophilus | Bacillus subtilis |
| | subtilis | | dehydrogenase, | (strain HB27/ATCC | |
| | | | mitochondrial | BAA-163/DSM 7039) | |
| Yl2OXAD_12 | Saccharomyces | Q72IW9 | Homoisocitrate | Thermus thermophilus | Saccharomyces |
| | cerevisiae | | dehydrogenase, | (strain HB27/ATCC | cerevisiae |
| | | | mitochondrial | BAA-163/DSM 7039) | |
| Yl2OXAD_13 | Yarrowia | Q72IW9 | Homoisocitrate | Thermus thermophilus | Yarrowia lipolytica |
| | lipolytica | | dehydrogenase, | (strain HB27/ATCC | |
| | | | | BAA-163/DSM 7039) | |
| Yl2OXAD_14 | Bacillus | P40495 | Homoisocitrate | Saccharomyces | Bacillus subtilis |
| | subtilis | | dehydrogenase, | cerevisiae (strain | |
| | | | mitochondrial | ATCC 204508/S288c) | |
| | | | | (Baker's yeast) | |
| Yl2OXAD15_ | Saccharomyces | P40495 | Homoisocitrate | Saccharomyces | Saccharomyces |
| | cerevisiae | | dehydrogenase, | cerevisiae (strain | cerevisiae |
| | | | mitochondrial | ATCC 204508/S288c) | |
| | | | | (Baker's yeast) | |
| Yl2OXAD_16 | Yarrowia | P40495 | Homoisocitrate | Saccharomyces | Yarrowia lipolytica |
| | lipolytica | | dehydrogenase, | cerevisiae (strain | |
| | | | mitochondrial | ATCC 204508/S288c) | |
| | | | | (Baker's yeast) | |
| Yl2OXAD_17 | Bacillus | P40495 | Homoisocitrate | Saccharomyces | Bacillus subtilis |
| | subtilis | | dehydrogenase, | cerevisiae (strain | |
| | | | mitochondrial | ATCC 204508/S288c) | |
| | | | | (Baker's yeast) | |
| Yl2OXAD_18 | Saccharomyces | P40495 | Homoisocitrate | Saccharomyces | Saccharomyces |
| | cerevisiae | | dehydrogenase, | cerevisiae (strain | cerevisiae |
| | | | mitochondrial | ATCC 204508/S288c) | |
| | | | | (Baker's yeast) | |
| Yl2OXAD_19 | Yarrowia | P40495 | Homoisocitrate | Saccharomyces | Yarrowia lipolytica |
| | lipolytica | | dehydrogenase, | cerevisiae (strain | |
| | | | mitochondrial | ATCC 204508/S288c) | |
| | | | | (Baker's yeast) | |

TABLE 5
Genetic engineering results in Bacillus subtilis

| | | | | E1 | | | | | | | | | | | |
|---|---|---|---|---|---|---|---|---|---|---|---|---|---|---|---|
| | | E1 | Enzyme 1- | Modi- | Enzyme 1- | | E2 | Enzyme 2- | E2 | Enzyme 2- | | E3 | Enzyme 3- | Enzyme 3- | |
| | Titer | Uniprot | activity | fica- | source | E1 Codon | Uniprot | activity | Modi- | source | E2 Codon | Uniprot | activity | source | E3 Codon |
| Strn | (μ/L) | ID | name | tions | organism | Optimization | ID | name | fications | organism | Optimization | ID | name | organism | Optimization |
| Bs2OXAD_ | | A0A0G9LF37 | Trans- | 0 | Clostridium | Yl | P49367 | Homo- | 0 | Saccharo- | Yl | P40495 | Homo- | Saccharo- | Yarrowia |
| 01 | | | Homo- | | sp. | | | aconitase, | | myces | | | isocitrate | myces | lipolytica |
| | | | aconitate | | C8 | | | mitochon- | | cerevisiae | | | dehydro- | cerevisiae | |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Bs2OXAD_ | | P48570 | Homo- | 0 | Saccharo- | Bs | P49367 | Homo- | 0 | Saccharo- | Bs | P40495 | Homo- | Saccharo- | Bacillus |
| 02 | | | citrate | | myces | | | aconitase, | | myces | | | isocitrate | myces | subtilis |
| | | | synthase, | | cerevisiae | | | mitochon- | | cerevisiae | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Bs2OXAD_ | | P48570 | Homo- | 0 | Saccharo- | modified | P49367 | Homo- | 0 | Saccharo- | modified | P40495 | Homo- | Saccharo- | modified |
| 03 | | | citrate | | myces | codon | | aconitase, | | myces | codon | | isocitrate | myces | codon |
| | | | synthase, | | cerevisiae | usage for | | mitochon- | | cerevisiae | usage for | | dehydro- | cerevisiae | usage for |
| | | | cytosolic | | (strain | Coryne- | | drial | | (strain | Coryne- | | genase, | (strain | Coryne- |
| | | | isozyme | | ATCC | bacterium | | | | ATCC | bacterium | | mitochon- | ATCC | bacterium |
| | | | | | 204508/ | glutami- | | | | 204508/ | glutami- | | drial | 204508/ | glutami- |
| | | | | | S288c) | cum and | | | | S288c) | cum and | | | S288c) | cum and |
| | | | | | (Baker's | Saccharo- | | | | (Baker's | Saccharo- | | | (Baker's | Saccharo- |
| | | | | | yeast) | myces | | | | yeast) | myces | | | yeast) | myces |
| | | | | | | cerevisiae | | | | | cerevisiae | | | | cerevisiae |
| Bs2OXAD_ | | P48570 | Homo- | 0 | Saccharo- | Sc | P49367 | Homo- | 0 | Saccharo- | Sc | P40495 | Homo- | Saccharo- | Saccharo- |
| 04 | | | citrate | | myces | | | aconitase, | | myces | | | isocitrate | myces | myces |
| | | | synthase, | | cerevisiae | | | mitochon- | | cerevisiae | | | dehydro- | cerevisiae | cerevisiae |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Bs2OXAD_ | | P48570 | Homo- | 0 | Saccharo- | Yl | P49367 | Homo- | 0 | Saccharo- | Yl | P40495 | Homo- | Saccharo- | Yarrowia |
| 05 | | | citrate | | myces | | | aconitase, | | myces | | | isocitrate | myces | lipolytica |
| | | | synthase, | | cerevisiae | | | mitochon- | | cerevisiae | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Bs2OXAD_ | | Q9Y823 | Homo- | D123N | Schizo- | Bs | P49367 | Homo- | 0 | Saccharo- | Bs | P40495 | Homo- | Saccharo- | Bacillus |
| 06 | | | citrate | | Saccharo- | | | aconitase, | | myces | | | isocitrate | myces | subtilis |
| | | | synthase, | | myces | | | mitochon- | | cerevisiae | | | dehydro- | cerevisiae | |
| | | | mitochon- | | pombe | | | drial | | (strain | | | genase, | (strain | |
| | | | drial | | (strain | | | | | ATCC | | | mitochon- | ATCC | |
| | | | | | 972/ | | | | | 204508/ | | | drial | 204508/ | |
| | | | | | ATCC | | | | | S288c) | | | | S288c) | |
| | | | | | 24843) | | | | | (Baker's | | | | (Baker's | |
| | | | | | (Fission | | | | | yeast) | | | | yeast) | |
| | | | | | yeast) | | | | | | | | | | |
| Bs2OXAD_ | | Q9Y823 | Homo- | D123N | Schizo- | modified | P49367 | Homo- | | Saccharo- | modified | P40495 | Homo- | Saccharo- | modified |
| 07 | | | citrate | | Saccharo- | codon | | aconitase, | | myces | codon | | isocitrate | myces | codon |
| | | | synthase, | | myces | usage for | | mitochon- | | cerevisiae | usage for | | dehydro- | cerevisiae | usage for |
| | | | mitochon- | | pombe | Coryne- | | drial | 0 | (strain | Coryne- | | genase, | (strain | Coryne- |
| | | | drial | | (strain | bacterium | | | | ATCC | bacterium | | mitochon- | ATCC | bacterium |
| | | | | | 972/ | glutami- | | | | 204508/ | glutami- | | drial | 204508/ | glutami- |
| | | | | | ATCC | cum and | | | | S288c) | cum and | | | S288c) | cum and |
| | | | | | 24843) | Saccharo- | | | | (Baker's | Saccharo- | | | (Baker's | Saccharo- |
| | | | | | (Fission | myces | | | | yeast) | myces | | | yeast) | myces |
| | | | | | yeast) | cerevisiae | | | | | cerevisiae | | | | cerevisiae |
| Bs2OXAD_ | 2.3183 | Q9Y823 | Homo- | D123N | Schizo- | Sc | P49367 | Homo- | 0 | Saccharo- | Sc | P40495 | Homo- | Saccharo- | Saccharo- |
| 08 | | | citrate | | Saccharo- | | | aconitase, | | myces | | | isocitrate | myces | myces |
| | | | synthase, | | myces | | | mitochon- | | cerevisiae | | | dehydro- | cerevisiae | cerevisiae |
| | | | mitochon- | | pombe | | | drial | | (strain | | | genase, | (strain | |
| | | | drial | | (strain | | | | | ATCC | | | mitochon- | ATCC | |
| | | | | | 972/ | | | | | 204508/ | | | drial | 204508/ | |
| | | | | | ATCC | | | | | S288c) | | | | S288c) | |
| | | | | | 24843) | | | | | (Baker's | | | | (Baker's | |
| | | | | | (Fission | | | | | yeast) | | | | yeast) | |
| | | | | | yeast) | | | | | | | | | | |
| Bs2OXAD_ | | Q9Y823 | Homo- | D123N | Schizo- | Yl | P49367 | Homo- | 0 | Saccharo- | Yl | P40495 | Homo- | Saccharo- | Yarrowia |
| 09 | | | citrate | | Saccharo- | | | aconitase, | | myces | | | isocitrate | myces | lipolytica |
| | | | synthase, | | myces | | | mitochon- | | cerevisiae | | | dehydro- | cerevisiae | |
| | | | mitochon- | | pombe | | | drial | | (strain | | | genase, | (strain | |
| | | | drial | | (strain | | | | | ATCC | | | mitochon- | ATCC | |
| | | | | | 972/ | | | | | 204508/ | | | drial | 204508/ | |
| | | | | | ATCC | | | | | S288c) | | | | S288c) | |
| | | | | | 24843) | | | | | (Baker's | | | | (Baker's | |
| | | | | | (Fission yeast) | | | | | yeast) | | | | yeast) | |
| Bs2OXAD_ | | A0A0G9LF37 | Trans- | 0 | Clostri- | Bs | P49367 | Homo- | 0 | Saccharo- | Bs | P40495 | Homo- | Saccharo- | Bacillus |
| 10 | | | Homo- | | dium sp. | | | aconitase, | | myces | | | isocitrate | myces | subtilis |
| | | | aconitate | | C8 | | | mitochon- | | cerevisiae | | | dehydro- | cerevisiae | |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Bs2OXAD_ | | A0A0G9LF37 | Trans- | 0 | Clostri- | modified | P49367 | Homo- | 0 | Saccharo- | modified | P40495 | Homo- | Saccharo- | modified |
| 11 | | | Homo- | | dium sp. | codon | | aconitase, | | myces | codon | | isocitrate | myces | codon |
| | | | aconitate | | C8 | usage for | | mitochon- | | cerevisiae | usage for | | dehydro- | cerevisiae | usage for |
| | | | synthase | | | Coryne- | | drial | | (strain | Coryne- | | genase, | (strain | Coryne- |
| | | | | | | bacterium | | | | ATCC | bacterium | | mitochon- | ATCC | bacterium |
| | | | | | | glutami- | | | | 204508/ | glutami- | | drial | 204508/ | glutami- |
| | | | | | | cum and | | | | S288c) | cum and | | | S288c) | cum and |
| | | | | | | Saccharo- | | | | (Baker's | Saccharo- | | | (Baker's | Saccharo- |
| | | | | | | myces | | | | yeast) | myces | | | yeast) | myces |
| | | | | | | cerevisiae | | | | | cerevisiae | | | | cerevisiae |
| Bs2OXAD_ | | A0A0G9LF37 | Trans- | 0 | Clostri- | Sc | P49367 | Homo- | 0 | Saccharo- | Sc | P40495 | Homo- | Saccharo- | Saccharo- |
| 12 | | | Homo- | | dium sp. | | | aconitase, | | myces | | | isocitrate | myces | myces |
| | | | aconitate | | C8 | | | mitochon- | | cerevisiae | | | dehydro- | cerevisiae | cerevisiae |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Bs2OXAD_ | | O87198 | Homo- | 0 | Thermus | Bs | P49367 | Homo- | 0 | Saccharo- | Bs | Q72IW9 | Homo- | Thermus | Bacillus |
| 13 | | | citrate | | thermo- | | | aconitase, | | myces | | | isocitrate | thermo- | subtilis |
| | | | synthase | | philus | | | mitochon- | | cerevisiae | | | dehydro- | philus | |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-163/ | | | | | S288c) | | | | BAA-163/ | |
| | | | | | DSM | | | | | (Baker's | | | | DSM | |
| | | | | | 7039) | | | | | yeast) | | | | 7039) | |
| Bs2OXAD_ | | O87198 | Homo- | 0 | Thermus | modified | P49367 | Homo- | 0 | Saccharo- | modified | Q72IW9 | Homo- | Thermus | modified |
| 14 | | | citrate | | thermo- | codon | | aconitase, | | myces | codon | | isocitrate | thermo- | codon |
| | | | synthase | | philus | usage for | | mitochon- | | cerevisiae | usage for | | dehydro- | philus | usage for |
| | | | | | (strain | Coryne- | | drial | | (strain | Coryne- | | genase | (strain | Coryne- |
| | | | | | HB27/ | bacterium | | | | ATCC | bacterium | | | HB27/ | bacterium |
| | | | | | ATCC | glutami- | | | | 204508/ | glutami- | | | ATCC | glutami- |
| | | | | | BAA-163/ | cum and | | | | S288c) | cum and | | | BAA-163/ | cum and |
| | | | | | DSM | Saccharo- | | | | (Baker's | Saccharo- | | | DSM | Saccharo- |
| | | | | | 7039) | myces | | | | yeast) | myces | | | 7039) | myces |
| | | | | | | cerevisiae | | | | | cerevisiae | | | | cerevisiae |
| Bs2OXAD_ | | O87198 | Homo- | 0 | Thermus | Sc | P49367 | Homo- | 0 | Saccharo- | Sc | Q72IW9 | Homo- | Thermus | Saccharo- |
| 15 | | | citrate | | thermo- | | | aconitase, | | myces | | | isocitrate | thermo- | myces |
| | | | synthase | | philus | | | mitochon- | | cerevisiae | | | dehydro- | philus | cerevisiae |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-163/ | | | | | S288c) | | | | BAA-163/ | |
| | | | | | DSM | | | | | (Baker's | | | | DSM | |
| | | | | | 7039) | | | | | yeast) | | | | 7039) | |
| Bs2OXAD_ | | O87198 | Homo- | 0 | Thermus | Yl | P49367 | Homo- | 0 | Saccharo- | Yl | Q72IW9 | Homo- | Thermus | Yarrowia |
| 16 | | | citrate | | thermo- | | | aconitase, | | myces | | | isocitrate | thermo- | lipolytica |
| | | | synthase | | philus | | | mitochon- | | cerevisiae | | | dehydro- | philus | |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-163/ | | | | | S288c) | | | | BAA-163/ | |
| | | | | | DSM | | | | | (Baker's | | | | DSM | |
| | | | | | 7039) | | | | | yeast) | | | | 7039) | |
| Bs2OXAD_ | | P48570 | Homo- | 0 | Saccharo- | Bs | Q4WUL6 | Homo- | 0 | Neo- | Bs | P40495 | Homo- | Saccharo- | Bacillus |
| 17 | | | citrate | | myces | | | aconitase, | | sartorya | | | isocitrate | myces | subtilis |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | | P48570 | Homo- | 0 | Saccharo- | modified | Q4WUL6 | Homo- | 0 | Neo- | modified | P40495 | Homo- | Saccharo- | modified |
| 18 | | | citrate | | myces | codon | | aconitase, | | sartorya | codon | | isocitrate | myces | codon |
| | | | synthase, | | cerevisiae | usage for | | mitochon- | | fumigata | usage for | | dehydro- | cerevisiae | usage for |
| | | | cytosolic | | (strain | Coryne- | | drial | | (strain | Coryne- | | genase, | (strain | Coryne- |
| | | | isozyme | | ATCC | bacterium | | | | ATCC | bacterium | | mitochon- | ATCC | bacterium |
| | | | | | 204508/ | glutami- | | | | MYA- | glutami- | | drial | 204508/ | glutami- |
| | | | | | S288c) | cum and | | | | 4609/ | cum and | | | S288c) | cum and |
| | | | | | (Baker's | Saccharo- | | | | Af293/ | Saccharo- | | | (Baker's | Saccharo- |
| | | | | | yeast) | myces | | | | CBS | myces | | | yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | | P48570 | Homo- | 0 | Saccharo- | Sc | Q4WUL6 | Homo- | 0 | Neo- | Sc | P40495 | Homo- | Saccharo- | Saccharo- |
| 19 | | | citrate | | myces | | | aconitase, | | sartorya | | | isocitrate | myces | myces |
| | | | synthase, | | cerevisiae | | | mitochon- | | fumigata | | | dehydro- | cerevisiae | cerevisiae |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | | P48570 | Homo- | 0 | Saccharo- | Yl | Q4WUL6 | Homo- | 0 | Neo- | Yl | P40495 | Homo- | Saccharo- | Yarrowia |
| 20 | | | citrate | | myces | | | aconitase, | | sartorya | | | isocitrate | myces | lipolytica |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | 7.03778 | P48570 | Homo- | 0 | Saccharo- | Bs | Q4WUL6 | Homo- | 0 | Neo- | Bs | P40495 | Homo- | Saccharo- | Bacillus |
| 21 | | | citrate | | myces | | | aconitase, | | sartorya | | | isocitrate | myces | subtilis |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | 2.67668 | P48570 | Homo- | 0 | Saccharo- | modified | Q4WUL6 | Homo- | del 2- | Neo- | modified | P40495 | Homo- | Saccharo- | modified |
| 22 | | | citrate | | myces | codon | | aconitase, | 41 | sartorya | codon | | isocitrate | myces | codon |
| | | | synthase, | | cerevisiae | usage for | | mitochon- | and | fumigata | usage for | | dehydro- | cerevisiae | usage for |
| | | | cytosolic | | (strain | Coryne- | | drial | del | (strain | Coryne- | | genase, | (strain | Coryne- |
| | | | isozyme | | ATCC | bacterium | | | 721- | ATCC | bacterium | | mitochon- | ATCC | bacterium |
| | | | | | 204508/ | glutami- | | | 777 | MYA- | glutami- | | drial | 204508/ | glutami- |
| | | | | | S288c) | cum and | | | | 4609/ | cum and | | | S288c) | cum and |
| | | | | | (Baker's | Saccharo- | | | | Af293/ | Saccharo- | | | (Baker's | Saccharo- |
| | | | | | yeast) | myces | | | | CBS | myces | | | yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | 2.27663 | P48570 | Homo- | 0 | Saccharo- | Sc | Q4WUL6 | Homo- | del 2- | Neo- | Sc | P40495 | Homo- | Saccharo- | Saccharo- |
| 23 | | | citrate | | myces | | | aconitase, | 41 | sartorya | | | isocitrate | myces | myces |
| | | | synthase, | | cerevisiae | | | mitochon- | and | fumigata | | | dehydro- | cerevisiae | cerevisiae |
| | | | cytosolic | | (strain | | | drial | del | (strain | | | genase, | (strain | |
| | | | isozyme | | ATCC | | | | 721- | ATCC | | | mitochon- | ATCC | |
| | | | | | 204508/ | | | | 777 | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | | P48570 | Homo- | 0 | Saccharo- | Yl | Q4WUL6 | Homo- | del 2- | Neo- | Yl | P40495 | Homo- | Saccharo- | Yarrowia |
| 24 | | | citrate | | myces | | | aconitase, | 41 | sartorya | | | isocitrate | myces | lipolytica |
| | | | synthase, | | cerevisiae | | | mitochon- | and | fumigata | | | dehydro- | cerevisiae | |
| | | | cytosolic | | (strain | | | drial | del | (strain | | | genase, | (strain | |
| | | | isozyme | | ATCC | | | | 721- | ATCC | | | mitochon- | ATCC | |
| | | | | | 204508/ | | | | 777 | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Bs2OXAD_ | | O87198 | Homo- | 0 | Thermus | Control | W1QJE4 | Homo- | 0 | Ogataea | Control | W1QLF1 | Homo- | Ogataea | Control |
| 25 | | | citrate | | thermo- | | | aconitase, | | para-poly- | | | isocitrate | para-poly- | |
| | | | synthase | | philus | | | mitochon- | | morpha | | | dehydro- | morpha | |
| | | | | | (strain | | | drial | | (strain | | | genase, | (strain | |
| | | | | | HB27/ | | | | | ATCC | | | mitochon- | ATCC | |
| | | | | | ATCC | | | | | 26012/ | | | drial | 26012/ | |
| | | | | | BAA-163/ | | | | | BCRC | | | | BCRC | |
| | | | | | DSM | | | | | 20466/ | | | | 20466/ | |
| | | | | | 7039) | | | | | JCM | | | | JCM | |
| | | | | | | | | | | 22074/ | | | | 22074/ | |
| | | | | | | | | | | NRRL Y- | | | | NRRL Y- | |
| | | | | | | | | | | 7560 / DL- | | | | 7560/DL- | |
| | | | | | | | | | | 1) (Yeast) | | | | 1) (Yeast) | |
| | | | | | | | | | | (Hanse- | | | | (Hanse- | |
| | | | | | | | | | | nula poly- | | | | nula poly- | |
| | | | | | | | | | | morpha) | | | | morpha) | |

Yl = Yarrowia lipolytica;
Bs = Bacillus subtilis;
Sc = Saccharomyces cerevisiae

TABLE 6
Additional genetic engineering results in Saccharomyces cerevisiae

| | | E1 | Enzyme 1- | | Enzyme 1- | | E2 | Enzyme 2- | E2 | Enzyme 2- | | E3 | Enzyme 3- | Enzyme 3- | |
|---|---|---|---|---|---|---|---|---|---|---|---|---|---|---|---|
| | Titer | Uniprot | activity | E1 | source | E1 Codon | Uniprot | activity | Modi- | source | E2 Codon | Uniprot | activity | source | |
| Strn | (μ/L) | ID | name | Modifications | organism | Optimization | ID | name | fications | organism | Optimization | ID | name | organism | E3 Codon Optimization |
| Sc2OXAD_ | 238.3 | P48570 | Homo- | | Saccharo- | Bs | P49367 | Homo- | | Saccharo- | Bs | P40495 | Homo- | Saccharo- | Bs |
| 76 | | | citrate | | myces | | | aconitase, | | myces | | | isocitrate | myces | |
| | | | synthase, | | cerevisiae | | | mitochon- | | cerevisiae | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Sc2OXAD_ | 302.5 | P48570 | Homo- | | Saccharo- | Sc | P49367 | Homo- | | Saccharo- | Sc | P40495 | Homo- | Saccharo- | Sc |
| 77 | | | citrate | | myces | | | aconitase, | | myces | | | isocitrate | myces | |
| | | | synthase, | | cerevisiae | | | mitochon- | | cerevisiae | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Sc2OXAD_ | 257.2 | P48570 | Homo- | | Saccharo- | Yl | P49367 | Homo- | | Saccharo- | Yl | P40495 | Homo- | Saccharo- | Yl |
| 78 | | | citrate | | myces | | | aconitase, | | myces | | | isocitrate | myces | |
| | | | synthase, | | cerevisiae | | | mitochon- | | cerevisiae | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Sc2OXAD_ | 80012.8 | Q9Y823 | Homo- | D123N | Schizo- | Yl | P49367 | Homo- | | Saccharo- | Yl | P40495 | Homo- | Saccharo- | Yl |
| 79 | | | citrate | | Saccharo- | | | aconitase, | | myces | | | isocitrate | myces | |
| | | | synthase, | | myces | | | mitochon- | | cerevisiae | | | dehydro- | cerevisiae | |
| | | | mitochon- | | pombe | | | drial | | (strain | | | genase, | (strain | |
| | | | drial | | (strain | | | | | ATCC | | | mitochon- | ATCC | |
| | | | | | 972/ | | | | | 204508/ | | | drial | 204508/ | |
| | | | | | ATCC | | | | | S288c) | | | | S288c) | |
| | | | | | 24843) | | | | | (Baker's | | | | (Baker's | |
| | | | | | (Fission | | | | | yeast) | | | | yeast) | |
| | | | | | yeast) | | | | | | | | | | |
| Sc2OXAD_ | 118.6 | A0A0G9LF37 | Trans- | | Clostri- | Bs | P49367 | Homo- | | Saccharo- | Bs | P40495 | Homo- | Saccharo- | Bs |
| 80 | | | Homo- | | dium sp. | | | aconitase, | | myces | | | isocitrate | myces | |
| | | | aconitate | | C8 | | | mitochon- | | cerevisiae | | | dehydro- | cerevisiae | |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Sc2OXAD_ | 38.3 | A0A0G9LF37 | Trans- | | Clostri- | Sc | P49367 | Homo- | | Saccharo- | Sc | P40495 | Homo- | Saccharo- | Sc |
| 81 | | | Homo- | | dium sp. | | | aconitase, | | myces | | | isocitrate | myces | |
| | | | aconitate | | C8 | | | mitochon- | | cerevisiae | | | dehydro- | cerevisiae | |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Sc2OXAD_ | 148.7 | A0A0G9LF37 | Trans- | | Clostri- | Yl | P49367 | Homo- | | Saccharo- | Yl | P40495 | Homo- | Saccharo- | Yl |
| 82 | | | Homo- | | dium sp. | | | aconitase, | | myces | | | isocitrate | myces | |
| | | | aconitate | | C8 | | | mitochon- | | cerevisiae | | | dehydro- | cerevisiae | |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Sc2OXAD_ | 185.6 | O87198 | Homo- | | Thermus | Bs | P49367 | Homo- | | Saccharo- | Bs | Q72IW9 | Homo- | Thermus | Bs |
| 83 | | | citrate | | thermo- | | | aconitase, | | myces | | | isocitrate | thermo- | |
| | | | synthase | | philus | | | mitochon- | | cerevisiae | | | dehydro- | philus | |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-1631 | | | | | S288c) | | | | BAA-163/ | |
| | | | | | DSM | | | | | (Baker's | | | | DSM | |
| | | | | | 7039) | | | | | yeast) | | | | 7039) | |
| Sc2OXAD_ | 207.9 | O87198 | Homo- | | Thermus | Sc | P49367 | Homo- | | Saccharo- | Sc | Q72IW9 | Homo- | Thermus | Sc |
| 84 | | | citrate | | thermo- | | | aconitase, | | myces | | | isocitrate | thermo- | |
| | | | synthase | | philus | | | mitochon- | | cerevisiae | | | dehydro- | philus | |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-163/ | | | | | S288c) | | | | BAA-163/ | |
| | | | | | DSM | | | | | (Baker's | | | | DSM | |
| | | | | | 7039) | | | | | yeast) | | | | 7039) | |
| Sc2OXAD_ | 191.5 | O87198 | Homo- | | Thermus | Yl | P49367 | Homo- | | Saccharo- | Yl | Q72IW9 | Homo- | Thermus | Yl |
| 85 | | | citrate | | thermo- | | | aconitase, | | myces | | | isocitrate | thermo- | |
| | | | synthase | | philus | | | mitochon- | | cerevisiae | | | dehydro- | philus | |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-163/ | | | | | S288c) | | | | BAA-163/ | |
| | | | | | DSM | | | | | (Baker's | | | | DSM | |
| | | | | | 7039) | | | | | yeast) | | | | 7039) | |
| Sc2OXAD_ | 202.9 | P48570 | Homo- | | Saccharo- | Bs | Q4WUL6 | Homo- | | Neo- | Bs | P40495 | Homo- | Saccharo- | Bs |
| 86 | | | citrate | | myces | | | aconitase, | | sartorya | | | isocitrate | myces | |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Sc2OXAD_ | 212.1 | P48570 | Homo- | | Saccharo- | modified | Q4WUL6 | Homo- | | Neo- | modified | P40495 | Homo- | Saccharo- | modified |
| 87 | | | citrate | | myces | codon | | aconitase, | | sartorya | codon | | isocitrate | myces | codon |
| | | | synthase, | | cerevisiae | usage for | | mitochon- | | fumigata | usage for | | dehydro- | cerevisiae | usage for |
| | | | cytosolic | | (strain | Coryne- | | drial | | (strain | Coryne- | | genase, | (strain | Coryne- |
| | | | isozyme | | ATCC | bacterium | | | | ATCC | bacterium | | mitochon- | ATCC | bacterium |
| | | | | | 204508/ | glutami- | | | | MYA- | glutami- | | drial | 204508/ | glutami- |
| | | | | | S288c) | cum and | | | | 4609/ | cum and | | | S288c) | cum and |
| | | | | | (Baker's | Saccharo- | | | | Af293/ | Saccharo- | | | (Baker's | Saccharo- |
| | | | | | yeast) | myces | | | | CBS | myces | | | yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Sc2OXAD_ | 177.3 | P48570 | Homo- | | Saccharo- | Sc | Q4WUL6 | Homo- | | Neo- | Sc | P40495 | Homo- | Saccharo- | Sc |
| 88 | | | citrate | | myces | | | aconitase, | | sartorya | | | isocitrate | myces | |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Sc2OXAD_ | 170.1 | P48570 | Homo- | | Saccharo- | modified | Q4WUL6 | Homo- | del 2- | Neo- | modified | P40495 | Homo- | Saccharo- | modified |
| 89 | | | citrate | | myces | codon | | aconitase, | 41 | sartorya | codon | | isocitrate | myces | codon |
| | | | synthase, | | cerevisiae | usage for | | mitochon- | and | fumigata | usage for | | dehydro- | cerevisiae | usage for |
| | | | cytosolic | | (strain | Coryne- | | drial | del | (strain | Coryne- | | genase, | (strain | Coryne- |
| | | | isozyme | | ATCC | bacterium | | | 721- | ATCC | bacterium | | mitochon- | ATCC | bacterium |
| | | | | | 204508/ | glutami- | | | 777 | MYA- | glutami- | | drial | 204508/ | glutami- |
| | | | | | S288c) | cum and | | | | 4609/ | cum and | | | S288c) | cum and |
| | | | | | (Baker's | Saccharo- | | | | Af293/ | Saccharo- | | | (Baker's | Saccharo- |
| | | | | | yeast) | myces | | | | CBS | myces | | | yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Sc2OXAD_ | | P48570 | Homo- | | Saccharo- | Sc | Q4WUL6 | Homo- | del 2- | Neo- | Sc | P40495 | Homo- | Saccharo- | Sc |
| 90 | | | citrate | | myces | | | aconitase, | 41 | sartorya | | | isocitrate | myces | |
| | | | synthase, | | cerevisiae | | | mitochon- | and | fumigata | | | dehydro- | cerevisiae | |
| | | | cytosolic | | (strain | | | drial | del | (strain | | | genase, | (strain | |
| | | | isozyme | | ATCC | | | | 721- | ATCC | | | mitochon- | ATCC | |
| | | | | | 204508/ | | | | 777 | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Sc2OXAD_ | 196.7 | P48570 | Homo- | | Saccharo- | Yl | Q4WUL6 | Homo- | del 2- | Neo- | Yl | P40495 | Homo- | Saccharo- | Yl |
| 91 | | | citrate | | myces | | | aconitase, | 41 | sartorya | | | isocitrate | myces | |
| | | | synthase, | | cerevisiae | | | mitochon- | and | fumigata | | | dehydro- | cerevisiae | |
| | | | cytosolic | | (strain | | | drial | del | (strain | | | genase, | (strain | |
| | | | isozyme | | ATCC | | | | 721- | ATCC | | | mitochon- | ATCC | |
| | | | | | 204508/ | | | | 777 | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |

Yl = Yarrowia lipolytica;
Bs = Bacillus subtilis;
Sc = Saccharo-myces cerevisiae

TABLE 7
Host evaluation-round genetic engineering results for Corynebacterium Wutamicum

| | | | | | | | | | E2 | | | | | | |
|---|---|---|---|---|---|---|---|---|---|---|---|---|---|---|---|
| | | E1 | Enzyme 1- | E1 | Enzyme 1- | | E2 | Enzyme 2- | Modi- | Enzyme 2- | | E3 | Enzyme 3- | Enzyme 3- | |
| | Titer | Uniprot | activity | Modi- | source | E1 Codon | Uniprot | activity | fica- | source | E2 Codon | Uniprot | activity | source | |
| Strn | (μ/L) | ID | name | fications | organism | Optimization | ID | name | tions | organism | Optimization | ID | name | organism | E3 Codon Optimization |
| Cg2OXAD_ | 0 | P48570 | Homo- | | Saccharo- | Bacillus | Q4WUL6 | Homo- | | Neo- | Bacillus | P40495 | Homo- | Saccharo- | Bacillus |
| 100 | | | citrate | | myces | subtilis | | aconitase, | | sartorya | subtilis | | isocitrate | myces | subtilis |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 1947.6 | P48570 | Homo- | | Saccharo- | modified | Q4WUL6 | Homo- | | Neo- | modified | P40495 | Homo- | Saccharo- | modified |
| 101 | | | citrate | | myces | codon | | aconitase, | | sartorya | codon | | isocitrate | myces | codon |
| | | | synthase, | | cerevisiae | usage for | | mitochon- | | fumigata | usage for | | dehydro- | cerevisiae | usage for |
| | | | cytosolic | | (strain | Coryne- | | drial | | (strain | Coryne- | | genase, | (strain | Coryne- |
| | | | isozyme | | ATCC | bacterium | | | | ATCC | bacterium | | mitochon- | ATCC | bacterium |
| | | | | | 204508/ | glutami- | | | | MYA- | glutami- | | drial | 204508/ | glutami- |
| | | | | | S288c) | cum and | | | | 4609/ | cum and | | | S288c) | cum and |
| | | | | | (Baker's | Saccharo- | | | | Af293/ | Saccharo- | | | (Baker's | Saccharo- |
| | | | | | yeast) | myces | | | | CBS | myces | | | yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 0 | P48570 | Homo- | | Saccharo- | Saccharo- | Q4WUL6 | Homo- | | Neo- | Saccharo- | P40495 | Homo- | Saccharo- | Saccharomyces cerevisiae |
| 102 | | | citrate | | myces | myces | | aconitase, | | sartorya | myces | | isocitrate | myces | |
| | | | synthase, | | cerevisiae | cerevisiae | | mitochon- | | fumigata | cerevisiae | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 2718.1 | P48570 | Homo- | | Saccharo- | Yarrowia | Q4WUL6 | Homo- | | Neo- | Yarrowia | P40495 | Homo- | Saccharo- | Yarrowia lipolytica |
| 103 | | | citrate | | myces | lipolytica | | aconitase, | | sartorya | lipolytica | | isocitrate | myces | |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 224.3 | P48570 | Homo- | | Saccharo- | Bacillus | Q4WUL6 | Homo- | | Neo- | Bacillus | P40495 | Homo- | Saccharo- | Bacillus |
| 104 | | | citrate | | myces | subtilis | | aconitase, | | sartorya | subtilis | | isocitrate | myces | subtilis |
| | | | synthase, | | cerevisiae | | | mitochon- | | fumigata | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 0 | P48570 | Homo- | | Saccharo- | modified | Q4WUL6 | Homo- | del 2- | Neo- | modified | P40495 | Homo- | Saccharo- | modified |
| 105 | | | citrate | | myces | codon | | aconitase, | 41 | sartorya | codon | | isocitrate | myces | codon |
| | | | synthase, | | cerevisiae | usage for | | mitochon- | and | fumigata | usage for | | dehydro- | cerevisiae | usage for |
| | | | cytosolic | | (strain | Coryne- | | drial | del | (strain | Coryne- | | genase, | (strain | Coryne- |
| | | | isozyme | | ATCC | bacterium | | | 721- | ATCC | bacterium | | mitochon- | ATCC | bacterium |
| | | | | | 204508/ | glutami- | | | 777 | MYA- | glutami- | | drial | 204508/ | glutami- |
| | | | | | S288c) | cum and | | | | 4609/ | cum and | | | S288c) | cum and |
| | | | | | (Baker's | Saccharo- | | | | Af293/ | Saccharo- | | | (Baker's | Saccharo- |
| | | | | | yeast) | myces | | | | CBS | myces | | | yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 0 | P48570 | Homo- | | Saccharo- | Saccharo- | Q4WUL6 | Homo- | del 2- | Neo- | Saccharo- | P40495 | Homo- | Saccharo- | Saccharo- |
| 106 | | | citrate | | myces | myces | | aconitase, | 41 | sartorya | myces | | isocitrate | myces | myces |
| | | | synthase, | | cerevisiae | cerevisiae | | mitochon- | and | fumigata | cerevisiae | | dehydro- | cerevisiae | cerevisiae |
| | | | cytosolic | | (strain | | | drial | del | (strain | | | genase, | (strain | |
| | | | isozyme | | ATCC | | | | 721- | ATCC | | | mitochon- | ATCC | |
| | | | | | 204508/ | | | | 777 | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 295.7 | P48570 | Homo- | | Saccharo- | Yarrowia | Q4WUL6 | Homo- | del 2- | Neo- | Yarrowia | P40495 | Homo- | Saccharo- | Yarrowia |
| 107 | | | citrate | | myces | lipolytica | | aconitase, | 41 | sartorya | lipolytica | | isocitrate | myces | lipolytica |
| | | | synthase, | | cerevisiae | | | mitochon- | and | fumigata | | | dehydro- | cerevisiae | |
| | | | cytosolic | | (strain | | | drial | del | (strain | | | genase, | (strain | |
| | | | isozyme | | ATCC | | | | 721- | ATCC | | | mitochon- | ATCC | |
| | | | | | 204508/ | | | | 777 | MYA- | | | drial | 204508/ | |
| | | | | | S288c) | | | | | 4609/ | | | | S288c) | |
| | | | | | (Baker's | | | | | Af293/ | | | | (Baker's | |
| | | | | | yeast) | | | | | CBS | | | | yeast) | |
| | | | | | | | | | | 101355/ | | | | | |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 1310.4 | P48570 | Homo- | | Saccharo- | Bacillus | P49367 | Homo- | | Saccharo- | Bacillus | P40495 | Homo- | Saccharo- | Bacillus |
| 86 | | | citrate | | myces | subtilis | | aconitase, | | myces | subtilis | | isocitrate | myces | subtilis |
| | | | synthase, | | cerevisiae | | | mitochon- | | cerevisiae | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Cg2OXAD_ | 0 | P48570 | Homo- | | Saccharo- | Saccharo- | P49367 | Homo- | | Saccharo- | Saccharo- | P40495 | Homo- | Saccharo- | Saccharo- |
| 87 | | | citrate | | myces | myces | | aconitase, | | myces | myces | | isocitrate | myces | myces |
| | | | synthase, | | cerevisiae | cerevisiae | | mitochon- | | cerevisiae | cerevisiae | | dehydro- | cerevisiae | cerevisiae |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Cg2OXAD_ | 5737.3 | P48570 | Homo- | | Saccharo- | Yarrowia | P49367 | Homo- | | Saccharo- | Yarrowia | P40495 | Homo- | Saccharo- | Yarrowia |
| 88 | | | citrate | | myces | lipolytica | | aconitase, | | myces | lipolytica | | isocitrate | myces | lipolytica |
| | | | synthase, | | cerevisiae | | | mitochon- | | cerevisiae | | | dehydro- | cerevisiae | |
| | | | cytosolic | | (strain | | | drial | | (strain | | | genase, | (strain | |
| | | | isozyme | | ATCC | | | | | ATCC | | | mitochon- | ATCC | |
| | | | | | 204508/ | | | | | 204508/ | | | drial | 204508/ | |
| | | | | | S288c) | | | | | S288c) | | | | S288c) | |
| | | | | | (Baker's | | | | | (Baker's | | | | (Baker's | |
| | | | | | yeast) | | | | | yeast) | | | | yeast) | |
| Cg2OXAD_ | 96982.2 | Q9Y823 | Homo- | D123N | Schizo- | Bacillus | P49367 | Homo- | | Saccharo- | Bacillus | P40495 | Homo- | Saccharo- | Bacillus |
| 89 | | | citrate | | Saccharo- | subtilis | | aconitase, | | myces | subtilis | | isocitrate | myces | subtilis |
| | | | synthase, | | myces | | | mitochon- | | cerevisiae | | | dehydro- | cerevisiae | |
| | | | mitochon- | | pombe | | | drial | | (strain | | | genase, | (strain | |
| | | | drial | | (strain | | | | | ATCC | | | mitochon- | ATCC | |
| | | | | | 972/ | | | | | 204508/ | | | drial | 204508/ | |
| | | | | | ATCC | | | | | S288c) | | | | S288c) | |
| | | | | | 24843) | | | | | (Baker's | | | | (Baker's | |
| | | | | | (Fission | | | | | yeast) | | | | yeast) | |
| | | | | | yeast) | | | | | | | | | | |
| Cg2OXAD_ | 0 | Q9Y823 | Homo- | D123N | Schizo- | modified | P49367 | Homo- | | Saccharo- | modified | P40495 | Homo- | Saccharo- | modified |
| 90 | | | citrate | | Saccharo- | codon | | isocitrate | | myces | codon | | isocitrate | myces | codon |
| | | | synthase, | | myces | usage for | | hydro- | | cerevisiae | usage for | | dehydro- | cerevisiae | usage for |
| | | | mitochon- | | pombe | Coryne- | | lyase | | (strain | Coryne- | | genase, | (strain | Coryne- |
| | | | drial | | (strain | bacterium | | | | ATCC | bacterium | | mitochon- | ATCC | bacterium |
| | | | | | 972/ | glutami- | | | | 204508/ | glutami- | | drial | 204508/ | glutami- |
| | | | | | ATCC | cum and | | | | S288c) | cum and | | | S288c) | cum and |
| | | | | | 24843) | Saccharo- | | | | (Baker's | Saccharo- | | | (Baker's | Saccharo- |
| | | | | | (Fission | myces | | | | yeast) | myces | | | yeast) | myces |
| | | | | | yeast) | cerevisiae | | | | | cerevisiae | | | | cerevisiae |
| Cg2OXAD_ | 72083.5 | Q9Y823 | Homo- | D123N | Schizo- | Saccharo- | P49367 | Homo- | | Saccharo- | Saccharo- | P40495 | Homo- | Saccharo- | Saccharo- |
| 91 | | | citrate | | Saccharo- | myces | | aconitase, | | myces | myces | | isocitrate | myces | myces |
| | | | synthase, | | myces | cerevisiae | | mitochon- | | cerevisiae | cerevisiae | | dehydro- | cerevisiae | cerevisiae |
| | | | mitochon- | | pombe | | | drial | | (strain | | | genase, | (strain | |
| | | | drial | | (strain | | | | | ATCC | | | mitochon- | ATCC | |
| | | | | | 972/ | | | | | 204508/ | | | drial | 204508/ | |
| | | | | | ATCC | | | | | S288c) | | | | S288c) | |
| | | | | | 24843) | | | | | (Baker's | | | | (Baker's | |
| | | | | | (Fission | | | | | yeast) | | | | yeast) | |
| | | | | | yeast) | | | | | | | | | | |
| Cg2OXAD_ | 5042.8 | Q9Y823 | Homo- | D123N | Schizo- | Yarrowia | P49367 | Homo- | | Saccharo- | Yarrowia | P40495 | Homo- | Saccharo- | Yarrowia |
| 92 | | | citrate | | Saccharo- | lipolytica | | aconitase, | | myces | lipolytica | | isocitrate | myces | lipolytica |
| | | | synthase, | | myces | | | mitochon- | | cerevisiae | | | dehydro- | cerevisiae | |
| | | | mitochon- | | pombe | | | | | (strain | | | genase, | (strain | |
| | | | drial | | (strain | | | | | ATCC | | | mitochon- | ATCC | |
| | | | | | 972/ | | | | | 204508/ | | | drial | 204508/ | |
| | | | | | ATCC | | | | | S288c) | | | | S288c) | |
| | | | | | 24843) | | | | | (Baker's | | | | (Baker's | |
| | | | | | (Fission | | | | | yeast) | | | | yeast) | |
| | | | | | yeast) | | | | | | | | | | |
| Cg2OXAD_ | 713 | A0A0G9LF37 | Trans- | | Clostri- | Bacillus | P49367 | Homo- | | Saccharo- | Bacillus | P40495 | Homo- | Saccharo- | Bacillus |
| 93 | | | Homo- | | dium sp. | subtilis | | aconitase, | | myces | subtilis | | isocitrate | myces | subtilis |
| | | | aconitate | | C8 | | | mitochon- | | cerevisiae | | | dehydro- | cerevisiae | |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Cg2OXAD_ | 228.6 | A0A0G9LF37 | Trans- | | Clostri- | modified | P49367 | Homo- | | Saccharo- | modified | P40495 | Homo- | Saccharo- | modified |
| 94 | | | Homo- | | dium sp. | codon | | isocitrate | | myces | codon | | isocitrate | myces | codon |
| | | | aconitate | | C8 | usage for | | hydro- | | cerevisiae | usage for | | dehydro- | cerevisiae | usage for |
| | | | synthase | | | Coryne- | | lyase | | (strain | Coryne- | | genase, | (strain | Coryne- |
| | | | | | | bacterium | | | | ATCC | bacterium | | mitochon- | ATCC | bacterium |
| | | | | | | glutami- | | | | 204508/ | glutami- | | drial | 204508/ | glutami- |
| | | | | | | cum and | | | | S288c) | cum and | | | S288c) | cum and |
| | | | | | | Saccharo- | | | | (Baker's | Saccharo- | | | (Baker's | Saccharo- |
| | | | | | | myces | | | | yeast) | myces | | | yeast) | myces |
| | | | | | | cerevisiae | | | | | cerevisiae | | | | cerevisiae |
| Cg2OXAD_ | 201.4 | A0A0G9LF37 | Trans- | | Clostri- | Saccharo- | P49367 | Homo- | | Saccharo- | Saccharo- | P40495 | Homo- | Saccharo- | Saccharo- |
| 95 | | | Homo- | | dium sp. | myces | | aconitase, | | myces | myces | | isocitrate | myces | myces |
| | | | aconitate | | C8 | cerevisiae | | mitochon- | | cerevisiae | cerevisiae | | dehydro- | cerevisiae | cerevisiae |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Cg2OXAD_ | 520.2 | A0A0G9LF37 | Trans- | | Clostri- | Yarrowia | P49367 | Homo- | | Saccharo- | Yarrowia | P40495 | Homo- | Saccharo- | Yarrowia |
| 96 | | | Homo- | | dium sp. | lipolytica | | aconitase, | | myces | lipolytica | | isocitrate | myces | lipolytica |
| | | | aconitate | | C8 | | | mitochon- | | cerevisiae | | | dehydro- | cerevisiae | |
| | | | synthase | | | | | drial | | (strain | | | genase, | (strain | |
| | | | | | | | | | | ATCC | | | mitochon- | ATCC | |
| | | | | | | | | | | 204508/ | | | drial | 204508/ | |
| | | | | | | | | | | S288c) | | | | S288c) | |
| | | | | | | | | | | (Baker's | | | | (Baker's | |
| | | | | | | | | | | yeast) | | | | yeast) | |
| Cg2OXAD_ | 213.4 | O87198 | Homo- | | Thermus | Bacillus | P49367 | Homo- | | Saccharo- | Bacillus | Q72IW9 | Homo- | Thermus | Bacillus |
| 97 | | | citrate | | thermo- | subtilis | | aconitase, | | myces | subtilis | | isocitrate | thermo- | subtilis |
| | | | synthase | | philus | | | mitochon- | | cerevisiae | | | dehydro- | philus | |
| | | | | | (strain | | | drial | | (strain | | | genase | (strain | |
| | | | | | HB27/ | | | | | ATCC | | | | H B27/ | |
| | | | | | ATCC | | | | | 204508/ | | | | ATCC | |
| | | | | | BAA-163/ | | | | | S288c) | | | | BAA-163/ | |
| | | | | | DSM | | | | | (Baker's | | | | DSM | |
| | | | | | 7039) | | | | | yeast) | | | | 7039) | |
| Cg2OXAD_ | 756.4 | O87198 | Homo- | | Thermus | Saccharo- | P49367 | Homo- | | Saccharo- | Saccharo- | Q72IW9 | Homo- | Thermus | Saccharo- |
| 98 | | | citrate | | thermo- | myces | | aconitase, | | myces | myces | | isocitrate | thermo- | myces |
| | | | synthase | | philus | cerevisiae | | mitochon- | | cerevisiae | cerevisiae | | dehydro- | philus | cerevisiae |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-163/ | | | | | S288c) | | | | BAA-163/ | |
| | | | | | DSM | | | | | (Baker's | | | | DSM | |
| | | | | | 7039) | | | | | yeast) | | | | 7039) | |
| Cg2OXAD_ | 78777.8 | O87198 | Homo- | | Thermus | Yarrowia | P49367 | Homo- | | Saccharo- | Yarrowia | Q72IW9 | Homo- | Thermus | Yarrowia |
| 99 | | | citrate | | thermo- | lipolytica | | aconitase, | | myces | lipolytica | | isocitrate | thermo- | lipolytica |
| | | | synthase | | philus | | | mitochon- | | cerevisiae | | | dehydro- | philus | |
| | | | | | (strain | | | drial | | (strain | | | genase | (strain | |
| | | | | | HB27/ | | | | | ATCC | | | | HB27/ | |
| | | | | | ATCC | | | | | 204508/ | | | | ATCC | |
| | | | | | BAA-1631 | | | | | S288c) | | | | BAA-163/ | |
| | | | | | DSM | | | | | (Baker's | | | | DSM | |
| | | | | | 7039) | | | | | yeast) | | | | 7039) | |

Yl = Yarrowia lipolytica;
Bs = Bacillus subtilis;
Sc = Saccharo-myces cerevisiae

TABLE 8
Improvement-round genetic engineering results for Corynebacterium glutamicum

| | | | | | | | | | E2 | | | | | | |
|---|---|---|---|---|---|---|---|---|---|---|---|---|---|---|---|
| | | E1 | Enzyme 1- | E1 | Enzyme 1- | E1 Codon | E2 | Enzyme 2- | Modi- | Enzyme 2- | | E3 | Enzyme 3- | Enzyme 3- | E3 Codon |
| | Titer | Uniprot | activity | Modi- | source | Optimi- | Uniprot | activity | fica- | source | E2 Codon | Uniprot | activity | source | Optimi- |
| Strn | (μ/L) | ID | name | fications | organism | zation | ID | name | tions | organism | Optimization | ID | name | organism | zation |
| Cg2OXAD_ | 0 | Q9Y823 | Homo- | D123N | Schizo- | modified | P49367 | Homo- | | Saccharo- | modified | P40495 | Homo- | Saccharo- | modified |
| 50 | | | citrate | | Saccharo- | codon | | isocitrate | | myces | codon | | isocitrate | myces | codon |
| | | | synthase, | | myces | usage for | | hydro- | | cerevisiae | usage for | | dehydro- | cerevisiae | usage for |
| | | | mitochon- | | pombe | Coryne- | | lyase | | (strain | Coryne- | | genase, | (strain | Coryne- |
| | | | drial (EC | | (strain | bacterium | | | | ATCC | bacterium | | mitochon- | ATCC | bacterium |
| | | | 2.3.3.14) | | 972/ | glutami- | | | | 204508/ | glutami- | | drial | 204508/ | glutami- |
| | | | | | ATCC | cum and | | | | S288c) | cum and | | (HIcDH) | S288c) | cum and |
| | | | | | 24843) | Saccharo- | | | | (Baker's | Saccharo- | | (EC | (Baker's | Saccharo- |
| | | | | | (Fission | myces | | | | yeast) | myces | | 1.1.1.87) | yeast) | myces |
| | | | | | yeast) | cerevisiae | | | | | cerevisiae | | | | cerevisiae |
| Cg2OXAD_ | 596.4 | Q9Y823 | Homo- | E222Q | Schizo- | modified | P49367 | Homo- | | Saccharo- | modified | P40495 | Homo- | Saccharo- | modified |
| 51 | | | citrate | | Saccharo- | codon | | isocitrate | | myces | codon | | isocitrate | myces | codon |
| | | | synthase, | | myces | usage for | | hydro- | | cerevisiae | usage for | | dehydro- | cerevisiae | usage for |
| | | | mitochon- | | pombe | Coryne- | | lyase | | (strain | Coryne- | | genase, | (strain | Coryne- |
| | | | drial (EC | | (strain | bacterium | | | | ATCC | bacterium | | mitochon- | ATCC | bacterium |
| | | | 2.3.3.14) | | 972/ | glutami- | | | | 204508/ | glutami- | | drial | 204508/ | glutami- |
| | | | | | ATCC | cum and | | | | S288c) | cum and | | (HIcDH) | S288c) | cum and |
| | | | | | 24843) | Saccharo- | | | | (Baker's | Saccharo- | | (EC | (Baker's | Saccharo- |
| | | | | | (Fission | myces | | | | yeast) | myces | | 1.1.1.87) | yeast) | myces |
| | | | | | yeast) | cerevisiae | | | | | cerevisiae | | | | cerevisiae |
| Cg2OXAD_ | 47667 | Q9Y823 | Homo- | R288K | Schizo- | modified | P49367 | Homo- | | Saccharo- | modified | P40495 | Homo- | Saccharo- | modified |
| 52 | | | citrate | | Saccharo- | codon | | isocitrate | | myces | codon | | isocitrate | myces | codon |
| | | | synthase, | | myces | usage for | | hydro- | | cerevisiae | usage for | | dehydro- | cerevisiae | usage for |
| | | | mitochon- | | pombe | Coryne- | | lyase | | (strain | Coryne- | | genase, | (strain | Coryne- |
| | | | drial (EC | | (strain | bacterium | | | | ATCC | bacterium | | mitochon- | ATCC | bacterium |
| | | | 2.3.3.14) | | 972/ | glutami- | | | | 204508/ | glutami- | | drial | 204508/ | glutami- |
| | | | | | ATCC | cum and | | | | S288c) | cum and | | (HIcDH) | S288c) | cum and |
| | | | | | 24843) | Saccharo- | | | | (Baker's | Saccharo- | | (EC | (Baker's | Saccharo- |
| | | | | | (Fission | myces | | | | yeast) | myces | | 1.1.1.87) | yeast) | myces |
| | | | | | yeast) | cerevisiae | | | | | cerevisiae | | | | cerevisiae |
| Cg2OXAD_ | 258.2 | Q9Y823 | Homo- | R275K | Schizo- | modified | P49367 | Homo- | | Saccharo- | modified | P40495 | Homo- | Saccharo- | modified |
| 53 | | | citrate | | Saccharo- | codon | | isocitrate | | myces | codon | | isocitrate | myces | codon |
| | | | synthase, | | myces | usage for | | hydro- | | cerevisiae | usage for | | dehydro- | cerevisiae | usage for |
| | | | mitochon- | | pombe | Coryne- | | lyase | | (strain | Coryne- | | genase, | (strain | Coryne- |
| | | | drial (EC | | (strain | bacterium | | | | ATCC | bacterium | | mitochon- | ATCC | bacterium |
| | | | 2.3.3.14) | | 972/ | glutami- | | | | 204508/ | glutami- | | drial | 204508/ | glutami- |
| | | | | | ATCC | cum and | | | | S288c) | cum and | | (HIcDH) | S288c) | cum and |
| | | | | | 24843) | Saccharo- | | | | (Baker's | Saccharo- | | (EC | (Baker's | Saccharo- |
| | | | | | (Fission | myces | | | | yeast) | myces | | 1.1.1.87) | yeast) | myces |
| | | | | | yeast) | cerevisiae | | | | | cerevisiae | | | | cerevisiae |
| Cg2OXAD_ | 0 | P48570 | Homo- | | Saccharo- | modified | P49367 | Homo- | | Saccharo- | modified | P40495 | Homo- | Saccharo- | modified |
| 54 | | | citrate | | myces | codon | | isocitrate | | myces | codon | | isocitrate | myces | codon |
| | | | synthase, | | cerevisiae | usage for | | hydro- | | cerevisiae | usage for | | dehydro- | cerevisiae | usage for |
| | | | cytosolic | | (strain | Coryne- | | lyase | | (strain | Coryne- | | genase, | (strain | Coryne- |
| | | | isozyme | | ATCC | bacterium | | | | ATCC | bacterium | | mitochon- | ATCC | bacterium |
| | | | (EC | | 204508/ | glutami- | | | | 204508/ | glutami- | | drial | 204508/ | glutami- |
| | | | 2.3.3.14) | | S288c) | cum and | | | | S288c) | cum and | | (HIcDH) | S288c) | cum and |
| | | | | | (Baker's | Saccharo- | | | | (Baker's | Saccharo- | | (EC | (Baker's | Saccharo- |
| | | | | | yeast) | myces | | | | yeast) | myces | | 1.1.1.87) | yeast) | myces |
| | | | | | | cerevisiae | | | | | cerevisiae | | | | cerevisiae |
| Cg2OXAD_ | 6121 | Q9Y823 | Homo- | D123N | Schizo- | modified | P49367 | Homo- | | Saccharo- | modified | P40495 | Homo- | Saccharo- | modified |
| 55 | | | citrate | | Saccharo- | codon | | isocitrate | | myces | codon | | isocitrate | myces | codon |
| | | | synthase, | | myces | usage for | | hydro- | | cerevisiae | usage for | | dehydro- | cerevisiae | usage for |
| | | | mitochon- | | pombe | Coryne- | | lyase | | (strain | Coryne- | | genase, | (strain | Coryne- |
| | | | drial (EC | | (strain | bacterium | | | | ATCC | bacterium | | mitochon- | ATCC | bacterium |
| | | | 2.3.3.14) | | 972/ | glutami- | | | | 204508/ | glutami- | | drial | 204508/ | glutami- |
| | | | | | ATCC | cum and | | | | S288c) | cum and | | (HIcDH) | S288c) | cum and |
| | | | | | 24843) | Saccharo- | | | | (Baker's | Saccharo- | | (EC | (Baker's | Saccharo- |
| | | | | | (Fission | myces | | | | yeast) | myces | | 1.1.1.87) | yeast) | myces |
| | | | | | yeast) | cerevisiae | | | | | cerevisiae | | | | cerevisiae |
| Cg2OXAD_ | 270.5 | P48570 | Homo- | | Saccharo- | modified | Q4WUL6 | Homo- | | Neo- | modified | P40495 | Homo- | Saccharo- | modified |
| 56 | | | citrate | | myces | codon | | aconitase, | | sartorya | codon | | isocitrate | myces | codon |
| | | | synthase, | | cerevisiae | usage for | | mitochon- | | fumigata | usage for | | dehydro- | cerevisiae | usage for |
| | | | cytosolic | | (strain | Coryne- | | drial (EC | | (strain | Coryne- | | genase, | (strain | Coryne- |
| | | | isozyme | | ATCC | bacterium | | 4.2.1.36) | | ATCC | bacterium | | mitochon- | ATCC | bacterium |
| | | | (EC | | 204508/ | glutami- | | (Homo- | | MYA- | glutami- | | drial | 204508/ | glutami- |
| | | | 2.3.3.14) | | S288c) | cum and | | aconitate | | 4609/ | cum and | | (HIcDH) | S288c) | cum and |
| | | | | | (Baker's | Saccharo- | | hydratase) | | Af293/ | Saccharo- | | (EC | (Baker's | Saccharo- |
| | | | | | yeast) | myces | | | | CBS | myces | | 1.1.1.87) | yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 5171.9 | P48570 | Homo- | | Saccharo- | modified | P49367 | Homo- | | Saccharo- | modified | P40495 | Homo- | Saccharo- | modified |
| 57 | | | citrate | | myces | codon | | aconitase, | | myces | codon | | isocitrate | myces | codon |
| | | | synthase, | | cerevisiae | usage for | | mitochon- | | cerevisiae | usage for | | dehydro- | cerevisiae | usage for |
| | | | cytosolic | | (strain | Coryne- | | drial (EC | | (strain | Coryne- | | genase, | (strain | Coryne- |
| | | | isozyme | | ATCC | bacterium | | 4.2.1.36) | | ATCC | bacterium | | mitochon- | ATCC | bacterium |
| | | | (EC | | 204508/ | glutami- | | (Homo- | | 204508/ | glutami- | | drial | 204508/ | glutami- |
| | | | 2.3.3.14) | | S288c) | cum and | | aconitate | | S288c) | cum and | | (HIcDH) | S288c) | cum and |
| | | | | | (Baker's | Saccharo- | | hydratase) | | (Baker's | Saccharo- | | (EC | (Baker's | Saccharo- |
| | | | | | yeast) | myces | | | | yeast) | myces | | 1.1.1.87) | yeast) | myces |
| | | | | | | cerevisiae | | | | | cerevisiae | | | | cerevisiae |
| Cg2OXAD_ | 5063.5 | P48570 | Homo- | | Saccharo- | modified | P49367 | Homo- | | Saccharo- | modified | P40495 | Homo- | Saccharo- | modified |
| 58 | | | citrate | | myces | codon | | aconitase, | | myces | codon | | isocitrate | myces | codon |
| | | | synthase, | | cerevisiae | usage for | | mitochon- | | cerevisiae | usage for | | dehydro- | cerevisiae | usage for |
| | | | cytosolic | | (strain | Coryne- | | drial (EC | | (strain | Coryne- | | genase, | (strain | Coryne- |
| | | | isozyme | | ATCC | bacterium | | 4.2.1.36) | | ATCC | bacterium | | mitochon- | ATCC | bacterium |
| | | | (EC | | 204508/ | glutami- | | (Homo- | | 204508/ | glutami- | | drial | 204508/ | glutami- |
| | | | 2.3.3.14) | | S288c) | cum and | | aconitate | | S288c) | cum and | | (HIcDH) | S288c) | cum and |
| | | | | | (Baker's | Saccharo- | | hydratase) | | (Baker's | Saccharo- | | (EC | (Baker's | Saccharo- |
| | | | | | yeast) | myces | | | | yeast) | myces | | 1.1.1.87) | yeast) | myces |
| | | | | | | cerevisiae | | | | | cerevisiae | | | | cerevisiae |
| Cg2OXAD_ | 261.5 | P48570 | Homo- | | Saccharo- | modified | | | | | | | | | |
| 59 | | | citrate | | myces | codon | | | | | | | | | |
| | | | synthase, | | cerevisiae | usage for | | | | | | | | | |
| | | | cytosolic | | (strain | Coryne- | | | | | | | | | |
| | | | isozyme | | ATCC | bacterium | | | | | | | | | |
| | | | (EC | | 204508/ | glutami- | | | | | | | | | |
| | | | 2.3.3.14) | | S288c) | cum and | | | | | | | | | |
| | | | | | (Baker's | Saccharo- | | | | | | | | | |
| | | | | | yeast) | myces | | | | | | | | | |
| | | | | | | cerevisiae | | | | | | | | | |
| Cg2OXAD_ | 276.6 | P48570 | Homo- | | Saccharo- | modified | | | | | | | | | |
| 60 | | | citrate | | myces | codon | | | | | | | | | |
| | | | synthase, | | cerevisiae | usage for | | | | | | | | | |
| | | | cytosolic | | (strain | Coryne- | | | | | | | | | |
| | | | isozyme | | ATCC | bacterium | | | | | | | | | |
| | | | (EC | | 204508/ | glutami- | | | | | | | | | |
| | | | 2.3.3.14) | | S288c) | cum and | | | | | | | | | |
| | | | | | (Baker's | Saccharo- | | | | | | | | | |
| | | | | | yeast) | myces | | | | | | | | | |
| | | | | | | cerevisiae | | | | | | | | | |
| Cg2OXAD_ | 0 | D5Q163 | Homo- | | Clostridioides | modified | | | | | | | | | |
| 61 | | | citrate | | difficile | codon | | | | | | | | | |
| | | | synthase | | NAP08 | usage for | | | | | | | | | |
| | | | (EC | | | Coryne- | | | | | | | | | |
| | | | 2.3.3.14) | | | bacterium | | | | | | | | | |
| | | | | | | glutami- | | | | | | | | | |
| | | | | | | cum and | | | | | | | | | |
| | | | | | | Saccharo- | | | | | | | | | |
| | | | | | | myces | | | | | | | | | |
| | | | | | | cerevisiae | | | | | | | | | |
| Cg2OXAD_ | 51691.5 | O87198 | Homo- | | Thermus | modified | P49367 | Homo- | | Saccharo- | modified | P40495 | Homo- | Saccharo- | modified |
| 62 | | | citrate | | thermo- | codon | | isocitrate | | myces | codon | | isocitrate | myces | codon |
| | | | synthase | | philus | usage for | | hydro- | | cerevisiae | usage for | | dehydro- | cerevisiae | usage for |
| | | | (EC | | (strain | Coryne- | | lyase | | (strain | Coryne- | | genase | (strain | Coryne- |
| | | | 2.3.3.14) | | HB27/ | bacterium | | | | ATCC | bacterium | | | ATCC | bacterium |
| | | | | | ATCC | glutami- | | | | 204508/ | glutami- | | | 204508/ | glutami- |
| | | | | | BAA-163/ | cum and | | | | S288c) | cum and | | | S288c) | cum and |
| | | | | | DSM | Saccharo- | | | | (Baker's | Saccharo- | | | (Baker's | Saccharo- |
| | | | | | 7039) | myces | | | | yeast) | myces | | | yeast) | myces |
| | | | | | | cerevisiae | | | | | cerevisiae | | | | cerevisiae |
| Cg2OXAD_ | 825.3 | G8NBZ9 | Homo- | | Thermus | modified | P49367 | Homo- | | Saccharo- | modified | P40495 | Homo- | Saccharo- | modified |
| 63 | | | citrate | | sp. | codon | | isocitrate | | myces | codon | | isocitrate | myces | codon |
| | | | synthase | | CCB_US3_ | usage for | | hydro- | | cerevisiae | usage for | | dehydro- | cerevisiae | usage for |
| | | | | | UF1 | Coryne- | | lyase | | (strain | Coryne- | | genase | (strain | Coryne- |
| | | | | | | bacterium | | | | ATCC | bacterium | | | ATCC | bacterium |
| | | | | | | glutami- | | | | 204508/ | glutami- | | | 204508/ | glutami- |
| | | | | | | cum and | | | | S288c) | cum and | | | S288c) | cum and |
| | | | | | | Saccharo- | | | | (Baker's | Saccharo- | | | (Baker's | Saccharo- |
| | | | | | | myces | | | | yeast) | myces | | | yeast) | myces |
| | | | | | | cerevisiae | | | | | cerevisiae | | | | cerevisiae |
| Cg2OXAD_ | 255.1 | F2NL20 | Homo- | | Marini- | modified | P49367 | Homo- | | Saccharo- | modified | P40495 | Homo- | Saccharo- | modified |
| 64 | | | citrate | | thermus | codon | | isocitrate | | myces | codon | | isocitrate | myces | codon |
| | | | synthase | | hydro- | usage for | | hydro- | | cerevisiae | usage for | | dehydro- | cerevisiae | usage for |
| | | | (EC | | thermalis | Coryne- | | lyase | | (strain | Coryne- | | genase | (strain | Coryne- |
| | | | 2.3.3.14) | | (strain | bacterium | | | | ATCC | bacterium | | | ATCC | bacterium |
| | | | | | DSM | glutami- | | | | 204508/ | glutami- | | | 204508/ | glutami- |
| | | | | | 14884/ | cum and | | | | S288c) | cum and | | | S288c) | cum and |
| | | | | | JCM | Saccharo- | | | | (Baker's | Saccharo- | | | (Baker's | Saccharo- |
| | | | | | 11576/ | myces | | | | yeast) | myces | | | yeast) | myces |
| | | | | | T1) | cerevisiae | | | | | cerevisiae | | | | cerevisiae |
| Cg2OXAD_ | 0 | A0A0F7TVK2 | Homo- | | Penicillium | modified | P49367 | Homo- | | Saccharo- | modified | P40495 | Homo- | Saccharo- | modified |
| 65 | | | citrate | | brasilianum | codon | | isocitrate | | myces | codon | | isocitrate | myces | codon |
| | | | synthase, | | | usage for | | hydro- | | cerevisiae | usage for | | dehydro- | cerevisiae | usage for |
| | | | mitochon- | | | Coryne- | | lyase | | (strain | Coryne- | | genase | (strain | Coryne- |
| | | | drial | | | bacterium | | | | ATCC | bacterium | | | ATCC | bacterium |
| | | | (Putative | | | glutami- | | | | 204508/ | glutami- | | | 204508/ | glutami- |
| | | | Homo- | | | cum and | | | | S288c) | cum and | | | S288c) | cum and |
| | | | citrate | | | Saccharo- | | | | (Baker's | Saccharo- | | | (Baker's | Saccharo- |
| | | | synthase) | | | myces | | | | yeast) | myces | | | yeast) | myces |
| | | | | | | cerevisiae | | | | | cerevisiae | | | | cerevisiae |
| Cg2OXAD_ | 797 | A0A0L1I0C1 | Homo- | | Stemphylium | modified | P49367 | Homo- | | Saccharo- | modified | P40495 | Homo- | Saccharo- | modified |
| 66 | | | citrate | | lycopersici | codon | | isocitrate | | myces | codon | | isocitrate | myces | codon |
| | | | synthase | | | usage for | | hydro- | | cerevisiae | usage for | | dehydro- | cerevisiae | usage for |
| | | | (EC | | | Coryne- | | lyase | | (strain | Coryne- | | genase | (strain | Coryne- |
| | | | 2.3.3.14) | | | bacterium | | | | ATCC | bacterium | | | ATCC | bacterium |
| | | | | | | glutami- | | | | 204508/ | glutami- | | | 204508/ | glutami- |
| | | | | | | cum and | | | | S288c) | cum and | | | S288c) | cum and |
| | | | | | | Saccharo- | | | | (Baker's | Saccharo- | | | (Baker's | Saccharo- |
| | | | | | | myces | | | | yeast) | myces | | | yeast) | myces |
| | | | | | | cerevisiae | | | | | cerevisiae | | | | cerevisiae |
| Cg2OXAD_ | 498.5 | Q2IHS7 | Homo- | | Anaeromy- | modified | P49367 | Homo- | | Saccharo- | modified | B3LTU1 | Homo- | Saccharo- | modified |
| 67 | | | citrate | | xobacter | codon | | isocitrate | | myces | codon | | isocitrate | myces | codon |
| | | | synthase | | dehalogenans | usage for | | hydro- | | cerevisiae | usage for | | dehydro- | cerevisiae | usage for |
| | | | (EC | | (strain | Coryne- | | lyase | | (strain | Coryne- | | genase | (strain | Coryne- |
| | | | 2.3.3.14) | | 2CP-C) | bacterium | | | | ATCC | bacterium | | | RM11-1a) | bacterium |
| | | | | | | glutami- | | | | 204508/ | glutami- | | | (Baker's | glutami- |
| | | | | | | cum and | | | | S288c) | cum and | | | yeast) | cum and |
| | | | | | | Saccharo- | | | | (Baker's | Saccharo- | | | | Saccharo- |
| | | | | | | myces | | | | yeast) | myces | | | | myces |
| | | | | | | cerevisiae | | | | | cerevisiae | | | | cerevisiae |
| Cg2OXAD_ | 0 | A0A1F8TP88 | Homo- | | Chloroflexi | modified | Q4WUL6 | Homo- | | Neo- | modified | B3LTU1 | Homo- | Saccharo- | modified |
| 68 | | | citrate | | bacterium | codon | | aconitase, | | sartorya | codon | | isocitrate | myces | codon |
| | | | synthase | | RIFCSPLOWO2_ | usage for | | mitochon- | | fumigata | usage for | | dehydro- | cerevisiae | usage for |
| | | | | | 12_FULL_71_ | Coryne- | | drial (EC | | (strain | Coryne- | | genase | (strain | Coryne- |
| | | | | | 12 | bacterium | | 4.2.1.36) | | ATCC | bacterium | | | RM11-1a) | bacterium |
| | | | | | | glutami- | | (Homo- | | MYA- | glutami- | | | (Baker's | glutami- |
| | | | | | | cum and | | aconitate | | 4609/ | cum and | | | yeast) | cum and |
| | | | | | | Saccharo- | | hydratase) | | Af293/ | Saccharo- | | | | Saccharo- |
| | | | | | | myces | | | | CBS | myces | | | | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 4961.1 | P48570 | Homo- | | Saccharo- | modified | Q4WUL6 | Homo- | | Neo- | modified | B3LTU1 | Homo- | Saccharo- | modified |
| 69 | | | citrate | | myces | codon | | aconitase, | | sartorya | codon | | isocitrate | myces | codon |
| | | | synthase, | | cerevisiae | usage for | | mitochon- | | fumigata | usage for | | dehydro- | cerevisiae | usage for |
| | | | cytosolic | | (strain | Coryne- | | drial (EC | | (strain | Coryne- | | genase | (strain | Coryne- |
| | | | isozyme | | ATCC | bacterium | | 4.2.1.36) | | ATCC | bacterium | | | RM11-1a) | bacterium |
| | | | (EC | | 204508/ | glutami- | | (Homo- | | MYA- | glutami- | | | (Baker's | glutami- |
| | | | 2.3.3.14) | | S288c) | cum and | | aconitate | | 4609/ | cum and | | | yeast) | cum and |
| | | | | | (Baker's | Saccharo- | | hydratase) | | Af293/ | Saccharo- | | | | Saccharo- |
| | | | | | yeast) | myces | | | | CBS | myces | | | | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 334.7 | Q75A20 | ADR107Wp | | Ashbya | modified | Q4WUL6 | Homo- | | Neo- | modified | B3LTU1 | Homo- | Saccharo- | modified |
| 70 | | | | | gossypii | codon | | aconitase, | | sartorya | codon | | isocitrate | myces | codon |
| | | | | | (strain | usage for | | mitochon- | | fumigata | usage for | | dehydro- | cerevisiae | usage for |
| | | | | | ATCC | Coryne- | | drial (EC | | (strain | Coryne- | | genase | (strain | Coryne- |
| | | | | | 10895/ | bacterium | | 4.2.1.36) | | ATCC | bacterium | | | RM11-1a) | bacterium |
| | | | | | CBS | glutami- | | (Homo- | | MYA- | glutami- | | | (Baker's | glutami- |
| | | | | | 109.51/ | cum and | | aconitate | | 4609/ | cum and | | | yeast) | cum and |
| | | | | | FGSC | Saccharo- | | hydratase) | | Af293/ | Saccharo- | | | | Saccharo- |
| | | | | | 9923/ | myces | | | | CBS | myces | | | | myces |
| | | | | | NRRL Y- | cerevisiae | | | | 101355/ | cerevisiae | | | | cerevisiae |
| | | | | | 1056) | | | | | FGSC | | | | | |
| | | | | | (Yeast) | | | | | A1100) | | | | | |
| | | | | | (Eremothecium | | | | | (Asper- | | | | | |
| | | | | | gossypii) | | | | | gillus | | | | | |
| | | | | | | | | | | fumigatus) | | | | | |
| Cg2OXAD_ | 280.8 | E4VIM0 | Homo- | | Arthroderma | modified | Q4WUL6 | Homo- | | Neo- | modified | B3LTU1 | Homo- | Saccharo- | modified |
| 71 | | | citrate | | gypseum | codon | | aconitase, | | sartorya | codon | | isocitrate | myces | codon |
| | | | synthase | | (strain | usage for | | mitochon- | | fumigata | usage for | | dehydro- | cerevisiae | usage for |
| | | | | | ATCC | Coryne- | | drial (EC | | (strain | Coryne- | | genase | (strain | Coryne- |
| | | | | | MYA- | bacterium | | 4.2.1.36) | | ATCC | bacterium | | | RM11-1a) | bacterium |
| | | | | | 4604/ | glutami- | | (Homo- | | MYA- | glutami- | | | (Baker's | glutami- |
| | | | | | CBS | cum and | | aconitate | | 4609/ | cum and | | | yeast) | cum and |
| | | | | | 118893) | Saccharo- | | hydratase) | | Af293/ | Saccharo- | | | | Saccharo- |
| | | | | | (Microsporum | myces | | | | CBS | myces | | | | myces |
| | | | | | gypseum) | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 280.8 | F2PSY4 | Homo- | | Trichophyton | modified | Q4WUL6 | Homo- | | Neo- | modified | J8Q3V7 | Homo- | Saccharo- | modified |
| 72 | | | citrate | | equinum | codon | | aconitase, | | sartorya | codon | | isocitrate | myces | codon |
| | | | synthase | | (strain | usage for | | mitochon- | | fumigata | usage for | | dehydro- | arboricola | usage for |
| | | | | | ATCC | Coryne- | | drial (EC | | (strain | Coryne- | | genase | (strain H-6/ | Coryne- |
| | | | | | MYA- | bacterium | | 4.2.1.36) | | ATCC | bacterium | | | AS | bacterium |
| | | | | | 4606/ | glutami- | | (Homo- | | MYA- | glutami- | | | 2.3317/ | glutami- |
| | | | | | CBS | cum and | | aconitate | | 4609/ | cum and | | | CBS | cum and |
| | | | | | 127.97) | Saccharo- | | hydratase) | | Af293/ | Saccharo- | | | 10644) | Saccharo- |
| | | | | | (Horse | myces | | | | CBS | myces | | | (Yeast) | myces |
| | | | | | ringworm | cerevisiae | | | | 101355/ | cerevisiae | | | | cerevisiae |
| | | | | | fungus) | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 233.7 | P12683 | 3-hydroxy- | del 1- | Saccharo- | modified | Q4WUL6 | Homo- | | Neo- | modified | J8Q3V7 | Homo- | Saccharo- | modified |
| 73 | | | 3- | 527; | myces | codon | | aconitase, | | sartorya | codon | | isocitrate | myces | codon |
| | | | methyl- | Y528 | cerevisiae | usage for | | mitochon- | | fumigata | usage for | | dehydro- | arboricola | usage for |
| | | | glutaryl- | M; | (strain | Coryne- | | drial (EC | | (strain | Coryne- | | genase | (strain H-6/ | Coryne- |
| | | | coenzyme | T529A | ATCC | bacterium | | 4.2.1.36) | | ATCC | bacterium | | | AS | bacterium |
| | | | A | | 204508/ | glutami- | | (Homo- | | MYA- | glutami- | | | 2.3317/ | glutami- |
| | | | reductase | | S288c) | cum and | | aconitate | | 4609/ | cum and | | | CBS | cum and |
| | | | 1 (HMG- | | (Baker's | Saccharo- | | hydratase) | | Af293/ | Saccharo- | | | myces | Saccharo- |
| | | | CoA | | yeast) | myces | | | | CBS | myces | | | 10644) | (Yeast) |
| | | | reductase | | | cerevisiae | | | | 101355/ | cerevisiae | | | | cerevisiae |
| | | | 1) (EC | | | | | | | FGSC | | | | | |
| | | | 1.1.1.34) | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 0 | A0A117DXK2 | Homo- | | Asper- | modified | Q4WUL6 | Homo- | | Neo- | modified | J8Q3V7 | Homo- | Saccharo- | modified |
| 74 | | | citrate | | gillus | codon | | aconitase, | | sartorya | codon | | isocitrate | myces | codon |
| | | | synthase | | niger | usage for | | mitochon- | | fumigata | usage for | | dehydro- | arboricola | usage for |
| | | | | | | Coryne- | | drial (EC | | (strain | Coryne- | | genase | (strain H-6/ | Coryne- |
| | | | | | | bacterium | | 4.2.1.36) | | ATCC | bacterium | | | AS | bacterium |
| | | | | | | glutami- | | (Homo- | | MYA- | glutami- | | | 2.3317/ | glutami- |
| | | | | | | cum and | | aconitate | | 4609/ | cum and | | | CBS | cum and |
| | | | | | | Saccharo- | | hydratase) | | Af293/ | Saccharo- | | | 10644) | Saccharo- |
| | | | | | | myces | | | | CBS | myces | | | (Yeast) | myces |
| | | | | | | cerevisiae | | | | 101355/ | cerevisiae | | | | cerevisiae |
| | | | | | | | | | | FGSC | | | | | |
| | | | | | | | | | | A1100) | | | | | |
| | | | | | | | | | | (Asper- | | | | | |
| | | | | | | | | | | gillus | | | | | |
| | | | | | | | | | | fumigatus) | | | | | |
| Cg2OXAD_ | 436.5 | A0A0E4HH64 | Homo- | | Paenibacillus | modified | | | | | | | | | |
| 75 | | | citrate | | riograndensis | codon | | | | | | | | | |
| | | | synthase | | SBR5 | usage for | | | | | | | | | |
| | | | 1 (EC | | | Coryne- | | | | | | | | | |
| | | | 2.3.3.14) | | | bacterium | | | | | | | | | |
| | | | | | | glutami- | | | | | | | | | |
| | | | | | | cum and | | | | | | | | | |
| | | | | | | Saccharo- | | | | | | | | | |
| | | | | | | myces | | | | | | | | | |
| | | | | | | cerevisiae | | | | | | | | | |
| Cg2OXAD_ | 226.6 | A5UL49 | 2- | | Methanob- | modified | | | | | | | | | |
| 76 | | | isopropyl | | revibacter | codon | | | | | | | | | |
| | | | malate | | smithii | usage for | | | | | | | | | |
| | | | synthase, | | (strain | Coryne- | | | | | | | | | |
| | | | LeuA (EC | | ATCC | bacterium | | | | | | | | | |
| | | | 2.3.3.13) | | 35061/ | glutami- | | | | | | | | | |
| | | | | | DSM 861/ | cum and | | | | | | | | | |
| | | | | | OCM | Saccharo- | | | | | | | | | |
| | | | | | 144/PS) | myces | | | | | | | | | |
| | | | | | | cerevisiae | | | | | | | | | |
| Cg2OXAD_ | 215.5 | A0A150JKI3 | Putative | | Arc I | modified | | | | | | | | | |
| 77 | | | Homo- | | group | codon | | | | | | | | | |
| | | | citrate | | archaeon | usage for | | | | | | | | | |
| | | | synthase | | ADurb1113_ | Coryne- | | | | | | | | | |
| | | | AksA (EC | | Bin01801 | bacterium | | | | | | | | | |
| | | | 2.3.3.14) | | | glutami- | | | | | | | | | |
| | | | | | | cum and | | | | | | | | | |
| | | | | | | Saccharo- | | | | | | | | | |
| | | | | | | myces | | | | | | | | | |
| | | | | | | cerevisiae | | | | | | | | | |
| Cg2OXAD_ | 278 | V5IKX8 | Homo- | | Neurospora | modified | | | | | | | | | |
| 78 | | | citrate | | crassa | codon | | | | | | | | | |
| | | | synthase | | (strain | usage for | | | | | | | | | |
| | | | (Homo- | | ATCC | Coryne- | | | | | | | | | |
| | | | citrate | | 24698/ | bacterium | | | | | | | | | |
| | | | synthase, | | 74-OR23- | glutami- | | | | | | | | | |
| | | | variant 1) | | 1A/CBS | cum and | | | | | | | | | |
| | | | | | 708.71/ | Saccharo- | | | | | | | | | |
| | | | | | DSM | myces | | | | | | | | | |
| | | | | | 1257/ | cerevisiae | | | | | | | | | |
| | | | | | FGSC | | | | | | | | | | |
| | | | | | 987) | | | | | | | | | | |
| Cg2OXAD_ | 205.2 | A4G035 | 2- | | Methanococcus | modified | | | | | | | | | |
| 79 | | | isopropyl | | maripaludis | codon | | | | | | | | | |
| | | | malate | | (strain | usage for | | | | | | | | | |
| | | | synthase | | C5/ | Coryne- | | | | | | | | | |
| | | | (EC | | ATCC | bacterium | | | | | | | | | |
| | | | 2.3.3.13) | | BAA- | glutami- | | | | | | | | | |
| | | | | | 1333) | cum and | | | | | | | | | |
| | | | | | | Saccharo- | | | | | | | | | |
| | | | | | | myces | | | | | | | | | |
| | | | | | | cerevisiae | | | | | | | | | |
| Cg2OXAD_ | 0 | P05342 | Homo- | | Azotobacter | modified | | | | | | | | | |
| 80 | | | citrate | | vinelandii | codon | | | | | | | | | |
| | | | synthase | | | usage for | | | | | | | | | |
| | | | (EC | | | Coryne- | | | | | | | | | |
| | | | 2.3.3.14) | | | bacterium | | | | | | | | | |
| | | | | | | glutami- | | | | | | | | | |
| | | | | | | cum and | | | | | | | | | |
| | | | | | | Saccharo- | | | | | | | | | |
| | | | | | | myces | | | | | | | | | |
| | | | | | | cerevisiae | | | | | | | | | |
| Cg2OXAD_ | 0 | Q5KIZ5 | Homo- | | Cryptococcus | modified | | | | | | | | | |
| 81 | | | citrate | | neoformans | codon | | | | | | | | | |
| | | | synthase, | | var. | usage for | | | | | | | | | |
| | | | putative | | neoformans | Coryne- | | | | | | | | | |
| | | | | | serotype | bacterium | | | | | | | | | |
| | | | | | D | glutami- | | | | | | | | | |
| | | | | | (strain | cum and | | | | | | | | | |
| | | | | | JEC21/ | Saccharo- | | | | | | | | | |
| | | | | | ATCC | myces | | | | | | | | | |
| | | | | | MYA-565) | cerevisiae | | | | | | | | | |
| | | | | | (Filobasidiella | | | | | | | | | | |
| | | | | | neoformans) | | | | | | | | | | |
| Cg2OXAD_ | 237.8 | S6KZZ1 | NifV | | Pseudomonas | modified | | | | | | | | | |
| 82 | | | protein, | | stutzeri | codon | | | | | | | | | |
| | | | encodes a | | B1SMN1 | usage for | | | | | | | | | |
| | | | Homo- | | | Coryne- | | | | | | | | | |
| | | | citrate | | | bacterium | | | | | | | | | |
| | | | synthase | | | glutami- | | | | | | | | | |
| | | | | | | cum and | | | | | | | | | |
| | | | | | | Saccharo- | | | | | | | | | |
| | | | | | | myces | | | | | | | | | |
| | | | | | | cerevisiae | | | | | | | | | |
| Cg2OXAD_ | 289.6 | I2DYU9 | Homo- | | Burkholderia | modified | | | | | | | | | |
| 83 | | | citrate | | sp. | codon | | | | | | | | | |
| | | | synthase | | KJ006 | usage for | | | | | | | | | |
| | | | | | | Coryne- | | | | | | | | | |
| | | | | | | bacterium | | | | | | | | | |
| | | | | | | glutami- | | | | | | | | | |
| | | | | | | cum and | | | | | | | | | |
| | | | | | | Saccharo- | | | | | | | | | |
| | | | | | | myces | | | | | | | | | |
| | | | | | | cerevisiae | | | | | | | | | |
| Cg2OXAD_ | 411.2 | A0A126T608 | Homo- | | Methylomonas | modified | | | | | | | | | |
| 84 | | | citrate | | denitrificans | codon | | | | | | | | | |
| | | | synthase | | | usage for | | | | | | | | | |
| | | | | | | Coryne- | | | | | | | | | |
| | | | | | | bacterium | | | | | | | | | |
| | | | | | | glutami- | | | | | | | | | |
| | | | | | | cum and | | | | | | | | | |
| | | | | | | Saccharo- | | | | | | | | | |
| | | | | | | myces | | | | | | | | | |
| | | | | | | cerevisiae | | | | | | | | | |

Yl = Yarrowia lipolytica;
Bs = Bacillus subtilis;
Sc = Saccharomyces cerevisiae

What is claimed is:

1. An engineered microbial cell that expresses a heterologous homocitrate synthase, wherein the engineered microbial cell produces 2-oxoadipate.

2. The engineered microbial cell of claim 1, wherein the engineered microbial cell also expresses a heterologous homoaconitase.

3. The engineered microbial cell of claim 1 or claim 2, wherein the engineered microbial cell also expresses a heterologous homoisocitrate dehydrogenase.

4. The engineered microbial cell of any one of claims 1-3, wherein the engineered microbial cell expresses one or more additional enzyme(s) selected from an additional heterologous homocitrate synthase, an additional heterologous homoaconitase, or an additional heterologous homoisocitrate dehydrogenase.

5. An engineered microbial cell that expresses a non-native homocitrate synthase, wherein the engineered microbial cell produces 2-oxoadipate.

6. The engineered microbial cell of claim 5, wherein the engineered microbial cell also expresses a non-native homoaconitase.

7. The engineered microbial cell of claim 5 or claim 6, wherein the engineered microbial cell also expresses a non-native homoisocitrate dehydrogenase.

8. The engineered microbial cell of any one of claims 5-7, wherein the engineered microbial cell expresses one or more additional enzyme(s) selected from an additional non-native homocitrate synthase, an additional non-native homoaconitase, or an additional non-native homoisocitrate dehydrogenase.

9. The engineered microbial cell of 8, wherein the additional enzyme(s) are from a different organism than the corresponding enzyme in claims 5-7.

10. The engineered microbial cell of any of claims 5-9, wherein the engineered microbial cell comprises increased activity of one or more upstream 2-oxoadipate pathway enzyme(s), said increased activity being increased relative to a control cell.

11. The engineered microbial cell of any one of claims 5-10, wherein the engineered microbial cell comprises reduced activity of one or more enzyme(s) that consume one or more 2-oxoadipate pathway precursors, said reduced activity being reduced relative to a control cell.

12. The engineered microbial cell of claim 11, wherein the one or more enzyme(s) that consume one or more 2-oxoadipate pathway precursors comprise alpha-ketoglutarate dehydrogenase or citrate synthase.

13. The engineered microbial cell of claim 11 or claim 12, wherein the reduced activity is achieved by replacing a native promoter of a gene for the one or more enzymes that consume one or more 2-oxoadipate pathway precursors with a less active promoter.

14. An engineered microbial cell, wherein the engineered microbial cell comprises means for expressing a heterologous homocitrate synthase, wherein the engineered microbial cell produces 2-oxoadipate.

15. The engineered microbial cell of claim 14, wherein the engineered microbial cell also comprises means for expressing a heterologous homoaconitase.

16. The engineered microbial cell of claim 14 or claim 18, wherein the engineered microbial cell also comprises means for expressing a non-native homoisocitrate dehydrogenase.

17. An engineered microbial cell, wherein the engineered microbial cell comprises means for expressing a non-native homocitrate synthase, wherein the engineered microbial cell produces 2-oxoadipate.

18. The engineered microbial cell of claim 17, wherein the engineered microbial cell also comprises means for expressing a non-native homoaconitase.

19. The engineered microbial cell of claim 17 or claim 18, wherein the engineered microbial cell also comprises means for expressing a non-native homoisocitrate dehydrogenase.

20. The engineered microbial cell of any one of claims 14-19, wherein the engineered microbial cell comprises means for increasing the activity of one or more upstream 2-oxoadipate pathway enzyme(s), said increased activity being increased relative to a control cell.

21. The engineered microbial cell of any one of claims 14-20, wherein the engineered microbial cell comprises means for reducing the activity of one or more enzyme(s) that consume one or more 2-oxoadipate pathway precursors, said reduced activity being reduced relative to a control cell.

22. The engineered microbial cell of claim 21, wherein the one or more enzyme(s) that consume one or more 2-oxoadipate pathway precursors comprise alpha-ketoglutarate dehydrogenase or citrate synthase.

23. The engineered microbial cell of claim 21 or claim 22, wherein the reduced activity is achieved by means for replacing a native promoter of a gene for said one or more enzymes with a less active promoter.

24. The engineered microbial cell of any one of claims 5-23, wherein the engineered microbial cell comprises a fungal cell.

25. The engineered microbial cell of claim 24, wherein the engineered microbial cell comprises a yeast cell.

26. The engineered microbial cell of claim 25, wherein the yeast cell is a cell of the genus Saccharomyces.

27. The engineered microbial cell of claim 26, wherein the yeast cell is a cell of the species cerevisiae.

28. The engineered microbial cell of any one of claims 5-27, wherein the non-native homocitrate synthase comprises a homocitrate synthase having at least 70% amino acid sequence identity with a homocitrate synthase from Komagataella pastoris or Thermus thermophilus.

29. The engineered microbial cell of claim 28, wherein the engineered microbial cell comprises a non-native homocitrate synthase having at least 70% amino acid sequence identity with the homocitrate synthase from Komagataella pastoris and a non-native homocitrate synthase having at least 70% amino acid sequence identity with the homocitrate synthase from Thermus thermophilus.

30. The engineered microbial cell of claim 25, wherein the engineered microbial cell comprises a homocitrate synthase having at least 70 percent amino acid sequence identity to a homocitrate synthase from Schizosaccharomyces pombe (strain 972/ATCC 24843) (Fission yeast) (Uniprot ID No. Q9Y823; SEQ ID NO:90), having amino acid substitution D123N; a homoaconitase having at least 70 percent amino acid sequence identity to a homoaconitase from Saccharomyces cerevisiae (strain ATCC 204508/S288c) (Baker's yeast) (Uniprot ID No. P49367; SEQ ID NO:33); and a homoisocitrate dehydrogenase having at least 70 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11).

31. The engineered microbial cell of claim 30, wherein the engineered microbial cell is a Saccharomyces cerevisiae cell or a Yarrowia lipolytica cell.

32. The engineered microbial cell of any one of claims 7-23, wherein the engineered microbial cell is a bacterial cell.

33. The engineered microbial cell of claim 32, wherein the bacterial cell is a cell of the genus Corynebacterium.

34. The engineered microbial cell of claim 33, wherein the bacterial cell is a cell of the species glutamicum.

35. The engineered microbial cell of claim 34, wherein the non-native homocitrate synthase comprises a homocitrate synthase having at least 70% amino acid sequence identity with a homocitrate synthase selected from the group consisting of Thermus thermophilus, Saccharomyces cerevisiae, Candida dubliniensis, Ustilaginoidea virens, Schizosaccharomyces cryophilus, and Komagataella pastoris.

36. The engineered microbial cell of claim 35, wherein the non-native homocitrate synthase comprises a homocitrate synthase having at least 70% amino acid sequence identity with a homocitrate synthase from Thermus thermophilus or Saccharomyces cerevisiae.

37. The engineered microbial cell of claim 36, wherein the engineered microbial cell comprises a non-native homocitrate synthase having at least 70% amino acid sequence identity with the homocitrate synthase from Thermus thermophilus and a non-native homocitrate synthase having at least 70% amino acid sequence identity with the homocitrate synthase from Saccharomyces cerevisiae.

38. The engineered microbial cell of any one of claims 34-37, wherein the engineered microbial cell also expresses a non-native homoaconitase having at least 70% amino acid sequence identity with a homoaconitase selected from the group consisting of Ogataea parapolymorpha, Komagataella pastoris, Ustilaginoidea virens, Ceratocystis fimbriata f. sp. Platani, and Gibberella moniliformis.

39. The engineered microbial cell of claim 38, wherein the non-native homoaconitase comprises a homoaconitase having at least 70% amino acid sequence identity with a homoaconitase from Ogataea parapolymorpha.

40. The engineered microbial cell of any one of claims 34-39, wherein the wherein the engineered microbial cell also expresses a non-native homoisocitrate dehydrogenase having at least 70% amino acid sequence identity with a homoisocitrate dehydrogenase selected from the group consisting of Ogataea parapolymorpha, Candida dubliniensis, and Saccharomyces cerevisiae.

41. The engineered microbial cell of any one of claims 1-40, wherein the wherein the engineered microbial cell also expresses a non-native homoisocitrate dehydrogenase having at least 70% amino acid sequence identity with a homoisocitrate dehydrogenase from Ogataea parapolymorpha.

42. The engineered microbial cell of claim 34, wherein the engineered microbial cell comprises a homocitrate synthase having at least 70 percent amino acid sequence identity to a homocitrate synthase from Schizosaccharomyces pombe (strain 972/ATCC 24843) (Fission yeast) (Uniprot ID No. Q9Y823; SEQ ID NO:90), having amino acid substitution D123N; a homoaconitase having at least 70 percent amino acid sequence identity to a homoaconitase from Saccharomyces cerevisiae (strain ATCC 204508/S288c) (Baker's yeast) (Uniprot ID No. P49367; SEQ ID NO:33); and a homoisocitrated dehydrogenase having at least 70 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11).

43. The engineered microbial cell of claim 32, wherein the bacterial cell is a Bacillus subtilis cell.

44. The engineered microbial cell of claim 43, wherein the engineered microbial cell comprises a homocitrate synthase having at least 70 percent amino acid sequence identity to a homocitrate synthase from Saccharomyces cerevisiae (strain ATCC 204508/S288c) (Baker's yeast) (Uniprot ID No. P48570; SEQ ID NO:35); a homoaconitase having at least 70 percent amino acid sequence identity to a homoaconitase from Neosartorya fumigata (strain ATCC MYA-4609/Af293/CBS 101355/FGSC A1100) (Aspergillus fumigatus) (Uniprot ID No. Q4WUL6; SEQ ID NO:83), which includes a deletion of amino acid residues 2-41 and 721-777, relative to the full-length sequence; and a homoisocitrate dehydrogenase having at least 70 percent amino acid sequence identity to a homoisocitrate dehydrogenase from Saccharomyces cerevisiae (strain ATCC 204508/S288c) (Baker's yeast) (Uniprot ID No. P40495; SEQ ID NO:11).

45. The engineered microbial cell of any one of claims 5-41, wherein, when cultured, the engineered microbial cell produces 2-oxoadipate at a level at least 100 μg/L of culture medium.

46. The engineered microbial cell of claim 45, wherein, when cultured, the engineered microbial cell produces 2-oxoadipate at a level at least 20 mg/L of culture medium.

47. The engineered microbial cell of claim 46, wherein, when cultured, the engineered microbial cell produces 2-oxoadipate at a level at least 75 mg/L of culture medium.

48. A culture of engineered microbial cells according to any one of claims 5-47.

49. The culture of claim 48, wherein the substrate comprises a carbon source and a nitrogen source selected from the group consisting of urea, an ammonium salt, ammonia, and any combination thereof.

50. The culture of claim 48 or claim 49, wherein the engineered microbial cells are present in a concentration such that the culture has an optical density at 600 nm of 10-500.

51. The culture of any one of claims 48-50, wherein the culture comprises 2-oxoadipate.

52. The culture of any one of claims 48-51, wherein the culture comprises 2-oxoadipate at a level at least 100 μg/L of culture medium.

53. A method of culturing engineered microbial cells according to any one of claims 5-46, the method comprising culturing the cells under conditions suitable for producing 2-oxoadipate.

54. The method of claim 53, wherein the method comprises fed-batch culture, with an initial glucose level in the range of 1-100 g/L, followed controlled sugar feeding.

55. The method of claim 53 or claim 54, wherein the fermentation substrate comprises glucose and a nitrogen source selected from the group consisting of urea, an ammonium salt, ammonia, and any combination thereof.

56. The method of any one of claims 53-55, wherein the culture is pH-controlled during culturing.

57. The method of any one of claims 53-56, wherein the culture is aerated during culturing.

58. The method of any one of claims 53-57, wherein the engineered microbial cells produce 2-oxoadipate at a level at least 100 μg/L of culture medium.

59. The method of any one of claims 53-58, wherein the method additionally comprises recovering 2-oxoadipate from the culture.

60. A method for preparing 2-oxoadipate using microbial cells engineered to produce 2-oxoadipate, the method comprising:
(a) expressing a non-native homocitrate synthase in microbial cells;
(b) cultivating the microbial cells in a suitable culture medium under conditions that permit the microbial cells to produce 2-oxoadipate, wherein the 2-oxoadipate is released into the culture medium; and
(c) isolating 2-oxoadipate from the culture medium.


SEQUENCE LISTING (121 sequences)

SEQ ID NO: 1 - protein, 693 aa, Saccharomyces cerevisiae
MLRSTTFTRSFHSSRAWLKGQNLTEKIVQSYAVNLPEGKVVHSGDYVSIKPAHCMSHDNSWPVALKFMGLGATKIKNPSQIVTTLDHDIQNKSEKNLTKYKNIENFAKKHHIDHYPAGRGIGHQIMIEEGYAFPLNMTVASDSHSNTYGGLGSLGTPIVRTDAAAIWATGQTWWQIPPVAQVELKGQLPQGVSGKDIIVALCGLFNNDQVLNHAIEFTGDSLNALPIDHRLTIANMTTEWGALSGLFPVDKTLIDWYKNRLQKLGTNNHPRINPKTIRALEEKAKIPKADKDAHYAKKLIIDLATLTHYVSGPNSVKVSNTVQDLSQQDIKINKAYLVSCTNSRLSDLQSAADVVCPTGDLNKVNKVAPGVEFYVAAASSEIEADARKSGAWEKLLKAGCIPLPSGCGPCIGLGAGLLEPGEVGISATNRNFKGRMGSKDALAYLASPAVVAASAVLGKISSPAEVLSTSEIPFSGVKTEIIENPVVEEEVNAQTEAPKQSVEILEGFPREFSGELVLCDADNINTDGIYPGKYTYQDDVPKEKMAQVCMENYDAEFRTKVHPGDIVVSGFNFGTGSSREQAATALLAKGINLVVSGSFGNIFSRNSINNALLTLEIPALIKKLREKYQGAPKELTRRTGWFLKWDVADAKVVVTEGSLDGPVILEQKVGELGKNLQEIIVKGGLEGWVKSQL

SEQ ID NO: 2 - DNA, 2079 bp, Artificial
  mutated or codon-optimized sequence
atgcttcgttccaccacgttcactcgatcgttccactcttcccgtgcttggcttaaaggtcaaaacttgacagaaaaaattgtccagtcctatgcagttaatctgcccgaaggaaaggttgtgcattccggtgattacgtttcgatcaaaccagcacattgcatgtctcacgacaattcatggcccgtggcgctcaagttcatgggtctgggtgctaccaaaattaagaaccctagccagattgtgactaccctggaccatgatatccaaaataagtcggaaaaaaacttgacaaagtataaaaacatcgaaaactttgcaaaaaagcaccacatcgatcattatcccgcgggtcgcggtattggccaccagatcatgatcgaggaaggatacgcattcccacttaacatgactgtggcctctgattcacattccaacacatacggcggattgggctctttgggtacgcccatcgtccgcacggacgctgccgccatttgggctaccggacagacgtggtggcagatcccaccagttgcccaagttgagctcaagggacagctcccccagggagtgagcggtaaagatatcatcgtcgccctctgcggacttttcaataatgatcaagttctcaatcacgccatcgaattcaccggcgattccctcaacgctctcccaattgaccaccgtctcaccatcgcaaacatgactactgagtggggcgcattgagcggtcttttcccagtcgacaagactctgatcgattggtataaaaaccgtcttcagaagctgggaactaacaaccaccctcgaattaaccctaagaccatccgcgccctcgaagaaaaggcgaaaattccaaaagcagataaagatgcccactacgcaaaaaaacttatcatcgatcttgctactctgacccattacgtgtcaggccctaactccgtcaaagttagcaatacggtacaggatctctctcagcaagacattaaaatcaacaaagcgtatttggtctcctgtactaactcgcgcttgtcagatctgcagtccgccgcagatgtcgtgtgcccaaccggagaccttaacaaggttaacaaggtggcaccaggcgtcgaattttatgtggccgctgcgagcagcgagattgaggctgacgctcgaaagagcggagcctgggagaagctgcttaaagcaggttgcatccccctgccctccggctgcggtccgtgtatcggcctgggtgcgggcttgctcgaaccaggcgaagtaggtatttccgctacaaaccgcaacttcaaaggccgcatgggtagcaaagacgcactcgcgtacctcgcttccccagctgtcgttgccgcgtccgctgtgctcggtaagatttcatcacctgcggaagtcctgtcaacgagcgagatccccttttctggcgtcaagaccgagattatcgaaaaccccgtggtggaggaagaagtaaacgcacagactgaggcaccgaagcagtcggttgaaattttggaaggcttcccccgtgaattctctggagaacttgtcctttgcgatgcggacaatattaacaccgacggcatctatccaggaaagtatacttatcaagatgatgtcccgaaggaaaagatggcccaagtttgcatggaaaattacgatgctgaatttcgcacgaaggtccatcctggcgacattgtcgtttccggcttcaacttcggaactggtagctcccgtgagcaagcagcgacggccctcctggctaagggaatcaacttggttgtgtcgggctctttcggcaacatcttctctcgcaacagcatcaacaacgcgctccttaccctcgagattcctgctctgattaaaaaattgcgcgagaaataccagggcgcaccaaaggaactgacgcgccgcaccggatggtttctgaagtgggatgtggcagacgctaaagtcgttgtcacagagggctctctggacggcccagtcattttggagcagaaagtaggcgagctgggcaagaatctgcaggaaatcatcgtgaagggtggcttggaaggatgggtaaagagccaattg

SEQ ID NO: 3 - protein, 371 aa, Saccharomyces cerevisiae
MFRSVATRLSACRGLASNAARKSLTIGLIPGDGIGKEVIPAGKQVLENLNSKHGLSFNFIDLYAGFQTFQETGKALPDETVKVLKEQCQGALFGAVQSPTTKVEGYSSPIVALRREMGLFANVRPVKSVEGEKGKPIDMVIVRENTEDLYIKIEKTYIDKATGTRVADATKRISEIATRRIATIALDIALKRLQTRGQATLTVTHKSNVLSQSDGLFREICKEVYESNKDKYGQIKYNEQIVDSMVYRLFREPQCFDVIVAPNLYGDILSDGAAALVGSLGVVPSANVGPEIVIGEPCHGSAPDIAGKGIANPIATIRSTALMLEFLGHNEAAQDIYKAVDANLREGSIKTPDLGGKASTQQVVDDVLSRL

SEQ ID NO: 4 - DNA, 1113 bp, Artificial
  mutated or codon-optimized sequence
atgtttcgatccgtggcgacccgcctgtctgcatgccgtggcttggcaagcaacgctgctcgcaaatctttgacgatcggtctgatcccgggtgacggaattggcaaggaagtaattcccgctggtaagcaggtcctcgaaaatttgaactccaaacacggtctttcatttaatttcatcgatttgtacgccggctttcagacatttcaagagaccggcaaagctcttccagatgaaactgtgaaggtgctcaaggaacagtgtcaaggagctttgtttggagccgtacagtccccaaccacaaaggtggagggctactcatcccctatcgtcgctctccgccgcgaaatgggtctgttcgccaacgtgcgcccggtgaaaagcgtggagggtgagaaaggaaagccaatcgatatggttattgtgcgtgaaaataccgaggatttgtacattaagatcgaaaagacgtacatcgacaaagccaccggtacccgagtggccgatgcaacaaaacgcatctcagagatcgcaactcgccgtattgcaaccatcgctttggacattgcgctcaaacgcctccaaacccgcggtcaggcaactctgaccgttactcacaagagcaatgtgctgtcccagtccgacggtctgttccgcgagatctgcaaggaggtgtacgagtccaataaggataaatacggccaaattaagtacaatgaacagattgtggatagcatggtttatcgccttttccgcgaaccccaatgtttcgatgtcatcgtggcccctaacctgtacggagacatcctttcagacggtgctgcggctctggtgggctcgcttggcgtcgttccatctgccaacgtcggcccagaaatcgtcattggcgaaccatgccacggttccgcaccagacattgctggcaaaggcattgctaatcctattgccaccattcgttccactgcgctgatgttggagttcctgggacacaacgaagcggctcaggatatttacaaagcagtcgacgcaaacttgcgtgaaggatcgattaagaccccggatttgggcggaaaagctagcacacaacaagttgttgatgacgtgctttcccgcctt

SEQ ID NO: 5 - protein, 491 aa, Cryptococcus neoformans
MCPPADEPINAGADQDMVAIETNTPHISTSSANQVNGTTGTAEAQPPAVKTHKGLYGRASDFLSNTSNWKIIESTLREGEQFANAFFTLETKIKIAKMLDEFGVEYIELTSPAASPESRAHCEAICNLGLKRTKILTHIRCHMDDARLAVETGVDGVDVVIGTSSFLREHSHGKDMTWITKTAIEVIEFVKSKGIEIRFSSEDSFRSELVDLLSIYRTVDKIGVNRVGVADTVGCADARQVYELVRTLRGVVSCDIETHFHNDTGCAIANAYAALEAGATHVDTSILGIGERNGITPLGGLIARMMVADPEYVKSKYNLTMLRELENFVAEAVEVQVPFNNYITGFCAFTHKAGIHAKAILANPSTYEILNPADFGMTRYVSIGHRLTGWNAVKSRVEQLNLKLNDEQVKDATAKIKELADVRTQSMEDVDMILRIYHTGIQSGDLKVGQSAVLDRLLEKHMPSRDNSPSGRSSNADGTPAKRQRVGEPSA

SEQ ID NO: 6 - DNA, 1473 bp, Artificial
  mutated or codon-optimized sequence
atgtgcccaccagcagacgagccaattaacgcaggagcggatcaggatatggtggcgatcgaaacaaataccccacacatctctacctcgagcgcaaaccaggtgaacggcaccactggaaccgccgaggcccagccaccagccgtgaaaacgcataagggcctgtacggccgcgcgtccgacttcttgtctaacacatcgaactggaagattatcgagtctaccttgcgagagggtgaacagttcgccaacgctttcttcacattggagacgaagatcaagatcgcaaagatgttggatgagttcggcgttgagtatattgaactgacgagcccggcagccagccctgagtcccgtgcccactgcgaagctatttgcaacctgggattgaagcgcaccaaaatcttgacccacatccgctgtcatatggatgacgcacgtcttgctgttgagactggtgttgacggagtggatgtggtgatcggcacctcatccttcttgcgcgagcactctcatggtaaggacatgacgtggattaccaagaccgcaatcgaggtcatcgagttcgtgaagtccaagggtattgagattcgcttcagcagcgaagattccttccgttccgagttggtagatctgctttccatttaccgcaccgtggataaaatcggtgttaatcgtgtgggtgtggcagataccgtgggctgcgccgacgctcgtcaggtgtacgagctcgtacgcacattgcgcggcgtcgtgagctgcgatattgaaacccatttccacaacgataccggttgtgccattgctaatgcgtacgcagcactggaagcgggagccacgcacgtggatacctccattttgggcattggcgaacgcaacggtattactcctcttggcggcttgatcgcgcgcatgatggtcgccgatccagaatacgttaaatcaaagtataacctcaccatgctgcgcgaattggaaaactttgttgcagaggcagtcgaagttcaagtgcctttcaacaattacatcaccggcttctgcgcattcacgcacaaagcaggtattcatgctaaagctattttggccaacccgtctacctacgagattctgaacccagctgacttcggcatgacccgttacgtatcaatcggtcatcgactcacaggttggaatgcggtcaagtctcgcgtggagcaattgaacctgaagctcaacgacgaacaagtgaaagatgcaacagccaaaattaaagagctggcagacgtccgtacccagtccatggaagacgtggacatgattcttcgcatctaccataccggtatccaatcgggcgatctcaaagtgggacagagcgcagttcttgatcgccttctggaaaaacacatgccttcccgagataacagcccttcgggacgctcatccaacgccgatggaactcccgcaaagcgccagcgtgttggtgagccatctgcg

SEQ ID NO: 7 - protein, 392 aa, Unknown
  Arc I group archaeon ADurb1113_Bin01801
MKSFVSPYNFVENVNLKVDPKDIIVYDTTLRDGEQTPGICFTPDEKIDIAIKLDELGVNQIEAGFPVVSDGERRAIRNIMKQSLNSEILALTRTLKSDIDVALSCDVDGIITFIGASDLHLKYKLKMTREEALSKAVEAVEYGKSHGIFVAFTAEDSTRTDLGYLLELYKSTTEAGADRIHIADTTGSIRPMGMRYLVSQIKASIDNTIGVHCHDDFGLAVANSLAAFEAGARAISTSMNGLGERAGNASLEEIILGLRLLYGIEMPFKYEVIYELSRLVEKYTTMPVPKNKAVIGDNVFAHESGIHVLAVRAEPLTYEPYSPEFVGQKRRIILGKHCGMSCIDAKLEELRLSVPQSEKETLILKIKEMAERGAKVGDKEFKNMVQEILSKG

SEQ ID NO: 8 - DNA, 1176 bp, Artificial
  mutated or codon-optimized sequence
atgaagtccttcgtctcaccgtataactttgttgaaaacgtgaacctgaaggtggacccaaaggatatcattgtatatgacactactcttcgtgatggcgaacagacccctggcatctgctttacaccagatgagaaaatcgacatcgccattaaattggatgagctgggtgttaatcagatcgaggccggttttccagttgtctcggacggcgagcgacgtgctatccgcaacatcatgaaacaatccctgaactccgaaatcctggcgcttacgcgcacattgaagtctgatattgatgttgctttgtcatgcgatgtagatggcattatcacctttatcggagcgtcagacctccatctgaaatacaagttgaaaatgacccgagaagaagccctgtccaaggctgtcgaggcagtagaatacggcaagtcccacggaatcttcgttgcgtttaccgctgaggactctactcgcaccgatttgggctatctccttgaactttacaaatccaccaccgaggctggcgccgatcgcattcacatcgcggataccacaggcagcatccgccccatgggaatgcgctacctggtgtcacagatcaaagcctcaatcgataacacgattggcgttcactgccatgatgatttcggacttgcggtggcgaactccctggccgcttttgaggccggagcgcgcgccatctccaccagcatgaacggtttgggcgaacgtgccggcaacgccagccttgaggagatcattttgggcttgcgactgctgtacggcatcgagatgccattcaaatacgaggtcatttacgagctttcccgtctcgtggaaaagtacactactatgccagtcccgaagaacaaagctgtcatcggtgataacgttttcgcccatgaatctggcattcacgtacttgctgtgcgagcggaacccctcacctatgaaccatattcgccagagtttgttggtcaaaaacgccgcatcattctcggcaagcactgcggcatgtcatgcattgacgccaagttggaagaacttcgactgtccgtcccgcagtccgagaaggagaccttgattctcaagattaaagagatggccgaacgtggtgcaaaggtgggcgataaggagtttaagaacatggtgcaggaaatcttgtcgaaggga

SEQ ID NO: 9 - protein, 371 aa, Saccharomyces arboricola
MFRSVATRLSACRGLATNATRKSLTIGLIPGDGIGKEVIPAGRQVLENLNSKHGLNFDFIDLYAGFQTFQETGKALPDETIKVLKEQCQGALFGAVQSPTTKVEGYSSPIVALRREMGLFANVRPVKSVEGTKDKPIDMVIVRENTEDLYIKIEKTYIDKATGTRVADATKRISEIATRRIATIALDIALQRLQTRGQATLTVTHKSNVLSQSDGLFREICKEVYESNKDKYGQIKYNEQIVDSMVYRLFREPQCFDVIVAPNLYGDILSDGAAALVGSLGVVPSANVGPEIVIGEPCHGSAPDIAGKGIANPIATIRSTALMLEFLGHNDAAQDIYKAVDANLREGTITTPDLGGKASTQQVVDDVLSKL

SEQ ID NO: 10 - DNA, 1113 bp, Artificial
  mutated or codon-optimized sequence
atgttccgttccgttgccacccgtctgtccgcttgccgtggactcgcaaccaacgccacccgtaaatctctcacgattggtctgattccgggagatggaatcggcaaggaagtcatccccgccggacgtcaagtgcttgagaacttgaactctaagcacggccttaactttgacttcatcgatctgtacgccggatttcaaacctttcaggaaaccggtaaagctctgccagacgagaccatcaaggttttgaaggaacagtgccagggagcgctgtttggcgccgttcagtctcctaccaccaaagttgagggctattcgtctcctatcgtggcccttcgccgtgagatgggtttgttcgcaaatgttcgtcctgtcaaatctgtagaaggcaccaaggataaacccattgacatggttatcgtgcgagaaaacactgaagatctttatatcaagattgagaagacatacatcgacaaggcaaccggcacgcgcgttgccgatgcgacaaaacgcatttcggaaatcgccacccgacgcattgccaccatcgccctcgatatcgcacttcaacgcctgcaaacacgcggccaagctaccctcaccgtgacccacaagtccaacgtcttgtcacagtcagatggactgttccgcgaaatttgtaaagaagtttatgaatctaacaaagataaatatggtcaaatcaaatacaacgaacagattgtcgatagcatggtctaccgcctcttccgcgaacctcagtgtttcgatgttatcgtcgccccgaacctgtacggagacatcttgtccgacggtgcagccgcattggtgggctccctgggtgtggttcctagcgcaaacgtgggcccggaaattgtaatcggcgagccgtgccatggttctgctcccgatattgcgggtaagggcattgctaacccaattgctaccatccgctccaccgcactcatgctcgaattcctgggtcacaacgatgcagcccaggatatctacaaggcagtggatgcaaatctccgtgagggcaccatcaccaccccagacctgggtggcaaggcaagcacccagcaagtcgtggatgatgtcttgagcaagctt

SEQ ID NO: 11 - protein, 357 aa, Saccharomyces cerevisiae
MASNAARKSLTIGLIPGDGIGKEVIPAGKQVLENLNSKHGLSFNFIDLYAGFQTFQETGKALPDETVKVLKEQCQGALFGAVQSPTTKVEGYSSPIVALRREMGLFANVRPVKSVEGEKGKPIDMVIVRENTEDLYIKIEKTYIDKATGTRVADATKRISEIATRRIATIALDIALKRLQTRGQATLTVTHKSNVLSQSDGLFREICKEVYESNKDKYGQIKYNEQIVDSMVYRLFREPQCFDVIVAPNLYGDILSDGAAALVGSLGVVPSANVGPEIVIGEPCHGSAPDIAGKGIANPIATIRSTALMLEFLGHNEAAQDIYKAVDANLREGSIKTPDLGGKASTQQVVDDVLSRL

SEQ ID NO: 12 - DNA, 1071 bp, Artificial
  mutated or codon-optimized sequence
atggcctcgaacgctgcgcgtaagtccctgaccattggactcattcctggtgacggaatcggtaaggaagtgattccagcaggcaagcaggtcttggaaaatctgaactcgaagcacggtctgtcctttaactttattgacctttacgcgggattccagacctttcaggaaaccggtaaggccttgccagatgagaccgttaaagtcctgaaagaacaatgccagggcgcactttttggtgcggtgcaatcaccgaccacgaaggtggaaggttactcaagccctatcgttgcgcttcgtcgcgagatgggactgttcgcaaacgttcgccctgtgaagtccgttgagggagaaaaaggaaagcccatcgacatggtgattgtccgagaaaacacagaagatctctacatcaagatcgagaagacctacatcgataaggcaactggtacacgagtagcagatgcaactaagcgtatctctgaaattgcgactcgccgtatcgcaaccatcgcgcttgacatcgctctgaagcgcttgcagacccgtggccaggccaccctgaccgttacacacaagtctaatgtgctcagccagtccgatggtcttttccgagagatctgtaaggaggtgtatgaatccaacaaagacaaatacggccaaatcaagtataatgagcagatcgtggactcaatggtttatcgcctgtttcgcgaacctcaatgtttcgatgtgatcgtggcacctaacctgtatggagacattcttagcgacggcgctgccgccctggtgggttccctgggcgtggtgccatctgctaacgtgggccccgagatcgtcattggcgaaccttgccacggatcagcaccagatatcgcaggtaagggtatcgcaaaccccatcgctacgatccgatcaaccgcattgatgctcgaatttcttggtcacaatgaggcagctcaggatatctacaaggctgtggatgcaaaccttcgtgaaggaagcatcaaaaccccagacttgggtggaaaggcatcgacacaacaggtcgtggacgatgtcttgtcccgcctg

SEQ ID NO: 13 - protein, 386 aa, Methanococcus maripaludis
MDWKAVSPYNPKLNLKDCYLYDTTLRDGEQTPGVCFTHDQKLEIAKKLDELKIKQIEAGFPIVSENERKAIKSITGEGLNAQILALSRVLKEDIDKAIECDVDGIITFIAASPMHLKYKLHKSLDEVEEMGMKAVEYAKDHGLFVAFSAEDATRTPVEDLIRIHKNAEEHGANRVHIADTLGCATPQAMYHICSELSSNLKKAHIGVHCHNDFGFAVINSIYGLIGGAKAVSTTVNGIGERAGNAAIEEIVMALKVLYDHDMGLNTEILTEISKLVENYSKIRIPENKPLVGEMAFYHESGIHVDAVLENPLTYEPFLPEKIGQKRKIILGKHSGCRAVAHRLQELGLEASREELWEIVKKTKETREEGTEISDEVFKNIVDKIIK

SEQ ID NO: 14 - DNA, 1158 bp, Artificial
  mutated or codon-optimized sequence
atggattggaaagcagtgtctccatacaaccctaagctgaacttgaaggactgttacctttacgatacaaccctccgtgatggcgaacagactcctggagtctgtttcacacacgatcagaaacttgagattgcaaagaaactggacgaactgaaaattaagcagatcgaggcgggcttccctattgtctcggaaaatgaacgcaaagctattaaatcaatcaccggcgagggtttgaatgctcagatcttggcattgtcgcgtgtcctgaaggaagatattgataaggctatcgaatgtgacgtcgacggcatcattacgttcatcgccgcatcaccaatgcaccttaaatacaaactgcataagtccctggatgaagtcgaggaaatgggtatgaaggctgtggagtacgctaaggatcatggcctctttgtcgcctttagcgccgaagacgcgactcgtaccccagtggaggatctcatccgaatccacaagaatgccgaagaacatggagccaaccgcgtccacatcgcggacaccttgggttgcgctacgccgcaggcgatgtatcatatttgttccgagttgtcatcgaaccttaaaaaggctcacatcggcgttcactgccacaatgactttggcttcgctgtcatcaactcaatctacggcttgatcggcggcgccaaggcagtttcaactactgtcaacggtatcggtgaacgcgctggcaatgccgcaattgaagaaatcgtcatggccctcaaggtgctctacgatcacgatatgggcctcaacaccgagatcctgaccgagatcagcaaactggtggagaactactctaagatccgcattccagagaataagccacttgtcggcgaaatggccttttaccacgaatccggcattcatgtcgatgctgttcttgaaaacccgctcacatacgagcccttcttgcctgaaaaaatcggtcaaaagcgtaagatcatcctgggcaagcactccggctgtcgcgctgtcgcgcatcgcctccaggaattgggcttggaagcgtcccgcgaagaactctgggaaatcgtgaagaaaaccaaggaaacacgcgaagagggcacggaaatctcggatgaggtgtttaagaacattgttgacaaaatcatcaaa

SEQ ID NO: 15 - protein, 475 aa, Arthroderma gypseum
MATDTGSRPCCSSHTSDDNNTNGVTVAANGNHEGFTAVQTRQNPHPHVSRNPYGHNAGVTDFLSNVSRFKIIESTLREGEQFANAFFDTEKKIEIAKALDDFGVDYIELTSPCASEQSRKDCEAICKLGLKAKILTHIRCHMDDARIAVETGVDGVDVVIGTSSYLREHSHGKDMTYIKNTAIEVINFVKSKGIEVRFSSEDSFRSDLVDLLSIYSAVDQVGVNRVGIADTVGCASPRQVYELVRVLRGVVKCDIEIHLHNDTGCAIANAYCALEGGATHIDTSVLGIGERNGITPLGGLMARMIAADRDYVLSKYKLDKIKDIEDLVAEAVQVNIPFNNPITGFCAFTHKAGIHAKAILNNPSTYEIINPADFGMTRYVHFASRLTGWNAIKSRAQQLNLDMTDAQYKECTAKIKALADIRPIAVDDADSIIRAYHRNIKLGEDKPLLDLTEEQTAEFAAKQKEMAENGVEVSA

SEQ ID NO: 16 - DNA, 1425 bp, Artificial
  mutated or codon-optimized sequence
atggcaaccgacaccggctctcgcccgtgctgttcttcgcacacgtcggatgataataacaccaacggtgttaccgtggccgctaacggcaatcacgaaggcttcactgcagtgcagactcgccaaaacccccacccacacgtttcccgcaacccatacggtcacaacgctggtgtaaccgattttctctcgaacgtgtcccgattcaaaattatcgagtccacccttcgtgaaggcgaacagtttgccaacgctttcttcgacaccgagaagaagattgagatcgctaaggcattggatgatttcggcgtagactatatcgagctcacctcgccatgcgcatcagaacagtctcgtaaggattgtgaagctatttgcaagctgggcttgaaggccaagatcctcacccatattcgctgccacatggacgatgcacgtatcgcggttgagaccggtgttgatggtgtcgacgttgtgatcggcacaagcagctacctgcgcgaacactcccacggcaaggacatgacctacatcaagaacaccgccatcgaagtaatcaacttcgttaagtcaaaaggcattgaagtccgattctcctctgaagacagcttccgatcagatctggtggatctgcttagcatctattccgctgtcgaccaggtgggcgtgaaccgtgtgggcatcgcggatacggttggttgcgcttcccctcgtcaggtctacgagctggtacgcgtgttgcgtggagtggtgaagtgcgatattgagatccaccttcataacgacaccggctgcgcgatcgcgaacgcatattgtgccctcgaaggcggagccacacacattgacacctccgtgttgggtattggtgaacgcaatggcatcactcctcttggcggtttgatggcccgcatgatcgcagcagatcgcgactacgtcctttctaaatataaacttgacaagattaaagatatcgaggatcttgtcgcagaggccgtgcaggtaaacatcccattcaataacccgattaccggtttctgtgcgttcacacacaaggcaggaatccacgcgaaggccattttgaacaatccctccacatacgagatcatcaacccagccgacttcggcatgacccgttatgttcattttgcctcccgcctcaccggatggaacgcaattaaatcacgcgcacagcagcttaacttggacatgaccgacgcgcagtacaaggagtgcaccgcaaaaatcaaagcactggcagatatccgtccaatcgcagtcgacgatgcagacagcattatccgcgcataccaccgcaacatcaaactcggcgaggacaaacccctgcttgatcttaccgaagagcagaccgccgaattcgcagcgaagcaaaaagaaatggcggaaaacggagtggaagtgtccgca

SEQ ID NO: 17 - protein, 376 aa, Anaeromyxobacter dehalogenans
MTKSTWKLIDSTLREGEQFAHGTFRTEDKLEIARALDTFGVEYVEVTSPAASPQSQRDAVQIVKLGLGARVITHSRCVLDDVRAAIDTGVRGIGLLFATSRILRESSHGKSIQQIIDAMGPPIELALKAGLETRFSAEDAFRSEVTDLVAVYQAAERLGVHRVGIADTVGIATPRQVFALVREIRRAVRCDIGFHGHNDTGCAIANAYEALAAGATHVDVAVLGIGERVGITPLGGIIARMFSIEPQSVAERYRLGQLRELERLVARVTGIEVPFNNYVTGETAYSHKAGMHLKAMMANPGSYEIIPPEAFGLTRRLILGSRLTGRHAIAYRAREMGITFGESELKALTKRIKELADAGELSEEQIDRVLRDWVTA

SEQ ID NO: 18 - DNA, 1128 bp, Artificial
  mutated or codon-optimized sequence
atgaccaaatccacctggaaactcatcgattccacactccgtgaaggagaacagtttgcacatggaaccttccgtaccgaagacaaactggagattgctcgcgcactcgatactttcggagtggaatacgtagaggtgacctcaccggctgcgtcgccgcagagccagcgcgatgcagtccagattgtcaagctcggcttgggcgcacgcgtcatcactcactcgcgctgcgttctggacgacgtccgtgccgcgattgacaccggcgttcgaggcatcggcttgctgttcgctacctcacgcattttgcgtgaatctagccatggtaagagcatccaacagattattgacgcgatgggcccaccaatcgaactggccctcaaggcgggcctggaaactcgcttctccgcagaggacgcttttcgttctgaagtgacggatctggtggccgtttaccaggcagctgaacgtctgggcgtgcaccgcgttggcatcgcagacaccgttggcatcgctacgcctcgccaggtgttcgcgctggtgcgcgagatccgtcgcgctgttcgctgtgacatcggcttccacggccacaatgatactggctgtgccatcgcaaacgcctacgaggcactggcagcgggcgcgacacatgtggatgtcgctgttctcggtatcggagagcgcgtcggcatcaccccgttgggcggtatcattgcgcgcatgttttccatcgaacctcaatccgtcgcagagcgttaccgtctgggacagctccgtgagctcgagcgtctcgtcgcccgagtcacaggtatcgaagtgcccttcaacaactacgtcaccggcgaaacggcttactctcataaggcaggaatgcatctcaaagctatgatggctaacccgggttcgtatgaaattatccctcctgaagcattcggccttacccgtcgattgatcttgggcagccgccttaccggccgccacgctattgcctaccgtgcccgcgagatgggcatcaccttcggcgagtcggaactgaaggcattgaccaagcgtattaaagaactggccgacgccggcgaactgtctgaggagcagatcgatcgcgtgctccgcgattgggtgacggcg

SEQ ID NO: 19 - protein, 418 aa, Artificial
  mutated or codon-optimized sequence
MSVSEANGTETIKPPMNGNPYGPNPSDFLSRVNNFSIIESTLREGEQFANAFFDTEKKIQIAKALDNFGVDYIELTSPVASEQSRQDCEAICKLGLKCKILTHIRCHMDDARVAVETGVDGVDVVIGTSQYLRKYSHGKDMTYIIDSATEVINFVKSKGIEVRFSSEDSFRSDLVDLLSLYKAVDKIGVNRVGIADTVGCATPRQVYDLIRTLRGVVSCDIQCHFHNDTGMAIANAYCALEAGATHIDTSILGIGERNGITPLGALLARMYVTDREYITHKYKLNQLRELENLVADAVEVQIPFNNYITGMCAFTHKAGIHAKAILANPSTYEILKPEDFGMSRYVHVGSRLTGWNAIKSRAEQLNLHLTDAQAKELTVRIKKLADVRTLAMDDVDRVLREYHADLSDADRITKEASA

SEQ ID NO: 20 - DNA, 1254 bp, Artificial
  mutated or codon-optimized sequence
atgtccgtttccgaagccaatggcactgagactatcaagcctcctatgaacggaaacccctacggccctaacccatccgactttctctcccgcgtgaataacttctccatcatcgaatccaccctccgagaaggagagcaattcgcaaacgctttctttgacaccgagaagaaaatccaaatcgccaaggcactggataactttggagtggattacatcgagctgacctcacccgttgcttctgaacagtcccgccaagactgcgaggctatctgcaagctcggtctcaaatgtaagattctgacccacattcgctgccacatggacgatgcccgtgtggccgtcgaaactggcgtggatggcgttgacgtcgtcatcggcacatcccagtacctccgcaagtactcacacggcaaggatatgacctacatcatcgattccgcgacagaagtgattaactttgtcaagtcgaagggtatcgaagtgcgcttctcctcagaagactccttccgctcagatctggtggatcttctctcgctgtacaaggcggttgacaagatcggtgtcaatcgtgtcggaattgcagataccgtaggctgcgcaactccccgtcaggtttacgatctgatccgtactctccgtggcgtggtttcatgcgatatccaatgccatttccacaacgacacgggaatggctattgccaacgcatactgtgctttggaagcaggtgctacacacatcgataccagcatcctgggcatcggagaacgcaacggaattacgcccctgggtgctctcttggcgcgtatgtacgtgactgaccgcgaatacatcacccacaagtataaattgaatcaactgcgcgaactggaaaaccttgttgcggacgctgtggaagtccaaatcccgtttaataactatattaccggtatgtgcgcattcacccacaaggctggcatccacgcgaaggcaattctcgcaaacccatctacttacgagattttgaaaccggaagacttcggtatgtcacgatatgttcacgtgggatcccgcctgacgggctggaacgctatcaaatctcgtgccgaacagctcaatctgcacttgaccgatgcacaggcaaaggagctgaccgtgcgcattaagaaattggcggatgtccgtaccctggcgatggacgatgttgaccgcgttctccgcgaatatcacgctgatctgagcgacgccgatcgaattaccaaagaggcatctgct

SEQ ID NO: 21 - protein, 463 aa, Aspergillus niger
MCPGADHEPNGQANAANGRNGEHPGFTAVETRQNPHPSVSRNPYGHNVGVTDFLSNVSRFQIIESTLREGEQFANAFFDTEKKIEIAKALDEFGVDYIELTSPCASEQSRKDCEAICKLGLKAKILTHIRCHMDDARIAVETGVDGVDVVIGTSSYLREHSHGKDMTYIKNTAIEVIEFVKSKGIEIRFSSEDSFRSDLVDLLSIYSAVDKVGVNRVGIADTVGCASPRQVYELVRVLRGVVSCDIETHFHNDTGCAIANAYCALEAGATHIDTSVLGIGERNGITPLGGLMARMMVADPEYVKGKYRLEKLKDIEDLVAEAVEVNIPFNNYITGFCAFTHKAGIHAKAILNNPSTYEIINPADFGMSRYVHFASRLTGWNAIKSRAQQLKIEMTDDQYKECTAKIKALADIRPIAVDDADSIIRAYYRNLKLGENKPLLDLTADEQAQFAAKEKELAAQASA

SEQ ID NO: 22 - DNA, 1389 bp, Artificial
  mutated or codon-optimized sequence
atgtgcccgggcgccgaccatgagcctaacggccaggcaaacgctgcaaacggacgcaatggcgagcaccccggatttaccgctgttgagacgcgtcaaaacccacatccttctgttagccgtaacccgtacggtcacaacgtaggtgtcacagatttcttgtccaacgtaagccgctttcagattattgaatctacccttcgcgagggcgagcagttcgccaacgccttctttgacaccgaaaaaaaaattgagatcgccaaagcacttgatgagttcggcgtggattacatcgagctcacttctccttgcgcatccgaacagtctcgcaaagactgcgaggcaatttgcaagttgggtctcaaagcgaaaatccttacgcacatccgatgccacatggacgacgcacgaatcgcagttgaaaccggtgttgatggcgtggacgtagttatcggtacctccagctacctccgcgaacactcacacggcaaggatatgacctacatcaaaaacaccgcgattgaagtaatcgagtttgtcaaatccaagggaatcgagatccgcttctcatctgaggattccttccgcagcgatctcgttgatctgctttccatttattctgctgttgacaaggtaggtgtcaatcgcgtcggtatcgcggatacagtgggttgtgcctcccctcgacaagtgtacgaactcgtgcgtgtgttgcgcggtgtcgtgtcctgtgatattgagacccactttcacaacgataccggttgcgcaatcgcgaatgcttactgcgctctggaagccggagcgacccatattgatacctccgtgttgggcattggagaacgcaacggtattacacccctcggtggtctgatggcacgtatgatggtcgccgaccccgaatacgtgaaaggcaagtaccgcctggagaagctcaaagatatcgaggacctcgtggccgaagcagtggaggttaatattccctttaacaattatatcactggtttttgcgccttcactcacaaagccggtatccacgctaaggccatccttaacaacccgagcacgtacgaaattattaatccagctgacttcggcatgtcacgttacgttcacttcgcttcgcgccttacgggatggaacgctattaagtcccgtgcccagcagcttaagattgagatgacggacgaccaatataaggaatgcaccgctaagatcaaagctctggcggatattcgtccaatcgctgtggacgacgcggacagcattatccgcgcctactaccgaaacctgaaactcggcgaaaacaagccactgcttgatctgacggcggacgagcaggctcaattcgcggccaaagagaaggaactcgcggctcaagcatcagcg

SEQ ID NO: 23 - protein, 382 aa, Marinithermus hydrothermalis
MTNPRSLGSWAIIDSTLREGEQFERAHFSTGDKVEIARALDAFGIEYIEVTTPVASPQSARDAKVLANLNLKAKVITHIQCRKEAAWAALETGVQGIDLLFGTSKYLRAAHGRDIPRIIEEALEVIELIREQAPEVEVRFSAEDTFRSDEHDLLTVYRSVAPYVNRVGLADTVGVATPRQVYALVREVRRAVGPEVDIEFHGHNDTGCAVANAYEALEAGATHVDTSILGIGERNGITPLGGFLARMYTLQPEYVAGKYRLEMLPELDRMIARMTGIQIPFNNYITGESAFSHKAGMHLKAIYLNPQAYEAYPPEAFGLSRKLIIGSRLTGRAAIKKRAEELGLSFGEEELQRITHRIKALADQGTLTLEELDRILREWVLA

SEQ ID NO: 24 - DNA, 1146 bp, Artificial
  mutated or codon-optimized sequence
atgaccaatcctcgtagcctgggctcatgggcaatcattgactcgaccttgcgcgaaggtgaacagttcgagcgcgcacacttcagcaccggagataaggtggagattgcccgtgcgcttgacgcatttggtatcgaatacattgaggtaactactccggtagcatctccacagtccgcgcgtgatgcaaaggtgctcgcaaatctcaaccttaaagccaaggttatcacacacatccaatgccgcaaggaagcagcttgggccgccctcgaaaccggagtgcaaggtatcgacctcctgttcggtacctctaagtacctgcgcgcggcccacggacgtgatattccacgcattatcgaggaagccttggaagtcatcgagttgatccgcgagcaggcccccgaagtggaagtgcgattttcagcggaggataccttccgctccgatgagcacgaccttctgacagtttaccgtagcgttgctccttacgtgaaccgcgtgggcctggcggacacagtaggcgtagccactcctcgtcaagtctatgcattggtgcgtgaggtccgacgagccgtcggccctgaagtcgacattgaatttcacggccacaatgacaccggctgtgccgtggcgaacgcatacgaggccttggaagccggcgccacgcacgtggatactagcatcctgggcatcggagaacgtaacggaattactcctcttggcggctttttggcccgcatgtacaccctccagcctgagtatgttgcgggaaaataccgccttgaaatgttgcctgagttggatcgcatgatcgcccgtatgaccggcatccagattcccttcaataactacatcactggcgagagcgcattctcacacaaagcaggaatgcatctgaaggctatctacctgaacccacaggcgtacgaggcgtaccctccggaagccttcggcttgtcccgcaaattgattattggatcccgtttgactggtcgtgcagcaattaagaagcgcgccgaggaactgggattgtcttttggcgaagaggagttgcaacgaatcacccatcgtatcaaggctctcgctgatcagggtaccttgacactggaggaacttgatcgtattctgcgtgaatgggtacttgct

SEQ ID NO: 25 - protein, 418 aa, Artificial
  mutated or codon-optimized sequence
MSVSEANGTETIKPPMNGNPYGPNPSDFLSRVNNFSIIESTLREGEQFANAFFDTEKKIQIAKALDNFGVDYIELTSPVASEQSRQDCEAICKLGLKCKILTHIRCHMDDARVAVETGVDGVDVVIGTSQYLRKYSHGKDMTYIIDSATEVINFVKSKGIEVRFSSEDSFRSDLVDLLSLYKAVDKIGVNRVGIADTVGCATPRQVYDLIRTLRGVVSCDIECHFHNDTGMAIANAYCALEAGATHIDTSILGIGERNGITPLGALLARMYVTDREYITHKYKLNQLKELENLVADAVEVQIPFNNYITGMCAFTHKAGIHAKAILANPSTYEILKPEDFGMSRYVHVGSRLTGWNAIKSRAEQLNLHLTDAQAKELTVRIKKLADVRTLAMDDVDRVLREYHADLSDADRITKEASA

SEQ ID NO: 26 - DNA, 1254 bp, Artificial
  mutated or codon-optimized sequence
atgtcagtatctgaagctaatggcaccgaaacaatcaagcctccgatgaacggcaacccctatggccctaacccgtccgattttttgtcccgcgtcaataatttttcaattatcgaaagcacccttcgtgaaggcgaacagtttgctaacgcattcttcgataccgagaagaagatccagatcgcaaaggcactcgataactttggcgtcgattacatcgagctcactagcccagtggcaagcgagcagtcacgccaagattgtgaagcgatttgcaagctcggcctgaagtgcaagatcttgacccacatccgttgccatatggacgacgcacgcgtcgccgtagagactggcgttgacggtgttgacgtagtgattggcacatcacaatatctccgcaagtattcccatggaaaagatatgacatatattattgattccgctacagaggttattaactttgtcaaatccaagggaatcgaagtgcgcttttcgtccgaagattcgttccgttccgacttggtggatctcctgtccctctacaaggcggtcgacaagatcggtgttaaccgcgtcggcattgccgacacggttggttgtgcaacaccccgacaagtgtacgatcttatccgcaccctgcgcggcgtggtctcatgtgacatcgaatgtcatttccacaacgatacaggaatggcgatcgcaaacgcatattgcgcactcgaagccggagctacgcatatcgacactagcatcctcggtattggtgaacgcaacggcatcaccccattgggagcgcttttggcccgtatgtacgtgactgatcgcgaatacatcacccacaaatacaaactgaaccaactgaaggagctggaaaaccttgtcgctgacgctgttgaagtacagattccgttcaacaattatatcacaggcatgtgtgcattcacccacaaggctggaatccacgctaaagccatcctcgcgaacccatctacgtatgaaatcctcaagcctgaagacttcggaatgtctcgctacgtccatgttggctctcgcctcactggatggaacgcaattaaatctcgcgcagaacagttgaatttgcacctgacagacgcccaggccaaggaactcactgtgcgcattaagaagcttgctgatgtgcgtacattggcgatggacgacgtagatcgcgtccttcgagagtaccacgcagacctgagcgatgctgaccgaattacaaaagaggcgtccgct

SEQ ID NO: 27 - protein, 371 aa, Saccharomyces cerevisiae; identical to SEQ ID NO: 3

SEQ ID NO: 28 - DNA, 1113 bp, Artificial
  mutated or codon-optimized sequence
atgttccgtagcgttgcgacgcgcctgtcagcgtgtcgcggcctggcgtctaacgctgcccgcaaatcactgaccatcggtttgattcccggagacggaatcggcaaggaagttattcccgccggaaagcaagtccttgaaaaccttaactctaagcacggcctctcatttaattttattgatttgtatgcgggattccagactttccaggagaccggtaaggcattgcccgacgaaaccgtgaaagtcctgaaggaacagtgccaaggagcattgttcggtgcggtgcagtccccaaccacaaaggtggaaggttactcctcacccatcgtcgcgttgcgccgagaaatgggcctgttcgcgaatgtccgcccagtcaagtcagtggaaggcgagaagggtaaaccaattgacatggttatcgtgcgtgaaaacaccgaggacctctacatcaaaattgaaaagacttacatcgataaggcaactggaactcgcgtagcggatgcgacgaaacgtatcagcgaaatcgctacccgtcgcattgccactattgccctggacattgcgttgaagcgccttcaaacgcgcggccaagctactctcaccgtcacgcataaatccaacgtgctgtcccagagcgacggtctgttccgtgagatttgcaaggaagtgtatgaatctaacaaagataaatatggacaaatcaaatataacgagcagattgtggactcaatggtctatcgcttgttccgcgagcctcaatgcttcgacgtcattgttgctcccaatctgtacggagatatcctttccgatggtgccgccgcgcttgtaggctcactgggcgtggttccgtcggcaaacgtaggtccagaaatcgtgatcggagagccatgtcatggttctgcaccagatattgctggaaagggcatcgccaaccctatcgccaccatccgttccaccgccttgatgctcgagtttctgggacataatgaagctgctcaggacatttacaaagcggtagacgcaaacctgcgtgagggttcaattaagactcccgacctgggaggtaaagcaagcacacagcaagtggtcgatgatgtcctttctcgcctt

SEQ ID NO: 29 - protein, 475 aa, Trichophyton equinum
MATDTGSRPCCSSHTGDDNNTNGVTIAPNGNHEGFTAVQTRQNPHPHVSRNPYGHNAGVTDFLSNVSRFKIIESTLREGEQFANAFFDTEKKIEIAKALDDFGVDYIELTSPCASEQSRKDCEAICKLGLKAKILTHIRCHMDDARIAVETGVDGVDVVIGTSSYLREHSHGKDMTYIKNTAIEVINFVKSKGIEVRFSSEDSFRSDLVDLLSIYSAVDQVGVNRVGIADTVGCASPRQVYELVRVLRGVVKCDIEIHLHNDTGCAIANAYCALEGGATHIDTSVLGIGERNGITPLGGLMARMIAADRDYVLSKYKLEKIKDIEDLVAEAVQVNIPFNNPITGFCAFTHKAGIHAKAILNNPSTYEIINPADFGMTRYVHFASRLTGWNAIKSRAQQLNLDMTDAQYKECTAKIKALADIRPIAVDDADSIIRAYHRNIKLGENKPLLELTEEQTAEFAAKEKELAENGVEASA

SEQ ID NO: 30 - DNA, 1425 bp, Artificial
  mutated or codon-optimized sequence
atggctaccgacaccggttctcgaccttgctgcagcagccatactggcgatgacaacaacacaaatggcgttaccatcgcgcccaacggtaaccacgaaggttttactgctgtgcagacacgccagaatccacacccacatgtttctcgcaacccatacggtcataacgccggtgtgacagactttcttagcaatgtatctcgcttcaagatcatcgagtccacgctccgtgaaggtgagcaattcgcaaacgccttcttcgacaccgagaaaaaaatcgaaatcgccaaagcactggacgattttggagtggactacatcgaacttacttccccatgtgcgtctgagcagtcacgtaaggattgcgaagctatctgtaagttgggcctgaaggcaaagatccttacccacatccgctgccacatggatgatgcccgtatcgcggtggaaaccggagttgacggcgttgatgtggtgattggcacttcctcctacctccgagaacactcgcacggtaaggatatgacttacatcaaaaacaccgcgatcgaggtgattaacttcgttaaatccaagggcatcgaggttcgcttttcatccgaggactcctttcgatccgatctggtggatctgttgtcgatctactccgctgtcgaccaggttggagttaaccgtgtgggtattgcagacaccgttggttgtgcttcgccacgccaggtctacgagctcgtgcgcgtactgcgcggtgtcgtcaaatgtgatatcgaaatccacctccacaacgacaccggctgcgccatcgcgaacgcctactgtgcactcgagggtggtgcaacacacatcgacaccagcgtacttggcatcggcgaacgcaacggcatcacccccctcggaggcctgatggcccgcatgatcgcagcggatcgtgactacgttctttctaaatataagctggagaaaattaaagatatcgaagatctcgtagcagaggccgtacaggtcaatatcccgttcaacaaccctattaccggcttttgtgctttcacgcataaggctggcatccacgcgaaagctatcctcaacaacccatcgacctacgagatcatcaatcccgcggattttggcatgacccgttacgtgcattttgcctcccgcctgactggatggaatgcaattaaatcacgtgcacagcagctcaatttggatatgaccgatgcgcagtacaaggaatgcacagctaagatcaaggcactcgctgatattcgccctatcgcagttgacgacgcggactctatcatccgagcataccaccgaaatatcaagttgggagaaaataagcccctgctggagcttaccgaagaacagacagcggaattcgcggcaaaggagaaggagctcgcagagaacggtgtcgaagcatccgcg

SEQ ID NO: 31 - protein, 469 aa, Penicillium brasilianum
MCPGADSEPNGQVNGANGANGADHEGFTGIETRQNPHPSASRNPYGHNVGVTDFLSNVSRFKIIESTLREGEQFANAFFDTEKKIEIAKALDDFGVDYIELTSPCASEQSRADCEAICKLGLKAKILTHIRCHMDDARVAVETGVDGVDVVIGTSSYLREHSHGKDMTYIKNTAIEVIEFVKSKGIEIRFSSEDSFRSDLVDLLSIYSAVDKVGVQRVGIADTVGCASPRQVYELVRVLRGVVGCDIETHFHNDTGCAIANAYCALEAGATHIDTSVLGIGERNGITPLGGLMARMMVADPEYVKGKYKLEKLKDIEDLVAEAVQVNIPFNNYITGFCAFTHKAGIHAKAILNNPSTYEIINPADFGMTRYVHFASRLTGWNAIKSRCQQLKIDLTDAQAKECTAKIKALADIRPIAVDDADSIIRAYARNLKLGEDKPLMDLTAEEQAQFAAKEKEIALEAEKNGIAV

SEQ ID NO: 32 - DNA, 1407 bp, Artificial
  mutated or codon-optimized sequence
atgtgcccgggcgcagattcagagccaaatggacaggtgaacggcgccaacggtgccaacggcgcagatcatgaaggatttaccggcatcgagacccgacaaaacccacatccaagcgcttcacgcaacccctacggccataacgtgggagtcaccgactttttgtctaacgtgtctcgcttcaagattatcgaatccacactccgcgagggtgaacagttcgccaacgcattcttcgatactgaaaaaaagatcgaaattgccaaagcactggacgacttcggagttgactacattgaactgacatccccttgtgcgagcgaacagtcccgcgctgattgcgaagcgatctgcaagttgggcctgaaggcaaagatcttgactcatattcgctgccacatggacgacgcacgagtcgcggtcgagaccggagtggatggcgtcgacgtggtcatcggtacttcgtcctacttgcgcgagcatagccacggaaaggacatgacctatatcaagaacacggcaatcgaagtaatcgagttcgtgaagtccaaaggtatcgagatccgcttctcctcggaagactctttccgctccgatctggtagacctcctgtccatctactctgccgtggacaaagttggcgtacaacgtgtgggcatcgcggacactgtgggctgcgcttcccctcgccaggtgtacgagcttgttcgcgttctgcgcggcgtagtcggctgcgatattgagacccatttccataacgataccggatgcgcaatcgcgaatgcgtactgtgctcttgaagcgggtgctacccacatcgacacgagcgttctgggtatcggtgagcgtaacggcatcaccccgctgggtggtctcatggcgcgtatgatggtcgcggacccggaatacgtcaaaggcaagtacaaattggagaaactcaaggacatcgaggaccttgtggcggaagcggtacaagtgaacattccttttaacaattacatcacgggtttctgcgcttttactcacaaggctggtattcacgcaaaggcaatcttgaacaacccttctacctacgaaatcatcaatccggctgattttggcatgacccgctacgtacactttgcgtctcgcctgacaggttggaatgcgatcaagtcacgctgccaacagttgaagattgatcttacggatgcgcaggcaaaggaatgtacagcgaagattaaggcgcttgcagacatccgcccaattgcggtggacgacgccgactctatcatccgcgcctacgcacgcaacctcaagttgggcgaggataaaccactgatggatcttacagccgaagaacaggcacaatttgcagccaaagagaaggagattgctttggaggcagagaaaaatggaatcgcagtg

SEQ ID NO: 33 - protein, 684 aa, Saccharomyces cerevisiae
MFHSSRAWLKGQNLTEKIVQSYAVNLPEGKVVHSGDYVSIKPAHCMSHDNSWPVALKFMGLGATKIKNPSQIVTTLDHDIQNKSEKNLTKYKNIENFAKKHHIDHYPAGRGIGHQIMIEEGYAFPLNMTVASDSHSNTYGGLGSLGTPIVRTDAAAIWATGQTWWQIPPVAQVELKGQLPQGVSGKDIIVALCGLFNNDQVLNHAIEFTGDSLNALPIDHRLTIANMTTEWGALSGLFPVDKTLIDWYKNRLQKLGTNNHPRINPKTIRALEEKAKIPKADKDAHYAKKLIIDLATLTHYVSGPNSVKVSNTVQDLSQQDIKINKAYLVSCTNSRLSDLQSAADVVCPTGDLNKVNKVAPGVEFYVAAASSEIEADARKSGAWEKLLKAGCIPLPSGCGPCIGLGAGLLEPGEVGISATNRNFKGRMGSKDALAYLASPAVVAASAVLGKISSPAEVLSTSEIPFSGVKTEIIENPVVEEEVNAQTEAPKQSVEILEGFPREFSGELVLCDADNINTDGIYPGKYTYQDDVPKEKMAQVCMENYDAEFRTKVHPGDIVVSGFNFGTGSSREQAATALLAKGINLVVSGSFGNIFSRNSINNALLTLEIPALIKKLREKYQGAPKELTRRTGWFLKWDVADAKVVVTEGSLDGPVILEQKVGELGKNLQEIIVKGGLEGWVKSQL

SEQ ID NO: 34 - DNA, 2052 bp, Artificial
  mutated or codon-optimized sequence
atgttccactcttcacgcgcgtggctgaaaggtcaaaacctgaccgaaaagatcgtacagtcctacgccgtcaatttgccggaaggaaaggtggttcactctggcgattacgtttcaatcaaaccagcacactgtatgtctcacgacaattcctggccggtagcgcttaaatttatgggtcttggtgcgaccaagattaaaaacccaagccaaatcgtcaccaccctggaccatgatattcagaacaagtctgagaaaaatctgaccaagtacaagaacatcgagaactttgccaagaagcatcacattgatcactaccccgcgggtcgtggtatcggccatcagattatgatcgaagagggctacgcttttcccctcaacatgacagttgcttctgacagccactcaaatacgtacggcggtctcggctcactcggcactccgattgtgcgcacagacgcagcagctatttgggctacgggccagacatggtggcagatcccaccagttgcgcaggtggagctgaagggccaacttcctcagggtgtctccggtaaagacattatcgtggcactctgcggactgttcaacaacgatcaagtgcttaaccacgcgatcgagtttaccggtgattctctgaatgcacttccgatcgatcaccgcttgaccatcgcgaacatgacgaccgaatggggtgctcttagcggccttttcccggtcgacaagaccctgatcgattggtacaaaaatcgcctgcagaaactcggcacaaacaaccacccccgtatcaacccaaagacgatccgcgctcttgaagaaaaggcaaagatccctaaggccgataaagatgcccactacgcaaagaaactcattatcgatctcgcgacactcacgcactacgtatccggcccaaatagcgttaaagtcagcaatactgtccaggatttgtcccagcaggatatcaagatcaataaggcctacctggtatcatgtacaaatagccgcctctcggatctgcaatcagcggcagatgttgtgtgcccaacaggagacttgaataaagtcaataaggtggcccccggcgtcgaattctacgttgcggcagcatcgtccgaaattgaggctgatgcccgcaaatcaggtgcctgggaaaaattgctgaaggcaggctgtatccccctgccttctggttgtggaccttgtatcggtctgggtgcgggcttgcttgagcctggcgaagtgggtatctccgcgacaaaccgcaacttcaagggacgcatgggctccaaggatgccctggcataccttgcatctcctgcagtcgtggctgccagcgccgttctgggcaagatttcttcaccagccgaggtcctctcaacctctgagatccctttctccggtgttaaaacggaaatcatcgagaacccggttgtcgaagaggaggttaacgcccagaccgaagcacctaagcaatccgtagaaatcctggaaggctttccacgtgagttttctggagaattggtcctgtgtgatgcagataacatcaacaccgatggcatctaccccggcaagtacacataccaggatgacgtcccaaaagagaagatggctcaggtctgcatggaaaattacgatgctgaattccgcaccaaagttcacccgggcgacatcgtagtaagcggcttcaattttggcacgggcagctcccgcgaacaggccgcaacagccttgctggccaagggaattaacctcgttgtatcgggatccttcggtaatatcttttctcgaaacagcatcaacaatgctctgttgaccctcgagatccccgcgttgatcaaaaagctgcgcgaaaagtatcagggagcaccaaaggagctgacccgtcgtaccggctggttcctcaagtgggatgtcgccgatgccaaagttgtcgtaaccgagggtagcctcgatggcccggtgattcttgagcaaaaggttggagagctgggcaaaaacctccaggaaatcatcgtcaaaggaggactcgaaggctgggttaagtctcagctt

SEQ ID NO: 35 - protein, 428 aa, Saccharomyces cerevisiae
MTAAKPNPYAAKPGDYLSNVNNFQLIDSTLREGEQFANAFFDTEKKIEIARALDDFGVDYIELTSPVASEQSRKDCEAICKLGLKAKILTHIRCHMDDAKVAVETGVDGVDVVIGTSKFLRQYSHGKDMNYIAKSAVEVIEFVKSKGIEIRFSSEDSFRSDLVDLLNIYKTVDKIGVNRVGIADTVGCANPRQVYELIRTLKSVVSCDIECHFHNDTGCAIANAYTALEGGARLIDVSVLGIGERNGITPLGGLMARMIVAAPDYVKSKYKLHKIRDIENLVADAVEVNIPFNNPITGFCAFTHKAGIHAKAILANPSTYEILDPHDFGMKRYIHFANRLTGWNAIKARVDQLNLNLTDDQIKEVTAKIKKLGDVRSLNIDDVDSIIKNFHAEVSTPQVLSAKKNKKNDSDVPELATIPAAKRTKPSA

SEQ ID NO: 36 - DNA, 1284 bp, Artificial
  mutated or codon-optimized sequence
atgaccgccgcgaaacccaacccatacgcagcgaagccaggagactacctttccaacgttaacaactttcagctcattgattccaccctgcgcgaaggagaacagttcgcgaatgcgtttttcgacaccgagaagaagatcgaaatcgctcgtgccttggatgacttcggcgtggattacattgaactgacttccccggtggcctcggagcagagccgcaaggactgcgaggcgatctgcaaactgggcctgaaagccaagatccttacccatattcgctgccatatggatgatgctaaggttgcggtagagaccggagtggacggagttgacgtcgtgatcggaacgtcgaagtttttgcgccagtactctcacggcaaggatatgaattatatcgcaaagagcgctgtggaggtaattgaatttgtgaagtcaaagggcattgaaattcgcttttcgtccgaagattccttccgcagcgaccttgtagaccttctgaatatctataagaccgtggacaaaatcggcgtgaatcgagttggtatcgccgatacagtgggttgtgctaatccccgccaagtctatgaactcatccgaacccttaagagcgtcgtaagctgcgatatcgagtgtcactttcacaacgatactggctgcgcaatcgctaacgcatataccgctctcgaaggcggcgctcgtctgattgacgtatcggtcttgggtatcggcgaacgaaacggtatcacaccgctgggcggccttatggcacgcatgattgttgcagcaccagactacgttaagtccaagtacaaacttcacaagatccgagacattgagaacctggtcgccgatgccgtcgaagtgaatatcccattcaataatcccattaccggcttctgtgcgttcacccataaggcgggcatccacgccaaagccattttggccaacccgagcacgtacgagatccttgatccacacgactttggtatgaagcgttacatccacttcgcgaatcgtctcaccggctggaacgcaatcaaggcccgcgtagatcagctcaacctcaaccttaccgatgatcaaatcaaagaagtcaccgccaaaatcaagaagctcggtgacgttcgctcgcttaacatcgatgacgttgattcaattatcaagaacttccatgcggaagtgtcaactccccaagtactctccgctaagaagaataagaagaatgactcagacgtgccagaacttgcgaccattcctgccgccaaacgtactaaaccatccgcg

SEQ ID NO: 37 - protein, 487 aa, Stemphylium lycopersici
MCPPTEETPATNGHSNGTNGAPINKEGFTGVQTKQNPHPTHKSPYQPVGDFLSNVGRFKIIESTLREGEQFANAYFDLEAKIKIARALDNFGVDYIEVTSPAASEQSRRDCEALCKLGLKAKILTHVRCHMDDARIAVETGVDGLDVVIGTSAYLREHSHGKDMTYIKNTALEVIEFVKSKGKEIRFSSEDSFRSDLVDLLSIYSAVDKVGVDRVGIADTVGCASPRQVYDLVRTLRGVVSCDIETHFHNDSGCAIANAFCALEAGATHIDTSVLGIGERNGITPLGGLMARMIVADREYVTSKYNIKALKEVEDLVADLVEVNIPFNNYITGFCAFTHKAGIHAKAILNNPSTYEIINPQDFGMSRYVHFASRLTGWNAIKSRAEQLGLKMTDAQYKECTAKIKAIADIRKIALDDTDSIIRTYHNNLHATEEVPLLPGMTEEEKAKFAQAEAELNGMHEKRELDATADAQAEVPVAKKNKTATVA

SEQ ID NO: 38 - DNA, 1461 bp, Artificial
  mutated or codon-optimized sequence
atgtgcccacccacggaggagaccccggcaaccaacggtcactcaaacggtacaaacggtgccccaattaacaaagaaggatttaccggcgtgcaaacgaagcaaaatccccacccgacgcataagtccccctatcaacccgtcggagattttctgagcaacgttggtcgtttcaagatcattgagtcaacccttcgcgaaggtgagcagtttgcaaacgcatactttgatctggaggcgaagattaaaattgctcgtgcactcgataactttggcgttgactatatcgaggtaacctctccagccgcatcggagcaatcccgccgcgattgcgaggcgctctgcaaactgggtcttaaggcaaaaatcctcacccacgtccgttgccatatggacgatgcgcgtatcgctgtcgagacgggcgtggatggactggacgttgtgatcggtacttcagcctacctgcgcgaacattctcacggaaaggatatgacgtacatcaagaacaccgctctcgaggtgattgagttcgtaaaaagcaaaggaaaggagattcgcttttcctcggaagattccttccgcagcgatctggttgaccttctctccatctactctgcagttgataaagtgggtgtcgatcgagtgggtattgcggataccgtaggctgtgcctccccccgtcaggtctacgacctggtccgaaccctgcgcggagttgtgtcctgcgacatcgaaactcatttccacaacgactcgggctgcgcaatcgcgaatgccttctgcgcactcgaagccggcgccacccatattgatacttctgtcctgggtatcggtgaacgcaatggtattactccactgggaggactcatggcacgcatgatcgttgccgaccgcgaatatgttacatccaagtacaacatcaaggccctgaaagaggttgaggacctcgtcgcagacctggtcgaggtcaacatccccttcaacaactacattaccggtttctgtgcatttactcataaggccggcatccatgcaaaagccatcctgaacaacccgtccacgtacgagatcattaacccacaggattttggtatgtcccgctacgttcacttcgcctcacgactcacaggttggaacgcgatcaagtcccgcgcagaacaactcggattgaagatgactgatgcccagtacaaagagtgcaccgcaaaaatcaaagccattgcagatatccgtaaaatcgcactggacgacaccgactcgattatccgtacctaccacaacaacctgcacgccacagaagaggtgccattgcttcctggcatgactgaggaggaaaaggccaagtttgcccaggctgaagccgagcttaacggcatgcatgaaaagcgcgaacttgatgccactgcagacgcacaggccgaagttccggtcgcgaagaagaacaaaaccgcgacagtggct

SEQ ID NO: 39 - protein, 359 aa, Saccharomyces cerevisiae
MGLASNAARKSLTIGLIPGDGIGKEVIPAGKQVLENLNSKHGLSFNFIDLYAGFQTFQETGKALPDETVKVLKEQCQGALFGAVQSPTTKVEGYSSPIVALRREMGLFANVRPVKSVEGEKGKPIDMVIVRENTEDLYIKIEKTYIDKATGTRVADATKRISEIATRRIATIALDIALKRLQTRGQATLTVTHKSNVLSQSDGLFREICKEVYESNKDKYGQIKYNEQIVDSMVYRLFREPQCFDVIVAPNLYGDILSDGAAALVGSLGVVPSANVGPEIVIGEPCHGSAPDIAGKGIANPIATIRSTALMLEFLGHNEAAQDIYKAVDANLREGSIKTPDLGGKASTQQVVDDVLSRL

SEQ ID NO: 40 - DNA, 1077 bp, Artificial
  mutated or codon-optimized sequence
atgggtctcgccagcaacgcggcccgcaaatccctgaccatcggcctgatcccaggtgatggcattggcaaagaagttatccccgccggcaagcaagtgttggagaaccttaattccaaacacggcttgtcttttaatttcattgacttgtacgcgggctttcagaccttccaggaaaccggtaaagctctgcccgatgagaccgtcaaagtgctgaaagaacaatgtcaaggcgccctttttggagccgtccaatccccgaccactaaggttgagggttacagcagcccaatcgttgccttgcgtcgtgaaatgggactgttcgcgaacgtccgccctgtcaagtccgtggagggcgaaaagggaaagcctattgacatggttatcgtgcgtgaaaacactgaagatctctacatcaaaattgagaagacatacattgataaggccaccggcacacgcgtagcggatgcgaccaagcgtatctctgaaatcgcaacgcgccgcatcgccaccatcgcactcgatattgcgctgaaacgtctgcaaactcgaggacaggccaccctgaccgtcacgcacaagtctaacgttctgtcccagagcgatggactgtttcgcgagatttgtaaggaagtttacgaatccaataaggataagtacggacagatcaaatataatgaacaaattgtggactccatggtgtaccgcttgttccgcgaaccacagtgtttcgacgttatcgttgctccgaatctgtacggagatatcctctctgacggtgcagcggcgttggttggatcgctgggcgtggtacctagcgcaaacgttggtccagaaattgtgatcggcgagccatgtcacggctcggctccagatatcgcaggaaagggcatcgcaaatcccatcgccaccatccgctccactgctctgatgctggagttcttgggtcacaatgaagcggcgcaagacatctacaaggccgtggatgcaaaccttcgtgaaggctccatcaagactccagatcttggaggaaaagcgtcgacacaacaggtcgtcgatgatgtactttcacgcctg

SEQ ID NO: 41 - DNA, 1254 bp, Artificial
  mutated or codon-optimized sequence
atgtccgtgtccgaagcgaacggaaccgaaacgattaaacccccgatgaatggcaacccctatggtccaaacccctccgactttttgtctcgtgttaataacttctccatcatcgaatctaccttgcgtgagggcgaacaattcgcaaatgcttttttcgacaccgaaaagaagattcagatcgctaaggccctggataacttcggtgtcgattacatcgagttgacatctccagtggcttcagaacagtcccgtcaagattgcgaggctatctgtaagttgggtctgaaatgtaaaattttgacccacatccgatgtcacatggacgacgctcgcgtggcagtggaaactggtgtcgacggtgttaacgtggtgattggcacctcccagtacctgcgcaagtacagccacggtaaggacatgacctacatcattgactccgctaccgaagttatcaactttgtgaaatcaaagggcattgaagtgcgcttctcatcagaggattcgttccgttctgatttggtggatcttcttagcctctacaaagctgtggataagatcggtgttaaccgcgttggcatcgcagatacggtgggttgcgccactcctcgccaagtatacgaccttatccgcacacttcgtggcgttgtgtcctgcgatattgaatgccatttccacaacgacactggtatggccattgcaaatgcatactgcgcgttggaggccggtgctactcacattgacacatccattctgggtattggcgaacgaaatggaattaccccactgggtgccctcctggctcgtatgtacgtaacggaccgagaatatattacccacaaatacaagctgaaccagctgcgcgaattggaaaatcttgttgcggatgctgttgaagttcagattccattcaataactacattacgggcatgtgcgcttttacccacaaggcaggaatccacgcgaaggcaatccttgctaacccaagcacgtacgagatccttaaacccgaagatttcggcatgtcccgatacgtacatgttggatcccgtctgaccggttggaacgctatcaaatcacgcgctgaacagctcaacctccatctgacagatgctcaggcaaaggaactcactgtacgcatcaagaagctcgcggacgtccgaaccctggccatggatgatgtcgaccgcgtccttcgtgaatatcatgcggacctcagcgatgcagaccgcattactaaagaagcatccgcg

SEQ ID NO: 42 - protein, 380 aa, Paenibacillus riograndensis
MKSLKLCDTTLRDGEQAAGVSFTRTEKLEIAKLLSECGVEQAEVGIPAMGTREQEDIAAIAELNLPMKLMTWNRSLPGDIDKARATGVNWSHVSIPVSEIQLQGKLGLSTDEGLNKLLGAAEYALSLGMTVSVGMEDSSRADMGFLIRLVNSLHREGICRFRYADTVSAHHPGQIAERVSTLLGEVPGDVELEVHCHNDFGLACANTLSGIAAGAAWASTTVAGIGERTGNAAMEEVVMAWHHLYGGDSSVRFDLLKGLADKVIAASGRSVGDAKPIVGQLAFTHESGIHVDGLMKERATYQTFDPSEVGRSHRFVLGKHSGTGGVAHVLEQLGLAITPDTAGRLLVRVREYAESHKGTVPEYMLVQWLMEEQQRAQNVV

SEQ ID NO: 43 - DNA, 1140 bp, Artificial
  mutated or codon-optimized sequence
atgaagtccctgaaactttgcgataccaccctccgcgatggagaacaggcagcgggtgtttcgtttacacgcactgagaaactcgaaatcgctaaactgctctctgagtgtggagtagaacaggcggaggttggtatccctgccatgggtacgcgcgagcaagaggatattgctgcaatcgccgaactcaacttgccaatgaagttgatgacctggaatcgttcgctccctggcgacattgacaaggcacgcgcaaccggtgtcaattggtcccatgtctcgatccccgtctccgagattcagttgcagggtaagctcggcttgtccacagatgaaggccttaacaagttgctcggcgcggcggaatacgccctgtctttgggcatgaccgtatcggtcggtatggaggattcctcgcgcgctgatatgggcttcctcatccgcctcgtcaattcactgcatcgtgagggtatctgccgcttccgatacgccgatactgtatctgcgcaccatccaggacagatcgcggaacgcgtgtcgacccttttgggagaagttccgggcgacgttgaacttgaagtgcactgccataacgacttcggtctggcctgtgcgaataccctgtcgggaatcgccgcaggcgctgcctgggcatccacaaccgtggcgggcattggtgagcgcaccggtaacgcggcaatggaagaagtggtgatggcttggcaccatctgtacggtggcgattcctcggtgcgcttcgacttgctgaagggcctggcagacaaggttatcgctgcttctggtcgttccgtgggtgatgcaaaacccattgtcggtcagctggcattcacacacgaatccggtattcatgtcgatggcctcatgaaggagcgcgcaacctaccaaacgtttgatccttcagaggtaggccgttctcaccgcttcgtcctcggtaagcattccggaaccggcggagtcgcgcacgtactcgagcaactcggactcgctatcaccccagacaccgccggccgcttgctggttcgcgttcgagagtacgcagaaagccacaaaggtaccgttccggagtatatgttggtccagtggctcatggaagaacagcagcgtgcgcagaacgtcgtt

SEQ ID NO: 44 - protein, 673 aa, Neosartorya fumigata
MRYTEASSSTTQTSPSSSSWPAPDAAPRVPQTLTEKIVQAYSLGLAEGQYVKAGDYVMLSPHRCMTHDNSWPTALKFMAIGASKVHNPDQIVMTLDHDVQNKSEKNLKKYESIEKFAKQHGIDFYPAGHGVGHQIMIEEGYAFPGTVTVASDSHSNMYGGVGCLGTPMVRTDAATIWATGRTWWKVPPIAKVQFTGTLPEGVTGKDVIVALSGLFNKDEVLNYAIEFTGSEETMKSLSVDTRLTIANMTTEWGALTGLFPIDSTLEQWLRHKAATASRTETARRFAEERINELFANPTVADRGARYAKYLYLDLSTLSPYVSGPNSVKVATPLDELEKHKLKIDKAYLVSCTNSRASDIAAAAKVFKDAVARTGGPVRVADGVEFYVAAASKAEQKIAEEAGDWQALMDAGAIPLPAGCAVCIGLGAGLLKEGEVGISASNRNFKGRMGSPDAKAYLASPEVVAASALNGVISGPGIYKRPEDWTGVSIGEGEVVESGSRIDTTLEAMEKFIGQLDSMIDSSSKAVMPEESTGSGATEVDIVPGFPEKIEGEILFLDADNISTDGIYPGKYTYQDDVTKDKMAQVCMENYDPAFSGIARAGDIFVSGFNFGCGSSREQAATSILAKQLPLVVAGSIGNTFSRNAVNNALPLLEMPRLIERLREAFGSEKQPTR

SEQ ID NO: 45 - DNA, 2019 bp, Artificial
  mutated or codon-optimized sequence
atgcgctataccgaagcttccagctcgacaacacaaacctcccctagctcttcctcgtggcccgccccggacgctgcgccacgcgtgcctcagacgctcactgaaaaaattgtgcaagcgtattcactcggcctggcggaaggtcagtacgtcaaggcgggagattacgtgatgctctctccccaccgctgcatgacccacgacaattcgtggcccaccgcattgaaatttatggcgatcggcgcatcgaaagttcataaccctgatcaaatcgtgatgaccttggaccacgacgtccagaataaatccgaaaagaacctgaaaaaatacgaatcgatcgagaagtttgccaagcagcacggtatcgatttctacccggccggccacggagtgggccatcaaattatgattgaggaaggatacgctttcccaggaaccgtgaccgtcgcatctgattcacacagcaacatgtacggtggcgtgggctgccttggaactccaatggtacgaactgatgcagcaaccatctgggcaaccggccgcacgtggtggaaggtgccacctatcgcaaaagtgcagtttacaggtactcttccggaaggcgtcacaggcaaagatgtgattgttgcactgtcgggattgtttaacaaagacgaagtgttgaattacgcaatcgagttcacgggaagcgaagaaacaatgaaatctctctcggtcgatacacgcctgaccatcgctaatatgaccacggagtggggtgcgttgaccggtctgttccctatcgattccacgctggagcagtggcttcgacacaaggcagcaaccgcgtcccgaaccgaaactgcccgccgcttcgcggaggaacgcattaacgagctctttgccaacccaactgtggctgatcgaggcgcacgctacgcgaaatacctttatctggatttgtctaccctgtccccttacgtttctggtccaaacagcgtcaaggttgccacaccactggatgaactggaaaagcacaagttgaagatcgataaggcctacttggtctcttgcaccaactcccgagcaagcgatatcgctgcagcagctaaggtttttaaggatgcggttgcacgtactggcggccctgtccgtgttgcagatggcgttgagttttatgtcgctgcggcatccaaagcagaacaaaagatcgcggaagaagctggagattggcaagctcttatggacgccggagcaattcctcttccggcaggttgcgccgtatgcattggcctcggcgcgggcctcctcaaggaaggagaggtcggcatttcggcctccaaccgtaactttaagggccgcatgggttccccagacgcaaaggcataccttgcatctccagaagtggttgctgcgagcgcgctgaatggtgttatttcgggacctggtatttataaacgccctgaggattggaccggtgtctccatcggtgaaggtgaagttgtggaatccggctcgcgcattgacacaactctggaagccatggagaagtttatcggacaacttgattccatgatcgactcctcctctaaggctgtaatgccagaagaaagcactggttccggtgcaacggaggtggacatcgtcccgggttttccggaaaagattgaaggtgaaatcttgttcctggatgcggacaacattagcactgacggcatttacccgggtaagtacacttaccaagatgacgtgaccaaggataaaatggctcaagtgtgtatggagaactacgacccagcgttctcgggaatcgcgcgcgcaggcgacatcttcgtgtcaggcttcaatttcggatgcggttcttcccgcgaacaggcagcaacctccattctggcaaaacaattgcctctggtggttgcaggctcgatcggcaacaccttttcccgaaatgctgtcaataatgccctgcccttgctggaaatgccacgtcttatcgaacgcctccgtgaagcctttggctctgagaaacagccaacccgc

SEQ ID NO: 46 - protein, 376 aa, Chloroflexi bacterium
MPVERFAIIDSTLREGEQFSSAHFTTDQKVVVAKALDAFGVEYIEVTSPVASPQSFDDCKTIAALPLKAKVLTHVRCHMDDARVAVETCVDGIDILFGTSSFLREFSHGKSVDEIIAQAGEVITFIKDHGKEVRFSSEDSFRSTEGDLLRVYQAVDSMGVDRVGIADTVGIATPRQCYALAHELRRNVSCDIEFHGHNDTGCAIANSYSVLEGGATHIDTSILGIGERNGITPLGGFIARMYADDPAAIKRKYDLPRLRELDELVASMVGIDVPFDEYVTGKFAFHHKAGMHTKAIYLNPNSYEILDPADFGLERTINIAHRLTGYNAVGRRAQELGLQFGKDDLREVTKRIKAMADAGPLSMDQLDQILRGYVVA

SEQ ID NO: 47 - DNA, 1128 bp, Artificial
  mutated or codon-optimized sequence
atgccggtggagcgttttgcaatcatcgattcgaccttgcgcgagggtgagcaattttcctccgctcacttcacgaccgatcagaaagtggtggtggctaaggctctcgacgcttttggtgttgagtacattgaagtcacttctcctgtcgcctccccacagtcattcgatgactgtaagactattgcggcactccccttgaaagcaaaagtgttgactcacgttcgttgtcatatggatgatgctcgcgtggcggtggagacgtgcgttgacggcattgatatcctctttggcactagctcttttctgcgagaattttctcacggcaagtccgtggatgagatcattgcccaggcgggagaagttattacattcatcaaggatcacggaaaggaagttcgtttttcgtccgaagactccttccgctcaacagaaggagacctgctgcgcgtgtatcaggccgttgacagcatgggtgtcgatcgcgtgggtattgctgacacggtgggaattgctacaccccgtcaatgttacgctctcgcccatgagcttcgccgaaacgtgtcgtgtgacatcgaattccatggtcataacgataccggctgcgccatcgcaaattcgtactcggtcctggagggcggcgctactcacatcgacacgagcattctgggtatcggcgagcgcaacggcattacaccactgggaggattcatcgctcgtatgtacgcggacgacccagcggctatcaaacgcaaatatgatcttcctcgccttcgagagttggatgaactggttgcttccatggtcggtattgacgtgcctttcgacgaatacgtcaccggtaagtttgcgttccaccacaaggcaggtatgcacactaaggcgatttacctcaatccaaactcctacgaaatccttgacccagcggattttggtctcgagcgtaccattaacatcgcccaccgcctcacgggctacaacgcggtgggacgacgcgcccaggaactgggtctccaattcggtaaggacgacctccgagaagtgaccaaacgaatcaaggccatggcagatgctggaccactgtcaatggatcaattggaccaaatcttgcgcggctacgtggtggca

SEQ ID NO: 48 - protein, 442 aa, Ashbya gossypii
MSQGNEFHQVTEATTALNNFQQNPYGPNPADYLSNVGSFQLIDSTLREGEQFANAFFSTEKKIEIARALDDFGVDYIELTSPVASEQSLRDCQAICKLGLKAKILTHIRCHMDDAKVAVGTGVDGVDVVIGTSKFLRQYSHGKDMNYIAKSAIEVIEYVKSKGIEIRFSSEDSFRSDLVDLLNIYKTVDKIGVNRVGIADTVGCANPRQVYELVRTLKSVVSCDVECHFHNDTGCAIANAYTALEGGAKLVDVSVLGIGERNGITPLGGLMARMIVAAPDYVKSKYKLHKIRDIENLVAEAVEVNVPFNNPITGFCAFTHKAGIHAKAILANPSTYEILNPNDFGMTRYIHFANRLTGWNAIKSRVDQLNLHLTDDQVKEVTAKIKQLGDVRPLNIDDVDSIIKAFHAQIATPRVTARPANREEEINGFDLEAPPQKKTKIL

SEQ ID NO: 49 - DNA, 1326 bp, Artificial
  mutated or codon-optimized sequence
atgtcccaaggtaacgaatttcaccaagttaccgaggcaacgaccgcactcaacaattttcagcagaatccctacggcccgaatccggctgactacttgtcgaatgtcggctcttttcaacttatcgactcgaccctccgcgagggcgagcagttcgccaacgcgttcttttccactgagaagaaaatcgaaattgcccgtgctttggacgacttcggtgtcgattatattgagctcacctccccggtggcgtccgaacagagccttcgcgactgtcaggccatctgcaagcttggcctcaaggcaaagattctcacgcacatccgttgccacatggatgacgcaaaggtggctgtgggcaccggagtcgatggagtggatgtggtcatcggaacctctaagttccttcgtcaatactcgcacggtaaggacatgaactatatcgctaagtccgcgattgaagttatcgagtacgtcaagagcaaaggtattgagatccgtttctcctccgaggattcattccgctcagatctcgtggaccttctgaacatttataagaccgttgacaagattggtgtcaatcgtgttggaattgctgacaccgtcggatgcgcaaatccacgccaggtttacgagctcgtccgcaccctcaaatccgtggtgtcatgcgacgtggaatgtcattttcacaacgataccggctgcgccatcgcaaacgcatacaccgctctcgaaggaggcgccaaactcgtggacgtaagcgtgctgggcatcggtgagcgtaatggtattactccactcggtggcctgatggcccgtatgatcgtagcggcaccagactacgtaaaatcgaagtacaagcttcataagatccgcgacattgagaacttggtggctgaggcagtggaggtcaacgtgccatttaataacccaattaccggtttctgcgctttcacacataaagcaggaattcacgctaaggcaatcctcgctaacccctccacctatgaaatcctgaaccctaacgattttggtatgacacgctacatccacttcgccaaccgtcttaccggctggaatgcaatcaagtctcgcgtagaccagcttaacttgcacctcacggatgatcaggttaaagaggtgactgccaagattaaacaacttggcgatgttcgcccactcaacatcgatgatgttgatagcatcatcaaggcctttcacgcacagatcgcgacccctcgcgtgaccgctcgaccagctaaccgtgaagaagaaattaacggcttcgaccttgaagcgcctccgcagaagaagacgaagatcctc

SEQ ID NO: 50 - protein, 381 aa, Pseudomonas stutzeri
MSIVIDDTTLRDGEQSAGVAFSAEEKLAIARALAQLGVPELEIGIPSMGEEECEVMRAIAGLALPVRLLAWCRLCDADLLAAGGTGVGMVDLSLPVSDLMLQHKLGRDRDWALREAARLVGAARDAGLEVCLGCEDASRADPEFIVRVAEVAQAAGARRLRFADTVGVMEPFAMHARFRFLAERLDLELEVHAHDDFGLATANTLAAVRGGATHINTTVNGLGERAGNAALEECALALKHLHGIDCGIDVRGIPSISALVEQASGRQVAWQKSVVGAGVFTHEAGIHVDGLLKHRRNYEGLNPDELGRSHSLVLGKHSGAHMVELSYRELGIELQQWQSRALLGCIRRFSTQTKRSPQSADLQGFYQQLCEQGLALAGGAA

SEQ ID NO: 51 - DNA, 1143 bp, Artificial
  mutated or codon-optimized sequence
atgtcaattgtcattgatgataccaccctccgtgatggtgagcagtccgcgggcgtcgctttctctgcggaggaaaagttggccatcgctcgcgctctggcccaactgggtgtaccagaattggaaattggtatcccatctatgggcgaagaggaatgcgaagtgatgcgagctatcgcaggactggcactcccagtgcgccttctggcctggtgtcgtctctgcgacgcagacttgctcgctgcaggcggcactggcgtgggcatggtggatttgtcgttgccggtgtccgatcttatgctccaacacaaattgggtcgagatcgtgactgggctctgcgtgaggccgcacgactcgtcggagctgcgcgtgacgcaggtctggaggtctgcctgggttgcgaagatgccagccgtgcagatcccgagttcatcgtccgagtcgcagaagtggcacaagccgccggagcccgtcgtctgcgcttcgcagatactgtgggagtgatggaaccctttgccatgcatgcacgcttccgattcctggcagagcgactcgaccttgagctggaagttcacgcccacgatgacttcggcctggcgaccgccaacaccctcgcagcagttcgcggcggagcgacacacattaacaccacggttaacggactgggagaacgcgcgggtaatgctgctctcgaagaatgtgcactcgccctcaagcaccttcacggcattgactgcggtatcgacgtgcgtggcatcccatccatctccgcacttgtcgaacaggcgtccggacgccaggtcgcctggcagaaatctgtggtgggcgcaggtgtattcacccacgaggcaggaattcatgtcgacggcctcctgaaacaccgccgaaattatgagggtctcaatcctgatgagcttggccgtagccattcactcgttttgggcaagcactcgggcgctcatatggtggaactgtcatatcgcgaactgggcatcgaactccaacaatggcaaagccgcgcactgttgggctgcatccgacgcttttctacccaaacgaagcgctccccacagtccgcagacttgcagggcttttaccagcaactctgtgaacagggactcgccctcgcgggcggcgctgcc

SEQ ID NO: 52 - protein, 376 aa, Unknown
  Thermus sp. CCB_US3_UF1
MREWRIIDSTLREGEQFERANFTTQDKVLIAQALDEFGIEYIEVTTPMASPQSRKDAETLASLGLKAKVVTHIQTRLDAAEVAVETGVQGIDLLFGTSKYLRAAHGRDIPRIIEEAREVIQYIREKAPHVEVRFSAEDTFRSDEHDLLEIYGAIAPYVDRVGLADTVGIATPRQVYALVREVRRVVGPEVDIEFHGHNDTGCAIANAFEAIEAGATHVDTTVLGIGERNGITPLGGFLARMYTLQPEYVRGKYKLEMLPELDRMVARMVGVEIPFNNYITGETAFSHKAGMHLKAIYINPESYEPYPPEVFGVRRKLIIASKLTGRHAIRARAEELGLHYGEEELARITQHIKALADRGQLTLEELDRILREWITA

SEQ ID NO: 53 - DNA, 1128 bp, Artificial
  mutated or codon-optimized sequence
atgcgcgaatggcgcatcatcgattccacactgcgcgagggagaacagtttgaacgcgctaatttcactacacaagataaagtcttgatcgcccaggctctcgatgaattcggcatcgaatatatcgaggttaccacgccgatggcctctcctcaatcccgtaaagatgcagagactttggcgtcattgggcctcaaagctaaagttgtgactcatattcagacccgtcttgacgcagcggaagtcgcggttgaaacaggcgtgcagggcatcgatctccttttcggtacatccaagtacttgcgtgccgcccatggtcgcgacatcccccgcatcatcgaagaagcccgcgaagtgatccagtacattcgcgaaaaggccccgcacgtggaagtgcgtttctccgcggaagacacatttcgctcagatgaacacgacctgttggagatttacggcgccatcgcaccgtacgtggatcgcgtaggtttggccgacactgtgggcattgctacaccgcgacaggtctatgcgcttgtgcgagaagtgcgccgcgttgttggacccgaagtggacatcgagtttcacggacataatgacactggctgtgctatcgccaatgcattcgaagcgattgaggcaggcgcaacgcacgtcgacaccaccgtgctcggcatcggcgagcgtaacggaatcaccccgctcggtggcttcctggcccgtatgtacaccttgcaacccgagtacgttcgcggaaaatacaagctcgagatgctccctgaattggatcgcatggtcgctcgcatggtgggcgtcgaaattcccttcaacaactatattaccggcgaaaccgcgttttctcacaaagctggcatgcaccttaaagctatctacattaaccctgagtcatatgaaccataccctcctgaagtcttcggcgtgcgccgcaagctcattatcgcttccaagcttacgggtcgccacgctattcgcgcacgtgctgaagaactcggccttcattacggcgaggaagagctggctcgcatcactcagcacattaaagccctcgccgatcgcggacagcttacgctggaagaattggaccgtatcctccgagaatggatcaccgct

SEQ ID NO: 54 - protein, 390 aa, Methanobrevibacter smithii
MQYYISHYNKEPELNFPDEITVYDTTLRDGEQTPGVCFSPEEKLEIAKKLDEVKIKQIEAGFPIVSKKEQESVKAITSEGLNAQIISLSRTKKEDIDAALDCDVDGVITFMGTSDIHLEHKMHIGRQEALNTCMNAIEYAKDHGLFVAFSAEDATRTDLDFLKRIYNKAESYGADRVHIADTTGAITPQGITYLVKELKKDVNIDIALHCHNDFGLAVINSISGVLAGANGISTTVNGIGERAGNASLEEVIMSLKLLYGKDLGFKTKHIKELSELVSKASGLPVPYNKPVVGNNVFRHESGIHVDAVIEEPLCYEPYIPELVGQKRQLVLGKHSGCRAVRAKLNECDLDVSDDTLIEIVKKVKKSREEGTYINDDVFKEIVKSCNYKKE

SEQ ID NO: 55 - DNA, 1170 bp, Artificial
  mutated or codon-optimized sequence
atgcagtactacatttcccattacaataaagagccagagttgaactttcctgatgaaatcaccgtgtatgacacaactctgcgcgacggcgagcaaacgcctggcgtctgcttctcgccggaagaaaaactggaaatcgcgaaaaagctcgatgaggtgaaaatcaagcagatcgaggcaggcttcccaattgtgtctaaaaaggaacaagaatcagttaaagctattacctccgagggactcaacgctcaaattatttccttgtcacgcaccaaaaaggaagatatcgatgccgcgcttgattgcgatgtagacggcgtaattacattcatgggtactagcgacattcatttggaacacaagatgcatattggccgtcaggaggccctgaacacctgcatgaatgcaatcgaatacgccaaggaccatggtctctttgtggctttttccgcagaggatgcgacacgcacggatctggatttcctgaagcgtatttacaacaaggctgagtcgtatggtgcagatcgcgtacacatcgcggacaccactggcgcgatcacgccacaaggaatcacctatcttgttaaagaacttaagaaagacgtgaatattgatattgcacttcattgtcataacgattttggtcttgctgttatcaactcgatcagcggcgtactggccggtgctaatggcatcagcaccacggtcaacggaattggcgaacgcgccggtaacgcctcgctggaggaagttatcatgagcctcaagcttttgtacggaaaagacttgggattcaagacaaagcacattaaggaactctcggaattggtctcaaaagcttctggcctcccggtgccttataataaacccgtggtgggtaataacgttttccgtcatgaatccggtatccacgtcgacgcggtgattgaagagccactgtgctatgaaccgtacatccccgaacttgtcggacagaaacgccagttggttctcggaaaacacagcggctgccgtgctgttcgcgccaagctcaacgagtgcgatctcgatgtatccgatgataccctcatcgaaattgtgaagaaggttaagaaatcacgagaagaaggtacttacatcaatgacgacgttttcaaagaaattgtgaaatcctgtaactacaagaaagaa

SEQ ID NO: 56 - protein, 777 aa, Neosartorya fumigata
MFKRTGSLLLRCRASRVPVIGRPLISLSTSSTSLSLSRPRSFATTSLRRYTEASSSTTQTSPSSSSWPAPDAAPRVPQTLTEKIVQAYSLGLAEGQYVKAGDYVMLSPHRCMTHDNSWPTALKFMAIGASKVHNPDQIVMTLDHDVQNKSEKNLKKYESIEKFAKQHGIDFYPAGHGVGHQIMIEEGYAFPGTVTVASDSHSNMYGGVGCLGTPMVRTDAATIWATGRTWWKVPPIAKVQFTGTLPEGVTGKDVIVALSGLFNKDEVLNYAIEFTGSEETMKSLSVDTRLTIANMTTEWGALTGLFPIDSTLEQWLRHKAATASRTETARRFAEERINELFANPTVADRGARYAKYLYLDLSTLSPYVSGPNSVKVATPLDELEKHKLKIDKAYLVSCTNSRASDIAAAAKVFKDAVARTGGPVRVADGVEFYVAAASKAEQKIAEEAGDWQALMDAGAIPLPAGCAVCIGLGAGLLKEGEVGISASNRNFKGRMGSPDAKAYLASPEVVAASALNGVISGPGIYKRPEDWTGVSIGEGEVVESGSRIDTTLEAMEKFIGQLDSMIDSSSKAVMPEESTGSGATEVDIVPGFPEKIEGEILFLDADNISTDGIYPGKYTYQDDVTKDKMAQVCMENYDPAFSGIARAGDIFVSGFNFGCGSSREQAATSILAKQLPLVVAGSIGNTFSRNAVNNALPLLEMPRLIERLREAFGSEKQPTRRTGWTFTWNVRTSQVTVQEGPGGETWSQSVPAFPPNLQDIIAQGGLEKWVKKEISKA

SEQ ID NO: 57 - DNA, 2331 bp, Artificial
  mutated or codon-optimized sequence
atgttcaagcgcaccggttcacttcttttgcgatgccgtgcgtcccgcgtgcctgtgatcggacgtcccctcattagcctcagcacctcctccacgtccctctctctgtcgcgcccccgatccttcgcaactacttcgcttcgccgctataccgaagcatcctcatctactacacagacctcccccagctcgtcatcgtggccggctccagatgcagccccacgcgtgccacaaactctcacagaaaagatcgtgcaagcgtatagcctcggtctggcagaaggtcagtacgtcaaggcgggcgattacgtcatgctgtccccgcaccgttgtatgacacatgacaactcctggccaacggcactgaagttcatggcaatcggtgcttctaaggttcacaatcccgaccagatcgtgatgaccttggaccatgatgtccagaataagagcgagaagaacctgaagaaatatgaatctatcgaaaaatttgctaaacaacacggaattgatttttaccctgccggccatggcgtcggtcaccagatcatgatcgaggagggctacgcgttcccaggcaccgtgacagtcgcatcagactcccactctaacatgtatggaggcgtgggttgtttgggcaccccgatggttcgcaccgacgcggccaccatttgggctacaggtcgcacttggtggaaagttccccctatcgcaaaggtgcaattcactggtacccttcccgaaggagtgacgggtaaagacgttattgtggcattgtcaggtctcttcaataaggatgaggtgctgaattacgctattgagttcaccggctctgaagagaccatgaagagcctgtccgttgacacccgcttgactatcgccaacatgacgacagaatggggtgcgttgactggactcttccctatcgattcgaccctggagcagtggctgcgtcacaaagctgccactgcgtctcgtaccgaaactgcgcgccgcttcgccgaggaacgcattaatgagctgttcgcaaaccctaccgtcgcagaccgtggcgcacgttatgcaaagtacctttacctcgacctgtccactctgtccccatacgtgtccggaccgaactccgttaaggtcgcaacgcccctggacgagctggaaaagcacaagctcaagatcgataaagcctatctggtctcctgtactaactctcgtgcatcggatattgcggctgctgcaaaagtgttcaaggatgcggttgcccgaacgggcggacccgtccgagttgctgatggcgtggaattctatgtcgccgcagcctccaaagcggaacagaagatcgctgaggaggctggcgactggcaggctctgatggatgctggcgcgatcccgttgcctgcaggctgtgccgtgtgcatcggtcttggcgcaggtctcctcaaggagggcgaggtgggcatttccgcatcgaaccgcaattttaagggtcgcatgggctctccagatgcaaaggcatatttggctagccccgaagtcgtcgccgcaagcgccttgaacggcgtcatcagcggccccggcatctacaaacgcccagaggactggaccggtgtctcgatcggtgagggcgaggtcgttgaatccggttcccgtattgatacgaccctcgaagctatggaaaaatttatcggccaattggactccatgatcgatagcagctccaaggcagtcatgccagaggaaagcactggatccggtgcaacggaggtagatatcgtccccggattcccagaaaagatcgagggcgaaattctctttttggacgctgataacattagcactgatggcatctacccaggtaagtacacctaccaggacgacgtgacaaaggataaaatggctcaagtctgcatggaaaactacgatcccgcattctccggcattgcacgtgctggtgatattttcgtgtctggcttcaattttggctgcggttcctcccgtgaacaagcagcgacttccatccttgctaagcagctccccctcgtggtggcgggttccattggcaacacctttagccgcaacgcggtgaataacgctcttccccttctggagatgccgcgcctcatcgaacgcctccgagaggctttcggttctgaaaaacagccgacccgccgcaccggttggacatttacatggaacgttcgcacctcccaggtcaccgtgcaggaaggccccggaggcgagacctggtcccaatccgtgccagctttcccccctaacttgcaggacattattgcacagggtggtttggagaagtgggtcaaaaaagagatcagcaaagct

SEQ ID NO: 58 - protein, 378 aa, Unknown
  Burkholderia sp. KJ006
MSIPIINDTTLRDGEQTAGVAFTVEEKCAIATALSNAGVPELEIGIPSMGADEIADIRSIVDLNLDASVMVWGRLTPGDLAAALRSRPDIIHLSVPVSDIHLQYKLRQPRAWVFAQIKRVIGEAVRSDLKISLGAEDASRADPAFLADVARLAQQCGAQRIRFADTLGVLDPFSTYEAVARLRDAVDIEIEMHAHNDLGLATANTLAALRAGATHANTTVNGLGERAGNAALEEVVMCSRHLLGRDTGVDTTALVGISRLVEHASGRQVALNKSIVGGGVFTHESGIHTDGLAKNPTTYESFDPAELGRARSLVLGKHSGSHGVRHAYEALGLPVADQLVPLLLARIRQHATQTKQAPNAADLYRFLVETRELVEEWS

SEQ ID NO: 59 - DNA, 1134 bp, Artificial
  mutated or codon-optimized sequence
atgtccattcctatcattaacgatacgaccctccgcgacggcgaacagaccgcgggagtcgcattcactgtagaagagaagtgtgctattgcgaccgcactttccaacgccggtgttcccgaacttgagattggcattccatccatgggagcagacgaaattgctgacatccgaagcatcgtggacctcaacctggacgcgtcagttatggtatggggccgattgacccctggtgacctcgcagcggctctccgctcccgcccagacattatccacttgtccgtgccggtgtccgatatccatctgcagtataagttgcgacaaccgcgcgcctgggtgttcgcccaaattaaacgcgttatcggtgaggccgtccgttccgacctcaaaatctccctgggtgcagaagacgctagccgtgctgaccctgccttcctggccgacgtggctcgtttggcgcaacagtgcggtgcacaacgcatccgcttcgctgatacgctcggcgtgctcgaccccttttcaacgtacgaagcagtcgcacgcctccgtgacgctgtagacatcgagatcgaaatgcacgcacacaacgatctgggcttggcgaccgcgaacaccttggctgcgttgcgcgctggcgcaacccacgcaaataccacggtcaatggcctcggtgaacgtgcgggaaacgcagccttggaggaagtcgttatgtgttcacgccatcttcttggtcgagatacaggagtcgataccacggcactggtgggtatttcccgtctggtcgaacacgcatctggccgccaggttgcactgaacaagtcaatcgtcggtggaggtgtcttcacacatgaatccggcatccataccgacggtttggctaaaaatccaaccacgtacgagtcattcgacccagcagaactgggccgtgcacgctcccttgtcctcggcaagcacagcggctcccacggtgttcgacatgcatatgaggcacttggtctgccagttgcggatcagctggtgccccttctccttgcccgcatccgtcaacatgcgactcagaccaagcaagctccaaacgcggcggacttgtatcgcttcttggtggaaacccgcgaacttgtggaagagtggtca

SEQ ID NO: 60 - protein, 385 aa, Azotobacter vinelandii
MASVIIDDTTLRDGEQSAGVAFNADEKIAIARALAELGVPELEIGIPSMGEEEREVMHAIAGLGLSSRLLAWCRLCDVDLAAARSTGVTMVDLSLPVSDLMLHHKLNRDRDWALREVARLVGEARMAGLEVCLGCEDASRADLEFVVQVGEVAQAAGARRLRFADTVGVMEPFGMLDRFRFLSRRLDMELEVHAHDDFGLATANTLAAVMGGATHINTTVNGLGERAGNAALEECVLALKNLHGIDTGIDTRGIPAISALVERASGRQVAWQKSVVGAGVFTHEAGIHVDGLLKHRRNYEGLNPDELGRSHSLVLGKHSGAHMVRNTYRDLGIELADWQSQALLGRIRAFSTRTKRRSPQPAELQDFYRQLCEQGNPELAAGGMA

SEQ ID NO: 61 - DNA, 1155 bp, Artificial
  mutated or codon-optimized sequence
atggcaagcgtaattattgacgacacgactctccgtgatggtgaacagtctgcgggcgttgctttcaatgccgatgagaagattgccattgctcgtgccttggccgaattgggagtccctgaattggaaatcggaattccatctatgggcgaggaagagcgagaagtgatgcacgccatcgcgggactgggtttgtcatcccgtctcctcgcctggtgccgcctctgcgacgttgacctggcagctgcgcgctctaccggcgtgactatggtggacttgtcacttcctgtttctgacctgatgttgcaccataaactcaatcgcgaccgcgactgggcgttgcgtgaagtggcccgtcttgtcggagaagcgcgtatggcaggactcgaagtgtgtttgggttgcgaggacgcgtcccgtgcggatttggagtttgtggtccaggtgggcgaagtggcccaggccgccggtgcccgacgcttgcgtttcgcagacacggtcggtgtgatggagcctttcggtatgttggatcgcttccgatttctttcccgtcgcttggacatggaactggaggtccacgcacacgacgacttcggcctcgccaccgcaaataccctcgcagcagttatgggaggtgctacccacatcaacaccaccgtgaatggtcttggcgagcgcgcgggaaacgctgcgttggaagagtgcgttctggccttgaagaacctgcacggtattgatacaggcatcgacacccgtggcatcccggcgatttccgcgctggtcgaacgtgcgtcgggccgtcaggttgcctggcagaagtccgtggtaggcgccggtgtgttcactcatgaagcaggtattcacgtggatggcctgttgaagcaccgtcgtaactacgaaggccttaaccctgatgaactgggccgatcccactccctcgtgctgggcaaacacagcggagcacatatggtccgtaacacctatcgcgacctgggcattgagctggctgattggcagtcccaagcgcttctgggtcgtatccgtgcgttctccacacgtaccaagcgccgcagcccacaacccgctgaactgcaggatttctaccgccagttgtgtgagcaaggcaaccctgagctggcagcgggtggcatggct

SEQ ID NO: 62 - protein, 374 aa, Methylomonas denitrificans
MKSNSRTIVIDDTTLRDGEQSAGVVFSLEEKLAIAGQLSALGVPELEIGIPAMGAQERDEIKAIAALKLPSNLLVWSRMREEDLQHCLGLGVGTVDLSISASDQHIQHKLKQSRAWVLATIERCVKTAVDAGMQVCVGAEDASRADGDFLALMAEAAQAAGACRIRFADTVGIMEPFGVFEAIRKLRAVTDMDIEMHAHDDLGLATANTLAAAYAGATHVNTTVNGLGERAGNAALEEVVVGLKQLYGFETGVDLRNFPALSRQVATASGDTIGSRKSLIGRDVFSHEAGIHVDGLLKDPNNYQGVDPALVGRSHQLVLGKHSGSQGVMHAYKQLGIQINRWQAGRLLPLIREFVSLHKRAPQSTDLNQFLHSL

SEQ ID NO: 63 - DNA, 1122 bp, Artificial
  mutated or codon-optimized sequence
atgaagagcaactcccgcaccattgtgatcgacgataccacgttgcgtgatggcgagcagtcagccggcgtggtcttctctctggaagaaaagctcgctatcgctggccaactgagcgcgttgggtgtccctgaactcgaaatcggtattcccgccatgggagcgcaggagcgcgatgaaatcaaggccatcgcagcgctgaagcttccttcaaacctgctggtgtggtcacgcatgcgcgaagaggaccttcagcattgtctcggtttgggcgttggtactgtggatctgtccatctccgcatcagaccagcacatccagcacaagcttaaacaaagccgcgcatgggtgcttgcaacaattgagcgctgcgttaagacggccgtggacgcaggtatgcaggtgtgcgtaggcgctgaagacgccagccgcgcggatggcgacttcctcgcccttatggcagaagcggcacaagcggccggagcgtgccgtattcgcttcgccgatacagtcggcatcatggaaccttttggtgtcttcgaagccattcgtaagctgcgtgccgtgactgatatggacatcgaaatgcacgcacatgatgatcttggcttggctacagctaatacgctcgccgcagcgtacgccggtgcgacccacgtgaacacgacagtgaatggactgggtgagcgcgctggtaacgctgctcttgaggaagttgtggtcggccttaaacagctgtacggcttcgagaccggcgtcgacctgcgtaactttcctgctctgtcccgtcaagttgcaacggcttcgggtgataccatcggttctcgaaaatccctcattggtcgtgacgtgttctcccatgaggcaggaatccatgttgacggtctcctgaaagatccgaacaactaccaaggtgtggacccagccctggttggtcgcagccaccagcttgtgctgggcaagcactccggctcccagggcgttatgcatgcgtacaagcagcttggcattcaaatcaaccgttggcaagcaggccgcctcctccctctgattcgagaatttgtttctctccacaagcgtgcaccccagagcactgatctgaaccagtttctccactccctg

SEQ ID NO: 64 - protein, 418 aa, Artificial
  mutated or codon-optimized sequence
MSVSEANGTETIKPPMNGNPYGPNPSDFLSRVNNFSIIESTLREGEQFANAFFDTEKKIQIAKALDNFGVDYIELTSPVASEQSRQDCEAICKLGLKCKILTHIRCHMDDARVAVETGVDGVDVVIGTSQYLRKYSHGKDMTYIIDSATEVINFVKSKGIEVRFSSEDSFRSDLVDLLSLYKAVDKIGVNRVGIADTVGCATPRQVYDLIRTLRGVVSCDIECHFHNDTGMAIANAYCALEAGATHIDTSILGIGERNGITPLGALLARMYVTDKEYITHKYKLNQLRELENLVADAVEVQIPFNNYITGMCAFTHKAGIHAKAILANPSTYEILKPEDFGMSRYVHVGSRLTGWNAIKSRAEQLNLHLTDAQAKELTVRIKKLADVRTLAMDDVDRVLREYHADLSDADRITKEASA

SEQ ID NO: 65 - DNA, 1254 bp, Artificial
  mutated or codon-optimized sequence
atgtccgtcagcgaggccaacggaacggagacgatcaaaccgccaatgaacggaaatccgtatggccctaacccctcagatttcctctcacgcgtcaataacttctcaattatcgaatctactttgcgcgaaggcgaacaatttgccaacgctttcttcgataccgagaagaagatccaaattgccaaggcgcttgataacttcggcgtggattacatcgaacttaccagcccggtagcctctgaacagtcccgccaggactgtgaagcaatctgtaagctcggcctcaagtgcaaaatccttacccatattcgctgccacatggatgacgcacgcgtcgcagtggagacaggtgtggacggcgtcgatgtggtaatcggcacttcccagtatctgcgcaagtactctcatggtaaagatatgacctatattattgattctgcaaccgaagtgatcaacttcgtcaaatccaaaggtattgaggtccgtttttcttccgaggactcattccgctcggatctcgtcgatctgctgtccctgtacaaggcagttgataagatcggcgtgaaccgcgtgggcattgccgataccgtgggatgcgcgacccctcgacaagtgtatgatttgatccgaacccttcgcggcgtggtatcctgcgacatcgagtgccacttccacaacgatactggaatggcgattgcgaatgcctactgcgccctggaagcaggagcaactcatattgatacttccattttgggcatcggcgaacgcaacggtattacgccacttggagccctcctcgcacgaatgtacgtaaccgacaaggagtatattacccacaagtacaaactcaatcagctgcgtgaactggagaacttggtggcggatgccgtggaggttcagatcccattcaataactacattacgggcatgtgcgccttcacacacaaggcaggtatccacgccaaggcaatcctggcaaacccgtctacctacgagatcctgaagcccgaagatttcggaatgtcccgatacgtgcacgttggctcacgcctcaccggctggaatgctattaaatcccgcgccgaacagcttaacctccacttgacggacgctcaagcgaaggaacttaccgtacgcattaaaaagctcgccgacgtccgtaccctggctatggacgatgttgatcgcgttttgcgtgagtatcacgcggatctgtctgatgctgatcgtattacgaaggaggcctccgcc

SEQ ID NO: 66 - protein, 425 aa, Neurospora crassa
MCGTDAPNGTNGASNGVNPANFRSNPYQPVGDFLSNVGSFKIIESTLREGEQFANAYFDTETKIKIAKALDDFGVDYIELTSPAASEQSRRDCEAICKLGLKAKILTHVRCDMRDAQLAVETGVDGLDVVIGTSSFLREHSHGKDMAYIEKTAVEVIEYVKSKGLEVRFSSEDSFRSDLVDLLSLYRAVDKVGVHRVGIADTVGCASPRQVYDLVRTLRGVVSCDIETHFHDDTGCAIANAYCALEAGATHIDTSVLGIGERNGITPLGGLMARMIVTSPDYVKSKYKLHKLKELEDLVAEAVEINTPFNNPITGFCAFTHKAGIHAKAILNNPSTYEILNPADFGLTRYVHFASRLTGWNAVKTRVGQLGLEMTDDQVKECTAKIKALADVRPIAIDDADSIIRTFHLGLHEQNKVQPPAVVEN

SEQ ID NO: 67 - DNA, 1275 bp, Artificial
  mutated or codon-optimized sequence
atgtgtggaaccgatgctcccaacggtactaacggagcctccaatggcgtcaaccccgcaaatttccgctccaacccgtatcagcctgttggtgattttctgtcgaatgtgggtagctttaagatcatcgaatccacccttcgcgagggcgaacagtttgcgaacgcttacttcgataccgagacgaagatcaagattgcaaaggccttggacgatttcggcgttgactacattgagcttacttctcctgccgcgtcagaacaatcacgccgcgattgtgaggctatctgcaagcttggcttgaaagcgaaaatcttgacccatgttcgctgcgacatgcgcgatgctcaactggcagtggaaacaggcgtggatggtctggacgtcgtcatcggcacctcgtcgttcctgcgcgaacactcccacggaaaggacatggcgtacattgagaaaacagcagtggaagtgatcgagtatgtgaaatcgaaaggactggaagtgcgattctcctccgaagactcattccgttctgacctcgtcgaccttctctccctgtaccgcgcggtcgataaagtgggagttcaccgcgtcggtatcgcggacacggttggctgtgcctcgccacgccaggtatacgatcttgttcgtactcttcgcggcgtcgtctcgtgcgacatcgagacacactttcatgatgacacaggctgtgcaatcgctaacgcatattgtgctctggaggcaggtgctacccacatcgatacgagcgtgcttggcattggcgaacgcaacggtattacgcccctcggtggacttatggcgcgcatgattgtgacttcccctgactatgtgaagtccaaatacaagcttcacaagctgaaagagctggaagatttggttgccgaggcggtggagattaacaccccttttaataacccgattaccggtttttgcgcgttcacccataaagccggaatccacgcaaaagcaatcctgaacaatccgtctacgtacgaaatccttaacccggccgactttggtttgacccgctatgtacacttcgcctcgcgcctcaccggttggaacgcagttaagacccgcgtcggtcagttgggtctcgaaatgactgacgatcaggtaaaggagtgcactgcaaagattaaggcactcgccgacgttcgcccaattgcgatcgatgacgcagactctatcattcgcacattccacctcggtttgcacgaacagaacaaagttcagcctccggcggttgttgagaac

SEQ ID NO: 68 - protein, 397 aa, Clostridioides difficile
MCVISKDRAKEIKIVDTTLRDGEQTAGVVFANREKIMIAEMLSDLGVDQIEVGIPTMGGDEKNVIKHICSRNLKSDIMAWNRAVIKDVEESISCGVDAVAISISVSDIHIENKLRTSRGWVLENMAKTVEFAKKNGLYVSVNGEDASRADIDFLTEFINVGKQAGADRFRYCDTVGVMNPFSIKNAIETLYERTNFDIEMHTHNDFGMATANALAGIAAGANYVGVTVNGLGERAGNAALEEVLMALKCVYKCDLNNIDTRKFRGICEYVAQASGRILPTWKPVVGDNMFIHESGIHADGALKDPHNYEPFDPSEVNLERKIVIGKHSGRAAVVNKLSEYEMYISPENATKLLNAIRATSIRLKRSLMDKEILQLYCDILAHEKGTTEEEAVRGSYI

SEQ ID NO: 69 - DNA, 1191 bp, Artificial
  mutated or codon-optimized sequence
atgtgtgttatcagcaaggatcgtgctaaggagatcaagatcgtggacacaacactgcgcgacggcgagcagaccgccggcgtcgtcttcgccaaccgagaaaaaattatgatcgcagaaatgttgtctgacctgggagttgaccaaatcgaagtgggaatccccaccatgggcggcgacgagaagaacgtgattaagcacatttgctctcgcaatctgaagtcggacatcatggcatggaaccgcgcagtcatcaaagatgtagaggagtccatcagctgcggcgttgacgctgtggcaatctccatctccgtctctgatatccatatcgagaacaaacttcgcacctcgcgtggttgggttctggaaaacatggctaagaccgtggagttcgctaagaaaaacggtctgtacgtttccgtgaacggagaagatgcaagccgcgcagatatcgattttctgaccgaatttatcaacgtaggtaagcaggccggagccgaccgcttccgctactgcgataccgtcggcgtgatgaaccccttttccattaagaacgcaatcgagacgctctacgagcgcacaaacttcgacattgagatgcatacccacaatgacttcggcatggccaccgcaaatgcgctggcgggaatcgccgccggcgcaaattacgttggcgtgacagtcaacggcctgggcgaacgtgccggcaacgcagccctcgaagaagtactgatggcgcttaagtgtgtttacaaatgcgatctcaataacattgatacgcgcaagttccgtggtatctgcgaatacgtagctcaagcatccggccgcatcctgcccacctggaaacctgttgtcggagacaacatgttcatccacgaaagcggcattcatgccgatggagcgctgaaggatcctcacaattatgaaccgttcgacccgtccgaggtcaaccttgagcgaaaaatcgtcatcggcaagcactcaggacgtgccgccgtcgtcaataagctttccgagtacgagatgtacatctcccctgagaacgcaaccaagctgctcaacgctatccgtgcaaccagcatccgactgaagcgttcgcttatggacaaggaaatcctgcagctctactgtgacatcttggctcacgaaaagggtaccaccgaggaagaagcggtacgcggctcctacatt

SEQ ID NO: 70 - protein, 527 aa, Artificial
  mutated or codon-optimized sequence
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 71 - DNA, 1581 bp, Artificial
  mutated or codon-optimized sequence
atggcggctgatcagctcgttaaaacggaagtaacaaagaaatccttcaccgctcccgtacagaaagcttctactcctgtcttgaccaacaagaccgttatctccggctctaaagtgaaatcgctctcttccgcccagagcagctcatccggtccgtcgtcttcctccgaagaggacgactcacgtgatattgaatcactggataaaaagattcgaccgctcgaagagcttgaggcgcttttgagctccggcaataccaaacagttgaagaacaaggaggttgccgcccttgtgattcacggcaaacttccgctctatgccttggagaagaagttgggagacaccacgcgcgcagtggccgtgcgccgcaaggcactgtctattctcgcggaggcacccgtcctggcttccgaccgcttgccctacaagaactacgattacgaccgtgtcttcggcgcatgttgcgagaatgtaatcggatatatgccccttcccgttggcgtgatcggcccacttgtcatcgatggcacatcataccatattccaatggcgaccaccgagggttgcctcgtggcgtcggccatgcgcggttgtaaggccatcaacgccggaggtggagcaactaccgtacttactaaggacggcatgactcgtggccccgtggtgcgtttcccgactcttaaacgctccggtgcgtgcaagatctggttggattccgaggagggacagaacgcaatcaaaaaagctttcaacagcacttctcgcttcgctcgacttcagcacattcagacctgtctcgctggtgacttgttgttcatgcgttttcgcaccaccactggagacgcgatgggcatgaatatgattagcaaaggagtggaatactccctcaaacaaatggtggaagaatatggctgggaagacatggaagtggtttccgtgtctggtaattattgtaccgacaagaagccggcagccatcaactggattgagggccgtggcaagtccgtggtggcggaggcgacaattccgggtgatgtcgtgcgcaaagttctcaagtctgacgtgtcagcgttggtagaactgaacatcgcaaaaaaccttgtaggatcggcaatggctggttcagtgggcggttttaatgcgcacgcagccaacctcgtcaccgctgtgttccttgccctgggtcaggatccagcacagaatgtggaaagctcgaactgcatcaccctgatgaaagaagtggatggcgacttgcgtatctccgtctcgatgccatcaatcgaagtgggtaccatcggtggcggaaccgtgttggaaccacagggagcaatgcttgatctcctcggcgttcgaggtccgcacgccacggcgcccggcaccaacgcacgtcagctggcacgcatcgtggcttgcgcggttctcgcgggtgaattgagcctctgcgcagccctggcagcgggtcacctggtgcaatctcacatgacacataaccgtaaaccagccgagcccaccaagccgaataacctcgacgccactgatattaaccgcttgaaggatggttccgtcacctgcatcaaatct

SEQ ID NO: 72 - DNA, 2052 bp, Artificial
  mutated or codon-optimized sequence
atgtttcactcctcccgagcttggctcaagggacaaaacctgacagagaaaatcgtccagtcttacgcagtcaatctccctgaaggcaaggtcgtccactcgggtgactacgtgtcgatcaagcccgcccattgtatgtcccacgataactcctggccagtggccctcaagttcatgggactgggtgcaaccaagatcaagaacccctcccagattgtgactaccctcgatcatgacatccagaacaagtccgagaagaacctcacaaagtacaagaatattgaaaacttcgctaagaaacaccacatcgatcactacccagccggccgaggaattggccaccagattatgatcgaggagggctatgcctttcccctgaacatgaccgtggccagcgactcccactccaacacctacggcggactgggtagcctgggtacacccatcgtgcgaacggacgccgccgctatttgggctaccggccaaacgtggtggcagattccacccgtcgcccaggtggagctgaagggacagctgccccagggagtgtcgggtaaagacatcattgttgcactctgcggcctctttaacaacgaccaggtcctcaatcacgccattgaattcacgggagactccctgaatgccctgcccatcgaccatagactcaccatcgccaacatgacaaccgagtggggtgccctgtctggacttttccccgttgacaagacacttatcgactggtacaagaaccgactccagaagctcggaactaataaccatccccgaatcaaccccaagaccattcgtgctcttgaggagaaagccaagatccccaaggccgacaaggacgcccactacgccaagaagcttattatcgaccttgccactcttacccactatgtctccggtcctaactcggtgaaagtttcgaacaccgttcaggatctgtcccaacaagatattaaaattaacaaggcttacctcgtgtcttgcaccaattcccgactctctgacctccagtctgctgcagacgtcgtctgccctaccggtgacctcaacaaagttaacaaggtcgctcccggagtcgagttttacgtggcagccgcttcctccgagattgaagcagatgctagaaagagcggagcttgggagaagctcctgaaggctggctgcattccccttccctccggctgcggtccttgcatcggcctgggtgccggcctcctcgagcccggtgaggttggtatctctgctacgaatagaaacttcaagggaagaatgggctctaaggacgccctcgcttacctcgcctcgcccgccgtggttgctgcttctgccgtcctgggcaagatctccagccctgccgaggtgctgtccacttccgagatccctttttcgggcgtcaagactgagatcatcgagaaccccgtcgtggaggaggaggtcaacgcacagactgaggcccccaaacagtccgtggagatccttgagggcttcccccgagagttttccggcgagctcgttctctgcgacgctgacaatatcaacactgacggcatctaccctggcaagtacacctaccaagacgacgtccccaaggagaagatggcccaggtctgtatggaaaactacgacgcagagttccggactaaagtccaccccggcgacatcgtggttagcggattcaacttcggtaccggctcctctcgagagcaggccgccacagccctcctggctaaaggcatcaacctggtcgtgtccggatccttcggcaacatctttagccgaaactccattaacaacgctctccttacactcgagattcctgccctcattaagaagctgcgtgaaaagtaccagggcgctcctaaggagcttacgcgacgaaccggatggtttctcaagtgggatgtcgctgatgctaaggtcgtggtcacggaaggatcgctggacggtcccgtgatcctcgagcagaaggttggtgaactgggcaagaacctccaggagatcatcgtgaagggaggacttgaaggttgggtgaagtcccagctg

SEQ ID NO: 73 - protein, 679 aa, Ogataea parapolymorpha
MSRHFSTSLARCRGQNLTEKIVQKYAVGLAPDKLVYSGDYVSIRPAHVMSHDNSWPVALKFKGLGAKKVKDNRQIVNTLDHDVQNKTEKNLAKYHNIEQFAKEQGIDFYPAGRGIGHQIMIEEGYAFPSNLTVASDSHSNTYGGIGSLGTPIVRTDAASIWATGQTWWQIPPVAKVELKGSLPKGVTGKDVIVSLCGLFNNDEVLNHAIEFVGDEIHKLPIDYRLTIANMTTEWGALSGLFPIDETLINFYTNRLLRLGPNHPRINHETVEKLRQNKLAADPDAYYAKTLTIDLSTLSPYISGPNSVKVSNSLSDLSAKNMKVDKAYLVSCTNSRLSDIKAAADIVRGHKIAPGVKFYIAAASSEVQADAEAAGAWQTLLDAGCIPLPAGCGPCIGLGTGLLEEGEVGISATNRNFKGRMGSKDALAFLASPEVVAASAVVGKIAAPEEVDGKPCPPTREVKKTIVVNEKPPSTDDTAKSSGSVLPGFPEEITGELVLCDADNINTDGIYPGKYTYQDDVPREKMAEVCMENYDSEFGKKTKAGDIIISGFNFGTGSSREQAATCILARDIKLVVAGSFGNIFSRNSINNALLTLEIPDLINMLRDRYRDSEPELTRRTGWFLKWNVPKSLVTVTDSSGAVVLEQKVGELGTNLQEIIIQGGLEGWVRSELQKEAQQQQ

SEQ ID NO: 74 - DNA, 2037 bp, Ogataea parapolymorpha
atgagccgccattttagcacttcactggcgcgctgtcgtggccagaacctcaccgaaaagattgtgcagaaatacgcggttggcctggcaccggataagctcgtatactcaggcgactacgtatctatccgcccagctcacgtaatgtcgcatgataatagctggcccgtggctcttaaatttaagggtctgggtgcgaaaaaggtgaaggacaaccgtcaaatcgtcaatacccttgaccacgacgttcagaacaagactgagaagaacctggcaaaataccacaacattgaacaattcgcgaaggagcagggcattgacttttaccccgcaggacggggaatcggccatcagattatgatcgaggaaggctacgcttttccaagcaatctcactgttgcttccgattcgcactccaatacctatggaggcattggctctcttggcaccccgattgtccgtacggatgctgcctctatttgggcgaccggccagacgtggtggcaaatcccacctgtggctaaggtcgagttgaagggatctttgccaaagggcgtaacgggcaaggacgtaatcgtgtccctctgcggtctctttaacaatgatgaggtattgaaccacgcaatcgagttcgtcggcgatgagatccataaactgccgattgattaccggctcacgatcgcaaacatgacgactgaatggggcgccttgtctggtttgttccccattgacgagactctgattaatttttatacgaaccgcctccttcgcttgggtccaaatcacccacggattaatcacgaaaccgtagaaaagctccgccagaataagcttgctgcagaccccgacgcatattacgcaaaaactcttactattgacctctccaccctttctccatatatttccggtccgaactctgtcaaagtgagcaacagcctcagcgacctgtcggcaaaaaatatgaaagttgacaaggcctatctggtgtcctgcaccaattcgcgcctttctgacattaaagcggcagccgatatcgtgcgtggccataagatcgccccgggtgtaaagttctatatcgcggccgcatcgtccgaagtgcaagccgatgcggaggcggctggagcctggcaaactctcctggacgctggttgcattccgcttcccgcgggatgcggtccttgtatcggactcggaacgggcctcttggaggagggcgaagtcggtattagcgccacgaatcgcaacttcaaaggtcgcatgggatccaaagacgccctcgcttttttggcgtccccagaggtcgtagcagctagcgcggtcgtcggcaagatcgcagcacccgaagaggtagatggaaagccatgccctccaacccgcgaagtaaagaaaacgattgtcgtcaacgagaaacccccatcaacggatgacacggctaaaagctctggctctgtgttgccaggattccccgaagagatcactggagagttggtcctgtgtgacgcggacaatattaataccgacggtatttatcctggaaaatatacgtatcaggacgatgtcccccgcgaaaagatggccgaggtgtgcatggaaaattatgactcagagtttggtaagaaaaccaaggctggcgatatcatcatttccggctttaatttcggtacgggttctagccgtgaacaggcggctacttgtattttggcgcgggatattaaacttgtggtggccggatcctttggaaacattttttcccggaactcaatcaataacgctctgctgacgctcgaaattccagaccttatcaatatgctgcgcgatcgctaccgtgattcggaaccggagctgactcgccgcactggctggtttttgaaatggaatgtccccaaatcactggttactgtcacggattcgtcgggcgcggtggttttggagcagaaggttggtgagttgggcactaatctccaagaaattatcatccagggtggtctcgagggctgggttcgctcagagcttcagaaggaggcacagcaacaacaa

SEQ ID NO: 75 - DNA, 2052 bp, Artificial
  mutated or codon-optimized sequence
atgtttcactcttcccgtgcgtggctcaaaggacagaatctgaccgaaaagattgtgcagtcatatgctgtcaatctccctgaaggcaaggtggtccactcgggcgattacgtgtcgattaagccggctcactgcatgtctcatgataattcttggcccgtagcactcaaatttatgggactgggcgccaccaagatcaaaaatccgtctcagattgtgaccaccctggaccatgatatccaaaacaaatccgaaaaaaatctgactaagtacaagaacattgaaaacttcgctaaaaaacaccacatcgatcactaccctgcgggccgcggtatcggccaccaaattatgatcgaggaaggttatgcctttcctctcaacatgaccgtggcatcagactcccactcaaacacgtatggtggtctgggctcgctcggcactccgattgtccgcactgatgcagctgccatctgggctacgggtcagacctggtggcagatcccccctgtggcccaggttgaactgaagggccagcttccccagggtgtgtccggcaaggacatcatcgtagccctttgcggtctctttaacaacgaccaggttttgaaccacgcgattgagttcaccggcgattcgctgaacgcgcttcctattgatcatcgccttaccatcgcgaacatgaccacagaatggggtgccctctccggcctctttcctgtcgataaaaccttgatcgattggtacaaaaatcgtctccagaagttgggaaccaataaccatccgcgtatcaacccgaaaaccattcgagcattggaagagaaggccaaaatccccaaggctgacaaggatgctcattacgctaagaagttgatcatcgacctcgccacccttacccactacgtgtcgggacctaattctgttaaggtgtccaacactgtgcaggacctgagccagcaagatattaaaatcaacaaagcttatttggtttcttgcacaaattcgcgcctgtccgatttgcagtccgcggccgacgttgtctgtccaaccggtgacttgaacaaggttaacaaggttgcaccaggcgttgaattctacgtcgcggcagcatccagcgaaatcgaagccgatgcgcgcaaatccggtgcttgggaaaagctgctgaaggcaggctgcatccccctgccgtctggatgcggcccttgcatcggcttgggcgcaggcttgttggaaccgggcgaggtgggcatctccgcgactaatcgcaactttaagggacgcatgggttcgaaggacgcgcttgcatacttggcgtctcctgcagtcgtagcggcatccgctgttctcggaaagatcagctcgcccgctgaggtcttgagcacctctgaaattcctttctccggcgtgaaaaccgaaatcatcgaaaaccctgttgtggaagaggaagtgaacgcgcagaccgaagcgcctaaacaatccgttgaaatcctcgagggttttccacgcgaattctctggcgaacttgtgctgtgcgacgctgataacatcaacaccgatggtatctacccgggtaaatatacgtaccaagacgacgtcccaaaagaaaaaatggctcaggtttgcatggagaattatgacgccgagtttcgcaccaaggtccaccctggagatattgttgtcagcggtttcaatttcggtaccggttcaagccgcgagcaggccgcaacagcacttctggccaagggtattaacttggttgtatctggcagcttcggtaatattttctcacgaaactcaatcaataacgcactgcttaccctcgagatcccagcgcttattaagaagcttcgtgagaaataccaaggagctcccaaggaattgacgcgccgcacaggttggtttcttaaatgggatgtagcagatgcgaaggtggtggttaccgagggtagcctggacggcccagtgatccttgaacaaaaggtgggcgaacttggcaagaaccttcaggaaattattgttaaaggcggcctcgaaggctgggttaagtctcaactg

SEQ ID NO: 76 - DNA, 1161 bp, Artificial
  mutated or codon-optimized sequence
atgtcaatcaagaatctaaataacggcaaagaggtctttatcgtagacaccacattgagagatggtgagcaaaccgccggtgttgtttttgctaatgaggagaaaatcgcaattgccagtatgttatctgaactgggtgttgatcagttagaagtgggaatcccaaccatgggtggtgatgagaaagaagctattaaacagattgtgaagaggaaccagaaatcttcaatcatggcctggaatagagctgtcattaaggatattgaagaatcaatagattgcggggtcgacgctgtggccattagcataagtgtgtcagatatacatatccagcataaattgaaaacgtctagagggtgggttttggaaaatatggtaaaatcagtcgaatttgctaagaaaaacggattgtacgtatcagtaaatggtgaggatgctagtagagccgatagagagtttcttatccaatttatcgaagtagctaaacaagcgggagcagaccgtttccgttactgcgataccgttggtattatggagccattcaaaattaatgaggatattagatacctacatgcgaagaccaattttgatatcgaaatgcatacacacaacgattttggtatggcgacagccaacgcaattgcgggaattatgggtggcgcttctcacgtgggcgtgacagtaaacggactgggtgaacgtgctggtaacgctgccttagaagaggtacttatggcgctgatgttcgtttacaattacggtggcaatatctctacaacaatgttcagagaagtatctgaatatgtcagtagagcatcaggccgtattttgccagcatggaaagctatagtcggagataacatgttcgctcacgaatcaggcatacatgccgatggtgccatcaaaaatcctaaaaattatgaggctttcgatccaggtatcgttggattagaaagacaaattgtaatcggcaaacactccggtaaagcgggcatcatcaacaaatttaaggaatacggaatcgacctggacaacaattctgctaaagatattttggagatggtgcgttctacttcagtaagactaaaaagatcactgttcgataaagagcttgtccagatctataaggaatacttaagattgagggaagaaaaagtcaac

SEQ ID NO: 77 - DNA, 1284 bp, Artificial
  mutated or codon-optimized sequence
atgacggctgctaagcccaatccatacgcagcaaagcccggcgattacctgagcaacgtgaacaacttccagctcatcgattcaaccctccgtgaaggcgaacaattcgcaaacgccttttttgataccgagaagaagatcgaaatcgcacgagcactggatgatttcggtgtcgattacattgagctgacctctccggtcgcgtctgaacagtctcgaaaagattgtgaagccatctgtaagctcggtctcaaagctaaaatcctgacccacattcgctgccacatggatgatgctaaagtagcagtggagactggagtggacggcgttgatgttgtcattggcacctcgaaatttttgcgtcagtactcccacggtaaggatatgaactacatcgcaaagtctgcggtcgaggtaattgaattcgttaaatccaagggtatcgaaattcgcttctcaagcgaagattctttccgtagcgatctggtggatctgctgaatatctacaagaccgtggataaaatcggcgtgaaccgcgttggcatcgccgacaccgtaggttgtgctaatccccgccaggtctacgaactgattcgcacgcttaaatccgtagtctcgtgtgatatcgaatgtcactttcacaatgacaccggctgtgccatcgcgaacgcctacaccgcactggagggtggcgctcgcttgattgacgtctcggttctgggcatcggtgagcgcaatggtattaccccactcggtggtctcatggcccgtatgatcgtcgctgctcctgactacgttaagtctaagtacaaattgcacaagattcgcgatatcgagaatcttgtcgcagacgcggtggaagtgaacatcccctttaacaacccgatcaccggattttgtgctttcacccataaggctggtatccatgcgaaagcaatcttggcgaaccctagcacctacgagatcttggatcctcacgatttcggaatgaagcgctacattcattttgctaaccgtctgaccggctggaacgctatcaaggcacgtgttgaccagctgaacctgaacttgactgacgatcagatcaaagaggtcacagcgaagatcaaaaaactgggtgacgtgcgctctcttaacattgatgacgtggattccattattaagaacttccacgccgaggtctccacccctcaggttctctctgcgaagaagaataagaagaacgactccgatgtgccagaattggccaccatcccagcggccaagcgtacaaagccgtccgct

SEQ ID NO: 78 - DNA, 1113 bp, Artificial
  mutated or codon-optimized sequence
atgtttcgctcggttgcaacacggttgtcagcttgtagaggtctcgcatcaaacgccgcacgaaagtctttaacgatcggtttaattccaggtgacggaataggcaaagaagttattccagccggcaagcaggtactggaaaatttaaacagcaaacatggtttgtcttttaattttatcgatctctatgcggggtttcagacgtttcaagagacaggtaaagcccttcctgatgagacggtcaaagttcttaaagaacaatgccaaggtgccttgtttggtgcagtacaatcgcctacgacaaaggttgagggctattcaagcccaattgtagctttacgacgcgaaatgggattatttgctaacgttcggccggtcaaatctgttgaaggtgagaaaggcaaacctatagacatggtgattgtccgcgaaaataccgaagatttatacatcaaaattgagaaaacttatattgataaagcgacaggaacacgagtagctgacgccacaaaacggatcagcgaaatcgctacacggcgcattgcaaccatcgctctggacattgcgctgaagcgattacaaacacgtggacaggctacgttgacagtaacccataagagcaatgttctgtcacaatcggatggcctgttcagagaaatctgcaaagaagtgtatgaaagtaacaaagataagtatgggcaaatcaaatacaacgaacaaattgttgattcaatggtgtatagactctttagagagccacagtgctttgatgttatcgtggccccgaatttatatggggatattttatccgacggagcggcagccttagttggctccctgggtgttgtcccgtcagctaacgtcggcccggaaatcgtaattggcgaaccgtgccatggcagtgctccggatattgccggtaaaggaattgccaatcctatcgcgacgattcggtccacagcgttaatgttagaattcctgggtcacaatgaagcagcgcaagatatttataaagcggtggacgcaaatctgcgggagggaagcattaaaacgcctgatctgggcggtaaagccagcactcagcaggtggttgacgacgtactttcaagatta

SEQ ID NO: 79 - DNA, 1113 bp, Artificial
  mutated or codon-optimized sequence
atgtttcgttctgttgccactcggctgtccgcctgccgaggactcgcctctaacgctgccagaaagtctctgaccatcggactcattcccggtgatggaattggaaaggaagtgatccctgctggaaagcaggtcctggagaaccttaactccaaacacggcctgtccttcaacttcatcgatctctacgccggctttcagaccttccaggagaccggaaaggcacttccagacgagacagttaaagtcctgaaggagcaatgccagggcgctctcttcggtgctgttcagtctcccactacgaaggtggagggatacagctcccctattgtggcccttcggcgagagatgggactgttcgcaaacgtgcggcccgtcaaatccgtcgaaggtgaaaagggcaagcccatcgacatggtgatcgtccgggagaacactgaggacctttacatcaagatcgagaagacttacatcgataaggccactggcacccgagtcgctgacgctactaagcgaatctcggaaattgccacccgacgaatcgccacgatcgcacttgacatcgccctcaagcgactccagacccggggtcaagccacgctgacagtgacccacaaatctaacgtgctctcccagtcggacggactcttccgagagatttgcaaggaggtgtatgaatcgaacaaggataaatacggccagatcaagtataacgaacagatcgttgactccatggtctaccgactgttccgagagcctcagtgctttgacgtcatcgttgcccctaacctctacggcgacatcctctcggacggagccgctgctctggtcggctctctgggtgtcgtgccttctgccaacgtgggtcccgagattgtcattggagagccatgccacggttctgcccctgatattgcaggaaagggaatcgctaaccccatcgccactattcgaagcacagctctcatgcttgagttcctcggccacaacgaggccgcccaggacatttacaaggccgtcgacgccaacctccgagagggaagcatcaagaccccagacctgggtggcaaggcctccacccagcaagtggtggacgacgtcctttcgcggctg

SEQ ID NO: 80 - DNA, 1284 bp, Artificial
  mutated or codon-optimized sequence
atgactgccgctaagcctaacccttacgccgccaagcccggcgactacctgtctaacgtgaacaattttcagcttatcgatagcaccctccgagagggagaacagttcgccaacgccttctttgataccgagaagaagattgaaatcgcccgagccctggacgattttggcgtcgactacatcgaactgacatctcccgttgcttcggagcagtcgcggaaggattgcgaggctatttgcaagctgggtcttaaggctaagatcctcacccacattcgatgtcatatggacgacgctaaggtggccgtcgagactggtgtggacggtgtcgacgttgtcatcggcacatccaaatttcttcgacaatattcccacggcaaagacatgaactacatcgccaagtctgcagttgaggtcattgaatttgtgaagagcaagggtattgaaattcggttctcttccgaggactctttccgttctgatcttgttgacctgctcaacatctacaagactgtggataaaatcggcgtgaacagagttggaattgcagacactgttggttgtgccaaccctcggcaggtctacgagctcatccgtacgctcaagtctgtcgtgagctgtgacattgagtgtcactttcacaatgatactggatgcgcaatcgccaatgcatacactgctctcgaaggcggagctcggctgattgacgttagcgtgctgggtattggagagcgtaacggcattacacccctcggcggcctcatggctcgtatgatcgtcgccgcccctgattacgtcaagtctaagtacaagctgcacaagattcgagacattgagaaccttgttgctgacgccgtcgaagtgaatatccctttcaataaccctatcaccggtttctgcgcctttacccacaaggctggcatccacgccaaggctattctggctaacccctctacctacgagattctcgacccccatgactttggaatgaaacgatacatccactttgctaaccgactgaccggctggaatgcaattaaggcccgtgtggaccagctcaacctcaaccttacggacgatcagatcaaagaggttaccgccaagattaagaagcttggagacgtgcggtccctcaacatcgacgacgtggattcgattatcaagaacttccatgctgaagtctctacccctcaagtcctgtccgctaagaagaacaagaagaacgactctgatgtccctgaactcgccactattcctgctgctaaaagaactaagccttccgca

SEQ ID NO: 81 - DNA, 1113 bp, Artificial
  mutated or codon-optimized sequence
atgtttagaagcgttgctactaggctatctgcctgtaggggactagcttcaaacgctgcaagaaaaagcttgactattggtttgataccaggtgatggtataggaaaggaagtaatcccggctggaaagcaagttttggaaaacttaaattctaaacacggtttgtcatttaatttcatagacttgtatgctggcttccaaactttccaagaaacaggaaaggccttgccagacgaaacagtcaaagtcttaaaggagcaatgccagggtgctctttttggcgcagtacagtctcctaccaccaaagtagagggttattcttctccaatagttgccttgagaagggaaatgggcctttttgcaaacgttaggcctgtaaaatccgtcgagggtgaaaaaggtaagccaattgatatggttattgttagagaaaacaccgaggatttgtatattaagatagaaaagacctatatagacaaagccaccggaaccagagttgcagatgcaactaaaagaatttccgagattgcgacgagaaggatagctaccattgccctagatattgccctaaagagattgcaaactagggggcaagctacgttaacagtcacacacaaaagcaatgtgctgtcccaatctgatggtttgttccgtgaaatatgcaaggaagtatacgaaagcaataaagataagtatggtcagataaagtataacgaacaaatcgtcgattcaatggtatataggctgtttagagaacctcagtgctttgatgtaattgttgcccctaacttgtatggtgacatcctatccgatggcgctgcggctctagtaggttccttgggtgtggtcccatcagctaacgttggaccagaaattgtcattggtgagccatgccatggcagtgcgccggatatcgcgggtaagggtatcgcgaaccccatcgccaccattagaagtacagcattaatgcttgaattcctaggccacaatgaggcagcacaggatatctataaggcagtcgacgccaatcttcgtgaaggttctattaaaacaccagatttgggtggcaaggcgtctacacaacaggtcgttgacgacgttttatcaagactt

SEQ ID NO: 82 - DNA, 2079 bp, Artificial
  mutated or codon-optimized sequence
atgcttcgatccacaactttcacccgttcttttcactcttcccgagcttggctcaagggtcagaacctgactgagaagattgtccagagctacgccgtgaacctgcctgagggcaaagtggtccactccggtgactacgtttccatcaagcccgcacactgtatgtctcacgataactcctggcctgttgctcttaagttcatgggtctcggagctactaagattaaaaacccttcgcagattgtcaccacactcgatcacgacatccagaacaagtctgaaaagaaccttaccaagtacaagaatatcgaaaatttcgccaaaaagcaccacattgatcactaccccgctggtcgaggtattggtcatcagattatgatcgaggagggatacgccttcccccttaacatgaccgttgctagcgactcccactctaatacttatggcggactgggatcgctgggtactcccatcgtgcgaaccgatgccgctgctatttgggctacgggccaaacttggtggcagatccctccagtggcacaggtggagctcaagggccagctcccccaaggcgtgagcggaaaggacattatcgtcgccctgtgcggcctgttcaacaacgaccaggtgctgaaccacgctattgagttcactggcgactccctgaacgcactccccattgatcatcgacttaccatcgctaacatgactaccgagtggggtgcactgtccggcctcttccccgttgacaagaccctgattgattggtataagaaccgactccagaagctgggtaccaacaaccacccccgtattaaccccaagaccatccgagccctggaggagaaggccaagattcccaaggctgacaaggacgctcactacgccaagaagctcatcattgaccttgctaccctgacgcactacgtctccggtccaaactccgtcaaggtctcgaatactgtccaagatcttagccaacaggacattaagatcaacaaggcttatctggtgtcgtgtaccaactcccgactctctgatctgcagtctgcagctgatgtggtctgccccacgggtgaccttaataaggtcaacaaggtggcaccaggtgtggagttctacgtcgccgcagcttcctccgagattgaggctgacgccagaaaaagcggtgcttgggagaaacttctgaaggccggttgcatccctcttccctctggctgcggcccttgcatcggtctcggcgctggtctcctggagcccggtgaggtcggaatttccgcaacaaaccgaaacttcaagggacgaatgggcagcaaggacgccctggcttacctggccagccccgctgttgtggccgcctccgccgtcctgggtaagatcagctcccccgctgaggtgctcagcacttccgaaatcccattttccggtgttaagaccgagatcattgaaaaccccgtggtcgaggaggaagttaacgcacaaaccgaagctcctaaacagtctgtcgagatcctggaaggttttccccgagaattttcgggcgagctcgtcctctgcgacgctgataacatcaacaccgatggcatttaccccggtaagtacacctaccaagacgacgtcccaaaggagaagatggcacaggtttgtatggagaactacgatgccgagttccgaacgaaggtccatcctggagatatcgtggtttccggttttaatttcggaactggctcgtctcgagagcaggccgccacagcactgctcgccaagggcattaaccttgtcgtctcgggctcgttcggaaacattttttcccggaactccatcaacaacgccctgctgactcttgagatccctgccctgatcaagaagctgagagagaagtatcagggcgctcctaaggaacttacacgacgtaccggatggtttcttaagtgggacgtggctgacgccaaggtcgtggtgaccgagggatcccttgacggaccagtcatcctggaacagaaggtcggcgagctgggcaagaacctccaggagatcatcgtcaagggaggtctggagggttgggtgaagtctcagctg

SEQ ID NO: 83 - protein, 680 aa, Neosartorya fumigata
MFATTSLRRYTEASSSTTQTSPSSSSWPAPDAAPRVPQTLTEKIVQAYSLGLAEGQYVKAGDYVMLSPHRCMTHDNSWPTALKFMAIGASKVHNPDQIVMTLDHDVQNKSEKNLKKYESIEKFAKQHGIDFYPAGHGVGHQIMIEEGYAFPGTVTVASDSHSNMYGGVGCLGTPMVRTDAATIWATGRTWWKVPPIAKVQFTGTLPEGVTGKDVIVALSGLFNKDEVLNYAIEFTGSEETMKSLSVDTRLTIANMTTEWGALTGLFPIDSTLEQWLRHKAATASRTETARRFAEERINELFANPTVADRGARYAKYLYLDLSTLSPYVSGPNSVKVATPLDELEKHKLKIDKAYLVSCTNSRASDIAAAAKVFKDAVARTGGPVRVADGVEFYVAAASKAEQKIAEEAGDWQALMDAGAIPLPAGCAVCIGLGAGLLKEGEVGISASNRNFKGRMGSPDAKAYLASPEVVAASALNGVISGPGIYKRPEDWTGVSIGEGEVVESGSRIDTTLEAMEKFIGQLDSMIDSSSKAVMPEESTGSGATEVDIVPGFPEKIEGEILFLDADNISTDGIYPGKYTYQDDVTKDKMAQVCMENYDPAFSGIARAGDIFVSGFNFGCGSSREQAATSILAKQLPLVVAGSIGNTFSRNAVNNALPLLEMPRLIERLREAFGSEKQPTR

SEQ ID NO: 84 - DNA, 2040 bp, Artificial
  mutated or codon-optimized sequence
atgttcgcaactacatctttgcgtagatacaccgaagcatcttcttcaacaacgcagacgagcccttcttcatcatcatggccagcaccagatgcggccccaagagtacctcaaacgttaactgagaagatcgttcaagcttattcactgggtcttgctgaaggtcaatatgttaaagcgggtgattatgtaatgttgtcacctcataggtgtatgactcatgacaattcctggccaaccgctttgaaatttatggcgatcggcgcaagtaaagtgcataatcccgatcaaattgttatgacactggatcacgacgttcaaaacaaaagcgaaaaaaatcttaaaaaatacgaatccattgagaagttcgctaaacagcatggtattgacttctatcccgccggccatggtgttggtcatcaaattatgattgaagaaggttacgctttccctggtaccgtgaccgtggcatctgattcacatagcaatatgtatggcggcgtagggtgcttggggacccctatggtccgtacagacgcagctacgatctgggcaactggtagaacttggtggaaggttccacccattgcaaaggtacagttcactggcacattaccggagggtgttactggaaaagatgttattgtagcattaagtggtctattcaataaagacgaagtgttgaattatgcgatcgaatttactggttccgaagagaccatgaaatctctgtctgttgacaccagattaaccatagcaaatatgacgacggaatggggcgcattaacaggcttattcccaatcgattctactcttgagcagtggctgagacacaaagcagctacagcctcaaggacagagacggctaggaggtttgcggaagaaagaattaacgagttatttgcaaaccctaccgttgctgatagaggagccaggtatgctaaatacctttatcttgacttaagtacattatcaccttacgtgtctggtccaaactctgttaaggttgccacccctttagatgaactggagaaacacaagttaaaaatcgataaagcctatcttgtatcttgtacgaattcaagagcatctgatatagcggccgctgcgaaggtatttaaagacgctgttgcaaggactggtggacccgtaagggttgctgatggtgttgagttctatgtagccgccgcttcaaaagctgagcaaaaaattgcagaagaagcaggggattggcaagcattgatggatgcaggtgcaattcccttgccggctggctgcgcggtgtgtataggtttgggtgcgggccttctgaaagagggtgaagttggtatatcagcttcaaacagaaatttcaagggcagaatggggtctccggacgcaaaagcatatttggctagcccggaagttgttgccgcgtcagccctgaacggagtgataagcggtcctggaatttataagagacccgaagattggacgggtgttagtatcggggaaggtgaagtagtcgaatctggttccagaattgataccactcttgaggcaatggagaaattcattggtcaactggattccatgattgatagtagctctaaggctgttatgccggaagaaagcacagggtctggcgcaactgaggtggatatcgtacctggtttccctgaaaaaatcgagggtgagattctttttttagatgctgataatatcagtacggacggcatttaccccggtaaatatacataccaggacgacgtcaccaaggacaagatggcacaagtttgcatggaaaattacgatccggcttttagcggcatcgcaagagctggagacatttttgttagtggattcaactttggatgtggatctagtagagagcaggcagccacatcaatcttggctaaacagctgcccttggttgttgctggtagtatcggtaatacattctctcgtaacgctgtcaataacgcgttgccgcttttggagatgcctagattgatcgaaaggttgagagaggcatttggttccgaaaagcaacctaccagg

SEQ ID NO: 85 - DNA, 2331 bp, Artificial
  mutated or codon-optimized sequence
atgtttaagcgcacaggttcgcttctcctccgttgtcgcgcatcccgcgtgccagtcattggtcgccctttgattagcttgtccaccagcagcacgtccctcagcttgtctcgaccgcgctccttcgcaacaacctccctgcgacgctacactgaggctagctcgtcaaccacgcaaaccagcccatcctccagctcgtggccagcaccagacgccgccccccgcgttccacaaacacttactgagaaaatcgttcaggcttactccctgggattggccgaaggacaatatgtgaaggccggagactatgttatgttgagcccgcaccgttgcatgacgcatgataattcttggcctaccgcactgaagtttatggctatcggagcgtcgaaagttcacaatcccgatcagattgttatgacactcgatcacgatgttcaaaacaaaagcgaaaagaacttgaagaagtacgaatcgatcgagaagttcgccaagcagcacggcatcgacttctaccctgcgggccacggcgttggtcaccaaatcatgatcgaagaaggctacgccttcccaggtaccgtaacagtcgcatctgactctcacagcaatatgtacggcggtgtgggatgtctgggtacaccaatggtgcgtaccgatgcagctacaatctgggcgaccggccgtacctggtggaaggtaccccccattgcgaaagttcagttcacgggaacgctgcctgaaggcgtaacgggaaaagatgttattgtggcactgtcgggtcttttcaacaaggacgaagtcctgaactatgctatcgaatttacaggttccgaagagacgatgaagtcgctgtccgttgatacccgtctcaccatcgcgaatatgactactgaatggggcgcgctgaccggtctcttcccgattgattctacactggaacaatggctgcgccacaaggctgccactgcttcgcgaaccgaaactgcccgccgtttcgcggaagagcgaatcaacgaactctttgcgaatcccacggtggctgaccgcggcgcacgctatgctaagtatctctacctggacctttctacccttagcccgtacgtgtctggacctaactccgttaaagtggcgacccctctggacgaattggaaaagcataaacttaagatcgataaggcatatcttgtgtcatgcaccaactctcgcgcatcagatatcgccgctgcggctaaagttttcaaagatgcagtggcacgcactggcggccctgtgcgagtggccgacggcgttgaattttacgtcgccgccgcatctaaagctgagcaaaagatcgcggaagaagcaggtgactggcaagctcttatggacgcgggcgcaatccccttgcctgccggctgtgcagtttgtatcggcctcggtgccggcctgctcaaggagggagaagtcggcatcagcgcaagcaatcgaaactttaaaggccgcatgggttctcctgacgcaaaggcctatctcgcttccccagaagttgttgcggccagcgcactcaacggagtaatctccggtccaggaatctacaagcgaccagaggactggactggcgtatccattggagaaggtgaagtcgtggaatccggctcacgcattgacaccacccttgaagcaatggagaagttcattggccaactggattcaatgattgattcctcctctaaggctgtcatgccggaggaatccactggatccggtgctaccgaggtcgacatcgttccaggtttccctgagaagatcgaaggtgagatccttttcctggacgcggataacatcagcaccgatggaatttatccaggaaaatatacttaccaggacgacgtgactaaagataagatggcccaagtgtgcatggaaaactatgatccggcattctccggcatcgcccgcgctggagatatctttgtctcaggctttaatttcggctgcggatcctcgcgtgagcaggccgccacctccattcttgccaagcagctcccgctggtggtcgctggatccatcggaaacacgttctctcgcaacgctgttaacaacgcccttcccttgctcgaaatgccacgcttgatcgagcgccttcgtgaagctttcggttctgagaagcagcctactcgccgtacaggttggacatttacctggaacgtgcgcacttcccaggttaccgtccaggagggccccggcggcgaaacctggagccagtccgttccagcatttccacccaatttgcaggacatcatcgctcagggcggactggagaagtgggtgaaaaaggaaatttccaaggcg

SEQ ID NO: 86 - DNA, 1254 bp, Artificial
  mutated or codon-optimized sequence
atgtctgtctcagaggccaatggaactgaaaccatcaaaccaccgatgaacggtaacccatacggaccaaacccaagtgatttccttagcagagtcaacaacttttctattattgagtctacattaagagaaggtgaacaatttgcaaacgccttcttcgatacagaaaagaagatccaaatcgcgaaggctttggataattttggcgtggattacattgaattgactagtcctgtcgcatctgagcagagcaggcaagattgcgaagccatatgtaagttaggtttaaagtgcaaaattttaacacatatacgttgtcacatggacgatgctagagtagcagttgagactggcgttgatggagtaaatgtggttatcggtacatctcaatatctaagaaagtactctcatggcaaggacatgacgtacataattgattcagctactgaagtaataaatttcgttaagtccaaaggcattgaagttcgtttcagctctgaagatagcttcagatccgatttggttgatctgctatccctgtacaaggcagttgataagattggggtgaatagagttgggatcgctgatacagtaggctgcgctactccaagacaggtttacgacttaatccgtacattgagaggtgtggttagttgcgacattgaatgtcattttcataatgatactggtatggctatcgcaaatgcatattgcgcattggaagccggtgccactcacattgatacttcaattttaggtattggagagagaaacggaatcactcccctgggtgctttattggcaagaatgtatgttacagatagagaatacatcacccacaagtacaagttgaatcaacttagggagttagaaaacttagttgctgacgctgtagaagttcagatcccatttaataattatatcacaggtatgtgtgctttcacgcataaagcgggaattcatgctaaagccattttggctaacccttcaacgtacgaaattttaaagcccgaagattttggcatgtccaggtacgtccacgttggttctagattgacaggctggaatgccataaagtcacgtgctgaacagttaaatttgcacttaactgatgcacaagcgaaagaattaacggttagaatcaagaaacttgccgatgtaaggacattggccatggatgatgttgatagagtcttaagagaatatcacgccgatttgtccgacgctgacagaatcacgaaggaggcctcagca

SEQ ID NO: 87 - DNA, 2040 bp, Artificial
  mutated or codon-optimized sequence
atgtttgcaacaacctcattacggcggtacacggaggcgtcaagttctacgacacagacgagtccatcttcatcgtcttggccagctccggatgctgcaccacgggttccgcaaactcttacggagaagattgttcaagcgtattcattaggactcgccgaaggacagtatgttaaagctggagattatgttatgcttagtcctcaccggtgtatgacccatgacaattcttggcctacggctttaaagtttatggctattggcgcatctaaggtgcataatcctgatcagattgtcatgacattggaccatgatgtacagaacaagagcgagaagaacctcaagaaatatgaatccattgaaaaatttgctaagcaacatgggatcgacttttatcctgcgggccatggcgtcggacatcagatcatgatagaagagggatatgcctttccgggcacggtcactgtcgcctcagattcacattcgaatatgtatggtggtgtaggatgtttgggaacccctatggtcagaactgacgcggcgacaatttgggctacaggacgtacttggtggaaagtcccgcctatcgcaaaagtccaatttactggtactttgccggaaggcgttacgggcaaggatgtaattgtcgctctgagcggccttttcaacaaagatgaagtgttgaactatgccatagaatttacagggtctgaagaaacgatgaagagtctctcagtagatactcgtcttacaattgccaatatgacaacggagtggggtgctctgacgggcctgttccctatcgacagcaccctggaacaatggcttcggcataaagcggccacggcgtcccgcacagaaaccgcgagacgcttcgcagaagaacggatcaatgaactgtttgctaacccgacggtagcagatcgcggcgcgagatatgcgaaatatttatacctggatctttccacgctttctccgtatgtttcaggaccgaattctgtgaaggtagctacaccgttagatgagcttgaaaagcataagttaaaaatcgataaagcgtaccttgtttcgtgcacaaatagcagagcgagcgatatcgctgcggctgcgaaggtatttaaggacgctgttgccagaacaggcggaccagttagagttgccgatggcgtagagttttatgtggcggcagcgagcaaagcagaacagaagattgccgaagaagctggagattggcaagcgttaatggatgcgggagccatccctttaccggcaggttgcgcagtatgtattgggcttggggctggactgcttaaagaaggagaagtcggaatcagcgcttctaaccgcaacttcaaaggacgcatggggtcacctgatgcgaaagcttatcttgcatcaccggaggtggttgccgcaagtgctttaaacggagtaattagtggcccgggaatttacaaacggccggaagactggacgggagtttcgatcggggaaggtgaagtagtagaaagcgggtctcgcattgataccactctggaagcgatggaaaagtttatcgggcagcttgactcgatgattgatagctcatctaaagccgtgatgcctgaggaatcaactgggtcaggagccacggaagtggatatcgtcccgggattcccggaaaaaatagaaggcgaaatcttgtttcttgatgctgataacatttctacagatggtatctatccgggaaaatatacgtaccaggacgatgtcacgaaagataaaatggcgcaggtttgcatggagaattatgacccggcctttagcggaatagcgcgagcgggcgatatctttgtctctggttttaactttggttgtggctcctcacgggaacaggcagcaacgtctattttggcaaaacaactcccgcttgttgtcgcgggctctattggaaacacattttctcggaacgcggtgaataacgctctgcctctgctggaaatgccgagattaatcgagcgtttgcgcgaagccttcggctcggaaaagcagccaacccgc

SEQ ID NO: 88 - protein, 334 aa, Thermus thermophilus
MAYRICLIEGDGIGHEVIPAARRVLEATGLPLEFVEAEAGWETFERRGTSVPEETVEKILSCHATLFGAATSPTRKVPGFFGAIRYLRRRLDLYANVRPAKSRPVPGSRPGVDLVIVRENTEGLYVEQERRYLDVAIADAVISKKASERIGRAALRIAEGRPRKTLHIAHKANVLPLTQGLFLDTVKEVAKDFPLVNVQDIIVDNCAMQLVMRPERFDVIVTTNLLGDILSDLAAGLVGGLGLAPSGNIGDTTAVFEPVHGSAPDIAGKGIANPTAAILSAAMMLDYLGEKEAAKRVEKAVDLVLERGPRTPDLGGDATTEAFTEAVVEALKSL

SEQ ID NO: 89 - DNA, 1002 bp, Artificial
  mutated or codon-optimized sequence
atggcatatcgcatttgccttatagaaggagatggtattgggcacgaagtcattccagcggctagacgtgtcctcgaggccaccggtctgccgcttgagtttgtagaagcggaagccggatgggagaccttcgaacgccgtggcacaagcgtccctgaggaaacggtggaaaagatccttagctgccacgccacgctgtttggggctgccacttcaccgacccgtaaggtgccgggttttttcggagccattcggtatttgcggcgaagactggatctctacgctaatgtaagaccggctaaatcgcgccctgtgccgggtagccgcccaggcgtggatctggtaattgtcagagagaacactgaaggcttgtacgttgagcaagagcgccgttacctggatgtggcgattgcggatgcggtaatttctaagaaagctagcgaaagaattggaagagcggccttgagaatcgctgaaggacggcctagaaaaacactgcacattgcacataaagcaaatgtgttgccgctgacacagggactgttcttggataccgttaaagaagttgcaaaagactttccgctggtaaatgtgcaggacattatagtggataattgtgccatgcagctggttatgcgcccagaacgttttgatgtcatcgttacaacaaaccttcttggcgatatcttgtcggatctggcagccggattagtagggggattggggctggccccttccggcaacattggggataccacagcagtctttgaaccggttcatggttcagccccagatattgctggcaaggggatcgcgaacccgacagccgcaattctgtctgcagcgatgatgcttgattatttgggagaaaaggaagcagcaaagcgcgtcgaaaaagcggttgatttagtcttagaacgtgggccacgtactccggacctggggggagacgcgaccaccgaggcctttacggaagccgtcgttgaagctctgaaatccctg

SEQ ID NO: 90 - protein, 418 aa, Artificial
  mutated or codon-optimized sequence
MSVSEANGTETIKPPMNGNPYGPNPSDFLSRVNNFSIIESTLREGEQFANAFFDTEKKIQIAKALDNFGVDYIELTSPVASEQSRQDCEAICKLGLKCKILTHIRCHMDDARVAVETGVDGVNVVIGTSQYLRKYSHGKDMTYIIDSATEVINFVKSKGIEVRFSSEDSFRSDLVDLLSLYKAVDKIGVNRVGIADTVGCATPRQVYDLIRTLRGVVSCDIECHFHNDTGMAIANAYCALEAGATHIDTSILGIGERNGITPLGALLARMYVTDREYITHKYKLNQLRELENLVADAVEVQIPFNNYITGMCAFTHKAGIHAKAILANPSTYEILKPEDFGMSRYVHVGSRLTGWNAIKSRAEQLNLHLTDAQAKELTVRIKKLADVRTLAMDDVDRVLREYHADLSDADRITKEASA

SEQ ID NO: 91 - DNA, 1254 bp, Artificial
  mutated or codon-optimized sequence
atgtctgtatcagaggcaaacggcaccgagactattaaaccaccaatgaacggaaatccatatgggccgaacccttcagactttctttcacgcgtcaacaatttttccatcatcgaatcgacgctgcgcgaaggtgaacaatttgcaaatgctttcttcgatacagaaaagaaaatccaaattgcgaaggcgcttgacaattttggagtcgattatattgagttaaccagccctgtcgcgtctgagcaatcacggcaagattgcgaagcaatttgtaagttagggctcaaatgtaaaatccttactcacatacggtgccatatggatgatgcccgggttgcagttgaaactggcgtagatggtgtcaacgttgtgattggcacatctcaatacctccggaaatacagtcacggaaaggacatgacatatatcattgacagtgcaactgaagtaattaacttcgtgaaatcaaaaggtattgaagtgcgctttagttcagaagattcgttcagatcagacctggttgacctcctttcattgtataaagccgttgataaaattggcgtaaaccgcgtaggaatcgctgatacggtggggtgtgctacaccgcggcaggtttacgaccttattagaacgctcagaggggttgtttcatgcgatattgaatgccattttcataacgacacggggatggcgatcgcgaatgcttactgcgcacttgaagccggcgccacacacatcgacactagtattttaggcattggcgaacgtaacggcatcacaccacttggcgcgcttttggcacgtatgtatgtgactgaccgagaatatattacccataaatataaactgaatcagctgcgcgaactggagaatcttgtcgccgacgccgtggaagtgcagatcccattcaataactatataactggaatgtgcgctttcacccacaaagccggcattcacgctaaagcaattcttgctaaccctagtacatatgaaatcctgaaaccggaagatttcggaatgtctcgatatgttcatgtgggatctagacttacgggctggaatgctattaaatcccgggccgagcagctgaatctccatcttacggatgcacaggcaaaggagttaacggttcgtattaaaaaattagctgacgtaagaacgttggcaatggatgatgtcgatcgggttctccgcgaatatcatgctgatttaagcgacgcggaccgcattacaaaagaagcatccgcc

SEQ ID NO: 92 - DNA, 1128 bp, Artificial
  mutated or codon-optimized sequence
atgagagagtggaagatcatcgattccactctgagagaaggagagcaatttgaaaaagctaatttttccacccaagataaggtggaaattgcaaaagcacttgatgaatttggaatcgagtacatcgaagttactacacctgttgcttcacctcaatccagaaaagatgcagaagtccttgcgagcttaggcttgaaagcgaaggtagtaactcatatacaatgtagattagatgccgccaaagtagctgtcgaaactggagttcaagggatagatctactatttggtacctccaaataccttagagctgcacatggtagagatattcccagaataatagaagaagcaaaggaagtcatagcatatatcagggaagcggctcctcacgttgaagtccgtttcagtgccgaagacacatttagatctgaagagcaagatttattggcagtttatgaggccgttgctccctacgttgacagagtcggcttagctgatactgttggtgttgctacaccaagacaagtttatgcgcttgtgcgtgaagtcagaagggttgtcggacctcgtgtcgatatagaattccatggtcataacgatactggctgcgccatcgctaacgcttatgaagccatcgaagctggggccacccacgttgatactaccatattaggtataggtgaaagaaacggtataacacctctgggaggttttttagccaggatgtatacgttgcaaccggaatatgtcagaagaaaatataaattggaaatgttgcctgaattggacagaatggtggccagaatggtgggagtcgaaatccctttcaacaactatataacgggagaaactgccttctcacataaagcaggcatgcacttaaaggctatttatattaatccagaggcatacgagccataccctccagaggtttttggagttaagagaaaattaattatcgcctcaaggctaacaggaagacatgcaattaaagcaagagctgaagaactgggattgcactatggggaagaagaattacacagggttacgcaacatattaaggctcttgcagatcgtgggcaattaacgctggaagaattggacaggattttaagagaatggattaccgca

SEQ ID NO: 93 - DNA, 2040 bp, Artificial
  mutated or codon-optimized sequence
atgttcgccactacttccctgcgacgttacacggaagcctcttcttccaccacccagacttccccctcgtcttcttcctggcctgctcccgacgccgccccccgagtccctcagactctgactgagaagatcgtccaagcttattctctcggactcgcagagggtcagtacgtcaaggccggagactacgttatgctctccccccaccggtgcatgacgcacgacaattcctggcctaccgctctgaagtttatggccatcggagcctctaaggtccataaccccgaccagattgtgatgacactggatcacgatgttcagaacaagtcggagaagaacctgaagaaatacgagtccatcgagaagttcgccaagcaacatggaattgacttctaccccgcaggtcacggcgtcggacaccaaattatgattgaggagggttacgctttccccggaaccgtcacagtggcctccgattcgcactcgaatatgtatggcggagtgggctgtctcggcacccctatggtccgaacagatgctgctacaatttgggccaccggcagaacgtggtggaaggtgccacccattgcaaaggttcagttcactggaacccttcctgagggcgtgaccggaaaggatgtcatcgttgctctcagcggtctgttcaacaaggacgaggtccttaactatgcaattgagttcaccggatccgaggaaactatgaagtctctttccgttgatacgcgactcactatcgccaacatgactaccgaatggggagccctgactggtctcttcccaatcgattccacgctcgagcagtggctgcgacacaaggccgctaccgcctcccggaccgagacagccagacggtttgcagaagagcgaattaacgagctgtttgctaaccccacggttgctgaccgaggcgccagatacgctaagtatctgtatctggacctgtctactctctctccttatgtctctggtcccaactccgtgaaggttgcaacacccctggatgaactggagaagcacaagctgaagatcgataaggcctacctcgtgtcttgcacaaactcgcgagcttcggatatcgccgctgccgccaaggtgtttaaggacgccgttgcccgtaccggcggccctgtccgagtcgccgacggtgtcgaattctacgtggcagccgcctccaaggccgaacagaagattgccgaggaagctggcgactggcaagccctcatggatgctggagctatccctctgcctgcaggttgcgctgtctgcatcggcctcggagccggactcctgaaggaaggtgaagtcggaatctcggccagcaaccgaaacttcaagggccgtatgggctcgccagacgccaaggcctatctggcctcccccgaggtggtcgccgcttctgccctcaacggtgtcatctctggaccaggaatctacaagcgacccgaggattggactggcgtctctattggagagggtgaggttgtcgagagcggttcgcgaatcgacactaccctcgaagccatggagaagtttatcggccagctggattccatgatcgactcgtcctccaaggctgtcatgcccgaggagtcgactggttccggagctacagaagttgacattgtccccggcttcccagagaagattgaaggcgagatcctcttcctcgacgctgacaacatttccactgacggcatctacccaggcaagtacacctaccaggatgacgtgaccaaggacaaaatggctcaggtgtgcatggagaactacgaccccgctttctcgggtattgcccgagccggtgacattttcgtgtctggatttaactttggttgtggctcctctagagagcaggccgctaccagcatcctcgccaagcagctgccccttgtggttgctggttccatcggcaacaccttctcgcgaaatgctgtgaacaacgccctcccccttctcgaaatgcctcggctcattgagcgactgagagaagctttcggctctgagaagcaacccactcgt

SEQ ID NO: 94 - DNA, 2331 bp, Artificial
  mutated or codon-optimized sequence
atgttcaagcgaactggctccctgctcctgcggtgtcgagcatcccgggtccctgtgattggacgaccccttatttcgctctctacttcgtcgacatctctgagcctgtcgcgacctcgatcgttcgcaaccacatctctccgacggtacaccgaggcttcgtcttctaccacccagacctctccctcctcctcgtcttggcccgcacccgatgccgccccccgagtcccccagaccctgaccgagaaaattgttcaggcctactcccttggcctcgccgagggtcagtatgtgaaggccggcgactacgtcatgctgtctccccatcgatgtatgacacacgacaactcttggcctaccgccctgaagttcatggccatcggcgcctctaaggttcacaaccccgatcaaattgtcatgaccctcgatcacgacgttcagaacaagtccgaaaagaacctgaagaagtacgaatcgatcgagaaattcgccaagcagcatggtatcgacttctaccctgctggccatggagttggtcaccaaatcatgattgaagagggctacgcctttccaggtactgtcaccgtggcctcggactcccactccaacatgtacggcggtgttggctgcctgggaacccccatggtgcgaactgatgctgctactatttgggctaccggcagaacgtggtggaaggttccccccatcgccaaggttcagttcaccggcactctccctgagggtgttaccggaaaggatgtcattgtcgccctttcgggactcttcaacaaggacgaggttcttaactacgctattgagtttactggtagcgaagagaccatgaagagcctcagcgtggataccagactgaccattgctaatatgacgaccgagtggggcgctcttaccggcctcttccccattgactcgaccctcgagcaatggctgagacataaggccgctaccgcttccagaactgaaacagcccgacgtttcgccgaggagcggattaatgagctttttgccaaccctacagttgccgaccgaggtgctcggtacgctaaatacctttacctcgacctctccacgctttccccttacgtctccggccccaacagcgtcaaggttgccactcccctcgacgagctcgagaagcataaactgaaaatcgacaaggcttacctcgtcagctgtactaactctcgagcttcggacatcgccgccgctgccaaggtctttaaggacgctgtcgcccggaccggcggccccgtgcgtgtcgccgacggtgttgaattctacgtcgccgctgcctctaaggctgagcagaagatcgccgaggaggctggtgattggcaagcactgatggatgcaggcgctattcccctgcctgccggatgcgccgtttgtatcggacttggtgctggcctgctcaaggagggtgaagttggcatttcggcctctaatcgaaactttaagggtcggatgggttcccccgacgctaaggcttacctggcttctcccgaggtggtggccgcatctgctctgaacggagttatctccggacctggtatctacaagcgacccgaggactggaccggagtttccatcggtgaaggtgaggtcgtcgagtccggcagccgtatcgacaccaccctggaggctatggaaaagttcattggacagctcgattctatgatcgattcgtcgtcgaaggctgtgatgccagaggagtccaccggaagcggtgctaccgaggtcgatatcgtccctggcttccctgagaagatcgaaggtgagattctctttctggacgccgacaacatttctactgatggaatttaccctggcaagtacacctaccaagacgacgttaccaaggacaagatggctcaggtgtgcatggagaactacgacccagctttctctggaatcgcccgtgccggcgacatctttgtctccggcttcaactttggttgcggttccagccgagagcaggccgctacttccattctggcaaagcaactgccactcgtggttgctggctctattggaaacaccttttctcgtaatgccgtgaacaacgctctccctctgcttgagatgcctcgactgattgaacgtctccgagaggcctttggatccgagaagcaacctacccggcgaaccggatggaccttcacatggaacgtgagaacctcccaggttaccgtccaggagggtcccggaggtgagacctggtctcagagcgtgcccgccttccccccaaacctccaggatattatcgcccaaggtggactcgagaagtgggtgaaaaaagagatctccaaggct

SEQ ID NO: 95 - DNA, 2040 bp, Artificial
  mutated or codon-optimized sequence
atgttcgcaaccacgtcattgcgacgttacacagaggctagctcaagcaccacccagacatcgccatcctcatcctcctggcctgccccagacgccgcaccacgtgtgccccagactctgactgagaaaatcgtgcaagcatactctctcggccttgccgagggtcaatacgttaaggccggtgactatgtcatgctgtccccacatcgctgtatgacccatgacaactcctggccaacagccctgaagttcatggctatcggtgcatctaaggtacacaacccagaccaaatcgttatgacactcgaccatgacgtgcagaacaagagcgaaaaaaacctcaaaaagtacgaatccatcgagaagtttgcgaagcagcatggcattgacttctacccggctggccacggcgtaggccatcaaatcatgatcgaagaaggttacgcctttcctggaaccgtcaccgttgcatcagattcccattccaatatgtatggtggtgtgggctgcctcggcacccctatggtgcgcactgacgcagccacaatctgggccacgggccgcacctggtggaaggtgcctcctatcgccaaggtgcagtttactggtactctgcctgagggcgttaccggtaaagatgtgatcgttgcgttgtcgggcctgtttaacaaagacgaagtgctcaactacgcgatcgagtttaccggcagcgaggaaaccatgaaaagcctgtctgttgacacccgtctgaccatcgcgaacatgaccacggaatggggtgctctgaccggtctgttccccattgattccaccctcgagcaatggctccgccacaaggcggcgactgcgtctcgaactgagacagcacgccgcttcgctgaggagcgcatcaacgaactcttcgccaatccaacagttgcagaccgtggcgcacgttacgccaaatacttgtatctggatttgtccaccctgtccccttatgtcagcggtccgaacagcgtaaaggtcgcaacacctctcgatgaactggagaaacacaaattgaaaatcgacaaggcatacctcgtctcctgcaccaacagccgtgcgagcgacattgcagcggccgctaaggtattcaaggatgccgtcgcccgtactggtggtcctgtgcgcgtggcagacggagtcgaattctacgtggccgcagcctcaaaggctgaacagaagatcgctgaagaggcgggagactggcaagccctgatggacgcaggtgcaatcccccttcccgcaggttgcgccgtgtgtattggcctcggagctggtctcctgaaagagggagaagtgggtatttccgcctcaaaccgcaatttcaagggccgcatgggttcgccagatgcaaaagcctacttggcctctcctgaagtggtcgctgcctctgcgcttaacggtgtgatctccggcccgggcatttataagcgcccagaggactggaccggcgtctcgatcggcgaaggcgaggttgtggaatcgggcagccgtattgacaccacacttgaggcaatggagaagtttattggccaacttgattctatgatcgattcaagctctaaggctgtcatgccagaagaaagcacaggatcaggtgcaactgaagtcgacattgttccaggattccctgaaaagattgaaggagagattctgtttcttgacgccgataacattagcaccgacggcatttatcctggcaagtatacctatcaagatgatgttacgaaggacaagatggcccaagtttgcatggagaactatgatcctgcattctcaggtatcgcccgcgctggcgacattttcgtaagcggcttcaacttcggttgcggctcttctcgtgaacaagctgcaacctccattcttgctaaacagcttccattggtggtcgcgggctctattggcaacaccttctcccgcaatgctgtgaacaacgcactcccgctccttgaaatgccacgtctgatcgagcgcttgcgagaggcattcggctctgagaagcaaccaacacgt

SEQ ID NO: 96 - protein, 387 aa, Unknown
  Clostridium sp. C8
MSIKNLNNGKEVFIVDTTLRDGEQTAGVVFANEEKIAIASMLSELGVDQLEVGIPTMGGDEKEAIKQIVKRNQKSSIMAWNRAVIKDIEESIDCGVDAVAISISVSDIHIQHKLKTSRGWVLENMVKSVEFAKKNGLYVSVNGEDASRADREFLIQFIEVAKQAGADRFRYCDTVGIMEPFKINEDIRYLHAKTNFDIEMHTHNDFGMATANAIAGIMGGASHVGVTVNGLGERAGNAALEEVLMALMFVYNYGGNISTTMFREVSEYVSRASGRILPAWKAIVGDNMFAHESGIHADGAIKNPKNYEAFDPGIVGLERQIVIGKHSGKAGIINKFKEYGIDLDNNSAKDILEMVRSTSVRLKRSLFDKELVQIYKEYLRLREEKVN

SEQ ID NO: 97 - DNA, 1161 bp, Artificial
  mutated or codon-optimized sequence
atgagcatcaaaaatttgaataacggaaaggaggtgtttatagtagataccactttaagagatggtgaacagacagcgggtgtggtgttcgcgaatgaggaaaaaatcgcgatcgcgagtatgctttccgagctcggagtggatcaactggaggtgggcattccgacaatgggcggagatgagaaggaggccatcaaacaaatcgtaaaacgtaatcagaaatcaagcattatggcctggaaccgcgctgttattaaggatatcgaagaatctatcgactgcggagtggacgcagtcgcgatctcaatttcagtaagtgatattcacattcagcataagctcaaaacaagccgggggtgggttctggaaaatatggtcaagtcagttgaatttgccaaaaagaatggcctgtatgtgtctgtcaatggcgaggacgcctctcgtgcggatagagaatttttaatccagttcattgaggtggcgaaacaggcaggtgctgatagatttagatactgtgacacagtcgggattatggagcctttcaagataaacgaagatattcggtatttgcatgctaaaaccaatttcgacatcgaaatgcacacgcacaacgattttggaatggctactgctaacgctatagctggaatcatgggcggtgcgagccatgtcggcgttacggtgaacgggctgggagagcgtgctggtaatgctgctttagaggaggtcctgatggctttgatgtttgtctataattacggcggaaacatttcgacaaccatgtttagagaagtttcagaatatgtgagccgagctagcggacgtattttgcctgcctggaaggcaattgtgggtgataacatgtttgcacatgaatctggaatccacgccgatggggcaataaagaatccgaaaaattatgaagcctttgatcctggtatcgtaggtttggagcgccagatcgtgataggaaagcacagtggtaaagcgggaattattaataaatttaaagaatatggtatcgatctggataacaactcggcgaaggacattttagaaatggttcgcagcacgtcagtgcgtttaaagcggtccctgttcgacaaagaattagtacaaatatacaaagaataccttcgtttgcgcgaggaaaaagtcaat

SEQ ID NO: 98 - DNA, 1002 bp, Artificial
  mutated or codon-optimized sequence
atggcttaccggatctgtctcattgagggcgacggcattggccacgaggtgatccctgccgcccgtcgagtcctcgaggcaaccggtcttcccctcgagtttgtcgaggcagaggctggctgggagacctttgagagaagaggtactagcgtgcctgaggagaccgttgagaagattctttcctgtcacgctaccctgtttggcgcagctacttctcccacgagaaaagttcctggattcttcggtgccattcgatacctgcgtagacgtctggacctgtacgccaacgttagacccgcaaagtctcgacccgttcccggctctcgtccaggcgttgacctggtcattgttcgagagaacaccgagggcctgtacgtcgaacaagaacgacgataccttgacgtcgctatcgctgacgccgtgatttctaaaaaggcttctgagcgtatcggtcgagcagctctgcgaatcgccgagggtcgaccccgaaagactctgcacattgcacataaggcaaatgtgcttccactcacacagggcctcttcctggacacagtcaaggaagtcgccaaggacttccccctcgtgaacgttcaggacattatcgtcgataactgcgccatgcagctggttatgcgaccagagcgatttgacgtcattgttaccacgaaccttctgggcgacattctctccgacctcgccgccggactggtgggcggactcggtctggcacccagcggtaacattggcgacaccaccgccgtgtttgagcctgttcacggttctgcccctgatattgccggtaagggcatcgccaaccccaccgccgctatcctgtccgccgccatgatgctcgactacctcggtgaaaaggaggctgctaagcgtgttgagaaggctgtcgatcttgtcctggaacgtggaccccgaacaccagacctgggcggcgatgctacgacagaagccttcacggaagccgtggtggaggccctcaagtcgctc

SEQ ID NO: 99 - DNA, 2052 bp, Artificial
  mutated or codon-optimized sequence
atgtttcattcctcaagagcgtggctaaaaggacaaaatctgacagaaaagatagtgcaatcttacgccgttaacttaccagaaggcaaagttgtacattctggcgattacgtctctattaaacctgctcactgtatgtctcatgataattcctggccagtggctttaaaatttatggggcttggcgcgaccaaaattaaaaacccctcacaaattgttactacgctggatcacgacattcagaataaatctgagaaaaacttgaccaaatacaagaacatcgagaactttgccaaaaagcaccatatagatcactatccagctggtagaggtataggacatcaaatcatgattgaagaaggctacgctttcccactgaacatgacagttgcttcagattcacatagtaatacttatgggggacttggttcattgggaacccccattgttagaacagacgctgctgctatctgggcaactggtcaaacatggtggcaaattcctccagtggcacaggtcgaacttaagggccaattacctcaaggtgttagtggaaaagacattattgttgctttgtgcggtttattcaataacgatcaagtattaaatcatgcgattgaattcactggggactctctgaatgctttgcctatagatcataggttaactattgccaatatgacaactgaatggggggctcttagcggtttgttcccagtggataagacccttatagactggtacaagaatcgtttgcaaaaattaggtacaaacaatcatcctagaattaatcctaaaaccataagagcgctagaagagaaggctaaaataccgaaagcggataaagacgcccattacgccaaaaaattgattattgacttagcaaccttgacccattatgtgtcaggtccaaattctgtaaaggtgtcaaatactgttcaagacctgtctcaacaagacatcaagattaacaaagcctatttggtgtcttgtacgaatagcagattaagtgacttgcaatcagcagcagatgttgtatgccctactggagatttaaataaagttaacaaggtcgctccaggcgttgaattttacgtggcggctgcttcatccgaaattgaagccgatgctaggaagagtggtgcttgggagaaactactaaaagctggttgtatcccattaccgagtggttgtggtccttgtataggtttaggagccggactgttggaaccgggagaagtgggtatctctgcgactaacagaaacttcaagggaaggatgggatctaaagatgccttggcctatctggcatctcctgctgtagttgcggcatcagccgtgttaggaaaaatatctagcccagctgaggttctttcaacttcagagatacctttctccggtgtcaagacagaaattattgagaatcctgtcgtcgaagaagaagtcaacgctcaaacagaagcaccaaagcaatctgttgagattcttgaaggttttccgagagaattcagtggagaattggtgctatgcgatgcagacaacattaacactgatggtatctacccagggaaatacacttatcaagacgacgttcctaaggaaaagatggctcaagtttgcatggaaaactatgacgcagaatttaggactaaagtacatcccggtgacattgttgtgagcggttttaatttcggtaccggttcatctagagagcaagcagccacagctttgttggctaagggcatcaatttggtggtgtctggtagctttgggaacattttctctcgtaattctattaacaatgctttgcttacacttgagattccggccttaattaaaaagttaagagagaaataccagggcgcgcctaaagaattgacaagacgtacgggctggttcctgaaatgggacgttgcggatgcaaaagtggttgttacagaaggttctttggacggcccggtgatcctggagcaaaaggttggagaactaggaaaaaatctgcaagaaattattgtcaagggtggcttagaaggttgggtgaagagtcaacta

SEQ ID NO: 100 - DNA, 1161 bp, Artificial
  mutated or codon-optimized sequence
atgtccattaagaacctgaataatggcaaggaagtgttcattgttgataccaccctccgagacggcgagcagactgccggcgtggttttcgccaacgaggaaaagattgcaattgcaagcatgcttagcgagcttggcgtcgatcagcttgaagtgggcattcctaccatgggaggcgacgagaaggaggccattaagcagatcgttaagcgtaatcagaagtcctcgatcatggcctggaaccgagcagttatcaaagacatcgaagaatccatcgactgcggcgtggacgccgttgccattagcatctcggtgtccgatattcacattcagcacaaacttaagacttctcgaggctgggttttggaaaatatggtaaagtccgtcgagttcgctaagaagaatggtttgtatgttagcgtaaacggcgaagatgcgtcgcgagcagaccgcgaatttctgatccagtttatcgaagtggcaaaacaggcgggcgccgatcgcttccgctactgtgacaccgttggaatcatggaaccatttaagatcaatgaggatatccgctacctccatgctaagaccaacttcgatatcgagatgcacacgcataacgattttggtatggctactgctaacgcaattgcaggtatcatgggcggagcctctcacgtgggtgtcacggtcaacggtctcggcgaacgcgctggcaacgctgcgttggaggaagtcttgatggctctcatgtttgtctataactacggtggcaacatctccactacgatgttccgagaagtctccgagtatgtgtcccgcgcaagcggccgtatcctgcccgcgtggaaggcgatcgttggcgataatatgtttgctcatgagtccggcatccacgccgacggagcgatcaagaatcctaaaaactacgaagcctttgatccaggcatcgtcggcttggagcgccagatcgttattggtaaacacagcggcaaggcgggcatcatcaacaagtttaaggaatacggcattgatttggataataactctgccaaagatattcttgaaatggtgcgctctacctcggtccgcctgaaacgtagcctttttgataaggaacttgttcaaatctacaaagaataccttcgtctccgagaagagaaggtgaac

SEQ ID NO: 101 - DNA, 1161 bp, Artificial
  mutated or codon-optimized sequence
atgtccatcaagaacctcaacaacggcaaagaggtgttcattgtggacactacactgcgagacggcgagcagaccgctggcgtcgtgttcgcaaacgaggagaagatcgccattgcttcgatgctctcggaactgggcgtcgatcaactggaggtgggtatccctaccatgggcggagacgaaaaggaggccattaaacaaattgtcaagcggaaccagaagtctagcatcatggcatggaaccgagctgttatcaaagacatcgaggagagcattgattgtggtgtcgatgccgtcgccatctcgatctctgtttctgacatccacattcagcacaaactgaagacctctcgaggttgggtcctggagaacatggtcaagtcggtggaattcgccaagaagaatggtctgtacgtgtccgtcaacggcgaggacgcctcccgagctgaccgtgagttccttatccagtttattgaagttgccaagcaagctggagctgatcgattccgatattgtgacaccgtgggaattatggagccctttaagattaacgaggacatccgatacctgcatgcaaagacgaatttcgacatcgagatgcacacccacaacgacttcggtatggctactgctaacgccattgctggcattatgggaggtgcctcgcacgtcggtgttaccgtgaacggtcttggcgagcgagccggcaacgctgccctggaggaggtcctgatggccctcatgtttgtttacaactacggcggcaacatctccaccactatgttccgagaagtttccgagtatgtttctcgagcatcgggacgaattctcccagcctggaaggccatcgttggtgataatatgttcgcccacgaatctggaatccacgccgacggagctattaagaaccccaagaactacgaggcttttgaccccggcattgtgggactggagcgacaaatcgttatcggtaagcattccggaaaggccggcatcatcaacaagtttaaggagtacggtattgacctcgacaacaactccgccaaagacattcttgaaatggtccgaagcacctccgtccgactgaagagatccctgttcgataaagagcttgtccagatttacaaagagtacctccgactcagagaggagaaggttaac

SEQ ID NO: 102 - DNA, 1002 bp, Artificial
  mutated or codon-optimized sequence
atggcgtaccgtatttgtttgatcgagggcgatggcatcggccacgaagtcatcccagctgcgcgccgcgtgctggaggctaccggacttcctctggaattcgttgaagcagaagcgggctgggaaaccttcgagcgccgcggcacctccgtgcccgaagaaactgtggaaaagatcctttcgtgtcatgcaacgctgttcggcgctgcaacctcaccaacccgcaaggtgcctggattcttcggcgccatccgctacctgcgccgccgcctcgatttgtacgctaacgtgcgccccgctaaatcccgtcctgtccctggctcgcgccctggtgtagacttggtcatcgtccgtgaaaacactgaaggcctgtacgtcgagcaagagcgacgctatctcgacgtggcaatcgcggatgccgttatctccaagaaggcatcagagcgtattggccgagccgcgctccgcatcgcggaaggccgaccgcgcaagaccctccacatcgctcacaaagccaacgtcttgccgctcacgcagggtttgtttctggacacggtgaaggaggtggcaaaggacttcccattggttaacgttcaagatatcattgttgacaattgcgcaatgcagctggtcatgcgacccgagcgatttgatgtgattgtcacgacgaatttgctgggtgatatcctctccgatttggcggctggcctggtgggcggtctcggcctggcaccaagcggcaacatcggtgatactaccgccgtgttcgagccggtccacggctcagcgcctgatattgcaggaaaaggtatcgccaacccgacggcagcgatcctttccgcagcaatgatgctggattaccttggcgaaaaggaagcggcgaaacgcgttgaaaaggcggtggacctcgtgctcgagcgcggaccacgaaccccggatttgggtggcgacgcaaccacggaagcttttaccgaagctgtcgttgaagctctgaagtctctc

SEQ ID NO: 103 - DNA, 2331 bp, Artificial
  mutated or codon-optimized sequence
atgttcaagagaaccgggagtctcctgctgcgttgtcgcgcgtcgcgagttccggtgattggtcgtcctttgatttctctgtcgaccagctcaacgtctcttagccttagcagacctcgcagtttcgccaccacgagcttgcgaagatacactgaagcgtcatcaagcaccactcagacatcgccaagtagctcttcttggcctgctccggatgcagcaccacgtgtgccacaaaccttgacagagaaaatcgttcaagcctacagcctgggacttgcagaggggcagtatgtgaaagcgggagactatgttatgctttctcctcatcgctgcatgactcacgataactcgtggcctacagcgttaaaatttatggcaattggagcatcgaaagtgcataacccggaccaaatagtgatgaccttggatcacgacgtccaaaacaaatctgaaaaaaatctcaaaaagtatgaatcgatcgaaaaattcgccaaacaacatggcatcgatttctaccctgcgggccacggagttgggcaccagattatgatcgaagaaggatatgcctttccgggtacagtaaccgtagcctctgacagccacagtaacatgtatgggggcgtcggttgcctgggaacgcctatggttcgcacggacgcagccacaatttgggcgacagggcgaacgtggtggaaagtaccacctatcgcgaaagtacagttcacgggcacgctgcctgaaggagtgacaggtaaagatgttattgttgcgcttagtggtctctttaataaggatgaagtcttgaattatgcgatagagtttacagggtccgaggaaacaatgaaaagcctgagtgttgatacccgattgacgattgctaacatgactactgaatggggtgcacttacaggtttgtttcctatagatagcactttagaacaatggctgcgacataaagcggcaacagcgtcacgcacggaaaccgcgagacgattcgctgaagaacgtatcaatgaactctttgccaatccaacagttgcggatcggggcgcccgctatgcaaaatatctttatctcgacctctccaccctgagcccatatgtttcaggaccgaatagcgtgaaggtcgcgactccgttagatgaattagaaaaacataaattaaaaattgacaaggcttaccttgttagctgtacaaacagccgtgcatcggatatagctgccgcggcgaaagtttttaaggatgcggtcgctcggaccggcggtccggtaagagttgctgacggggtggaattctacgtcgcagccgctagcaaagcggaacaaaaaatcgcggaggaagcaggggattggcaagcgttgatggatgcgggtgccatcccgctcccagcaggctgcgctgtttgtattggtttaggggctggactcttaaaagaaggggaggttggcattagcgcaagcaatcgcaattttaagggccgaatgggtagtccggatgcgaaagcgtatttggcatcaccggaggtggtcgccgctagtgccctcaatggggtcatttccgggccggggatctacaaaagaccagaagattggacaggagtcagtatcggcgaaggcgaagtagtcgaatcaggtagccgtattgatacaacacttgaagcgatggaaaagtttattggacaactcgatagcatgatcgatagctcaagcaaagctgttatgccggaagaatcaacggggtcaggagcaacagaagtggacattgtcccgggctttccggagaaaatcgaaggggagattttatttttagatgccgataatatttcaacggatggcatttacccaggcaaatatacgtatcaggatgacgtcacaaaggataaaatggcgcaggtatgtatggaaaactatgatcctgcattttctggcatcgcgcgggcgggcgacattttcgtcagcggctttaatttcggatgtggatcatcgagagagcaagcggcgacatcgattcttgctaaacagctgccgcttgtggtcgcggggagtattggaaacacatttagccgtaatgctgtgaacaatgccttgccactgttagaaatgccgagactgatcgaacgtctgcgagaagcttttgggagcgaaaagcaaccgacccgtagaaccggttggaccttcacgtggaatgtcagaacatcccaagtcacagtgcaagaaggaccgggcggcgagacatggtcgcaatctgttccagcatttcctccgaacctccaagatataattgcgcagggtggtctcgaaaagtgggttaagaaagaaataagtaaggca

SEQ ID NO: 104 - DNA, 1254 bp, Artificial
  mutated or codon-optimized sequence
atgtccgtatccgaggccaatggcaccgagacgatcaagccgccgatgaacggcaacccgtacggcccaaacccttccgatttcctgtcccgtgtgaataacttttccatcatcgaatcaactttgcgagaaggtgaacagtttgccaacgcattcttcgataccgagaaaaagattcaaatcgcgaaagctctcgacaatttcggtgtggattacattgagctcacttccccagtggctagcgagcaatctcgacaggattgcgaagcgatctgcaagttgggtctcaaatgcaaaatcctgacccacatccgctgtcacatggacgatgcccgcgtggcagtggaaactggtgtggacggcgtaaacgttgttatcggcacttctcagtatctccgaaaatactcccacggaaaagatatgacatacattatcgattccgcaaccgaagtaatcaactttgtgaaatcgaaaggcattgaggtgcgttttagctccgaagattcttttcgctccgatcttgtggatctgctttctttgtacaaagcagtggataagattggagtcaaccgagttggcatcgcagataccgtcggctgcgctacgcctcgtcaggtgtatgaccttattcgcaccctgcgaggtgtcgtctcctgtgatatcgaatgccatttccataacgataccggcatggcgatcgccaatgcatactgcgccctggaagccggtgcaacccacatcgatacctcgatcctcggcattggtgagcgaaacggtattacaccactcggagcgctgctcgcacgaatgtacgtgacagatcgcgaatacattacccataaatacaaactgaaccaacttcgcgagttggagaacttggtcgcagacgctgttgaggtgcagatcccctttaacaactacatcacaggcatgtgcgcattcacccacaaagcaggcatccacgcaaaggcgattctcgccaatccttctacatatgaaattctgaagcccgaggatttcggcatgtcccgctatgtccacgttggttcccgcctgaccggttggaacgcgatcaaatcccgagcagaacagctcaacttgcacttgaccgacgcccaggcaaaggaacttaccgtccgtatcaaaaaactggccgatgtacgcacattggcaatggatgatgtcgaccgtgtgctgcgcgagtatcacgcggatttgtccgatgcagatcgcatcaccaaggaagcctctgcc

SEQ ID NO: 105 - DNA, 1254 bp, Artificial
  mutated or codon-optimized sequence
atgtcggtgtctgaggccaacggtaccgagacgattaagcctcccatgaacggtaacccctacggtcccaatccttctgacttcctctctcgagttaataacttctccatcatcgagtcgaccctccgggaaggtgagcagttcgccaacgcattctttgacactgaaaagaagatccaaattgccaaggctcttgacaactttggcgtggattatatcgagctcacctcccctgttgcctccgagcagtcccgacaggattgcgaggctatttgcaaacttggccttaagtgcaagatcctcacccacatcagatgccacatggacgatgctagagtcgctgtcgagaccggagttgatggcgtcaacgttgttattggaacgtctcaatatctccgaaagtactctcatggcaaggacatgacctacattattgattccgctacggaggttattaactttgtcaagtccaaaggaatcgaggtccgattctcgtccgaggactccttccgaagcgatctcgtggacctcctttccctgtacaaggccgttgataagatcggagtcaacagagtcggcattgccgacaccgtcggttgcgccaccccacgacaggtgtatgatctcatccgtaccctgcgtggcgtggtctcctgtgatatcgaatgccactttcacaacgacactggtatggccatcgctaacgcctactgcgccctcgaggccggagcaacccacatcgacacgtccattctcggaatcggcgaacggaacggtattacaccactgggagctctcctcgctcggatgtacgtcaccgaccgggagtacatcacacataagtacaagctgaaccagcttcgagagcttgagaacctggttgctgacgctgtcgaggtccagatcccctttaataactacatcaccggcatgtgcgcttttacccacaaagcaggtattcatgccaaagcaatcctggctaaccccagcacatacgagattctgaagcccgaagacttcggaatgtcccgttacgtccacgtgggttcccgactcacgggctggaatgctattaagtccagagctgaacaactgaacctgcatcttaccgacgcccaggcaaaggaactgaccgtccgaatcaagaagctggctgatgtgcgaactctggccatggatgacgttgacagagttctgcgagaatatcacgccgacctgtctgatgctgatcgaattactaaggaagcctccgcc

SEQ ID NO: 106 - DNA, 2079 bp, Artificial
  mutated or codon-optimized sequence
atgttgaggtcaactacattcactaggtctttccactcttccagagcctggctgaaaggtcagaatttgaccgaaaaaatagttcagagctatgcggttaatttacccgaaggtaaggttgtccattcaggagattatgtttccattaaacccgcccattgcatgtctcatgataattcttggccagttgctcttaaatttatgggtttgggtgcaacgaaaataaagaacccctcacaaatcgttactactctagatcatgacattcaaaataagtccgaaaaaaatcttactaagtacaaaaatatagaaaactttgcaaaaaaacatcatatcgatcattaccctgcgggtagaggtataggtcatcagatcatgattgaagaaggatatgcgttccctttgaacatgactgttgcgagcgactctcatagcaacacttatggtggcttgggatctttaggaacgccaatcgtcagaaccgatgcggccgctatatgggcaacagggcaaacatggtggcagatcccccctgtggcgcaagtggagcttaagggtcaattgccccaaggtgtctccggtaaagatataatcgttgccttatgcggtttattcaataatgatcaagttctgaaccatgctattgaatttactggagattctcttaatgcattgccaatagatcataggctaaccattgcaaatatgacaacagaatggggtgctctgagtggtttgtttcctgtcgacaaaactttaattgattggtacaaaaatagattgcaaaaattgggaaccaacaaccatccaagaatcaatccaaaaactatcagggccctagaagaaaaggcgaagatccctaaagcagacaaggatgcccattatgctaaaaagcttataatagaccttgcaactttaacacattacgtgagtggtccaaacagcgtcaaggtctctaatacagtacaagatttgtcccagcaagatataaagataaacaaagcatatcttgtttcttgcacaaactctcgtttgtctgatttacaaagcgctgctgatgtagtctgccctacaggtgacttaaacaaggtgaataaagtggctcccggtgtagaattctatgtggctgctgcttcatcagaaatagaggcagatgcacgtaaatctggtgcctgggagaaacttctaaaggccggttgtatccctttacctagtggctgcggaccatgtattgggcttggagctggtttattggaaccgggggaggtgggaatctctgccacaaatagaaattttaaaggcagaatgggatcaaaggatgctctagcttatttggcctcccccgcggtagttgcagcatctgctgttttgggaaaaattagctcaccagctgaggtcttgtctacttccgagatccctttttctggtgtgaaaacagagattatagagaaccctgttgtagaagaggaagtaaacgcccagactgaagctcctaagcaatctgttgagattttagaaggttttcccagagagttcagcggggaattagtattgtgcgatgctgataatatcaatacagacggcatctaccctggtaaatatacatatcaagatgatgtccccaaagagaaaatggcccaagtatgcatggagaactatgacgccgaatttaggaccaaggttcatcctggtgacattgtcgtgtctggctttaactttggaaccggtagtagcagagaacaagccgcaactgcgttgttggcgaagggtatcaatttggttgtttcagggagtttcggtaatatctttagcaggaattctatcaataatgctctgcttacacttgaaataccagctttaataaaaaaacttagagagaagtatcagggtgcaccaaaagaacttaccagaagaaccggttggttcttgaagtgggacgtggctgacgctaaagttgtagttactgaaggttctcttgatggaccagtgatattggaacaaaaggtaggtgaattaggtaaaaatctacaggaaatcatagttaaaggcggcttagaaggttgggtgaaatcacaatta

SEQ ID NO: 107 - protein, 371 aa, Ogataea parapolymorpha
MASRTLSKQFARTYATKALKIGLIPGDGIGREVIPAGKQVLEALPSDLGLKFEFTELKAGFELFKQTGTALPDETVEVLQKSCDGALFGAVSSPTTKVEGYSSPIVALRKKLGLYANVRPVKSVEGIGRPVDMVIVRENTEDLYIKEEKLYEKDGQKVAEAIKRITERATTKIGAIALEIALQRQAIRELGGASLHSQPTLTVTHKSNVLSVSDGLFRETVRKLYDSNPAKYSGVQYKEQIVDSMVYRMFREPEIFDVVVAPNLYGDILSDGAAALVGSLGVVPSANVGDNFAIGEPCHGSAPDIEGKGIANPIATIRSTALMLEFMGHPEAASKIYAAVDANLKEDVIKTPDLGGKSSTQEVVADVIRRL

SEQ ID NO: 108 - DNA, 1113 bp, Ogataea parapolymorpha
atggcatcgcgcaccctctccaagcaatttgcacgcacctacgcaaccaaagctcttaagatcggccttatccctggcgacggcattggccgcgaggtgattcctgctggtaagcaggtgctcgaagctctcccctcagatctgggactcaagttcgaattcacggagcttaaggccggattcgagctgtttaaacaaaccggtaccgccctccctgatgaaactgtggaggtcctccagaaatcctgtgatggagcgctgttcggagcggtctcctcgcccaccactaaagttgagggctacagctctccaattgttgcactgcgtaagaagcttggcctctacgctaacgtacgtccagtgaaatccgttgaaggcatcggtcgcccggtggacatggtcatcgtgcgggagaacaccgaagacctttacattaaggaagaaaaattgtatgaaaaggacggccagaaggttgcagaggcaatcaaacgaatcaccgaacgcgccaccacaaaaatcggtgccatcgcattggagattgcgcttcagcgacaagctatccgcgagctcgggggtgcctccctccacagccagccaactctgaccgtgacccacaaatcaaatgtgttgtccgtgagcgatggtcttttccgggagacggttcgtaagctctacgattctaatccagctaagtatagcggcgttcaatataaggaacaaatcgtggattctatggtctaccgcatgttccgcgaacccgagattttcgatgtggttgtcgcaccgaacctctatggtgatatcctctcagacggagcagctgcccttgttggctccctgggtgtggtcccctcggctaacgttggagataacttcgcaatcggcgaaccgtgccacggtagcgctccagacatcgagggcaagggaatcgcaaaccccatcgcgacgatccggagcaccgcacttatgctcgaatttatgggccacccagaggcggcgtcaaagatttacgcggctgttgatgccaacctgaaagaggacgtaattaagaccccagacctgggtggaaagtcctccacccaagaagttgtggcggacgtgattcggcgactc

SEQ ID NO: 109 - DNA, 1284 bp, Artificial
  mutated or codon-optimized sequence
atgacagcagccaagccgaacccctatgcagccaagccaggagactacttatccaatgttaacaacttccagttgatcgatagtactttaagggaaggtgaacaatttgcaaatgctttctttgacaccgaaaagaaaattgaaattgcacgtgctctagatgactttggcgttgattacattgagttgacatctccagttgcctctgaacaatcaagaaaagactgcgaagctatatgcaagcttggcctgaaagctaaaatattgacccacattaggtgccacatggacgacgctaaagttgcagtagagacgggcgtggatggtgtagacgtagtgattggaaccagtaagttcttgagacagtactcccacgggaaggacatgaattatattgcgaagtctgctgtcgaagtcattgaatttgtcaaatccaaggggatagaaattagattttcctccgaagactcctttagaagcgatttagtagacttactgaatatatacaagaccgttgataagataggtgtcaatcgtgttggtattgctgatacggttggttgcgcgaatcccagacaagtctatgaattgattcgtaccctgaaatccgtagtgtcctgtgatatagaatgtcatttccataacgatacaggttgcgcaattgcaaacgcatatacagctcttgagggcggtgctagattgattgacgtatccgtccttggaattggtgaaagaaacggcattactccattgggcggattgatggctcgtatgatagtcgctgcccctgattatgtgaagtccaagtacaagttacataaaattagggatatcgaaaatttagtcgctgatgccgtggaagttaacattccattcaataatccgataacaggcttttgtgcattcacgcataaagcgggcatacatgccaaagctatattagctaaccctagcacctacgaaattttagatccccatgattttggtatgaaacgttacatacatttcgcaaacagacttaccggttggaacgcaataaaggcaagagttgaccagcttaacctaaatctgactgacgaccaaataaaagaagtgacagctaagattaaaaagctgggcgatgtaaggagtttgaacattgatgacgtagatagtataatcaagaactttcatgctgaagtaagtactccccaagtattgtccgcaaagaaaaataagaagaacgatagcgatgtgcctgaattggctaccataccagccgctaaaagaaccaaaccttctgca

SEQ ID NO: 110 - DNA, 1284 bp, Artificial
  mutated or codon-optimized sequence
atgactgctgcaaaaccgaacccgtacgcagccaaaccgggagattacttaagcaatgttaacaattttcagctcatcgatagtactttacgcgaaggggagcaatttgcaaatgcgtttttcgatacggaaaaaaaaatcgagatagcccgtgcgttagatgattttggcgtagactacattgaacttacgtccccggtcgctagcgaacaaagccggaaagactgtgaagccatttgcaagcttgggcttaaagcgaaaattcttacccatataagatgtcatatggacgacgccaaagtcgcggttgagaccggtgtcgatggcgtcgatgttgtcatcggcacttctaagtttttacgacaatattcgcacggaaaagacatgaattacatcgcaaagagtgcggtagaggtgatagaatttgtaaaatcaaaagggattgagatccgtttctcaagtgaggattcatttcgatcggatttggtggatctgctgaacatatacaaaacggtcgataaaatcggcgtgaatagagtgggaatagccgatactgttggctgcgcaaatccacgccaagtctacgaacttattcgcacgctcaaatcagtagtgtcatgcgacatagaatgccattttcacaatgacactggttgcgcgattgcaaacgcttatacggctttggaaggaggggctcgcttaattgatgtgtcagtcctggggattggcgagcgcaatggtattacgccattaggcgggcttatggcacgcatgatcgttgcggcgccggactacgttaaatcgaaatataaactgcataaaatccgggatatcgagaatttagtagccgatgctgtcgaagtcaacattccatttaacaaccctattacaggattttgcgcatttacgcataaggccggcatccacgccaaagcaatcttggccaatccttcaacatatgaaatattggaccctcacgactttggtatgaagcgctatattcattttgcgaatcgccttacaggatggaatgctatcaaggcgcgagttgaccagcttaaccttaaccttaccgacgatcaaattaaagaagtaaccgctaagattaaaaaactgggagacgttcgatccctgaatatcgacgacgtcgactccatcatcaagaacttccacgcagaagtgtctacgccgcaggtcctctcagctaaaaaaaataaaaaaaacgactctgatgttcctgaattagcaacaattccggcagctaagcgtacaaagccgtctgct

SEQ ID NO: 111 - DNA, 2079 bp, Artificial
  mutated or codon-optimized sequence
atgttaagaagtacgactttcacaagatcctttcatagctcccgggcgtggcttaaaggacagaatcttaccgaaaaaatcgtccaatcgtacgccgtcaatcttccggagggtaaagttgtacattcaggagactacgtttcaataaagccggcacattgtatgtctcatgacaattcctggcctgttgctctgaagtttatgggcctgggggccacaaagattaagaatccatcgcagatcgtcacaacattggatcatgacattcagaacaaatctgagaagaacctcacaaaatataaaaatatcgaaaactttgcaaaaaaacatcatattgaccactatcctgctgggagaggtattggtcaccaaattatgattgaggagggatacgcgttcccattaaatatgacggttgcgtcagattctcatagcaatacttacggtggcctggggagtttgggcacgccgattgtgcgtactgatgctgcggcaatttgggcaacagggcaaacatggtggcaaattccaccggttgcgcaggtcgagttgaaaggccagcttccgcaaggagtgagcggcaaagatattatcgtcgcattgtgtggtctctttaataatgaccaggtccttaatcacgctatcgagtttacaggagactcgctgaatgcactgcctatagatcatcgtttaacaatcgcaaatatgacaacagagtggggagccctgtctggcctgtttccggtagataaaactcttatcgattggtataaaaatcgtctgcagaagttaggtaccaataaccatccgcgcattaatcctaaaacaattcgagcattagaagagaaagccaaaattccgaaggcagacaaggatgcgcattatgcgaaaaaactgattattgatttggccacattaacacattatgtatcggggccgaactctgtaaaagtttccaatacggtccaagatttatcacaacaagacatcaaaatcaacaaggcatatctggttagctgcaccaatagccgattatccgatctgcaaagcgcggccgatgttgtatgtccaacaggtgatctcaataaggtaaataaagtcgctccgggagtagaattctacgtggccgccgcttccagtgagatcgaagcggacgccagaaaatcaggagcttgggaaaaactcttaaaagccggctgtattcctctgccgtctggctgcgggccatgcatcggattaggggcgggcctcctcgaaccgggcgaggtcggcatctcagctacaaatcgcaattttaaaggccgtatgggaagcaaagacgccttagcttatttggcttctccagctgttgtcgcagcatcagcagtcctgggaaagatttcttctcctgcagaagtattatccacatcagaaattccgttttctggggtaaaaaccgaaattatagaaaatccggtggtcgaggaagaggttaatgcgcagaccgaggctcctaaacagtctgtggaaatccttgaaggatttccgagagaatttagtggggaattagtgctgtgtgatgctgataacattaataccgatggcatctacccgggcaagtatacttaccaggacgacgtgcctaaagagaaaatggcacaagtttgcatggagaactacgatgctgaattcagaacgaaggtgcatccgggcgatatcgttgtctctggatttaatttcggtacgggctcgtccagagagcaggccgcaacggcacttcttgccaaaggcattaatctggttgtcagcggttcttttggcaatattttctcacgtaactcaatcaacaatgctctccttactcttgaaatcccggcacttattaaaaaactccgtgagaaatatcaaggcgctccaaaggaacttacaagacgtacaggttggttccttaaatgggatgttgccgatgcgaaagtggttgtcacggaaggatcccttgacgggccggtcattctggaacaaaaggtcggtgagctcggaaaaaatcttcaggagattattgttaaaggcggcttagaaggctgggttaaatcacaattg

SEQ ID NO: 112 - DNA, 1128 bp, Artificial
  mutated or codon-optimized sequence
atgcgtgagtggaagatcatcgactcgaccctgcgagaaggagagcagttcgagaaggccaacttctctacccaggacaaggttgagattgctaaggccctggacgagttcggaattgagtacattgaggtcactacacctgtcgcctcgccccagagccgtaaggacgctgaggtgctggcctctctcggcctcaaggctaaggtggttacccacatccagtgtagacttgatgctgcaaaggtcgccgtcgagactggcgtgcagggaatcgacctcctcttcggtacgagcaaataccttcgagccgcccatggacgagacatccctcgaatcattgaggaggccaaggaagtgattgcctacattagagaagctgcaccccatgtggaggtccgtttttccgctgaggatactttccgatctgaggagcaggatcttcttgctgtctacgaggccgtggccccctatgtcgacagagtgggcctggctgacaccgtgggtgtggctacgccacgtcaggtctacgccctggtccgagaggtgcgaagagttgtcggcccccgggtggacatcgagtttcatggacataacgacacgggctgtgctattgctaacgcttatgaagcaattgaagctggcgccacccacgtggataccactatcctcggaattggagaacgtaacggcatcacacccctgggtggtttccttgcacgaatgtataccctccaacccgagtacgtgcgacgtaagtacaagctcgaaatgctccccgagctggacagaatggttgctcgaatggtcggagtggaaattcccttcaacaactacatcaccggcgagaccgcctttagccacaaggctggcatgcacctgaaagctatctatatcaatcccgaggcatatgagccttacccccccgaggttttcggcgtgaagcgaaaactgatcatcgcatcgcggctgacaggtagacatgccatcaaagcacgtgccgaggaactgggtctccattacggagaggaggagctccatcgagtcacccagcacatcaaggccctggccgacagaggtcagctcaccctggaggagctggaccgaatcctgcgagagtggattaccgct

SEQ ID NO: 113 - DNA, 2052 bp, Artificial
  mutated or codon-optimized sequence
atgttccattcgtcacgcgcatggttaaaaggtcaaaatctgaccgaaaaaattgtgcagagctatgcagtaaacttacctgaagggaaagttgtccatagcggcgattacgtatcaattaaaccagcacattgcatgagccatgacaattcttggccggtggccttaaagtttatggggctgggggcaaccaaaattaaaaaccctagccagattgttaccacgcttgatcacgacatccagaacaaaagcgaaaaaaacctgaccaaatataagaacattgagaattttgcgaaaaaacatcacattgatcactatccagcgggtcgcggaatcggccaccaaattatgattgaggaaggttatgcttttcctcttaatatgaccgttgctagtgactcacattcaaatacatatggcgggctggggtcactgggcacaccgatagttcgtaccgatgcagccgcgatttgggctacagggcaaacatggtggcaaatccctccagtagcccaagttgagcttaaggggcaattacctcagggagtctcaggcaaagacattatcgtagctttatgcggtctgttcaataatgatcaagtacttaaccatgccattgaattcaccggcgacagcctcaacgcacttcctattgaccatcggctgacaatcgccaacatgacgacggagtggggagcattgtcaggactgtttccggttgacaagactcttatcgactggtacaaaaatcgccttcaaaaattaggaactaataaccatccacggatcaatccgaaaactatcagagcgttggaagaaaaagcgaaaattccgaaagctgataaagatgcccattacgctaagaaattgatcattgatctggctactttgacccattatgtgtcaggacctaactcagtaaaagtctcaaatacagttcaggatttatctcagcaagacatcaagatcaataaggcatacctggttagctgcactaattcacggttgtctgatcttcaatctgcagcggacgtagtttgccctacgggagatctgaataaagtaaacaaagtagctccgggcgtggaattttatgtagcggctgcgagcagcgagattgaagctgacgcacgcaagtcgggtgcctgggagaaacttttgaaagccggatgtataccgttgccgtccgggtgtggaccttgtatcggattaggcgcagggctgctcgaacctggcgaagtagggatctcagctacgaacagaaattttaaaggacgtatgggaagcaaagacgcgctggcctatttagccagtcctgcggtcgtcgcagcaagcgcagttctcgggaaaatctctagtccagcagaagtattgagcacaagcgaaattcctttttctggagtaaagacggaaattatcgaaaacccagttgttgaagaagaggttaatgcccagacagaagcgccgaagcaatccgtagagattctcgaaggttttcctcgcgaattttctggagaacttgtactgtgtgacgcggacaatattaacacagatgggatatatccgggaaaatatacttatcaagacgatgtacctaaggaaaagatggcacaggtatgtatggagaattatgacgccgaatttcgaactaaagtacaccctggcgatatcgtcgtgtcaggctttaactttggaaccggcagctctcgggagcaggctgcgacagcgctgctggccaaaggaatcaaccttgtcgtctctggctctttcggaaatatattcagccggaactccattaataacgcgcttttgaccctggaaattcctgcattgatcaaaaagttgcgggaaaaatatcaaggagcccctaaagaacttacccgacggacgggatggttccttaaatgggacgtcgcggacgcgaaagtggtagttacggagggttcactcgatgggcctgtgatccttgagcagaaagttggtgaattaggcaagaatcttcaggaaataattgtgaaaggtggcctggagggctgggtaaagagtcagctg

SEQ ID NO: 114 - DNA, 2331 bp, Artificial
  mutated or codon-optimized sequence
atgttcaaaagaaccggctcattgttgttaagatgtagagcatcaagagtacctgttataggtagacctttgatttcattgagcacatcctctacgagtctttctttgagtcgtcccagaagtttcgccaccaccagcttgagaaggtatactgaagcaagctcctctacgactcaaacatctccatcatctagttcttggccagcacccgacgctgcacccagggtaccacagactcttacagaaaaaattgtgcaggcttactcacttggtttggccgaaggtcaatacgttaaggcaggagattacgttatgttgtccccacatagatgtatgacccatgataactcttggccaacagcattgaagttcatggccattggagcatctaaggttcataaccccgaccagatagtcatgaccttagatcatgatgtccaaaataaatctgaaaaaaatctaaagaaatacgaaagcatagaaaaattcgccaagcagcatggtatagacttttatcctgctggacatggcgttggccaccaaatcatgatcgaagagggctacgcatttcccgggaccgttacagtggcgtctgattctcactcaaatatgtatggaggggtggggtgcttgggcacccccatggtaaggactgatgccgccaccatctgggccaccggaagaacatggtggaaagtgcctccaatcgcaaaagtccaattcacgggcacacttccagaaggtgttaccggaaaggatgtgattgtagcattatctggccttttcaataaagacgaagttttgaactacgctatcgagtttacaggttcagaagaaactatgaagtctttgtcagtagacactcgtcttacaattgcgaatatgacgacagaatggggtgctttgacgggcttgttccctattgattctacattagagcagtggctgagacataaagctgcaactgcgtctagaaccgaaacagccagaagattcgccgaagaaagaataaacgaattattcgccaacccaacggttgctgatcgtggtgctagatacgccaaatacttgtatttagatttgtctactttgtctccttatgttagtggtccaaactcagtcaaagtcgctaccccattggatgagctagaaaagcataagttaaagatcgacaaagcataccttgttagctgtacaaactcaagggcttctgatatcgcggcagctgccaaggtcttcaaggatgccgttgcaaggacagggggtcctgtcagagtcgcagatggggtggaattttacgtggcagctgcttccaaggccgagcagaaaattgcagaggaagctggggattggcaagctttgatggacgctggggcaattccattaccggcaggctgtgctgtttgcattggtctgggcgctggtttattaaaggaaggtgaagtgggtatatcagcctccaacagaaattttaaaggccgtatgggttctccagatgcgaaggcttatctagcctctccggaggtggttgctgccagtgcattgaacggggttatctcaggtccgggcatctacaagagaccagaggactggacaggagtatcaatcggagaaggcgaagttgttgaatctggatctagaatagacactactttagaggcaatggaaaagtttattggacaattagatagcatgattgactcctccagcaaagctgttatgccagaagaatcaacaggttctggcgctacagaagttgatattgttcctggtttccccgaaaaaatcgaaggcgaaatattgtttttggacgcggataatatctcaaccgatggaatttatccaggaaagtacacctaccaagacgacgttacaaaagacaaaatggctcaagtttgcatggaaaactacgatccagctttctccggtatagcaagggctggagacatttttgtttcaggtttcaactttggttgtggctcaagcagagaacaggctgctacatctattttggctaagcagttgcctttagttgtagcaggaagtattgggaatactttttccagaaacgcagttaacaatgcgttgccgttgttagaaatgccaagattaattgaaagactgagagaagcattcggttcagaaaaacagcctacaagaagaacaggatggacttttacttggaacgttaggacttctcaagtgacggtacaggaagggcctggcggtgagacctggtctcaatcagttccggctttccctccaaatttacaggatattattgctcagggaggtttagaaaaatgggttaaaaaagagatatctaaggca

SEQ ID NO: 115 - DNA, 1128 bp, Thermus thermophilus
atgcgggaatggaagattattgactcgacgcttcgcgaaggtgaacagtttgagaaagcgaatttcagcactcaagataaggttgaaatcgccaaggctttggatgagtttggcattgagtatatcgaggtaacgacccccgtggcaagcccccaatctcggaaggatgcagaggtgctcgcctctctcggactcaaggcgaaagtcgtgactcatattcaatgccgtctcgacgccgctaaggtggctgttgaaacgggagttcaaggcattgatctgcttttcggtacctctaaatacctccgcgcggcgcacggacgggatattccgcggattatcgaggaagctaaagaggtcattgcgtacattcgcgaagctgcgccacatgtagaagtgcgcttctccgcggaagacacgttccggtctgaggaacaggatttgctcgctgtttatgaagcggtggccccctacgttgatcgtgtgggacttgcggatacggttggagttgctacgcctcgccaagtatatgcccttgtacgcgaagttcggcgcgtggtgggtccacgggtggatattgaattccacggccataatgatactggttgcgccatcgccaacgcttatgaagcgatcgaggccggcgcaactcacgttgacacgactattctgggtattggcgaacggaacggaattactccgcttggaggttttctcgctcggatgtacacgcttcaaccggaatatgtacgccggaagtacaagctggaaatgctgcctgagctggatcgtatggtggcgcgcatggtcggagttgagatcccattcaataattacattaccggagaaaccgctttttcgcacaaagctggaatgcatctgaaagccatttacatcaatcccgaggcatacgaaccgtaccccccagaagtcttcggtgtcaagcggaaactcattattgcaagccgtcttacgggccggcacgctattaaagcgcgtgcggaggagcttggattgcattacggagaagaggaactccaccgtgtaacgcaacacatcaaagctctcgcagaccgtggccaattgactctcgaagaattggaccgtattctgcgggaatggatcaccgca

SEQ ID NO: 116 - protein, 376 aa, Thermus thermophilus
MREWKIIDSTLREGEQFEKANFSTQDKVEIAKALDEFGIEYIEVTTPVASPQSRKDAEVLASLGLKAKVVTHIQCRLDAAKVAVETGVQGIDLLFGTSKYLRAAHGRDIPRIIEEAKEVIAYIREAAPHVEVRFSAEDTFRSEEQDLLAVYEAVAPYVDRVGLADTVGVATPRQVYALVREVRRVVGPRVDIEFHGHNDTGCAIANAYEAIEAGATHVDTTILGIGERNGITPLGGFLARMYTLQPEYVRRKYKLEMLPELDRMVARMVGVEIPFNNYITGETAFSHKAGMHLKAIYINPEAYEPYPPEVFGVKRKLIIASRLTGRHAIKARAEELGLHYGEEELHRVTQHIKALADRGQLTLEELDRILREWITA

SEQ ID NO: 117 - DNA, 1128 bp, Artificial
  mutated or codon-optimized sequence
atgcgcgaatggaagattatcgacagtacgctccgtgagggtgaacaatttgaaaaggcgaacttttcaacgcaggacaaagtggagatagcaaaagctttggacgaatttggcatagaatatatcgaagtcacaacaccggtagcgtctccgcagagccgcaaggatgcagaagtattagctagcctcggattaaaagctaaggttgttacacatattcaatgccggctggacgcggcgaaggttgcggtggaaaccggtgttcaaggcattgatctgctttttggcacaagtaagtaccttagagctgctcatggccgtgatatcccacgtatcatcgaagaagccaaagaagtcatcgcatatattagagaggcggctccgcatgttgaagttcgtttttctgctgaggatacattccgttccgaagaacaggatcttctggctgtctatgaagcggttgcgccttacgtggaccgtgtgggtctggcggatactgttggtgttgcaacaccgcgccaggtatatgcgctggtccgtgaagttagacgcgtcgtaggcccgcgtgttgacatcgaatttcatggccacaatgataccggctgcgcaatcgcaaacgcttatgaagcaatagaagcgggcgcgacgcatgtggataccacaattctcgggattggtgaacgcaacggcataacaccacttggcggatttctcgcccggatgtatactttgcaaccggaatacgttagaagaaaatataaactggagatgctcccggagttagatcgtatggttgctcgtatggttggcgtcgaaattccgtttaacaactatattacaggagagacagcgttttctcataaagcgggtatgcacctgaaagccatctatatcaatccggaagcatacgaaccgtatccgccggaggtgttcggggtgaagagaaaattgatcattgcgtctagacttacgggccgccatgccattaaagcacgcgctgaggagctggggttacattacggcgaagaagaacttcatagagtcacgcagcatatcaaagccttggcagatagaggacagttgacgctggaagaattagatcggattttaagagaatggattacggct

SEQ ID NO: 118 - DNA, 1128 bp, Artificial
  mutated or codon-optimized sequence
atgcgtgaatggaaaattattgactctacgctgcgtgagggtgagcaatttgaaaaagcaaatttctccacccaggataaggtggaaatcgccaaggcgctggatgaatttggtatcgagtacatcgaagtgactactcccgtggcatccccacagtctcgaaaggacgccgaagtgttggcttccctcggccttaaggccaaggtcgttacccacatccagtgccgtctcgatgccgccaaggtggcggttgagactggcgttcagggaatcgacttgttgttcggaaccagcaaatacttgcgagctgctcatggccgcgatattccacgcattattgaagaggcaaaagaagtgattgcatacatccgagaagctgctccccatgtagaagtccgcttctcggctgaagacaccttccgttcagaagagcaagaccttttggctgtatacgaggctgttgctccgtacgtggatcgcgtcggtttggcagacaccgtgggcgtggccaccccacgccaggtatatgccttggttcgtgaggttcgtcgcgtagtaggcccacgtgtagacatcgaattccatggtcacaacgatacaggttgtgccatcgctaacgcatacgaggccatcgaagcaggcgctacccacgtcgacaccacaatcttgggcatcggcgaacgtaatggtatcaccccattgggtggcttcctggctcgcatgtacactcttcaacctgaatacgtgcgccgcaagtacaagttggaaatgttgcctgaactggatcgtatggttgcccgcatggtgggagtcgaaatccccttcaacaattacatcactggcgaaacggctttctcccacaaggctggaatgcacctgaaagcaatctacatcaatccagaggcgtatgagccatacccaccggaagtgtttggcgttaagcgaaaacttattattgccagccgactcaccggccgtcacgcaatcaaggctcgcgccgaagagctcggtctccattacggcgaagaggagcttcatcgcgttacccagcacatcaaagcactcgctgaccgcggccagctgacccttgaagaactggatcgcattctgcgcgaatggatcacggcg

SEQ ID NO: 119 - DNA, 1002 bp, Artificial
  mutated or codon-optimized sequence
atggcctaccgtatctgcttgattgaaggtgatggtattggtcatgaggtaattcctgctgccagaagagttttggaggcaactggtctacctttggaattcgtcgaagctgaggctggttgggaaacatttgaaaggagaggtacttcagtacccgaagaaacagttgagaaaatcttaagctgtcatgctaccttatttggtgcagctacatcccccacaagaaaggtacccggctttttcggcgcgatcagatatttgaggaggagactagacttatatgcgaacgtaagacctgcgaagtcaagacctgtccctggttcaagacctggcgttgatcttgtaattgtaagggagaacactgaaggtttgtacgtcgagcaagaaaggagatacctagacgtagcaattgccgatgctgttataagcaaaaaggctagtgagcgtattggaagagccgcgctaagaattgcagaaggaagacccagaaaaacgttgcatatcgcacataaagcgaacgtgttaccgttaactcaaggcctatttttggatacagttaaagaagtcgctaaagatttcccccttgttaatgttcaagacattatagttgacaattgcgctatgcagttggtaatgaggcctgaaagatttgatgtaatcgttactactaacctgctgggcgacatattgagtgacctagcagctggcttagttggtggattaggtctggctccatcaggaaatataggtgatactactgctgtttttgaacccgtgcatggttcagcacctgacatagctggaaagggtatcgcgaacccaactgcggctattctgtctgctgccatgatgttggactatctaggtgaaaaagaagctgccaagagagttgaaaaggccgtagatttggtgttagaaagaggtccgagaactcctgatctaggtggagatgctacgacagaagccttcacagaggcagtcgtagaagctttgaaatcacta

SEQ ID NO: 120 - protein, 435 aa, Komagataella pastoris
MSETNGQTNGSSNGAQPQQRNPYGPHPSDFLSNVSSFQLIESTLREGEQFANAFFTTEKKIEIAKALDDFGVDYIELTSPVASEQSRSDCEAICKLGLKAKILTHIRCHMDDARVAVETGVDGVDVVIGTSQFLRQFSHGKDMSYITNQAIEVIEFVKSKGIEIRFSSEDSFRSDIVDLLNIYRTVDKIGVNRVGIADTVGCANPRQVYELVKTLKSVVHCDIECHFHDDTGCAIANAYTALEAGAKLIDVSVLGIGERNGITPLGGLMARMIAADRDYVLSKYKLHKLRDLETLVAEAVQVNIPFNNPITGFCAFTHKAGIHAKAILANPSTYEILQPSDFGLTRYIHFANRLTGWNAIKSRVDQLNLHLTDAQCKEVTAKIKKMGDIRPLNIDDVDSIIKDFHADITTPAFPPVATNNDEDVEPATKKQRLDQ

SEQ ID NO: 121 - protein, 1305 aa, Artificial
  mutated or codon-optimized sequence
ATGTCGGAAACGAATGGTCAGACGAATGGAAGCTCAAATGGAGCCCAGCCGCAACAGCGCAATCCGTACGGTCCACACCCCAGCGATTTCCTCAGCAATGTTTCCAGCTTTCAGCTGATTGAGAGCACTCTGCGTGAGGGAGAGCAATTTGCGAATGCGTTCTTTACCACTGAAAAAAAAATTGAAATTGCGAAGGCGTTGGATGACTTTGGAGTTGATTATATCGAACTGACCTCACCTGTCGCATCCGAACAGTCACGTTCAGACTGTGAGGCCATCTGTAAACTGGGACTTAAAGCCAAAATTCTTACTCATATCCGTTGCCATATGGACGACGCTCGTGTGGCCGTCGAAACTGGAGTCGATGGCGTGGACGTGGTCATTGGAACCTCTCAATTCCTGCGTCAGTTTAGCCATGGCAAGGATATGAGCTATATCACTAATCAGGCTATTGAGGTGATCGAATTTGTAAAATCAAAAGGAATTGAAATTCGCTTCTCTTCCGAAGACTCGTTCCGTTCTGACATTGTCGATCTGCTTAATATTTACCGTACTGTTGATAAGATTGGTGTCAATCGGGTAGGCATCGCAGATACTGTTGGCTGCGCTAATCCTCGGCAGGTATATGAGCTTGTTAAGACCCTGAAGAGCGTAGTCCATTGTGATATTGAGTGCCACTTCCATGATGATACTGGCTGCGCCATCGCCAACGCCTACACGGCCCTCGAAGCAGGAGCGAAATTGATTGACGTATCTGTTTTGGGTATTGGTGAGCGCAACGGTATCACGCCTCTGGGCGGTTTGATGGCCCGGATGATTGCCGCAGACCGGGATTATGTCCTTAGCAAGTACAAGCTCCATAAGCTGCGTGACCTTGAAACGCTCGTCGCGGAAGCAGTTCAGGTTAACATCCCCTTTAACAACCCCATCACCGGCTTTTGTGCGTTCACGCATAAAGCCGGTATCCATGCGAAAGCTATCCTGGCAAATCCGTCAACTTACGAGATTCTGCAGCCAAGCGACTTCGGTCTGACTCGGTATATTCATTTCGCTAACCGCCTCACGGGCTGGAACGCTATTAAATCTCGGGTGGATCAGCTTAACTTGCACTTGACGGATGCTCAATGCAAAGAGGTCACGGCCAAAATCAAAAAGATGGGAGATATCCGGCCCCTTAATATTGATGACGTTGATTCAATCATCAAGGACTTTCACGCCGACATTACTACGCCAGCATTCCCTCCCGTAGCGACGAATAATGACGAGGATGTAGAACCAGCGACGAAGAAGCAACGTTTGGATCAA